(19) United States
(12) Patent Application Publication
(10) Pub. No.: US 2023/0037905 A1
(43) Pub. Date: Feb. 9, 2023

(54) TOPICAL COMPOSITION COMPRISING TOFACITINIB AND FINGOLIMOD

(71) Applicant: Vyne Therapeutics Inc., Bridgewater, NJ (US)

(72) Inventors: Russell ELLIOTT, Basking Ridge,, NJ (US); Yohan HAZOT, Kiryat Ekron (IL); Gareth WINCKLE, Biot (FR); Ariel MARGULIS, Jerusalem (IL)

(21) Appl. No.: 17/758,175

(22) PCT Filed: Dec. 31, 2020
(86) PCT No.: PCT/US2020/067612

§ 371 Date: Jun. 29, 2022

Related U.S. Application Data
(60) Provisional application No. 62/955,847, filed on Dec. 31, 2019.
(60) Provisional application No. 63/066,717, filed on Aug. 17, 2020.
(60) Provisional application No. 63/086,275, filed on Oct. 1, 2020.
(60) Provisional application No. 63/086,442, filed on Oct. 1, 2020.

(51) Int. Cl. A61K 31/519; A61K 31/137; A61K 47/14; A61K 9/00; A61K 47/44; A61K 9/107; A61K 9/12

(52) U.S. Cl. CPC A61K 31/519; A61K 31/137; A61K 47/14; A61K 9/0014; A61K 47/44; A61K 9/107; A61K 9/122

(57) ABSTRACT

The present disclosure relates to novel topical formulations containing a tofacitinib and or a fingolimod that are useful for treating dermatological conditions, such as atopic dermatitis, psoriasis, and eczema.

### BACKGROUND

Atopic dermatitis (AD) is a common, inflammatory skin condition affecting adults and children worldwide. Onset typically occurs during childhood. Diagnosis is based on clinical signs, morphology and distribution of skin lesions and historical features. AD is associated with a high socioeconomic burden, with an impact on the use of healthcare resources and patient health-related quality of life (HRQoL). Pruritus is a common symptom of AD and negatively affects patient HRQoL, particularly mental health and sleep quality.

Topical agents including emollients, corticosteroids, and calcineurin inhibitors (CNIs) are the mainstay of AD therapy. Other treatments include refined coal tar, topical and oral antibiotics, phototherapy, and systemic immunosuppressants. The possible limitations of current therapies include inadequate efficacy of nonsteroidal topical treatments, restrictions on application to particular body regions, “steroid and CNI phobia,” and application site reactions. Potential long-term safety concerns include systemic side-effects and skin atrophy (for striae and other atrophic changes) with topical corticosteroids and increased risk of infections with CNIs

Psoriasis is another chronic disease affecting skin and joints in at least 100 million individuals worldwide. There is no cure, and symptoms are managed by lifestyle measures, such as moisturizing and managing stress. The disease causes significant morbidity. Some of its main characteristics are inflamed, scaly and frequently disfiguring skin lesions, and arthritis of the joints in hands and feet. Typically, in the skin lesions, altered differentiation of epidermal keratinocytes accompanies keratinocyte hyperproliferation. Marked infiltrates of T-cells and neutrophils are characteristic of psoriatic skin and are directly involved in the inflammatory state of the affected tissue. In addition, a distinct increase in skin capillaries is a typical phenomenon in psoriasis.

In addition, the disease causes psoriatic skin lesions which are very itchy, and which can result in severe scratching and disfigurement. The various manifestations of the disease make it more than a dermatologic nuisance as it interferes with many daily activities of the afflicted. As a consequence, the disease also causes considerable psychological morbidity in many patients.

Current therapy for psoriasis includes anti-inflammatory agents such as steroids, specific anti-inflammatory cytokines and chemokines, and agents acting as anti-autoimmune therapies. While several of these therapies provide relief, many have undesirable side effects, and none provide a cure.

Eczema is a form of dermatitis or inflammation of the dermis. The term eczema is broadly applied to a range of persistent skin conditions characterized by one or more of the symptoms of redness, skin edema (swelling), itching and dryness, crusting, flaking, blistering, cracking, oozing, or bleeding.

As eczema has many leading causes, treatment can be varied. There is no cure for eczema. Some limited treatment options exist and include for example: moisturizers, topical corticosteroids, phototherapy and immunotherapy drugs. However, prolonged use of topical corticosteroids is thought to increase the risk of possible side effects, and high-strength steroids may be absorbed into the body. Their immunosuppressive action can also lead to secondary skin infections.

AD arises from the interaction between genetic, environmental, and immunological factors. In particular. T-helper cell (Th)2 cytokines interleukin (IL)-4, IL-5, IL-13 and IL-31 have been implicated in the pathogenesis of AD. The Janus kinase (JAK)-signal transducer and activator of transcription (STAT) pathway is utilized by numerous cytokines and growth factors for signal transduction.

Tofacitinib is a small-molecule JAK inhibitor. Tofacitinib has been shown to inhibit cytokines such as IL-4 directly and leads to rapid attenuation of JAK-STAT signaling in keratinocytes. Tofacitinib ointment was also shown to have a therapeutic effect in a phase II study in patients with mild-to-moderate chronic plaque psoriasis (Ports et al., 2013) and AD (Bissonette et al., 2016).

Fingolimod is an immunomodulating drug derived from the fungal metabolite myriocin. Fingolimod is a sphingosine-1-phosphate receptor modulator that sequesters lymphocytes in lymph nodes, preventing them from contributing to an autoimmune reaction. Fingolimod is one of several disease-modifying therapies used in the management of relapsing forms of MS (e.g., relapsing-remitting MS-RRMS). Fingolimod undergoes rapid phosphorylation in vivo by sphingosine kinase 2 to produce fingolimod-phosphate which binds to four of the five S1P receptors (S1P1 and S1P3-5) with high affinity (0.3-3.1 nM). Fingolimod-phosphate acts as a nonselective agonist for SIP, S1P3, S1P4, and S1P5 receptors (lacking activity on S1P2). It acts as a functional antagonist of SIP receptors, causing the irreversible internalization and degradation of bound S1P receptors (thus preventing their recycling back to the cell surface). Fingolimod is also a competitive inhibitor of sphingosine kinase 1 (SphK1).

Mast cells are believed to be involved in the pathogenesis of atopic dermatitis. Human mast cells are major interleukin 22 (IL-22) producers in patients with atopic dermatitis. Dermal mast cells contain and release interleukins, among which is TNF-α. Mast cells release the contents of their secretory granules to their surroundings upon degranulation. Many of these granule mediators or mediators synthesized de novo participate in the development of itch. Increased morphological contacts between mast cells and sensory nerves in the lesional skin in psoriasis and atopic dermatitis as well as experimental models in mice and rats support the role for mast cell-sensory nerve communication in consequent pruritus. As mentioned, Fingolimod is a competitive inhibitor of sphingosine kinase 1 (SphK1), and a functional antagonist of S1PR1, S1PR3, S1PR4, S1PR5 but not S1PR2. It can potentially downregulate mast cell infiltration and degranulation in atopic dermatitis.

Filaggrin expression is downregulated in patients with AD. S1P has been reported to induce Ca2+ signaling, a key process for epidermal and keratinocyte differentiation. The regulated release of Ca2+ from ER and Golgi stores, as well as the Ca2+ influx through Ca2+− permeable ion channels, induces the transcription of genes associated with keratinocyte differentiation, such as keratin 1 and 10, filaggrin, and loricrin. Fingolimod, a structural analogue of S1P can thus potentially upregulate filaggrin product through Ca2+ signaling. Fingolimod, through the reduction of inflammatory cells infiltration to the dermis, and consequent reduction in chemokines, can potentially prevent chemokines downregulation of filaggrin.

It is well described that some central dendritic cell functions (migration, cytokine secretion) can be modulated in vitro by S1P and fingolimod. Mature Dendritic Cells were found to migrate to S1P, a phenomenon that correlated with the up-regulation of S1P1 and S1P3 in maturing Dendritic Cells. S1P1 expression was massively induced (38-fold) in the matured Dentritic Cells in vivo. The migration-inducing effect of S1P could be severely hampered by application of the S1P analogue fingolimod in vitro and in vivo. Fingolimod can thus downregulate inflammation mediated by dendritic cells, and reduced antigen-capture by Langerhans cells in AD.

In many cases, the healing of a wound is imperfect; resulting in the formation of a scar. Attempts to accelerate the healing process may result in elevating the incidence of scar formation. When the wound is bacterially infected, the healing process becomes more challenging and may take longer. Scars are more often caused following improper treatment.

Many other topical disorders involve inflammation and a product which addresses the inflammation and treats or ameliorates the disorder while avoiding or minimizing systemic and skin-related side effects would be advantageous and could improve patient compliance with treatment.

A product that requires a short treatment period, which is safe, well-tolerated, and prevents occurrence and/or reduces the grade of severity or the incidences of AD, psoriasis, eczema-induced lesions and pruritus, and scarring, while avoiding systemic and skin-related side effects would be advantageous and could improve patient compliance with treatment.

### SUMMARY

In one or more embodiments, there is provided a composition to treat or lessen the symptoms of janus kinase (JAK) related conditions and or a sphingosine-1-phosphate receptor and or a CB1 receptor related conditions or disorders. In some embodiments the composition is applied topically, in some orally an in some both topically and orally. In one or more embodiments the composition comprises a carrier and one or more active pharmaceutical ingredients (active agents). In some embodiments the active agent comprises a JAK inhibitor. In some embodiments the active agent comprises a sphingosine-1-phosphate receptor agonist and or a CB1 receptor antagonist. In some embodiments the active agent is a combination of a JAK inhibitor (e.g., a tofacitinib) and a sphingosine-1-phosphate receptor agonist and or a CB1 receptor antagonist (e.g., a fingolimod).

In one or more embodiments, there is provided a topical composition comprising a tofacitinib or a pharmaceutically acceptable salt thereof and a carrier in which tofacitinib is suspended to treat or lessen the symptoms of janus kinase (JAK) related conditions, such as atopic dermatitis, psoriasis, and eczema.

In one or more embodiments, the effect of administering a composition comprising a tofacitinib is achieved by delivering the tofacitinib onto and into the skin or mucosa or follicles. In one or more embodiments, systemic penetration through the skin, mucosa or follicles is low. In one or more embodiments, systemic penetration through the skin, mucosa or follicles is less than about 10%, less than about 9%, less than about 8%, less than about 7%, less than about 6%, less than about 5%, less than about 4%, less than about 3%, less than about 2%, less than about 1.8%, less than about 1.7%, less than about 1.6%, less than about 1.5%, less than about 1.4%, less than about 1.3%, less than about 1.2%, less than about 1.1%, less than about 1%, less than about 0.8%, less than about 0.6%, less than about 0.5%, less than about 0.4%, or less than about 0.1% of the tofacitinib applied to the skin. In one or more embodiments, the average maximum plasma concentration following tofacitinib application to the skin, mucosa or follicles is less than 5 ng/mL or about 5 ng/mL. In one or more embodiments, the maximum plasma concentration following tofacitinib application to the skin mucosa or follicles is between about 1.5 ng/mL to about 6.2 ng/mL. In one or more embodiments, the skin penetration is between about 0.1 μg/cm2 to about 8 μg/cm2, or about 0.1 μg/cm2 to about 6 μg/cm2, or about 0.1 μg/cm2 to about 5 μg/cm2, or about 0.1 μg/cm2 to about 4 μg/cm2, or about 0.2 μg/cm2 to about 4 μg/cm2, or about 0.3 μg/cm2 to about 3.8 μg/cm2, or about 0.4 μg/cm2 to about 3.6 μg/cm2, or about 0.5 μg/cm2 to about 3.4 μg/cm2, or about 0.6 μg/cm2 to about 3.2 μg/cm2, or about 0.7 μg/cm2 to about 3 μg/cm2, or about 0.8 μg/cm2 to about 2.8 μg/cm2, or about 0.9 μg/cm2 to about 2.6 μg/cm2, or about 1 μg/cm2 to about 2.5 μg/cm2, or about 0.2 μg/cm2 to about 0.6 μg/cm2, or about 0.3 μg/cm2 to about 0.7 μg/cm2, or about 0.4 μg/cm2 to about 0.8 μg/cm2, or about 0.3 μg/cm2 to about 1.5 μg/cm2, or about 0.3 μg/cm2 to about 1 μg/cm2, or about 0.2 μg/cm2, or about 0.3 μg/cm2, or about 0.4 μg/cm2, or about 0.5 μg/cm2, or about 0.6 μg/cm2, or about 0.7 μg/cm2, or about 0.8 μg/cm2, or about 0.9 μg/cm2, or about 1 μg/cm2, or about 1.2 μg/cm2, or about 1.3 μg/cm2, or about 1.4 μg/cm2, or about 1.5 μg/cm2, or about 1.6 μg/cm2, or about 1.7 μg/cm2, or about 1.8 μg/cm2, or about 1.9 μg/cm2, or about 3 μg/cm2, or about 3.1 μg/cm2, or about 3.2 μg/cm2, or about 3.3 μg/cm2, or about 3.4 μg/cm2, or about 3.5 μg/cm2, or about 3.6 μg/cm2, or about 3.7 μg/cm2, or about 3.8 μg/cm2, or about 3.9 μg/cm2, or about 4 μg/cm2 or any other figure within these ranges. In one or more embodiments, the maximum plasma concentration following tofacitinib application to the skin, mucosa or follicles is between about 0.1% to about 8% by weight of applied dose, or about 0.1% to about 6% by weight of applied dose, or about 0.1% to about 5% by weight of applied dose, or about 0.1% to about 4% by weight of applied dose, or about 0.2% to about 4%, or about 0.3% to about 3.8%, or about 0.4% to about 3.6%, or about 0.5% to about 3.4%, or about 0.6% to about 3.2%, or about 0.7% to about 3%, or about 0.8% to about 2.8%, or about 0.9% to about 2.6%, or about 1% to about 2.5%, or about 0.2% to about 0.6%, or about 0.3% to about 0.7%, or about 0.4% to about 0.8%, or about 0.3% to about 1.5%, or about 0.3% to about 1%, or about 0.2%, or about 0.3%, or about 0.4%, or about 0.5%, or about 0.6%, or about 0.7%, or about 0.8%, or about 0.9%, or about 1%, or about 1.2%, or about 1.3%, or about 1.4%, or about 1.5%, or about 1.6%, or about 1.7%, or about 1.8%, or about 1.9%, or about 3%, or about 3.1%, or about 3.2%, or about 3.3%, or about 3.4%, or about 3.5%, or about 3.6%, or about 3.7%, or about 3.8%, or about 3.9%, or about 4% by weight of applied dose or any other figure within these ranges. In one or more embodiments, systemic delivery or systemic penetration through the skin, mucosa or follicles can supplement the effects produced by non-systemic delivery onto and into the skin, mucosa, or follicles.

In one or more embodiments, tofacitinib or a pharmaceutically acceptable salt thereof is micronized. In one or more embodiments, it is encapsulated. In one or more embodiments, the active agent is encapsulated in particles, microparticles, nanoparticles, microcapsules, microspheres, nanocapsules, nanospheres, liposomes, niosomes, polymer matrices, silica-gels, graphite, nanocrystals, or microsponges. Such particles can have various functions, such as (1) protection of the drug from degradation; (2) modification of the drug release rate from the composition; (3) control of skin penetration profile; and (4) mitigation of adverse effects, due to the controlled release of the active agent from the encapsulation particles. Encapsulation is described in U.S. Publication No. 2015/0209296, which is incorporated by reference. In one or more embodiments related to one or more of the foregoing, the active ingredient, such as tofacitinib, is associated with solid, porous microcarriers, each having a hydrophobic surface. In one or more additional embodiments, the solid, porous microcarriers comprise a material selected from the group consisting of hydrophobic surface-modified silicon dioxide, porous polystyrene, porous polyamide, porous hydrophobic cellulose, and porous polytetrafluoroethylene. In one or more embodiments, the microcarrier possesses a porous structure for retaining the active ingredient, a hydrophobic surface, and is chemically non-reactive with the active ingredient. In one or more additional embodiments, the hydrophobic encapsulant comprises a material selected from the group consisting of mineral oil, petrolatum jelly, synthetic waxes, natural waxes, and silicone oils. In one or more embodiments, the average encapsulant particle size is below about 95 microns, is below about 75 microns, is below about 50 microns, or is below about 25 microns.

In one or more embodiments the particle size for tofacitinib is expressed as D50. By D50 is meant that the portions of particles with a size smaller than the D50 value are 50%. In some embodiments the D50 for tofacitinib is about 2-7 micrometers e.g., about 2-3 micrometers.

In one or more other embodiments the particle size for tofacitinib is expressed as D90. By D90 is meant that the portion of particles with a size below the D90 value is 90%. In some embodiments the D90 for tofacitinib is about 3-20 micrometers e.g., about 4-6 micrometers.

In some embodiments, the D90 particle size of tofacitinib is below about 22 microns, about 20 microns, about 18 microns, about 16 microns, about 14, microns, about 12 microns, about 10 microns, about 8 microns, about 7 microns, or about 6 microns. In some embodiments, the D90 particle size of tofacitinib is about 10 microns, about 9 microns, about 8 microns, about 7 microns, about 6, microns, about 5 microns, about 4 microns, about 3 microns, or about 2 microns.

In one or more embodiments, there is provided a topical composition comprising a fingolimod or a pharmaceutically acceptable salt thereof and a carrier.

In one or more embodiments, the effect of administering a composition comprising a fingolimod is achieved by delivering the fingolimod onto and into the skin or mucosa or follicles. In one or more embodiments, systemic penetration through the skin, mucosa or follicles is low. In one or more embodiments, systemic penetration through the skin, mucosa or follicles is less than about 20%, less than about 15%, is less than about 10%, less than about 9%, less than about 8%, less than about 7%, less than about 6%, less than about 5%, less than about 4%, less than about 3%, less than about 2%, less than about 1.8%, less than about 1.7%, less than about 1.6%, less than about 1.5%, less than about 1.4%, less than about 1.3%, less than about 1.2%, less than about 1.1%, less than about 1%, less than about 0.8%, less than about 0.6%, less than about 0.5%, less than about 0.4%, or less than about 0.1% of the fingolimod applied to the skin.

In one or more embodiments the fingolimod is suspended in the carrier. In one or more embodiments, fingolimod or a pharmaceutically acceptable salt thereof is not micronized. In some embodiments the D90 for the fingolimod or fingolimod salt is between about 70 and about 25 microns, e.g., about 60-40 microns, or about 35-25 microns, such as about 50, or about 40, or about 30 microns.

In one or more embodiments, fingolimod or a pharmaceutically acceptable salt thereof is micronized. In some embodiments the D90 for micronized fingolimod or fingolimod salt is about 3-20 micrometers e.g., about 4-8 micrometers. In some embodiments, the D90 particle size of the fingolimod or fingolimod salt is below about 22 microns, e.g., about 20 microns, about 18 microns, about 16 microns, about 14, microns, about 12 microns or about 10 microns. In some embodiments it is below about 10 microns, e.g., about 9 microns, about 8 microns, about 7 microns, about 6 microns, about 5 microns, about 4 microns, about 3 microns, or about 2 microns.

In one or more embodiments, there is provided a composition comprising a fingolimod and a carrier in which the fingolimod is suspended or substantially suspended.

In some embodiments, the fingolimod is suspended as nanoparticles. In some embodiments, the carrier comprises nanoparticles of a fingolimod.

In some embodiments, at least about 95% of the fingolimod is not present as agglomerates. In some embodiments, less than about 5%, or 4%, or 3%, or 2%, or 1% of the composition comprises agglomerates with a fingolimod. In one or more embodiments, the carrier composition is free of or essentially free of, or substantially free of fingolimod agglomerates.

In one or more embodiments, fingolimod is encapsulated.

Fingolimod is soluble in organic solvents, such as ethanol, DMSO and dimethyl formamide and is sparingly soluble in water. In some embodiments the fingolimod is dissolved or partially dissolved in the composition. In some embodiments, the amount of a fingolimod that is dissolved in the carrier or composition as a proportion of the total amount of the fingolimod in the carrier or composition is not more than about 0.005%, or not more than about 0.05%, or not more than about 0.1%, or not more than about 0.2%, or not more than about 0.3%, not more than about 0.4%, or not more than about 0.5%, or not more than about 0.6%, or not more than about 0.7%, or not more than about 0.8%, or not more than about 0.9%, or not more than about 1%, or not more than about 2%, or not more than about 3%, or not more than about 4%, or not more than about 5%, or not more than about 7.5%, or not more than about 10%, or not more than about 12.5%, or not more than about 15%, or not more than about 20%.

In one or more embodiments, the fingolimod is chemically stable e.g., for at least one month, or at least 2 months, or at least 3 months, or at least 6 months, or at least 9 months, or at least 12 months, or at least 15 months, or at least 18 months, or at least 21 months or at least 24 months. For example, in some embodiments, the fingolimod is chemically stable for at least 3 months at 25° C. In some embodiments, at least 90% by mass of the fingolimod or salt thereof is present in the composition when stored for 3 months at 25° C. In some embodiments, at least about 95% by mass of the fingolimod or salt thereof is present in the composition when stored for 3 months at 25° C. In some embodiments, at least about 98% by mass of the fingolimod or salt thereof is present in the composition when stored for 3 months at 25° C.

In some embodiments, systemic exposure to a fingolimod applied topically is much less than when the same amount is applied orally. In some embodiments, the systemic exposure is at least about 20-fold less. In some embodiments. In some embodiments, the systemic exposure is at least about 70-fold less, at least about 100-fold less, at least about 200-fold less, at least about 400-fold less or is at least about 500-fold less.

In one or more embodiments, the composition is a gel, paste, lotion, cream, soap, spray, mask, patch, powder, pomade, ointment, oil, foam, or mousse. In one or more embodiments, the composition is hydrophobic. In one or more embodiments, the composition comprises hydrophobic oils and waxes. In one or more embodiments, the composition comprises fatty alcohols. In one or more embodiments, the composition comprises hydrophobic oils and waxes.

In one or more embodiments, the composition comprises fatty acids. In one or more embodiments, the composition is surfactant-free.

In one or more embodiments, the composition is given prophylactically before the onset of symptoms associated with a JAK-related condition (disorder) and or a sphingosine-1-phosphate receptor (S1PR) related condition and or a CB1 receptor (CB1R) related condition (hereinafter “a JAK/S1PR/CB1R related condition”). In one or more embodiments, the composition is administered at the beginning of symptoms related to a JAK/S1PR/CB1R-related condition. In one or more embodiments, the composition is administered during the first week, first two weeks, first three weeks, first month, first five weeks, first six weeks, first seven weeks, first eight weeks, first nine weeks, first ten weeks, first eleven weeks or first twelve weeks of symptoms related to a JAK/S1PR/CB1R-related condition or some similar period, which could include parts of a week, such as one day, two days, three days, four days, five days, or six days. In one or more embodiments, the composition is administered one, two, three, four, five, six, seven, or eight weeks prior to the beginning of symptoms related to a JAK/S1PR/CB1R-related condition. In some embodiments the composition is applied once daily. In some embodiments the composition is applied twice daily. In some embodiments the composition is applied at least once per day for at least 7 days. In some embodiments the composition is applied at least once per day for at least 14 days. In some embodiments the composition is applied at least once per day for at least 4 weeks. In some embodiments the composition is applied at least once per day for at least 8 weeks. In some embodiments the composition is applied at least once per day for at least 12 weeks, at least 16 weeks, at least 20 weeks, at least 6 months, at least 12 months. In some embodiments the composition is applied as a maintenance dose following an initial treatment period. In some embodiments the maintenance dose is applied on non-consecutive days. In some embodiments the maintenance dose is applied on alternative days. In some embodiments the maintenance dose is applied twice weekly.

In one or more embodiments, JAK-related conditions may include: an autoimmune disease, an immune system dysfunction, a viral disease, an allergic disease, a skin disease, an IL-6 pathway-related disease, an immune response, a hyperproliferative disorder, or a cancer.

In one or more embodiments, non-limiting examples of JAK-related conditions are alopecia, alopecia totalis, alopecia universalis, atopic dermatitis, psoriasis, vitiligo, autoimmune bullous skin disorder such as pemphigus vulgaris (PV) or bullous pemphigoid (BP), skin rash, skin irritation, skin sensitization (e.g., contact dermatitis or allergic contact dermatitis), chronic atypical neutrophilic dermatosis with lipodystrophy and elevated temperature (CANDLE), pustulosis palmoplantaris, ichtyosis, eczema, actinic keratosis, pruritus, rosacea and acne.

In one or more embodiments, non-limiting examples of JAK-related conditions are Crohn's disease, ulcerative colitis, Aicardi-Goutières syndrome, chilblain lupus, Stimulator of interferon genes-Associated Vasculopathy with onset in Infancy (SAVI), Singleton-Merten syndrome, retinal vasculopathy with cerebral leukodystrophy, autoimmune uveitis, multiple sclerosis, rheumatoid arthritis, juvenile arthritis, type I diabetes, lupus, systemic sclerosis, an inflammatory bowel disease, an autoimmune thyroid disease, an allograft rejection, a graft-versus-host disease, an allograft rejection reaction, or a graft-versus-host reaction, Epstein-Barr virus (EBV), hepatitis B, hepatitis C, HIV, HTLV 1, chickenpox, herpes zoster virus (VZV), or human papillomavirus (HPV) disease, myeloproliferative neoplasms, polycythemia vera, aicardi goutieres syndrome, systemic lupus erythematosus, solid tumors, advanced malignancies, metastatic cancer, Hodgkin's lymphoma, non-Hodgkin lymphoma, myelofibrosis, breast cancer, colorectal cancer, endometrial cancer, melanoma, acute myeloid leukemia, prostate cancer, kidney cancer, liver cancer, pancreatic cancer, gastric cancer, lung cancer, head and neck cancer, glioblastoma, leukemia, lymphoma, multiple myeloma, asthma, food allergy, rhinitis, Castleman's disease and Kaposi's sarcoma.

In one or more embodiments, JAK-related conditions may include a hyperproliferative disorder or skin cancer. In one or more embodiments, non-limiting examples of skin cancer are keratinocyte carcinomas, basal cell carcinoma, squamous cell carcinoma. Merkel cell cancer melanoma, cutaneous (skin) lymphomas, Kaposi sarcoma, skin adnexal tumors, and sarcomas.

In one or more embodiments, there is provided a method for preventing, treating or ameliorating symptoms related to a JAK-related condition in a subject, comprising topically administering prior to symptoms a JAK inhibitor. In some embodiments the administration of the JAK inhibitor is during the symptoms. In some embodiments, administration is continued for a period after alleviation of the symptoms, such as, one, two, three or four weeks afterwards. In some embodiments, the composition comprises a carrier and a JAK inhibitor e.g., a tofacitinib (as a base or a pharmaceutically acceptable salt thereof). In some embodiments, the composition comprises a carrier and a tofacitinib (as a base or a pharmaceutically acceptable salt thereof and an additional active agent. In some embodiments, the additional active agent is a fingolimod, an antihistamine, a corticosteroid, a retinoid, an antipruritic agent, an anaesthetic agent, a nonsteroidal anti-inflammatory drug (NSAID), an antibiotic, an anti-viral agent, an anti-fungal agent, a JAK-inhibitor, an ant-itching agent, an anti-irritant, or combinations thereof. In some embodiments the additional active agent is an immunosuppressive agent, a prodrug, an antineoplastic agent, a sphingosine-1-phosphate receptor agonist, a CB1 receptor antagonist or combinations thereof.

In one or more embodiments a JAK inhibitor e.g., a tofacitinib (either as a salt (e.g., tofacitinib citrate) or base) is used treat or ameliorate a disorder such as folliculitis, furunculosis, keratosis pilaris, hidradentitis suppurativa, pyoderma gangrenosum, a lichenification disorder e.g., lichen planus, sclerosus, lichen simplex chronicus, neurodermatitis, primary cicatricial alopecias, such as lichen planopilaris and frontal fibrosing alopecia, and cellulitis. The term lichenification is classed as a secondary skin lesion wherein the characteristic features of skin thickening, hvperpigmentation, and exaggerated skin lines are noted. Lichenification can be further divided into primary and secondary types. Primary lichenification signifies lichen simplex chronicus, also known as neurodermatitis circumscripta. Secondary lichenification occurs in atopic dermatitis, infective eczematous dermatoses, psoriasis, psoriasiform dermatosis, xerosis, pityriasis rubra pilaris, porokeratosis, vegetative growths, anxiety, and obsessive-compulsive disorders. In one or more embodiments the JAK inhibitor is used to treat or ameliorate any of these disorders in combination with an additional active agent.

In one or more embodiments, the antihistamine is, for example, astemizole, azatadine, azelastine, bromodiphenhydramine, brompheniramine, carbinoxamine, cetirizine, chlorcyclizine, clemastine, chlorothen, cyclizine, cyproheptadine, desloratadine, dexbrompheniramine, dimethindene, diphenylpyraline, doxylamine, fexofenadine, hydroxyzine, isothipendyl, loratadine, methapyrilene, montelukast, phenindamine, pheniramine, phenyltoloxamine, prophenpyridamine, pyrilamine, terfenadine, thenyldiamine, thonzylamine, trimeprazine, triprolidine and pharmaceutically acceptable salts thereof such as, e.g., azatadine maleate, fexofenadine HCl, hydroxyine HCl, isothipendyl HCl (theruhistin), methapyrilene HCl, montelukast sodium, tartrate, pheniramine maleate, phenyltoloxamine citrate, prophenpyridamine maleate, pyrilamine maleate, thenyldiamine HCl, trimeprazine, triprolidine HCl, buclizine, desloratidine, ebastine, emedastine, epinastine, ketotifen, levocabastine, levocetirizine, loratidine, mequitazine, mizolastine, olopatadine, oxatomide, terfenidine, pharmaceutically acceptable salts, isomers or prodrugs thereof, mepyramine, antazoline, dimenhydrinate, meclizine, thenaldine, alimemazine, ketotifen, acrivastine, embramine, dexchlorpheniramine, diphehydramine, misolastine, phenidamine, diphenhydramine, doxepin, phrilamine maleate, chlorpheniramine, tripelennamine, phenothiazine, promethazine hydrochloride, dimethindene maleate or mixtures of any two or more thereof.

In one or more embodiments, the corticosteroid is, for example, acetonide, aclometasone dipropionate, aldosterone, alpha-methyl dexamethasone, amcinafel, amcinafide, amcinonide, beclomethasone, beclomethasone dipropionates, betamethasone, betamethasone diproprionate, betamethasone sodium phosphate, betamethasone valerate, broncodialator, budesonide, chloroprednisone, chlorprednisone acetate, ciclesonide, clescinolone, clobetasol proprionate, clobetasol valerate, clobetasol valerate, clobetasol-17-propionate, clobetasone-17-butyrate, clocortelone, cortiso, cortisone, cortisone acetate, cortisone, dexamethasone, cortodoxone, deflazacort, defluprednate, desoxycorticosterone acetate, desoxymethasone, dexamethasone, dexamethasone sodium phosphate, dexamethasone-phosphate, dichlorisone, diflorasone, diacetate, diflucortolone valerate, diflurprednate, dipropionate HFA, fluadrenolone, flucetonide, fluclorolone acetonide, flucloronide, flucortine butylesters, flucortine butylesters, flucortolone, flucortolone caproate, fludrocortisone, flumethasone pivalate, flunisolide, fluocinolone acetonide, fluocinonide, fluocortolone, fluocortolone hydrocortisone-17-valerate, fluocortolone caproate, fluocortolone pivalate, fluoromethalone, fluosinolone acetonide, fluosinolone acetonide, fluperolone, fluprednidene (fluprednylidene) acetate, fluprednidene acetate, fluprednisolone, fluradrenolone, fluradrenolone acetonide, fluticasone, fluticasone furoate, fluticasone propionate, formoterol, halcinonide, hydrocortisone valerate, halobetasol proprionate, halometasone, hydrocortamate, hydrocortisone, hydrocortisone acetate, hydrocortisone butyrate, hydrocortisone cyclopentylpropionate, hydrocortisone valerate, hydrocortisone, budesonide, hydrocortisone-17-aceponate, hydrocortisone-17-buteprate, Hydrocortisone-17-butyrate, hydroxyl-triamcinolone, medrysone, meprednisone, methylprednisolone, mometasone. Mometasone furoate, paramethasone, prednicarbate, clobetasone-17-butyrate, prednisolone, prednisone, prednisone hydrocortisone acetat, rofleponide, Salmeterol, tixocortol, tixocortol pivalate, tixocortol prednisolone, triamcinolone, triamcinolone acetonide, triamcinolone alcohol, triamcinolone hexacatonide or mixtures of any two or more thereof.

In one or more embodiments, the retinoid is, for example, retinol, retinal, all trans retinoic acid and derivatives, isomers and analogs thereof, etretinate, actiretin, isotretinoin, adapalene, tazarotene, tretinoin, alitretinoin, seletinoid G or mixtures of any two or more thereof.

In one or more embodiments, there is provided a carrier composition suitable for providing delivery of an active agent topically to the skin or to a mucosal membrane or to a body cavity surface. In some embodiments, the active agent is suspended or substantially suspended. In some embodiments, the active agent is partly suspended and partly dissolved. In one or more embodiments, the active agent is provided in a pharmaceutically effective amount (“PEA”). A PEA will depend on multiple factors, including the disorder to be treated or prevented, the active agent, and the subject. In some embodiments, a PEA could range from as little as about 0.0001% or about 0.001% to as high as about 18%.

In one or more embodiments, the active agent is a JAK (Janus kinase) inhibitor. In some embodiments, the JAK inhibitor is provided in combination with one or more other active agents, which for example could be a second JAK inhibitor, or a S1PR modulator or agonist and or CB1R antagonist or may be an active agent useful for treating disorders of the skin, mucosa or body cavities, such as antibiotics, antifungals, antihistamines, anti-inflammatory agents, nonsteroidal anti-inflammatory drugs (NSAIDS), steroids, retinoids, antipruritic agents, anesthetic agents, and the like as will be appreciated by one skilled in the art.

JAKs include JAK1, JAK2, JAK3 and TYK2. They are cytoplasmic tyrosine kinases able to phosphorylate tyrosine residues either on themselves (autophosphorylation) or on adjacent molecules (transphosphorylation), including the STATs. The latter is a family of transcription factors acting downstream of JAKs. In some embodiments, the JAK inhibitor is a JAK 3 inhibitor. In some embodiments, it is a JAK 1 inhibitor. In some embodiments, it is a JAK 2 inhibitor. In some embodiments, it is a TYK2 inhibitor. In some embodiments, it is an inhibitor for any two or more JAK's, such as JAK 3 and JAK 1. In one or more embodiments, the JAK inhibitor is a tofacitinib. In some embodiments, tofacitinib is provided as the base. In some embodiments, tofacitinib is provided as a salt. In some embodiments, it may be provided as a combination of the salt and a combination of the base.

In one or more embodiments, there is provided a composition comprising a tofacitinib and a carrier in which the tofacitinib is suspended or substantially suspended. In some embodiments, at least about 99.9% of tofacitinib is suspended in the composition. In some embodiments, the tofacitinib is a pharmaceutically acceptable salt. In some embodiments, the tofacitinib salt includes one or more of a citrate salt, hydrochloride salt, hydrobromide salt, oxalate salt, nitrate salt, sulfate salt, phosphate salt, fumarate salt, succinate salt, maleate salt, besylate salt, tosylate salt, palmitate salt, tartrate salt, adipate salt, laurate salt and myristate salt. In some embodiments, the tofacitinib salt is tofacitinib citrate. In some embodiments, the tofacitinib salt is tofacitinib adipate. In some embodiments, the tofacitinib salt is tofacitinib laurate. In some embodiments, the tofacitinib salt is tofacitinib myristate. In some embodiments, the tofacitinib is a combination of two or more salts or a combination of one or more salts and tofacitinib base. In some embodiments, the tofacitinib is homogeneously suspended. In one or more embodiments, the tofacitinib is at least about 0.1% by weight of the composition. In some embodiments, it is at least 0.2%. In some embodiments, it is at least 0.3%. In some embodiments, it is about 0.1% to about 10%. In some embodiments, it is 0.2% to about 5%. In some embodiments, it is about 0.3% to about 3.5%. In some embodiments it is 0.3% to about 3%, or is about 0.3% to about 2%, or is about 0.3% to about 1.2%, or is about 0.4% to about 1.0%, or is about 0.45% to about 0.8% or is about 0.5% to about 0.75% by weight of the composition. In some embodiments it is about 0.1%, or about 0.15%, or about 0.2%, or about 0.25%, or about 0.3%, or about 0.35%, or about 0.4%, or about 0.45%, or about 0.5%, or about 0.55%, or about 0.6%, or about 0.65%, or about 0.7%, or about 0.75% or about 0.8%, or about 0.9% or about 1.0%, or about 1.1%, or about 1.2% by weight of the composition. In some embodiments it is about 1.3%, or about 1.4%, or about 1.5%, or about 1.6%, or about 1.7%, or about 1.8%, or about 1.9%, or about 2.0%, or about 2.25%, or about 2.5%, or about 2.75%, or about 3.0%, or about 3.5%, or about 4.0% or about 4.5%, or about 5.0%, or about 10.0% by weight of the composition. In some embodiments, it is about 0.4% to about 1.8% by weight of the composition. In some embodiments, it is about 0.5% to about 1.75%, or about 0.6% to about 1.7%, or about 0.7% to about 1.7%, or about 0.5% to about 1.6%, or about 0.5% to about 1.5%, or about 0.5% to about 1.4%, or about 0.5% to about 1.3%, about 0.5% to about 1.2%, by weight of the composition. In some embodiments, it is about 0.5% to about 0.7% by weight of the composition. In some embodiments, it is about 0.5%, or about 0.6% or about 0.7% by weight of the composition. In some embodiments, tofacitinib is about 0.6% by weight of the composition. For example, when the tofacitinib is tofacitinib citrate then about 1.1% by weight of the citrate salt would provide a dose of tofacitinib equivalent to about 0.6%. In one or more embodiments, the active agent is present in an amount of any figure within the ranges provided herein. In some embodiments a tofacitinib is applied topically in any of the aforesaid amounts together with at least one additional active agent e.g., a fingolimod. In one or more embodiments the aforesaid amounts of a tofacitinib when used in combination with a e.g., a fingolimod may be reduced by about 0.1%, by 0.25, by 0.3%, by 0.4%, by 0.5%, by 0.6%, by 0.7%, 0.8%, 0.9%, by 1%, by 2%, by 3%, by 4%, by 5%, by 6%, by 7%, by 8%, by 9%, by 10%, by 15%, by 20%, by 25%, by 30%, by 35%, by 40%, by 45%, by 50%, by 55%, by 60%, by 75%, or by 80%.

In one or more embodiments, the carrier is suitable for topical use, such as a gel, or a semi-solid, or a flowable semi-solid, or an ointment, or a liquid, or a foam, or a mousse, or a cream, or a lotion. In one or more embodiments, the carrier may be anhydrous. In one or more embodiments, the carrier may comprise water. In one or more embodiments, the carrier may be an emulsion. In some embodiments, the emulsion is with water and in some without. In other embodiments, the carrier is not an emulsion. In one or more embodiments, the carrier is a gel. In some embodiments, the gel comprises a silicone thickening agent. In some embodiments, the silicone thickening agent comprises a cross polymer and a silicone. In some embodiments, a gel comprises an elastomer-based formulation. In some embodiments, the gel comprises an oil or solvent and a polymeric agent, such as a gelling agent. In some embodiments, the gel is an oleogel formulation without elastomer. In some embodiments, tofacitinib is micronized. In some embodiments, tofacitinib is suspended as nanoparticles. In some embodiments, the carrier comprises nanoparticles of tofacitinib. In some embodiments, the size range is expressed as D90 between about 2 μm to about 50 μm. In some embodiments, the D90 is between about 5 μm to about 50 μm. In some embodiments, the D90 is less than about 25 μm, or is about 24 μm, or about 22 μm, or about 20 μm, or about 18 μm, or about 16 μm, or about 14 μm, or about 12 μm or about 11 μm. In some embodiments the D90 is less than about 10 μm, or is about 9 μm, or about 8 μm, or about 7.5 μm, or about 7 μm, or about 6 μm, or about 5 μm or about 4 μm, or about 3 μm. In one or more embodiments the average uniform size range expressed as D90 is less than about 1 μm, or less than about 0.75 μm, or less than about 0.5 μm, or less than about 0.25 μm, or less than about 0.2 μm, or is about 0.9 μm, or about 0.8 μm, or about 0.7 μm, or about 0.6 μm, or about 0.5 μm, or about 0.4 μm, or about 0.3 μm, or about 0.25 μm, or about 0.2 μm, or about 0.15 μm or about 0.1 μm.

In one or more embodiments, the carrier or carrier components can reduce the potential for agglomeration of suspended tofacitinib salt or base or fingolimod salt or base. In some embodiments, there is a reduction in the number of agglomerates. In some embodiments, there is a reduction in the size of the agglomerates. In some embodiments, there is a reduction in the frequency of agglomerates. In one or more embodiments there is provided a carrier composition in which the number and size of any agglomerates is considered not significant. For example, in some embodiments, the average number of tofacitinib particles in the size range between about 40 μm to about 100 μm is less than about 50 per mg. In some embodiments, the average number of particles in the size range between about 100 μm and 200 μm is less than about 10 per mg. In some embodiments, no or almost no particles larger than 200 μm are detected. In some embodiments, the average size of agglomerates is less than about 175 μm, or is less than about 150 μm, or is less than about 125 μm, or is less than about 100 μm, or is less than about 75 μm, or is less than about 50 μm. In some embodiments, at least about 95% of the tofacitinib or fingolimod is not present as agglomerates. In some embodiments, less than about 5% of the composition comprises agglomerates. In some embodiments, less than about 4% of the composition comprises agglomerates. In some embodiments, less than about 3% of the composition comprises agglomerates. In some embodiments, less than about 2% of the composition comprises agglomerates. In some embodiments, less than about 1% of the composition comprises agglomerates. In one or more embodiments, the carrier composition is free of or essentially free of, or substantially free of agglomerates.

In one or more embodiments, the carrier comprises at least one elastomer and at least one emollient. A detailed list of emollients is provided below. In some embodiments, emollient includes one or more of a glyceride oil, a branched-chain ester, and a branched hydrocarbon oil. In some embodiments, the emollient includes one or more of a triglyceride oil, an isopropyl ester, and a saturated and branched hydrocarbon oil.

In one or more embodiments, the carrier is not hydrophilic. In some embodiments, the carrier is free of or substantially free of hydrophilic compounds. In some embodiments, the carrier is free of or substantially free of volatile hydrophilic compounds, which in some embodiments includes a volatile hydrophilic propellant. In some embodiments, the carrier is free or substantially free of a surfactant. In some embodiments, the carrier is free or substantially free of water. In some embodiments, the carrier is free or substantially free of preservatives. In some embodiments, the carrier is free or substantially free of anti-oxidants. In some embodiments, the carrier is free or substantially free of scavengers. In some embodiments, the carrier is free or substantially free of additional stabilizers. In some embodiments, the carrier is free or substantially free of chelating agents.

In one or more embodiments, the carrier comprises a penetration enhancer that does not dissolve an active agent, e.g., a fingolimod or a JAK inhibitor, e.g., tofacitinib citrate. In one or more embodiments, the carrier comprises a penetration enhancer that only essentially dissolves the active agent. In one or more embodiments, the carrier comprises a penetration enhancer that only substantially dissolves the active agent. In one or more other embodiments, the carrier comprises a penetration enhancer that dissolves part of the active agent. In one or more embodiments, the carrier comprises a compound that does not dissolve an active agent, e.g. a fingolimod or a JAK inhibitor, e.g. tofacitinib citrate. In one or more embodiments, the carrier comprises a compound that only essentially dissolves the active agent. In one or more embodiments, the carrier comprises a compound that only substantially dissolves the active agent. In one or more other embodiments, the carrier comprises a compound that dissolves part of the active agent.

In one or more embodiments the carrier is free or substantially free of a penetration enhancer that dissolves a proportion of the active agent, e.g., a fingolimod or a JAK inhibitor, e.g. tofacitinib and in some embodiments the carrier is free or substantially free of a compound that essentially dissolves a proportion of the e.g., a JAK inhibitor, e.g. tofacitinib. In some embodiments, the proportion of the total active agent e.g., a fingolimod or a JAK inhibitor, e.g. tofacitinib that the penetration enhancer or the compound may dissolve is at least about 15%. In some embodiments, it is at least about 10%, or at least about 7.5%, or at least about 5%, or at least about 2.5%, or at least about 1%, or at least about 0.7%, or at least about 0.6%, or at least about 0.5%, or at least about 0.4%, or at least about 0.3%, or at least about 0.2%, or at least about 0.1%, or at least about 0.05%, or at least about 0.01%, or at least about 0.005%, or at least about 0.001%. In some embodiments, it is about 0.1% or more. In some embodiments, it is about 0.01% or more. In some embodiments, it is about 0.001% or more.

In one or more embodiments, the amount of fingolimod slat or base or tofacitinib salt or base that is dissolved in the carrier or composition as a proportion of the total amount of fingolimod salt or base or tofacitinib salt or base in the carrier or composition is not more than about 0.001%. In some embodiments, the amount oftofacitinib that is dissolved in the carrier or composition as a proportion of the total amount of tofacitinib in the carrier or composition is not more than about 0.01%. In some embodiments, the amount of tofacitinib that is dissolved in the carrier or composition as a proportion of the total amount of tofacitinib in the carrier or composition is not more than about 0.012%. In some embodiments, the amount of tofacitinib that is dissolved in the carrier or composition as a proportion of the total amount of tofacitinib in the carrier or composition is not more than about 0.015%. In some embodiments, the amount of tofacitinib that is dissolved in the carrier or composition as a proportion of the total amount of tofacitinib in the carrier or composition is not more than about 0.02%. In some embodiments, the amount of tofacitinib that is dissolved in the carrier or composition as a proportion of the total amount of tofacitinib in the carrier or composition is not more than about 0.03%. In some embodiments, the amount of tofacitinib that is dissolved in the carrier or composition as a proportion of the total amount of tofacitinib in the carrier or composition is not more than about 0.1%. In some embodiments, the amount of tofacitinib that is dissolved in the carrier or composition as a proportion of the total amount of tofacitinib in the carrier or composition is not more than about 0.005%, or not more than about 0.05%, or not more than about 0.2%, or not more than about 0.3%, not more than about 0.4%, or not more than about 0.5%, or not more than about 0.6%, or not more than about 0.7%, or not more than about 0.8%, or not more than about 0.9%, or not more than about 1%, or not more than about 2%, or not more than about 3%, or not more than about 4%, or not more than about 5%, or not more than about 7.5%, or not more than about 10%, or not more than about 12.5%, or not more than about 15%, or not more than about 20%.

In one or more embodiments, the total amount of tofacitinib that is dissolved in the carrier or composition is less than about 15% by weight of the total composition. In some embodiments, the total amount of tofacitinib that is dissolved in the carrier or composition is less than about 10%, or less than about 7.5%, or less than about 5%, or less than about 2.5%, or less than about 1%, or less than about 0.7%, or less than about 0.6%, or less than about 0.5%, or less than about 0.4%, or less than about 0.3%, or less than about 0.2%, or less than about 0.1%, or less than about 0.05%, or less than about 0.01%, or less than about 0.005%, or less than about 0.001%, or less than about 0.0001%, or less than about 0.00015%, or less than about 0.0002%, or less than about 0.0003%. In some embodiments, it is between about 0.1% and about 0.01%. In some embodiments, it is between about 0.01% and about 0.001%. In some embodiments, it is between about 0.1% and about 0.001%. In some embodiments, it is between about 0.001% and about 0.0002%.

In one or more embodiments a compound that can dissolve a portion of a tofacitinib includes one or more of water, HCl, transcutol, dimethyl isosorbide, a glycol, a polyethylene glycol, polyethylene glycol 200, polyethylene glycol 400, propylene glycol, glycerol, sulphoxides, dimethyl sulfoxide, dimethylacetamide, and dimethylformamide.

In one or more embodiments, the composition is non-occlusive or substantially non-occlusive. In one or more embodiments, the composition is partially occlusive. In one or more embodiments, the carrier is free or substantially free of an occlusive agent, such as petrolatum. In one or more embodiments, the carrier is free or substantially free of a solid wax having a melting temperature greater than about 45° C. In one or more embodiments, the carrier is free or substantially free of compounds to which tofacitinib is not inert. In one or more embodiments, the carrier is lipophilic. In one or more embodiments, the lipophilic carrier comprises at least one oil that is liquid at room temperature. In one or more embodiments, the lipophilic carrier comprises at least one oil that is solid at room temperature. In one or more embodiments, the lipophilic carrier comprises at least one oil that is liquid at room temperature and at least one oil that is solid at room temperature. In one or more embodiments, the carrier comprises a polymeric agent. In one or more embodiments, the polymeric agent is a gelling agent. In one or more embodiments, the carrier comprises a gelling agent and a hydrophobic agent or oil. In one or more embodiments, the carrier comprises at least one elastomer. In one or more embodiments the at least one elastomer comprises one or more of cyclopentasiloxane (and) polysilicone-11 (Grant MGS-Elastomer 1100), dimethicone (and) polysilicone-11 (Gransil DMG-3), a cyclopentasiloxane (and) petrolatum (and) polysilicone-11 (MGS-Elastomer 1148P), cyclopentasiloxane and dimethicone cross polymer (ST-Elastomer 10) and dimethicone (and) dimethicone crosspolymer (DOWSIL™ 9041). In some embodiments the elastomer is ST-Elastomer 10.

In one or more embodiments, there is provided a composition comprising a JAK inhibitor as a salt, such as a tofacitinib salt, e.g., tofacitinib citrate, wherein the salt is more stable than the base.

In one or more embodiments, there is provided a composition wherein the viscosity of the composition is stable or substantially stable from about 8° C. to about 40° C. In some embodiments, the viscosity of the composition is stable or substantially stable from about 10° C. to about 35° C. In some embodiments, the viscosity of the composition is stable or substantially stable from about 15° C. to about 30° C. In some embodiments, viscosity, of the composition is stable or substantially stable from about 20° C. to about 25° C.

In some embodiments, the carrier comprises a gelled oil. In some embodiments, the carrier comprises a gelled mineral oil. In some embodiments, the carrier comprises a gelled mineral oil and an elastomer. In some embodiments, the carrier comprises an elastomer and an emollient. In some embodiments, the carrier comprises a gelled oil and an emollient. In some embodiments, the carrier comprises an elastomer, a gelled oil and an emollient. In some embodiments, the gelled oil comprises a mineral oil. In some embodiments, the emollient is one or more of a glyceride oil, a branched alkyl ester, and a branched hydrocarbon oil. In some embodiments, if present, the glyceride oil comprises a triglyceride oil, the branched alky ester comprises an isopropyl ester, and the branched hydrocarbon oil is saturated. In some embodiments, the triglyceride oil comprises an MCT oil.

In one or more embodiments there is provided a topical composition comprising a tofacitinib and a carrier in which the tofacitinib is suspended or substantially suspended, wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and wherein the carrier is free or substantially free of a penetration enhancer that dissolves a proportion of the active agent, such as a JAK inhibitor, for example tofacitinib.

In some embodiments, at least about 99.9% of the active agent is suspended. In some embodiments, at least about 99%, about 98%, about 97%, about 96%, or about 95% of the active agent is suspended.

In some embodiments, the active agent, such as a JAK inhibitor, e.g., tofacitinib is a pharmaceutically acceptable salt. In some embodiments, the salt includes one or more of a citrate, an adipate, a laurate, or a myristate salt. In some embodiments, the JAK inhibitor is tofacitinib and the tofacitinib salt is tofacitinib citrate.

In some embodiments, the carrier or carrier base is a gel or comprises a gelled oil. In some embodiments, the oil is a silicone oil and the gelling agent is a cross polymer. In some embodiments, the oil is a mineral oil and the gelling agent is a copolymer, such as ethylene/propylene/styrene copolymer or butylene/ethylene/styrene copolymer. In some embodiments, the gelled mineral oil comprises a Versagel®.

In some embodiments, the carrier comprises an emollient. In some embodiments, it comprises a combination of two or more emollients. In some embodiments, the emollient comprises one or more of a glyceride, a triglyceride, a diglyceride, a monoglyceride, an MCT oil, a branched hydrocarbon oil, a saturated and branched hydrocarbon oil, squalene, squalane, a branched alkyl ester, isopropyl isostearate, isopropyl palmitate, isopropyl myristate, oleyl alcohol, a mineral oil, a vegetable oil, a liquid fatty acid, a liquid fatty alcohol, a branched liquid fatty acid, a branched liquid fatty alcohol, glyceryl monooleate, glyceryl isostearate, glyceryl dicaprate, a polypropylene glycerol alkyl ether, a polypropylene glycerol stearyl ether, polypropylene glycerol 15 stearyl ether, polypropylene glycerol 11 stearyl ether, glycerol behenate, diisopropyl adipate, cetearyl ethylhexanoate, and cetearyl isononanoate. In some embodiments, the emollient comprises one or more triglyceride oils. In some embodiments, the triglyceride oil comprises MCT oil. In some embodiments, the sole emollient is MCT oil. In other embodiments, it is combined with an alkyl ester. In some embodiments, the emollient comprises a branched alkyl ester. In some embodiments, the branched alkyl ester comprises an isopropyl ester or a glycerol iso-ester. In some embodiments, the isopropyl ester comprises isopropyl isostearate, isopropyl palmitate, isopropyl myristate or mixtures of two or more thereof. In some embodiments, the isopropyl ester comprises isopropyl isostearate. In some embodiments, the triglyceride oil is combined with a hydrocarbon oil. In some embodiments, the emollient comprises a branched hydrocarbon oil. In some embodiments, the branched hydrocarbon oil comprises squalene and or squalane. In some embodiments, the emollient comprises a branched and saturated hydrocarbon oil, such as squalane. In some embodiments, the emollient comprises at least two of a triglyceride oil, an isopropyl ester and a saturated and branched hydrocarbon oil. In some embodiments, the emollient comprises at least two of isopropyl isostearate, squalane and an MCT oil. In some embodiments, the emollient comprises a triglyceride oil, an isopropyl ester and a saturated and branched hydrocarbon oil. In some embodiments, the emollients comprise MCT oil, an isopropyl ester and squalane. In some embodiments, the isopropyl ester comprises isopropyl isostearate.

In one or more embodiments, a branched alkyl ester such as isospropyl isostearate may be substituted by or complemented with by the addition of one or more of the following: isostearyl isostearate, oleyl oleate, isocetyl stearate, hexyl laurate, isostearyl neopentanoate, ethylhexyl stearate, octyldodecyl neopentanoate, cetearyl octanoate, isodecyl neopentanoate, decyl oleate, isononyl ethylhexanoate, isononyl isononanoate, hexyldecyl ethylhexanoate, isotridecyl isononanoate, cetyl ethylhexanoate, octyldodecyl neodecanoate, octyldodecyl myristate, hexyldecyl isostearate, ethylhexyl hydroxystearate, octyldodecyl stearoyloxystearate, diisopropyl dilinoleate, octyl isopalmitate, isodecyl oleate, and octyl palmitate.

In one or more embodiments a branched hydrocarbon oil, such as squalene, may be substituted by or alternatively complemented with by the addition of one or more of the following: squalene, pristane, a mineral oil, a hydrogenated polyisobutene, isohexadecane, isodecane, or isododecane, and branched alkanes.

In one or more embodiments, triolein or lorenzo's oil may also be used.

In one or more embodiments, the composition comprising an elastomer and at least one emollient can provide two, three, or four of the following characteristics: an improvement in the chemical stability of the JAK inhibitor, e.g. a tofacitinib salt; a reduction or elimination of balling; when applied topically to skin or mucosa an increased delivery into the skin or mucosa; when applied topically to skin or mucosa a reduced delivery through the skin or mucosa; and when applied topically to skin an increased delivery into the epidermis and reduced delivery through the skin.

In some embodiments, such as when the tofacitinib salt is tofacitinib citrate, it can provide three, four or all of the aforesaid characteristics.

In one or more embodiments, the composition comprising an elastomer and at least one emollient can provide, when applied topically to skin or mucosa, an increased delivery into the dermis and a reduced delivery through the skin or mucosa.

In one or more embodiments, the carrier comprises a silicone oil in addition to the elastomer. In some embodiments, the silicone oil is a cyclomethicone or a dimethicone.

In one or more embodiments, the elastomer is about 75% to about 97% by weight of the composition. In some embodiments, the elastomer is about 80% to about 93% by weight of the composition. In some embodiments, the elastomer is about 86% to about 89% by weight of the composition. In some embodiments, the emollient is about 3% to about 25% by weight of the composition. In some embodiments, the emollient is about 7% to about 20% by weight of the composition. In some embodiments, the emollient is about 11% to about 14% by weight of the composition. In some embodiments, the emollient is about 12%, about 13%, or about 14% by weight of the composition.

In some other embodiments, the elastomer is about from 15% to about 75% and the emollient and or other components about 25% to 85% by weight of the composition.

In one or more embodiments the silicone oil is about 1% to about 75%, or about 5% to about 50%, or about 6% to about 40%, or about 7% to about 30%, or about 8% to about 20%, or about 10% to about 15%, or about 5% to about 10%, or about 1% to about 5%, by weight of the composition.

In one or more embodiments, the gelling agent is about 0.5% to about 15%, or about 1% to about 13%, or about 5% to about 12%, or about 8% to about 11%, by weight of the composition.

In one or more embodiments, the carrier comprises an elastomer, and at least one emollient or at least two emollients, and wherein the ratio of emollient to elastomer is about from about 1:30 to about 1:3. In one or more embodiments the carrier comprises, an elastomer, and at least two emollients; and wherein the ratio of emollient to elastomer is between about 1:9 to about 1:6, or is between about 1:8 and about 1:7, or is about 1:7, or is about 3:22, or is about 1:8. In one or more embodiments, the emollients are liquid at room temperature. In one or more embodiments, the emollients are liquid at about 25° C.

In one or more embodiments, the JAK inhibitor, e.g., tofacitinib is in an effective concentration sufficient to bind to Janus Kinase (JAK) receptors in the dermis or epidermis in the applied area of skin of a mammal. In one or more embodiments, the skin is of a human subject. In some embodiments, the receptors are JAK 3 receptors. In some embodiments, the receptors are JAK 1 receptors. In some embodiments, the receptors are JAK 2 receptors. In some embodiments, the receptors are TYK2 receptors. In some embodiments, the JAK inhibitor, e.g., tofacitinib is in an effective concentration sufficient to reach an apparent maximum inhibition of JAK receptors in the dermis or epidermis in the applied area of a mammal, as indicated when a significant additional increase in the JAK inhibitor, e.g., tofacitinib concentration by weight % in the composition does not result in a significant increase in efficacy in treating a disorder. In some embodiments, the JAK inhibitor, e.g., tofacitinib is in an effective concentration sufficient to reach an apparent maximum inhibition of JAK receptors in the dermis or epidermis in the applied area of a human subject, as indicated when a significant additional increase in tofacitinib concentration by weight % in the composition does not result in a significant increase in efficacy in treating a disorder. In some embodiments, the JAK inhibitor, e.g., tofacitinib is in an effective concentration sufficient to reach a plateau effect in the dermis or epidermis in the applied area of skin of a mammal, such as a human. In one or more embodiments, the disorder is atopic dermatitis and the effective concentration is about 0.6% by weight or more. In one or more embodiments, the tofacitinib is tofacitinib citrate. In one or more embodiments the effective concentration may be reduced by administering the tofacitinib with an S1PR receptor agonist e.g., a fingolimod.

In some embodiments, the carrier is free or substantially free of one or more of water, surfactants, hydrophilic compounds, preservatives, anti-oxidants, scavengers, chelating agents and additional stabilizers. In some embodiments, the composition is anhydrous or substantially anhydrous. In one or more embodiments, the composition has an Aw value of less than 0.9. In some embodiments, the composition has an Aw value of less than 0.8. In some embodiments, the composition has an Aw value of less than 0.7. In some embodiments, the composition has an Aw value of less than 0.6. In some embodiments, the composition has an Aw value of less than 0.5. In some embodiments, the composition has an Aw value of less than 0.4. In some embodiments, the composition has an Aw value of less than 0.3.

In one or more embodiments the active agent, such as a JAK inhibitor, e.g., a tofacitinib or S1PR modulator or agonist e.g., a fingolimod is chemically stable e.g., for at least one month, or at least 2 months, or at least 3 months, or at least 6 months, or at least 9 months, or at least 12 months, or at least 15 months, or at least 18 months, or at least 21 months or at least 24 months. For example, in some embodiments, the tofacitinib is chemically stable for at least 3 months at 25° C. In some embodiments, the tofacitinib is chemically stable for at least 6 months at 25° C. In some embodiments, at least 90% by mass of the tofacitinib or salt thereof is present in the composition when stored for 3 months at 25° C. In some embodiments, at least about 90% by mass of the tofacitinib or salt thereof is present in the composition when stored for 6 months at 25° C. In some embodiments, at least about 95% by mass of the tofacitinib or salt thereof is present in the composition when stored for 3 months at 25° C. In some embodiments, at least about 95% by mass of the tofacitinib or salt thereof is present in the composition when stored for 6 months at 25° C. In some embodiments, at least about 98% by mass of the tofacitinib or salt thereof is present in the composition when stored for 3 months at 25° C. In some embodiments, at least about 98% by mass of the tofacitinib or salt thereof is present in the composition when stored for 6 months at 25° C. In some embodiments, at least about 99% by mass of the tofacitinib or salt thereof is present in the composition when stored for 3 months at 25° C. In some embodiments, at least about 99% by mass of the tofacitinib or salt thereof is present in the composition when stored for 6 months at 25° C. In some embodiments, the composition is stored at 40° C., and the tofacitinib is chemically stable during the aforesaid periods. In some embodiments where the active ingredient comprises a tofacitinib, less than about 0.1% by mass of Impurity B is measured when the composition is stored for 3 months at 25° C. compared to time 0. In some embodiments, less than about 0.1% by mass of Impurity B is measured when the composition is stored for 6 months at 25° C. compared to time 0. In some embodiments, the composition is stored at 40° C., and the amount of Impurity B is less than about 0.1% during the aforesaid periods.

In some embodiments, such as where emollients like squalane, and or isopropyl isostearate, and or oleyl alcohol are present the level of adhesiveness, surface energy, or interfacial tension of the composition is reduced. In some embodiments, the reduction is sufficient to prevent significant adhesion of the active agent to a metal surface. In some embodiments, the reduction is sufficient to prevent significant adhesion of the active agent to a moving metal surface. In some embodiments, the metal is stainless steel. In some embodiments, the reduction is sufficient to prevent significant adhesion of the active agent to a plastic surface. In some embodiments, the reduction is sufficient to prevent significant adhesion of the active agent to a moving plastic surface. In some embodiments, the reduction is sufficient to bring the surface energy of the carrier to below that of the active agent with the metal or to that of a plastic. In some embodiments, the reduction is sufficient to bring the interfacial energy of the carrier to below that of the active agent with the metal or to that of a plastic. In one or more embodiments, the active agent is tofacitinib. In some embodiments, the interfacial tension (mN/m) of the carrier and tofacitinib is below that oftofacitinib with a metal. In some embodiments, the interfacial tension (mN/m) of the carrier and tofacitinib is at least about 5% below that of tofacitinib with a metal. In some embodiments, the interfacial tension (mN/m) of the carrier and tofacitinib is at least about 10% below that of tofacitinib with a metal. In some embodiments, the interfacial tension (mN/m) of the carrier and tofacitinib is at least about 15% below that of tofacitinib with a metal. In some embodiments, the interfacial tension (mN/m) of the carrier and tofacitinib is about 8% to about 25% below that oftofacitinib with a metal. In some embodiments, the interfacial tension (mN/m) of the carrier and tofacitinib is at least about 12% below that of tofacitinib with a metal. In some embodiments, the interfacial tension (mN/m) of the carrier and tofacitinib is at least about 20% below that of tofacitinib with a metal. In some embodiments, the interfacial tension (mN/m) of the carrier and tofacitinib is about 10% below that of tofacitinib with a metal. In some embodiments the interfacial tension (mN/m) of the carrier and tofacitinib is about 12% below that of tofacitinib with a metal. In some embodiments, the interfacial tension (mN/m) of the carrier and tofacitinib is about 15% below that of tofacitinib with a metal. In some embodiments, the interfacial tension (mN/m) of the carrier and tofacitinib is about 20% below that of tofacitinib with a metal. In some embodiments, the interfacial tension (mN/m) of the carrier and tofacitinib is about 22% below that oftofacitinib with a metal. In some embodiments, the interfacial tension (mN/m) of the carrier and tofacitinib is about 25% below that of tofacitinib with a metal. In some embodiments, the metal is stainless steel. In some embodiments, the surface energy of the carrier and tofacitinib is below that of tofacitinib with a plastic. In some embodiments, the interfacial tension (mN/m) of the carrier and tofacitinib is below that of tofacitinib with a plastic. In some embodiments, the interfacial tension (mN/m) of the carrier and tofacitinib is at least about 5% below that of tofacitinib with a plastic. In some embodiments, the interfacial tension (mN/m) of the carrier and tofacitinib is at least about 10% below that of tofacitinib with a plastic. In some embodiments, the interfacial tension (mN/m) of the carrier and tofacitinib is at least about 15% below that of tofacitinib with a plastic. In some embodiments, the interfacial tension (mN/m) of the carrier and tofacitinib is about 8% to about 25% below that of tofacitinib with a plastic. In some embodiments, the interfacial tension (mN/m) of the carrier and tofacitinib is at least about 12% below that of tofacitinib with a plastic. In some embodiments, the interfacial tension (mN/m) of the carrier and tofacitinib is at least about 20% below that of tofacitinib with a plastic. In some embodiments, the interfacial tension (mN/m) of the carrier and tofacitinib is about 10% below that of tofacitinib with a plastic. In some embodiments, the interfacial tension (mN/m) of the carrier and tofacitinib is about 12% below that of tofacitinib with a plastic. In some embodiments, the interfacial tension (mN/m) of the carrier and tofacitinib is about 15% below that of tofacitinib with a plastic. In some embodiments, the interfacial tension (mN/m) of the carrier and tofacitinib is about 20% below that of tofacitinib with a plastic. In some embodiments, the interfacial tension (mN/m) of the carrier and tofacitinib is about 22% below that of tofacitinib with a plastic. In some embodiments, the interfacial tension (mN/m) of the carrier and tofacitinib is about 25% below that of tofacitinib with a plastic. In some embodiments, the plastic is PTFE (polytetrafluorethylene). In some embodiments, the surface energy of the carrier and tofacitinib is below that of tofacitinib with a metal. In some embodiments, the interfacial tension between non-micronized tofacitinib and the composition is less than about 1.6 mN/m or between about 1.5 mN/m and about 1.1 nM/m. In some embodiments, the interfacial tension between micronized tofacitinib and the composition is less than about 2.5 mN/m or between about 1.8 mN/m and about 2.3 mN/m. In some embodiments, the surface tension of the composition is sufficient to discourage adhesion of tofacitinib to a surface. In some embodiments, the surface is a metal such as stainless steel, and in others it is a plastic. In one or more embodiments, the reduction in one or more of adhesiveness, surface energy, or interfacial tension of the composition may be facilitated by the presence of emollient. In some embodiments, the emollient comprises one or more of a branched hydrocarbon oil, a branched alkyl ester, a liquid fatty alcohol, and a liquid fatty acid. In some embodiments, the emollient comprises one or more of a branched and saturated hydrocarbon oil, an isopropyl ester, a liquid fatty alcohol, and a liquid fatty acid. In some embodiments, the emollient comprises one or more of squalane, isopropyl isostearate, and oleyl alcohol. In some embodiments, the ratio of carrier base to emollient is less than about 9:1. In some embodiments, the ratio of carrier base to emollient is between about 9:1 and about 6:1. In some embodiments, the ratio of carrier base to emollient is between about 8:1 and about 7:1, or is about 8:1, or about 22:3, or about 7:1. In some embodiments, the ratio of carrier base to emollient is less than about 30:1. In some embodiments, the ratio of carrier base to emollient is between about 30:1 and about 20:1. In some embodiments, the ratio of carrier base to emollient is between about 26:1 and about 22:1, or is about 23:1, or about 25:1.

In one or more embodiments, the interfacial tension is derived from a combination of surface tension and surface polarity.

In some embodiments, the carrier base is about 83% to about 90% by weight of the composition. In some embodiments, the carrier base is about 86% to about 88% by weight of the composition. In some embodiments, the carrier base is about 87% by weight of the composition. In some embodiments, the emollient is about 10% to about 16% by weight of the composition. In some embodiments, the emollient is about 11% to about 14% by weight of the composition. In some embodiments, the emollient is about 12% by weight of the composition.

In some embodiments, the active agent is tofacitinib citrate at about 0.5% to about 0.7%, or about 0.5%, or about 0.6% or about 0.7% by weight of the composition and the carrier base comprises an elastomer and is about 83% to about 90% by weight of the composition and the emollient is about 10% to about 16% by weight of the composition. In some embodiments, the carrier base comprises an elastomer and is about 86% to about 88% by weight of the composition, and the emollient is about 11% to about 14% by weight of the composition.

In some embodiments, the emollient comprises a triglyceride oil comprising an MCT oil, an olive oil, a coconut oil, a palm oil, a sunflower oil, a rapeseed oil, a soybean oil, a groundnut oil, a peanut oil, a corn oil, a walnut oil, a soya oil, a fish oil, a tallow, a fraction of any of the aforesaid, and mixtures of any two or more thereof.

In some embodiments, the tofacitinib is the sole active agent in the composition. In other embodiments, the composition further comprises a second active agent. By way of non-limiting examples, in some embodiments, the second active agent comprises a JAK inhibitor.

In some embodiments, the second active agent comprises an S1PR modulator or agonist e.g., a fingolimod. In some embodiments, the second active agent comprises an antipruritic agent. In some embodiments, the second active agent comprises an anaesthetic agent. In some embodiments, the second active agent comprises an antibiotic. In some embodiments, the second active agent comprises an antifungal. In some embodiments, the second active agent comprises an antiviral. In some embodiments, the second active agent comprises a steroid. In some embodiments, the second active agent comprises an NSAID. In some embodiments, the second active agent comprises a retinoid. In some embodiments, the second active agent comprises a dicarboxylic acid. In some embodiments, the second active agent comprises an antihistamine.

In one or more embodiments, the carrier or composition is a gel. In some embodiments, the carrier is a transparent gel. In some embodiments, the carrier is a translucent gel. In some embodiments, the transparent gel has a higher viscosity than the translucent gel. In some embodiments, transparency is an indicator that the excipients are compatible in the carrier and the active ingredient(s) is/are compatible in the composition. In some embodiments, the absence of transparency (e.g., presence of translucency) is an indicator that one or more of the excipients may have some incompatibility in the carrier. In some embodiments, a transparent gel has a higher viscosity than a translucent gel. In some embodiments, the translucent gel has a higher viscosity than the transparent gel. In some embodiments, a translucent gel has a higher ability to flow than a transparent gel. In some embodiments, a translucent composition may be flowable. In some embodiments, a translucent composition may be pourable. In some embodiments, upon addition of active ingredient(s), it is an opaque gel. In some embodiments, upon addition of active ingredient(s), it is a hazy gel. In some embodiments, a hazy or opaque composition may be flowable. In some embodiments, a hazy or opaque composition may be pourable. In some embodiments, the active agent provides color to the gel. In some embodiments, a coloring agent is added to the carrier or composition.

In some embodiments, the carrier or composition is at room temperature a semi-solid and in other embodiments is a liquid.

In some embodiments, the carrier or composition is foamable. In some embodiments, the carrier or composition comprises a foam adjuvant. In some embodiments the carrier or composition is not foamable. Oils are defoamers and silicone oils can be good defoamers. Elastomers comprise a mixture of a silicone oil and a silicone crosspolymer and elastomer based formulations are defoamers. In one or more embodiments it is challenging to achieve a foamable carrier or composition based on elastomers/silicone oils and other oils that can produce a foam. In some embodiments a foamable composition comprises a reduced amount of elastomer and or silicone oil and an increased amount of foam adjuvants and surfactants and other hydrophobic solvents. In one or more embodiments the surfactants are a combination of surfactants forming a complex emulgator and or having a difference in HLB values of at least 2, or at least 3. In one or more embodiments, polymeric agents which have surfactant properties are used such as poloxamers. In one or more embodiments the surfactants are silicone surfactants. In one or more embodiments the formulation is filled in an aerosol canister to which propellant is added. In one or more embodiments the formulation is adjusted so as to reduce the amounts of suspended solids that can potentially block the aerosol canister valve and to improve the shakability of the canister contents including propellant to a level that will allow repeated use of the canister without resulting in a block.

In some embodiments, the carrier or composition comprises a propellant. In some embodiments, the propellant is a hydrophobic propellant. In some embodiments, the propellant is a liquified or pressurized gas hydrophobic propellant. In some embodiments, the propellant includes one or more of propane, butane and isobutane. In some embodiments, the propellant is AP46, and in others, AP70. In some embodiments, the propellant is about 3% to about 25%, or about 5% to about 18%, or about 6% to about 15% by weight of the composition. In some embodiments, the ratio of propellant to composition is about 3:100 to about 25:100, or about 5:100 to about 18:100, or about 6:100 to about 15:100 by weight of the composition. In some embodiments, the foamable composition upon release from a pressurized canister forms a foam. In some embodiments the foam is quick breaking. In some embodiments, the foam is a breakable foam. In some embodiments the foam is thermolabile. In some embodiments, the foam is not thermolabile at 37° C. In some embodiments, it has a collapse time at 37° C. of at least about 30 secs, or at least about 60 secs, at least about 90 secs, of at least about 120 secs, or at least about 150 secs, at least about 180 secs, or at least about 240 secs, at least about 300 secs.

In one or more embodiments, the composition when applied to a surface does not run. In some embodiments, the composition is not a liquid. In some embodiments, the composition is not a runny liquid. In some embodiments, the composition is thixotropic. In some embodiments, it is shear thinning. By shear thinning is meant that on the application of stress such as extruding or squeezing through a restricted opening, the composition will act as a lower viscosity composition. So, by way of example upon application of a shear force to a gel composition, the composition may shear thin and become flowable or fluid.

In one or more embodiments, the carrier or composition when applied to a skin or mucosal surface, has a bioadhesive or mucoadhesive quality. In one or more embodiments, the composition forms a quasi-layer. In one or more embodiments, the quasi-layer facilitates the absorption of the active agent, such as tofacitinib into an epidermal and dermal layer of skin. In one or more embodiments, the quasi-film facilitates the absorption of the active agent, such as tofacitinib into a mucosal membrane. In one or more embodiments, the quasi-film facilitates the absorption of the active agent, such as tofacitinib into the lining of a body cavity. In one or more embodiments, having an interfacial tension of the composition and the active agent below that of the active agent with a metal or with a plastic may lead to a more effective delivery of the active agent. In one or more other embodiments, having an interfacial tension of the composition and the active agent above or similar to that of the active agent with a metal or with a plastic may lead to a more effective delivery of the active agent.

In one or more embodiments, having an interfacial tension of the composition and the active agent below that of the active agent with skin, or with a mucosal surface, or body cavity surface may lead to a more effective delivery of the active agent. In one or more other embodiments, having an interfacial tension of the composition and the active agent above or similar to that of the active agent with skin, or a mucosal surface, or body cavity surface may lead to a more effective delivery of the active agent.

In one or more embodiments, delivery of a JAK inhibitor salt, e.g., tofacitinib salt in the skin, mucosal and body cavity lining is higher than with a JAK inhibitor base, e.g., tofacitinib base. In one or more embodiments delivery of a JAK inhibitor, such as a tofacitinib salt in the skin, mucosal and body cavity lining is more than about 50%, or more than about 100% or more than about 200% higher than with tofacitinib base. In one or more embodiments delivery of a JAK inhibitor, such as a tofacitinib salt though the skin, mucosal or body cavity lining is comparable with or lower than with tofacitinib base. In one or more embodiments, the carrier base and emollient act synergistically to enhance delivery even though the JAK inhibitor, such as tofacitinib is not soluble or substantially not soluble in the carrier base and emollient.

In some embodiments, the carrier base comprises ST elastomer 10 and the emollient comprises MCT oil, or squalane, or isopropyl isostearate, or mixtures of any two or more thereof. In one or more embodiments the carrier composition further comprises a fragrance agent, a masking agent, a buffering agent, a pH agent, a preservative, a chelating agent, an antioxidant, a scavenger agent, a thickener, a diluent, an additional stabilizer and any mixtures of two or more thereof. In some embodiments, the carrier or composition further comprising at least one of a preservative, a chelating agent, an antioxidant, a scavenger agent, and any mixtures of two or more thereof. In other embodiments, the composition is free or substantially free of a preservative, a chelating agent, an antioxidant, a scavenger agent, and any mixtures of two or more thereof.

In one or more embodiments, there is provided a kit comprising a carrier/composition in a container and a disposable applicator connectable to the container. In some embodiments, the container is a tube. In some embodiments, it is a bottle with a pump. In some embodiments, it is an aerosol canister. In some embodiments, the container comprises a unit dose means suitable for delivery of a measured unit dose. In some embodiments, the unit dose is about 0.1 g, or about 0.2 g, or about 0.3 g, or about 0.4 g, or about 0.5 g, or about 0.6 g, or about 0.7 g, or about 0.8 g, or about 0.9 g. or about 1.0 g. In some embodiments, the disposable applicator is adapted for delivery of the composition to a body cavity. In some embodiments, the disposable applicator is adapted for delivery of the composition to a skin surface. In some embodiments, the disposable applicator is adapted for delivery of the composition to a mucosal surface.

In one or more embodiments, there is provided a method of treating a skin disorder comprising applying to the skin of a subject a composition described herein. In one or more embodiments, there is provided a method of treating a mucosal disorder comprising applying to the mucosa of a subject a composition described herein. In one or more embodiments, there is provided a method of treating a body cavity disorder comprising applying to the body cavity/body cavity surface of a subject a composition described herein.

In one or more embodiments, the method involves treating or preventing a JAK responsive dermatoses, e.g. a JAK 3 or JAK 1 responsive dermatoses. In one or more embodiments, the composition used in the method includes a JAK 3 and or a JAK 1 inhibitor, such as tofacitinib, e.g., tofacitinib citrate.

In one or more embodiments the skin disorder includes an eczema, a dermatitis, atopic dermatitis, or psoriasis

In some embodiments, the disorder is vitiligo. In some embodiments, the disorder is alopecia. In some embodiments, the disorder is alopecia totalis, alopecia universalis, atopic dermatitis, psoriasis, vitiligo, autoimmune bullous skin disorder such as pemphigus vulgaris (PV) or bullous pemphigoid (BP), skin rash, skin irritation, skin sensitization (e.g., contact dermatitis or allergic contact dermatitis), chronic atypical neutrophilic dermatosis with lipodystrophy and elevated temperature (CANDLE), pustulosis palmoplantaris, ichtyosis, eczema, actinic keratosis, pruritus, rosacea, lupus erythematosus, contact dermatitis, skin inflammation, skin itch, skin infection, acne, and acne vulgaris.

In one or more embodiments, the disorder is a mucosal disorder.

In one or more embodiments, the disorder is a body cavity disorder.

In one or more embodiments, there is provided a method of treating or preventing a dermatological disorder or a deterioration thereof comprising applying to the skin of a subject topical composition comprising a tofacitinib salt and a carrier in which the tofacitinib salt is suspended or substantially suspended, wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and wherein the carrier is free or substantially free of a penetration enhancer that dissolves a proportion of the tofacitinib salt; and wherein at least about 99.9% of the tofacitinib salt is suspended. In one or more embodiments the composition further comprises a fingolimod.

In one or more embodiments, the disorder treatable or preventable by the tofacitinib salt is a JAK responsive dermatoses e.g., a JAK 3 or JAK 1 responsive dermatoses. In one or more embodiments, the disorder treatable or preventable by the tofacitinib salt is a dermatological disorder, a mucosal disorder, or a body cavity disorder. In some embodiments, the dermatological disorder is an eczema. In some embodiments, the eczema is atopic dermatitis, contact dermatitis, dyshidrotic eczema, nummular eczema, seborrheic dermatitis or stasis dermatitis. In some embodiments the tofacitinib salt is tofacitinib citrate, and the dermatological disorder is a dermatitis, or is atopic dermatitis, or is psoriasis, or is rosacea.

In one or more embodiments, the tofacitinib is delivered into the epidermis and dermis. In some embodiments, the delivery to the epidermis is greater than to the dermis. In some embodiments, the delivery to the epidermis is at least about 20% or, at least about 50% or, at least about 100% or, at least about 150% or, at least about 200% or, at least about 250% or, at least about 300%, or at least about 400%, or at least about 500%, greater than to the dermis. In some embodiments, the delivery to the epidermis is expressed as a percentage of the applied dose. In some embodiments, the delivery to the epidermis as a percentage of applied dose is at least about 100% greater than to the dermis. In some embodiments, topical delivery of the tofacitinib to the dermis and epidermis is about or greater than 2-fold, or 3-fold, or 4-fold, or 5-fold, or 6-fold, or 7-fold, or 8-fold, or 9-fold, or 10-fold, or 15-fold than the delivery of the tofacitinib through the skin. In some embodiments, topical delivery of the tofacitinib to the dermis and epidermis is about or greater than 20-fold the delivery of the tofacitinib through the skin. In some embodiments, topical delivery of the tofacitinib to the dermis and epidermis is about or greater than 30-fold, or 40-fold, or 50-fold, than the delivery of the tofacitinib through the skin. In other words, the topical delivery with the carriers and compositions is advantageous as it will result in a low penetration through the skin into the blood and in consequence a lower systemic exposure of the active ingredient than if the same dose is given orally. In some embodiments, the tofacitinib is in an effective concentration sufficient to reach a plateau effect in the dermis or epidermis of a human subject to treat the disorder. In some embodiments, the concentration of the tofacitinib salt is about 0.5% to about 0.7% by weight of the composition. In some embodiments, the concentration of the tofacitinib salt is about 0.5%, or about 0.6%, or is about 0.7% by weight of the composition. In some embodiments, the carrier is free or substantially free of a penetration enhancer that dissolves a proportion of the tofacitinib, wherein that proportion is at least 0.1% by weight. In some embodiments, the carrier is free or substantially free of hydrophilic solvents. In some embodiments, the carrier base is about 83% to about 89% by weight of the composition, and the emollient is about 10% to about 16% by weight of the composition. In some embodiments, the carrier base comprises ST elastomer 10, and the emollient comprises MCT oil. In some embodiments, the emollient further comprises one or more of squalane, an isopropyl ester, and oleyl alcohol.

In one or more embodiments, the composition is applied to the area of the disorder. In some embodiments, the composition is applied to the area surrounding the area of the disorder. In some embodiments, the composition is applied to the area of the disorder and the area surrounding the disorder. In some embodiments, systemic exposure to tofacitinib applied topically is much less than when the same amount is applied orally. In some embodiments, the systemic exposure is at least about 20-fold less. In some embodiments. In some embodiments, the systemic exposure is at least about 70-fold less. In some embodiments, the systemic exposure is at least about 100-fold less. In some embodiments, the systemic exposure is at least about 200-fold less. In some embodiments, the systemic exposure is at least about 400-fold less. In some embodiments, the systemic exposure is at least about 500-fold less.

In one or more embodiments, the composition comprises a JAK inhibitor, e.g., tofacitinib citrate, and following treatment, the atopic dermatitis index is reduced significantly. In some embodiments, following treatment the atopic dermatitis index is less than three. In some embodiments, following treatment the atopic dermatitis index is about 2.5. In some embodiments wherein the carrier is free or substantially free of one or more of water, surfactants, hydrophilic compounds, preservatives, anti-oxidants, scavengers, chelating agents and additional stabilizers, following treatment the index is less than three. In some embodiments, following treatment the index is about 2.5. In some embodiments the reduction in the index is further improved when the composition comprises a therapeutically effective concentration of a fingolimod.

In one or more embodiments, there is provided a composition comprising a tofacitinib and a carrier in which part of the tofacitinib is suspended, wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and (iii) at least one compound in which the tofacitinib has some solubility; and wherein less than about 99.9% of the tofacitinib salt is suspended. In one or more embodiments the composition further comprises a fingolimod.

In one or more embodiments, less than about 99.8%, or less than about 99.7%, or less than about 99.6%, or less than about 99.5%, or less than about 99.3%, or less than about 99% of the tofacitinib salt is suspended. In one or more embodiments, the tofacitinib is a salt. In some embodiments, the tofacitinib is about 0.5% to about 0.7% by weight of the composition, or is about 0.5%, or about 0.6%, or is about 0.7% by weight of the composition. In one or more embodiments there is provided a method of treating or preventing a dermatological disorder or a deterioration thereof comprising applying to the skin of a subject the topical composition of a tofacitinib, e.g., tofacitinib citrate, wherein part is dissolved, and part is suspended, including in amounts described elsewhere herein. In one or more embodiments, a disorder responsive to treatment or prevention with a topical composition of a tofacitinib, e.g., tofacitinib citrate, wherein part is dissolved, and part is suspended, includes an eczema, a dermatitis or psoriasis, wherein the eczema is atopic dermatitis, contact dermatitis, dyshidrotic eczema, nummular eczema, seborrheic dermatitis or stasis dermatitis.

In one or more embodiments there is provided a topical composition comprising a JAK inhibitor and a carrier in which a JAK inhibitor is suspended or substantially suspended, wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and wherein the carrier is free or substantially free of a penetration enhancer that dissolves a proportion of the JAK inhibitor; and wherein at least about 99.9% of the JAK inhibitor is suspended. In one or more embodiments the composition further comprises a S1PR modulator or agonist e.g., a fingolimod.

In one or more embodiments, the JAK inhibitor is a salt, e.g., a citrate salt. In some embodiments, the JAK inhibitor is about 0.5% to about 0.7% by weight of the composition, or is about 0.5%, or about 0.6%, or is about 0.7% by weight of the composition. In some embodiments, the JAK inhibitor is about 0.3% to about 1.5% by weight of the composition. In one or more embodiments there is provided a method of treating or preventing a dermatological disorder or a deterioration thereof comprising applying to the skin of a subject the topical composition of a JAK inhibitor, e.g., a citrate salt, wherein it is suspended or substantially suspended, including in amounts described elsewhere herein. In one or more embodiments a disorder responsive to treatment or prevention with a topical composition of a tofacitinib, e.g., tofacitinib citrate, wherein, includes, an eczema, a dermatitis or psoriasis, wherein the eczema is atopic dermatitis, contact dermatitis, dyshidrotic eczema, nummular eczema, seborrheic dermatitis or stasis dermatitis.

In one or more embodiments there is provided a composition comprising a JAK inhibitor and a carrier in which part of the JAK inhibitor is suspended, wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and (iii) at least one compound in which the JAK inhibitor has some solubility; and wherein less than about 99.9% by weight of the JAK inhibitor is suspended. In other words, part is dissolved, and part is suspended. In one or more embodiments the composition further comprises a S1PR modulator or agonist e.g., a fingolimod. In some embodiments, less than about 99.8%, or less than about 99.7%, or less than about 99.6%, or less than about 99.5%, or less than about 99.3%, or less than about 99% by weight of the JAK inhibitor is suspended. In some embodiments, the JAK inhibitor is a salt. In some embodiments, the JAK inhibitor is about 0.5% to about 0.7% by weight of the composition, or is about 0.5%, or about 0.6%, or is about 0.7% by weight of the composition. In some embodiments, the JAK inhibitor is about 0.3% to about 1.5% by weight of the composition. In one or more embodiments there is provided a method of treating or preventing a dermatological disorder or a deterioration thereof comprising applying to the skin of a subject the topical composition of a JAK inhibitor, e.g., a citrate salt, wherein part is dissolved and part is suspended, including in amounts described elsewhere herein. In one or more embodiments a disorder responsive to treatment or prevention with topical composition of a tofacitinib, e.g., tofacitinib citrate, wherein part is dissolved, and part is suspended, includes, an eczema, a dermatitis or psoriasis, wherein the eczema is atopic dermatitis, contact dermatitis, dyshidrotic eczema, nummular eczema, seborrheic dermatitis or stasis dermatitis.

In one or more embodiments there is provided a topical composition comprising an active agent in a pharmaceutically effective amount and a carrier in which the active agent is suspended or substantially suspended, wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and wherein the carrier is free or substantially free of a penetration enhancer that dissolves a proportion of the active agent; and wherein at least about 99.9% by weight of the active agent is suspended. In some embodiments the active agent is a salt, e.g., a citrate salt. In some embodiments the active agent is a combination of two or more active agents. In one or more embodiments there is provided a method of treating or preventing a dermatological disorder or a deterioration thereof comprising applying to the skin of a subject the topical composition of an active agent, e.g., a citrate salt, that is suspended or substantially suspended, including in amounts described elsewhere herein. In one or more embodiments a disorder responsive to treatment or prevention with a topical composition of a tofacitinib, e.g., tofacitinib citrate, suspended or substantially suspended, includes an eczema, a dermatitis or psoriasis, wherein the eczema is atopic dermatitis, contact dermatitis, dyshidrotic eczema, nummular eczema, seborrheic dermatitis or stasis dermatitis. In one or more embodiments the composition further comprises a S1PR modulator or agonist e.g., a fingolimod.

In one or more embodiments there is provided a composition comprising an active agent and a carrier in which part of the active agent is suspended, wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and (iii) at least one compound in which the active agent has some solubility; and wherein less than about 99.9% by weight of the active agent is suspended. In other words, part is dissolved, and part is suspended. In some embodiments the active agent is a combination of two or more active agents. In some embodiments, less than about 99.8%, or less than about 99.7% less than about 99.6%, or less than about 99.5% less than about 99.3%, or less than about 99% by weight of the active agent is suspended. In some embodiments, the active agent is a salt, e.g., a citrate salt. In one or more embodiments there is provided a method of treating or preventing a dermatological disorder or a deterioration thereof comprising applying to the skin of a subject the topical composition of an active agent, e.g., a citrate salt, wherein part is dissolved, and part is suspended, including in amounts described elsewhere herein. In one or more embodiments a disorder responsive to treatment or prevention with a topical composition of a tofacitinib, e.g., tofacitinib citrate, wherein part is dissolved, and part is suspended, includes, an eczema, a dermatitis or psoriasis, wherein the eczema is atopic dermatitis, contact dermatitis, dyshidrotic eczema, nummular eczema, seborrheic dermatitis or stasis dermatitis. In one or more embodiments the composition further comprises a S1PR modulator or agonist e.g., a fingolimod.

In one or more embodiments, there is provided a topical carrier composition for suspending or substantially suspending at least about 99.9% by weight of an active agent, wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and wherein the carrier is free or substantially free of a penetration enhancer that can dissolve a proportion of the active agent.

In one or more embodiments the carrier composition further comprising an active agent, e.g., wherein the active agent comprises a JAK inhibitor, e.g., wherein the JAK inhibitor is a salt, e.g., wherein the JAK inhibitor is a tofacitinib, e.g., wherein the tofacitinib is a salt, e.g., wherein the salt is tofacitinib citrate, e.g., wherein the tofacitinib is about 0.5% to about 0.7% by weight of the composition, or is about 0.5%, or about 0.6%, or is about 0.7% by weight of the composition. In some embodiments, the JAK inhibitor is about 0.3% to about 1.5% by weight of the composition. In one or more embodiments, there is provided a method of treating or preventing a dermatological disorder or a deterioration thereof comprising applying to the skin of a subject the topical carrier composition for suspending or substantially suspending at least about 99.9% by weight of an active agent. In one or more embodiments the composition further comprises a S1PR modulator or agonist e.g., a fingolimod.

In one or more embodiments a disorder is responsive or partially responsive to treatment or prevention with a topical carrier composition without an active agent.

wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and herein the carrier is free or substantially free of a penetration enhancer that can dissolve a proportion of the active agent, wherein the disorder includes, an eczema, a dermatitis or psoriasis, wherein the eczema is atopic dermatitis, contact dermatitis, dyshidrotic eczema, nummular eczema, seborrheic dermatitis or stasis dermatitis.

In one or more embodiments, there is provided a topical carrier for suspending part and dissolving part of an active agent, wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and (iii) at least one compound in which the active agent has some solubility; and wherein the carrier is able to suspend less than about 99.9% by weight of the active agent and to dissolve at least about 0.1%. In some embodiments, the carrier has the capacity to dissolve up to about 15% by weight of the active agent. In some embodiments, the carrier has the capacity to dissolve more than about 0.2%, or more than about 0.3% or more than about 0.4%, or more than about 0.5%, or more than about 0.7%, or more than about 1% by weight of the active agent. In some embodiments, the carrier has the capacity to dissolve between about 0.1% and about 0.2%, or between about 0.3% and about 0.4%, or between about 0.4% and about 0.5%, or between about 0.5% and about 0.7%, or between about 0.7% and about 1.0%, or between about 1.0% and about 15% by weight of the active agent, e.g., wherein the active agent is a salt. In one or more embodiments, there is provided a method of treating or preventing a dermatological disorder or a deterioration thereof comprising applying to the skin of a subject the topical carrier composition capable of dissolving part and suspending part of an active agent. In one or more embodiments the active agent is a tofacitinib, a fingolimod or a combination thereof.

In one or more embodiments, a disorder is responsive or partially responsive to treatment or prevention with topical carrier composition without an active agent;

wherein the carrier comprises: (i) a carrier base comprising at least one elastomer, a gelled mineral oil, at least one gelling agent in at least one lipophilic solvent or oil, and mixtures of any two or more thereof; and (ii) at least one emollient; and (iii) at least one compound in which the active agent has some solubility; and wherein the carrier can suspend less than about 99.9% by weight of the active agent and to dissolve at least about 0.1%, wherein the disorder includes, an eczema, a dermatitis or psoriasis, and wherein the eczema is atopic dermatitis, contact dermatitis, dyshidrotic eczema, nummular eczema, seborrheic dermatitis, or stasis dermatitis. In one or more embodiments the active agent is a tofacitinib, a fingolimod or a combination thereof.

It should be noted that topical compositions disclosed herein can be applied to the target site as a gel or a semi-solid gel. In certain other embodiments, it can be applied as an ointment, or a liquid, or a foam or a breakable foam.

Application of the claimed compositions can be, for example, hourly, twelve hourly (e.g., twice daily), daily, alternate-day or intermittent, according to the condition of the patient. For reasons of compliance, less frequent applications, where possible, are preferable, e.g., daily single applications. In certain cases, where prolonged or long-term treatment is required, an initial dose is provided, followed by a gradual reduction to a lower maintenance dose, which can be increased if further outbreaks occur.

In one or more embodiments, the initial dose of a tofacitinib is about 0.1% to about 1.2% by weight of the composition. In one or more embodiments, the initial dose of a tofacitinib is about 0.5% to about 0.7% by weight of the composition. In one or more embodiments, the initial dose of a tofacitinib is about 0.5%, about 0.6%, or about 0.7% by weight of the composition. In one or more embodiments, the initial dose of a tofacitinib is about 0.6% tofacitinib by weight of the composition.

In one or more embodiments, the topical composition comprises a tofacitinib salt. The D90 particle size of the tofacitinib in one or more embodiments is less than or about 25 microns, or less than about 22 microns, or less than about 19 microns, or less than or about 16 microns, or less than or about 13 microns, or less than about 10 microns, or less than or about 9 microns, or less than or about 8 microns, or less than or about 7 microns, or less than or about 6 microns, or less than or about 5 microns. In one or more certain embodiments, 90% of the tofacitinib particles are less than or about one of the aforesaid amounts in size. In an embodiment, the D90 particle size ranges from about 6 microns to about 11 microns, or from about 7 microns to about 9 microns or from about 7.5 microns to about 8.5 microns. Skin penetration may be enhanced by having a smaller particle size.

In one or more embodiments, the carrier is at a concentration of about 40% to about 95% by weight. In one or more embodiments, the carrier is at a concentration of about 42% to about 93% by weight. In one or more embodiments, the carrier is at a concentration of about 44% to about 91% by weight. In one or more embodiments, the carrier is at a concentration of about 50% to about 90% by weight. In one or more embodiments, the carrier is at a concentration of about 55% to about 90% by weight. In one or more embodiments, the carrier is at a concentration of about 60% to about 90% by weight. In one or more embodiments, the carrier is at a concentration of about 65% to about 90% by weight. In one or more embodiments, the carrier is at a concentration of about 70% to about 90% by weight. In one or more embodiments, the carrier is at a concentration of about 75% to about 90% by weight. In one or more embodiments, the carrier is at a concentration of at least about 40% by weight, or at least about 45% by weight, or at least about 50% by weight, or at least about 55% by weight, or at least about 60% by weight, or at least about 65% by weight, or at least about 70% by weight, or at least about 75% by weight, or at least about 80% by weight, or at least about 85% by weight, or at least about 90% by weight, or at least about 92% by weight, or at least about 94% by weight and any ranges between any two figures listed for example from about 55% to about 94%. In some embodiments, the carrier is at a concentration of less than about 95% by weight, or is at a concentration of less than about 90% by weight, or is at a concentration of less than about 85% by weight, or less than about 80% by weight, or less than about 70% by weight, or less than about 60% by weight, or less than about 50% by weight. In one or more embodiments, the carrier is at a concentration of about 70% by weight, or about 72% by weight, or about 74% by weight, or about 76% by weight, or about 78% by weight, or about 80% by weight, or about 82% by weight, or about 84% by weight, or about 86% by weight, or about 88% by weight, or about 90% by weight, or about 92% by weight, or about 94% by weight, or about 96% by weight, or about 98% by weight. In one or more embodiments, the carrier is at a concentration of about 79.3% by weight, or about 82.4% by weight, or about 87% by weight, or about 87.4% by weight, or about 88% by weight, or about 88.24% by weight, or about 88.6% by weight.

In one or more embodiments, the carrier comprises at least one hydrophobic agent. In one or more embodiments, the hydrophobic agent or at least one hydrophobic agent comprises or is selected from the group consisting of an oil, a mineral oil, a hydrocarbon oil, an ester oil, an ester of a dicarboxylic acid, a triglyceride oil, an oil of plant origin, an oil from animal origin, an unsaturated or polyunsaturated oil, a diglyceride, a PPG alkyl ether, an essential oil, a silicone oil, a liquid paraffin, an isoparaffin, a polyalphaolefin, a polyolefin, a polyisobutylene, a synthetic isoalkane, isohexadecane, isododecane, alkyl benzoate, alkyl octanoate. C12-C15 alkyl benzoate, C12-C15 alkyl octanoate, arachidyl behenate, arachidyl propionate, benzyl laurate, benzyl myristate, benzyl palmitate, bis(octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, dimethylhexanoate, diethylhexyl adipate, diethylhexyl malate, diethylhexyl succinate, diisopropyl adipate, diisopropyl dimerate, diisopropyl sebacate, diisostearyl dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystearate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isostearyl citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, propyl myristate, propylene glycol myristyl ether acetate, propylene glycol dicaprate, propylene glycol dicaprylate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, alexandria laurel tree oil, an avocado oil, an apricot stone oil, a barley oil, a borage seed oil, a calendula oil, a canelle nut tree oil, a canola oil, a caprylic/capric a triglyceride castor oil, a coconut oil, a corn oil, a cotton oil, a cottonseed oil, an evening primrose oil, a flaxseed oil, a groundnut oil, a hazelnut oil, glycereth triacetate, glycerol triheptanoate, glyceryl trioctanoate, glyceryl triundecanoate, a hempseed oil, a jojoba oil, a lucerne oil, a maize germ oil, a marrow oil, a millet oil, a neopentylglycol dicaprylate/dicaprate, an olive oil, a palm oil, a passionflower oil, pentaerythrityl tetrastearate, a poppy oil, propylene glycol ricinoleate, a rapeseed oil, a rye oil, a safflower oil, a sesame oil, a shea butter, a soya oil, a soybean oil, a sweet almond oil, a sunflower oil, a sysymbrium oil, a syzigium aromaticum oil, a tea tree oil, a walnut oil, wheat germ glycerides, a wheat germ oil, PPG-2 butyl ether, PPG-4 butyl ether, PPG-5 butyl ether. PPG-9 butyl ether, PPG-12 butyl ether, PPG-14 butyl ether, PPG-15 butyl ether, PPG-15 stearyl ether, PPG-16 butyl ether, PPG-17 butyl ether, PPG-18 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether. PPG-10 cetyl ether, PPG-28 cetyl ether, PPG-30 cetyl ether, PPG-50 cetyl ether, PPG-30 isocetyl ether, PPG-4 lauryl ether, PPG-7 lauryl ether, PPG-2 methyl ether, PPG-3 methyl ether, PPG-3 myristyl ether, PPG-4 myristyl ether, PPG-10 oleyl ether, PPG-20 oleyl ether, PPG-23 oleyl ether, PPG-30 oleyl ether, PPG-37 oleyl ether, PPG-40 butyl ether, PPG-50 oleyl ether, PPG-11 stearyl ether, a herring oil, a cod-liver oil, a salmon oil, a cyclomethicone, a dimethyl polysiloxane, a dimethicone, an epoxy-modified silicone oil, a fatty acid-modified silicone oil, a fluoro group-modified silicone oil, a methylphenylpolysiloxane, phenyl trimethicone, a polyether group-modified silicone oil and mixtures of any two or more thereof. In some embodiments, the hydrophobic agent comprises or is selected from the group consisting of a soybean oil, a coconut oil, a cyclomethicone, a light mineral oil, a heavy mineral oil and mixtures thereof. In one or more embodiments, the solvent is tested individually for compatibility with an active agent, such as tofacitinib and is only used if it passes a compatibility test as described below in the Methods.

In one or more embodiments, the hydrophobic agent is at a concentration of about 75% to about 90% by weight. In one or more embodiments, the hydrophobic agent is at a concentration of about 55% to about 90% by weight. In one or more embodiments, the hydrophobic agent is at a concentration of about 50% to about 90% by weight. In one or more embodiments, the hydrophobic agent is at a concentration of at least about 40% by weight, at least about 45% by weight, at least about 50% by weight, at least about 55% by weight, at least about 60% by weight, at least about 65% by weight, at least about 70% by weight, at least about 75% by weight, at least about 80% by weight, at least about 85% by weight, at least about 90% by weight at least about 92% by weight, or at least about 94% by weight and any ranges between any two figures listed for example from about 55% to about 94%. In some embodiments, the hydrophobic agent is at a concentration of less than about 90% by weight, less than about 80% by weight, less than about 70% by weight, less than about 60% by weight, less than about 50% by weight. In one or more embodiments, the hydrophobic agent is at a concentration of about 70% by weight, or about 72% by weight, or about 74% by weight, or about 76% by weight, or about 78% by weight, or about 80% by weight, or about 82% by weight, or about 84% by weight, or about 86% by weight, or about 88% by weight, or about 90% by weight, or about 92% by weight, or about 94% by weight, or about 96% by weight, or about 98% by weight. In one or more embodiments, the hydrophobic agent is at a concentration of about 79.3% by weight, or about 82.4% by weight, or about 87% by weight, or about 87.4% by weight, or about 88% by weight, or about 88.24% by weight, or about 88.6% by weight.

In one or more embodiments, the hydrophobic composition comprises a gelled oil. In one or more embodiments, the gelled oil is a gelled mineral oil. In one or more embodiments, the gelled mineral oil is a VERSAGEL®. VERSAGELs® are gelled oils or emollients that can come in different product forms including, for example, the VERSAGEL® m, VERSAGEL® p, VERSAGEL® r and VERSAGEL® s series, and provide various viscosity grades. There are also VERSAGELs®, with isohexadecane, or with isododecane, or with hydrogenated polyisobutene, or with isopropylpalmitate. In an embodiment, it is VERSAGEL® 750 m. In an embodiment, it is VERSAGEL® 200 m. In an embodiment, it is VERSAGEL® 500 m. In an embodiment, it is VERSAGEL® 1600 m. VERSAGEL® m contains a mixture of mineral oil plus one or two or more of e.g., Ethylene/Propylene/Styrene Copolymer plus e.g., Butylene/Ethylene/Styrene Copolymer plus e.g., butylated hydroxyl toluene or similar gelling agents. In one or more embodiments, the gelled oil is at a concentration of about 55% to about 85% by weight. In one or more embodiments, the gelled oil is at a concentration of about 60% to about 80% by weight. In one or more embodiments, gelled oil is at a concentration of about 65% to about 75% by weight. In one or more embodiments, the gelled oil is at a concentration of about 55% to about 95% by weight. In one or more embodiments, the hydrophobic agent is at a concentration of about 75% to about 90% by weight. In one or more embodiments, the hydrophobic agent is at a concentration of about 21% to about 39% by weight. In one or more embodiments, the hydrophobic agent is at a concentration of about 26% to about 34% by weight. In one or more embodiments, the hydrophobic agent is at a concentration of about 9% to about 24% by weight. In one or more embodiments, the hydrophobic agent comprises a petrolatum at a concentration of about 9% to about 24% by weight, or about 26% to about 34% by weight or about 21% to about 39% by weight, or about 45% by weight, or about 50% by weight or about 55% by weight or about 60% by weight.

In one or more embodiments, the emollient comprises or is selected from the group consisting of isostearic acid derivatives, isopropyl palmitate, lanolin oil, diisopropyl dimerate, diisopropyl adipate, dimethyl isosorbide, maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, hydrogenated coco-glycerides, isononyl isononanoate, isotridecyl isononanoate, myristyl myristate, triisocetyl citrate, octyl dodecanol, octyl hydroxystearate and mixtures thereof. Other examples of other suitable emollients can also be found in the Cosmetic Bench Reference, pp. 1.19-1.22 (1996), which is incorporated herein by reference for emollients.

In one or more embodiments, the fatty alcohol and/or fatty acid have a melting point of at least about 40° C.

In one or more embodiments, the fatty alcohol comprises or is selected from the group consisting of lauryl alcohol, myristyl alcohol, cetyl alcohol, stearyl alcohol, arachidyl alcohol, behenyl alcohol, tetracosanol, hexacosanol, octacosanol, triacontanol, and tetratriacontanol. In one or more embodiments, the fatty acid comprises or is selected from the group consisting of dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid, and pentatriacontanoic acid.

In one or more embodiments, the fatty alcohol or the fatty acid is about 3% to about 10% by weight. For example, about 3% by weight, or about 4% by weight, or about 5% by weight, or about 6% by weight, or about 7% by weight, or about 8% by weight, or about 9% by weight, or about 10% by weight. For example, about 4.1% by weight, or about 4.4% by weight, or about 4.5% by weight, or about 5% by weight, or about 5.6% by weight, or about 8.6% by weight.

In one or more embodiments, the fatty alcohol is less than about 8% by weight. For example, less than about 7% by weight, or less than about 6% by weight, or less than about 5% by weight, or less than about 4% by weight.

In one or more embodiments, the carbon chain of the fatty alcohol or the fatty acid is substituted with a hydroxyl group.

In one or more embodiments, the fatty acid is 12-hydroxy stearic acid.

In one or more embodiments the composition comprises a modifying agentx. In one or more embodiments, the modifying agent is a wax comprising or selected from the group consisting of a plant wax, carnauba wax, candelilla wax, ouricury wax, sugarcane wax, retamo wax, jojoba oil, an animal waxes, beeswax, a petroleum derived wax, a paraffin wax, polyethylene, and derivatives thereof.

In one or more embodiments, the modifying agent is a combination comprising (i) at least one fatty alcohol and at least one fatty acid; or (ii) at least one fatty alcohol and at least one wax; or (iii) at least one fatty acid and at least one wax; or (iv) at least one fatty alcohol, at least one fatty acid, and at least one wax.

In one or more embodiments, the at least one modifying agent comprises or is selected from the group consisting of a fatty alcohol, a fatty acid and a wax, wherein the fatty alcohols and/or fatty acids have at least 12 carbon atoms in their carbon backbone. In certain embodiments the modifying agent is a combination of a fatty alcohol and a fatty acid and/or a wax.

In some embodiments, the fatty alcohol and/or fatty acid and/or wax are solid at ambient temperature. In certain embodiments, the fatty alcohol and/or the fatty acid and/or the wax or the mixture of them have a melting point of more than about 40° C.

In one or more embodiments, the wax is about 0% to about 6% by weight. For example, about 1% by weight, or about 2% by weight, or about 3% by weight, or about 4% by weight, or about 5% by weight, or about 6% by weight. In one or more embodiments, the wax is about 0.2% by weight.

In one or more embodiments, the wax is less than about 4% by weight. For example, less than about 3% by weight, or less than about 2% by weight, or less than about 1% by weight, or less than about 0.5% by weight.

In one or more embodiments, the fatty acid is about 1% to about 10% by weight. For example, about 1% by weight, or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

In one or more embodiments, the total amount of fatty acid fatty alcohol and wax, if present is about 1% to about 10% by weight. For example, about 1% by weight, or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

Where embodiments of the present invention are discussed herein in terms of a method of treatment involving the administration of a formulation or composition, it will be understood that the invention also provides that formulation, composition or active ingredient(s) thereof for use in that method, as well as the use of the formulation, composition or active ingredient(s) thereof in the manufacture of a medicament for use in that method.

### BRIEF DESCRIPTION OF THE DRAWINGS

FIG. 1 shows tofacitinib amounts in the epidermis, dermis and receptor fluid 24 hours following skin administration of emulsion-based (FIG. 1A) and ointment (petrolatum)-based (FIG. 1B) tofacitinib citrate formulations.

FIGS. 2A and 2B show glass bottle images of binary MCT oil-alternative oil/emollient mixtures at different ratios, combined with ST-elastomer 10.

FIG. 3 shows tofacitinib amounts (FIG. 3A) or percentage amount out of applied dose (FIG. 3B) in the epidermis, dermis and receptor fluid 24 hours following skin administration of Elastomer-based tofacitinib citrate formulations.

FIG. 4 shows tofacitinib amounts in the epidermis, dermis and receptor fluid 24 hours following skin administration of Elastomer-based tofacitinib citrate formulations with and without MCT oil (FIG. 4A) or a petrolatum-based formulation (+OCC—including petrolatum as an occlusive agent); (FIG. 4B).

FIG. 5 shows tofacitinib amounts (FIG. 5A) or percentage amount of applied dose (FIG. 5B) in the epidermis, dermis and receptor fluid 24 hours following skin administration of elastomer-based tofacitinib citrate formulations comprising MCT oil or MCT oil in combination with alternative emollients or a tofacitinib PEG-ointment-based formulation.

FIG. 6 shows results from in-vivo atopic dermatitis animal model study. FIG. 6A shows atopic dermatitis index for animals treated with elastomer-based tofacitinib citrate formulations comprising MCT oil at different tofacitinib strengths, a PEG ointment-based formulation and Triamcinolone 0.1% cream. FIG. 6B shows atopic dermatitis index for elastomer-based formulations at different tofacitinib strengths. Other parameters tested are visual parameters (FIG. 6C), behavioral parameters (FIG. 6D), inflammatory biomarkers (FIG. 6E and FIG. 6F) and epidermis thickness, mast cell numbers and microscopic atopic dermatitis score (FIG. 6G).) FIG. 6H shows mean body weight of animals at Day 39 (last day of treatment). FIGS. 6I-6P show results from a second in-vivo atopic dermatitis animal model study wherein animals were treated with formulations containing squalane and isopropyl isostearate with different strengths of tofacitinib citrate, a PEG ointment-based formulation and Triamcinolone 0.1% cream. FIG. 6I shows atopic dermatitis index for animals treated with the different formulations. Other parameters tested are body weight (FIG. 6J), IgE (FIG. 6K), inflammatory biomarkers (FIG. 6L, 6NP), histamine (FIG. 6M).

FIG. 7 shows cell viability (FIG. 7A) and skin irritation (IL-1α release; FIG. 7B) data for elastomer-based and emulsion-based formulations.

FIG. 8 shows cell viability (FIG. 8A) and skin irritation (IL-1α release; FIG. 8B) for elastomer-based and oleogel-based formulations.

FIG. 9 shows HET-CAM assay results measuring irritation of elastomer-based and emulsion-based formulations.

FIG. 10 shows photomicrographs of product homogeneity. FIG. 10A shows a photomicrograph of Fingolimod dispersed in the oil phase. FIG. 10B shows a photomicrograph of the combination of Fingolimod+Tofacitinib dispersed in the oil phase. FIG. 10C shows a photomicrograph of the combination of Fingolimod+Tofacitinib dispersed in the final formulation, when oil phase is added to ST Elastomer-10 and mixed for 10 minutes.

FIG. 11 shows tofacitinib citrate sticking to metal surfaces during the process of manufacturing of formulation OT1.0016A.

FIG. 12 shows a representative microscope image of an elastomer-based formulation with a micronized tofacitinib citrate (OT1.0031A).

FIG. 13 shows results for in-vivo psoriasis animal model study. FIG. 13A shows Psoriasis Index (PASI) for animals treated with different elastomer-based formulations with MCT oil, isopropyl isostearate and squalane, at different tofacitinib strengths compared to three control arms and FIG. 13B is a pictorial representation thereof.

FIG. 14 shows results for skin penetration study for elastomer-based formulations comprising different oils. FIG. 14A shows the skin to systemic delivery ratio. FIG. 14B shows the mean amount oftofacitinib recovery in epidermis, dermis and systemic.

FIGS. 15A and 15B show StratiCELL immunofluorescence skin barrier function results.

FIGS. 16A and 16B show StratiCELL skin histology results.

FIG. 17 shows atopic dermatitis index for AD mice treated with elastomer based formulations comprising different concentrations of fingolimod versus placebo.

### DETAILED DESCRIPTION

Unless defined otherwise, all technical and scientific terms used herein have the same meaning as is commonly understood by one of ordinary skill in the art to which this disclosure belongs. All ranges disclosed herein include the endpoints. The use of the term “or” shall be construed to mean “and/or” unless the specific context indicates otherwise. All patents, applications, published applications, and other publications are incorporated by reference in their entirety. In the event that there is a plurality of definitions for a term herein, those in this section prevail unless stated otherwise.

All % values are provided on a weight (w/w) basis.

Various carriers and compositions or formulations are described herein. They are often described for use in a method. A reference to or example of a carrier, composition or formulation for use in one method does not in any way limit the carrier, composition or formulation for use just in that method, but it can be for use in any other method or embodiment described herein. The carriers, compositions or formulations described herein are in one or more embodiments provided as carriers, compositions or formulations and are in one or more embodiments provided as a product even where they are described only in relation to their use in a method.

As used herein, the term “about” has its usual meaning in the context of pharmaceutical and cosmetic formulations to allow for reasonable variations in amounts that can achieve the same effect, typically plus or minus up to 30%. For example, if an amount of “about 1” is provided, then the amount can be up to 1.3 or from 0.70. In cases where “about X” will lead to a figure of above 100%, the term in one or more embodiments can be read as reflecting up to 100% by weight less the total of the minimum amount of the other ingredients. Likewise, it will be appreciated by one skilled in the art to the extent X is reduced from that upper level the amounts of the other ingredients are increased appropriately. As will be appreciated by one of skill in the art, there is some reasonable flexibility in formulating compositions such that where one or more ingredients are varied, successful formulations can still be made even if an amount falls slightly outside the range. Therefore, to allow for this possibility, amounts are qualified by about. In one or more embodiments, the examples e.g., amounts of formulation ingredients can be read as if prefixed with the term “about.” In one or more other embodiments, the examples can be read without the term “about.” In one or more embodiments, the figures can be read with the term “about.” In one or more other embodiments, the figures can be read without the term “about.”

As used herein, the terms “composition(s)” and “formulation(s)” can be used interchangeably depending on the context in which they are used as would be appreciated by a person skilled in the art.

The term “room temperature” as used herein, means 20° C. to 25° C. In an embodiment it is 20° C. In an embodiment it is 21° C. In an embodiment it is 22° C. In an embodiment it is 23° C. In an embodiment it is 24° C. In an embodiment it is 25° C. The term “ambient conditions” as used herein means room temperature, pressure and humidity. Ambient temperature and room temperature are used interchangeably herein.

The term “thixotropic,” as used herein, means that the formulation shows a decrease in viscosity upon application of a shear force. The structure of the formulation breaks down, leading to a reduction in viscosity. When the formulation is left standing without shear force, the viscosity is recovered.

As used herein, the term “gel”, refers, inter alia, to a carrier or formulation or composition that is not flowable at room temperature, such that when subjected to normal gravity at room temperature, it will retain its form. The term “flowable semi-solid”, as used herein refers, inter alia, to a base carrier or formulation that is slowly flowable when subjected to normal gravity at room temperature, and over time can adapt to and adopt the shape of a container. The term “liquid”, refers, inter alia, to a base carrier or formulation at room temperature, which is easily or readily flowable and can be poured into a container and can adapt to and adopt the shape of a container practically immediately.

As used herein, “foam” has its ordinary meaning to one of skill in the art, e.g., it may refer to an object or substance formed by trapping gas pockets within a solid or liquid. The gas pockets may comprise a gas, e.g., oxygen, nitrogen, or a mixture of gases, e.g., helium and xenon, or atmospheric air. The gas pockets within the foam may be connected to each other, e.g., closed-cell foams or discrete, e.g., open-cell foams. As used herein, “foamable compositions” refers to any composition that has the ability to form a foam. In some embodiments, foamable compositions comprise a carrier with or without a liquefied or compressed gas propellant, that forms a foam when the carrier is brought in contact with the propellant or by mechanical means, such as an air pump. In some embodiments, a foamable composition is packaged in an aerosol container together with a pressurized propellant. In some embodiments, the foamable composition is separate from the propellant such as in a bag in can system. In some embodiments, a valve on the aerosol container is actuated to release the foamable composition to form a foam.

In some embodiments, a formulation disclosed herein comprises water. In some embodiments, a formulation disclosed herein is water-free. As used herein, the terms “waterless” or “water-free,” or “anhydrous” or “nonaqueous” refer to compositions that contain no free or unassociated or absorbed water. In some embodiments, a waterless or water-free or anhydrous or nonaqueous composition comprises 0.0% added water by weight. Such a composition may contain trapped, bound, associated or otherwise unfree water, e.g., within its higher order crystal structure and in some embodiments such water may be about 1% or less. The terms “essentially waterless” or “essentially water-free” or “essentially anhydrous” or “essentially nonaqueous” refer to compositions that comprise less than 0.05% of water by weight. In some embodiments, an essentially water-free or anhydrous or nonaqueous composition comprises 0.04%, 0.03%, 0.02%, or 0.01% water by weight. The terms “substantially water-free” or “substantially waterless” or “substantially anhydrous” or “substantially nonaqueous” refer to compositions that comprise less than 0.5% of water by weight. In some embodiments, a substantially water-free or anhydrous or nonaqueous composition comprises 0.4%, 0.3%, 0.2%, or 0.1% water by weight. As used herein, “low water” refers to a composition that contains about or less than 1% of water by weight. In some embodiments, a composition with low water comprises 0.9%, 0.8%, 0.7%, 0.6% or 0.5% of water by weight.

The term “single phase” as used herein means that, after preparation the liquid components of the composition or carrier are fully miscible, and the solid components, if any, are either dissolved or homogeneously suspended in the composition so that only one phase is visible. In the context of a foamable composition “single phase” means that, after addition of propellant to the composition or carrier, the liquid components of the foamable composition or carrier are fully miscible, and the solid components, if any, are either dissolved or homogeneously suspended in the composition so that only one phase is visible. In some embodiments, a composition has a single phase before the addition of propellant. In some embodiments, a composition has a single phase after the addition of propellant.

By the term “substantially a single phase” it is meant that the composition or carrier, after preparation, is primarily or essentially a single phase as explained above, but can also have present a small amount of material which is capable of forming a separate phase amounting to less than about 5% by weight of the composition or carrier after the addition of propellant, e.g., less than about 3% by weight, or less than about 1% by weight of the composition. In the context of a foamable composition by the term “substantially a single phase” it is meant that the composition or carrier, after addition of propellant, is primarily or essentially a single phase as explained above, but can also have present a small amount of material which is capable of forming a separate phase amounting to less than about 5% by weight of the composition or carrier after the addition of propellant, e.g., less than about 3% by weight, or less than about 1% by weight of the composition. In some embodiments a composition may be a single phase before addition of propellant and a single phase after addition of propellant. In some embodiments a composition may be substantially a single phase before addition of propellant and a substantially single phase after addition of propellant. In some embodiments a composition may be substantially a single phase before addition of propellant and a single phase after addition of propellant. In some embodiments a composition may be a single phase before addition of propellant and substantially a single phase after addition propellant.

The terms “surfactant,” “surface active agent,” and “emulsifier” in the context used herein, refer to compounds that on their own, can both reduce surface tension between two substances or phases, and also stabilize an emulsion of water and oil. Reduction of surface tension in foam technology changes a material's ability to create small stable bubbles.

Surfactants include non-ionic, ionic, anionic, cationic, zwitterionic, amphoteric and amphiphilic surfactants. Surfactants may be derivatives of fatty alcohols or fatty acids, such as ethers or esters formed from such fatty alcohols or fatty acids with hydrophilic moieties, such as polyethylene glycol (PEG).

“Surfactant,” “emulsifier,” and “surface active agent,” as used herein, do not include compounds which do not function effectively on their own to reduce surface tension between two substances or phases and stabilize an emulsion of water and oil. For instance, a native (non-derivatized) fatty alcohol or fatty acid, as well as a wax, generally does not reduce surface tension between two substances or phases or stabilize an emulsion of water and oil on its own, and therefore is not considered a surfactant in the context used herein. Likewise, propoxylated lanolin oil derivatives are not themselves surfactants or emulsifiers. These excipients may be used in combination with or in lieu of a surfactant in some embodiments of the formulations disclosed herein. In some embodiments, foam adjuvants in formulations disclosed herein comprise fatty acids and/or fatty alcohols. In some embodiments, formulations disclosed herein comprise emollients comprising propoxylated lanolin oil derivatives.

As used herein, the term “emollient” refers to a material or agent that, when placed in contact with the human skin, is able to soften, smoothen, reduce scaling and itching, reduce inflammation, improve skin barrier function, and/or act as a carrier for active agents. Examples of emollients include but are not limited to avocado oil, isopropyl myristate, mineral oil, capric triglycerides, caprylic triglyceride, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohols, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oil, an essential oil, a silicone oil, dimethicone, cyclomethicone, polyalkyl siloxane, polyaryl siloxane, polyalkylaryl siloxane, a polyether siloxane copolymer, and poly(dimethylsiloxane)-(diphenyl-siloxane).

“Standard surfactant,” “customary surfactant” or “stand-alone surfactant” refer to customary non-ionic, anionic, cationic, zwitterionic, amphoteric and amphiphilic surfactants. Many standard surfactants are derivatives of fatty alcohols or fatty acids, such as ethers or esters formed from such fatty alcohols or fatty acids with hydrophilic moieties, such as polyethylene glycol (PEG). However, a native (non-derivatized) fatty alcohol or fatty acid, as well as waxes are not regarded as a standard surfactant.

The term “co-surfactant” as used herein means a molecule which on its own is not able to form and stabilize satisfactorily an oil-in-water emulsion, but when used in combination with a surfactant as defined herein, the co-surfactant has properties which can allow it to help a surfactant create an emulsion and can boost the stabilizing power or effect of the surfactant. Examples of co-surfactants include fatty alcohols, such as cetyl alcohol, or fatty acids, such as stearic acid. Cetyl alcohol is a waxy hydrophobic substance that can be emulsified with water using a surfactant. Some substances can have more than one function and for example, fatty alcohols can in some formulations act as a co-solvent. In certain circumstances, a co-surfactant can itself be converted into a surfactant or soap by, for example, adding a base, such as triethanolamine to a fatty acid like stearic acid.

The term “modifying agent” as used herein is an agent which, when added to a hydrophobic oil, facilitates the creation of a hydrophobic breakable vehicle in the form of a breakable gel or breakable foam. In one or more embodiments, it can facilitate the formation of a thixotropic gel or an elastic gel.

As used herein, a “foamer complex,” a “foam stabilizer” or a “foam adjuvant”, in relation to a foamable composition can comprise, e.g., a fatty alcohol, a fatty acid and/or a wax. In some embodiments, the foam adjuvant is a fatty alcohol and a wax or a fatty acid and a wax. In some embodiments, it is a wax. In some embodiments, the foam adjuvant or modifying agent comprises at least one of a fatty alcohol, a wax or a fatty acid. In some embodiments, the foam adjuvant or the modifying agent is selected from a group consisting of a fatty alcohol, a wax and a fatty acid. In some embodiments, the foam adjuvant is a fatty alcohol. In some embodiments, the foam adjuvant is a fatty acid. In some embodiments, the foam adjuvant is a wax. In some embodiments, a wax has the properties of a foam adjuvant. In some embodiments, a fatty alcohol, and/or a fatty acid and/or a wax is an adjuvant.

As used herein, a formulation disclosed herein may additionally include one or a combination of waxes. In some embodiments, a wax may have a melting point temperature of about 36° C. or higher. In some embodiments, a wax may have a melting point temperature of about 40° C. or higher. In some embodiments, a wax may have a melting point temperature of about 49° C. or higher. In some embodiments, a wax may have a melting point temperature of about 81° C. or higher. In some embodiments, a wax may have a melting point temperature of about 83° C. or higher. In some embodiments, a wax may have a melting point temperature of about 88° C. or higher. In some embodiments, a wax may have a melting point temperature of about 61° C. or higher. In some embodiments, a wax may have a melting point temperature of about 65° C. or higher. In some embodiments, a wax may have a melting point temperature of about 50° C. or higher. In some embodiments, a wax may have a melting point temperature of about 54° C. or higher. In some embodiments, a wax may have a melting point temperature of about 57° C. or higher. In some embodiments, a wax may have a melting point temperature of about 60° C. or higher. In one or more embodiments, the formulations provided herein comprise a wax, wherein the wax within the formulation has a melting point of 68-69° C. In one or more embodiments, the formulations provided herein comprise a wax, wherein the wax within the formulation has a melting point of 42-44° C. In some embodiments, a wax may have a melting point temperature of about 83-88° C. In some embodiments, a wax may have a melting point temperature of about 61-65° C. In some embodiments, a wax may have a melting point temperature of about 50-54° C. In some embodiments, a wax may have a melting point temperature of about 57-60° C.

The term “breakable” refers to a property of a gel or foam wherein the gel or foam is stable upon dispensing from a container yet breaks and spreads easily upon application of shear or mechanical force, which can be mild, such as a simple mechanical rub.

The term “water activity” as used herein represents the hygroscopic nature of a substance, or the tendency of a substance to absorb water from its surroundings. Microorganisms require water to grow and reproduce, and such water requirements are best defined in terms of water activity of the substrate. The water activity of a solution is expressed as Aw=P/Po, where P is the water vapor pressure of the solution and Po is the vapor pressure of pure water at the same temperature. Every microorganism has a limiting Aw, below which it will not grow; e.g., for Streptococci, Klebsiella spp, Escherichia coli, Clostridium perfringens, and Pseudomonas spp, the Aw value is 0.95. Staphylococcus aureus is most resistant and can proliferate with an Aw as low as 0.86, and fungi can survive at an Aw of at least 0.7.

The identification of a “solvent”, as used herein, is not intended to characterize the solubilization capabilities of the solvent for any specific active agent or any other component of the composition or foamable composition. Rather, such information is provided to aid in the identification of materials suitable for use as a component of the composition or foamable composition described herein.

The terms “hydrophobic gel composition” or “hydrophobic flowable semi-solid composition” or “hydrophobic liquid composition” or “hydrophobic foamable composition” or “hydrophobic foam composition” or “hydrophobic composition” as used herein refer to compositions that have a low solubility in water. In some embodiments, 100 to 1000 parts of water are needed to dissolve or render miscible 1 part of the composition. In some embodiments, 1000 to 10,000 parts of water are needed to dissolve or render miscible 1 part of the composition. In some embodiments, more than 10,000 parts of water are needed to dissolve or render miscible 1 part of the composition.

It should be noted that the term “substantially free of” an ingredient as used herein, is intended to mean that the composition comprises less than about 0.5% by weight of the ingredient unless specifically indicated otherwise.

As used herein, the term “essentially free of” an ingredient as used herein, is intended to mean that the composition comprises less than about 0.05% by weight of the ingredient, unless specifically indicated otherwise.

As used herein, the term “free of” an ingredient used herein, is intended to mean that the composition does not comprise any amount of the ingredient, unless specifically indicated otherwise e.g., where the ingredient is present in a trapped, bound, associated or otherwise unfree state. In one or more embodiments an ingredient will be considered as containing constituents normally found present in a trapped, bound, associated or otherwise unfree state, all in accordance with the grade of purity of the ingredient.

The terms “surfactant-free” or “emulsifier-free” or “non-surfactant” refer to compositions that comprise no or negligible levels of surfactants, emulsifiers, or surface-active agents. Where a formulation includes insignificant or de minimis amounts of surfactants, emulsifiers, or surface-active agents it is considered to be essentially surfactant-free. As used herein, “essentially free of surfactant” indicates less than about 0.05% by weight of a surfactant, e.g., a surfactant selected from the group consisting of non-ionic, ionic, anionic, cationic, zwitterionic, amphoteric and ampholytic surfactants. The term “substantially surfactant-free” relates to a composition that contains a total of about or less than 0.5% by weight of surfactant, e.g., a surfactant selected from the group consisting of non-ionic, ionic, anionic, cationic, zwitterionic, amphoteric and ampholytic surfactants. In some embodiments, the composition comprises about or less than 0.2% by weight of a surfactant; about or less than 0.15% by weight; about or less than 0.1% by weight; about or less than 0.05% by weight; or about or less than 0.01% by weight.

As used herein, the term “preventing” refers to avoiding the onset of a disorder or condition from occurring in a subject that has not yet been diagnosed as having the disorder or condition, but who may be susceptible to it.

The term “polyol” as used herein is an organic substance that contains at least two hydroxy groups in its molecular structure.

As used herein, the term “treatment” or “treating” refers to inhibiting, reversing, ameliorating, or reducing the disorder or condition, e.g., arresting its development; relieving the disorder or condition, e.g., causing regression of the disorder or condition or reversing the progression of the disorder or condition; slowing progression, or relieving or reducing one or more symptoms of the disorder or condition. In some embodiments, it can also mean preventing or helping to prevent the disorder or condition or one or more symptoms thereof.

The term “a method of preventing or treating a disease or a disorder” as provided throughout the specification is interchangeable with the term “use of the composition as a medicament for preventing or treating a disease.” It should be noted that the term “disease” is used interchangeably with the term “disorder.”

By “de minimis” it is meant to be so minor that its effect is to be disregarded, e.g., having no functional impact on a formulation or method.

As used herein, “shakability” refers to the degree to which the user can feel or hear the presence of a foamable composition when a pressurized canister filled with the foamable composition and propellant is shaken. Shaking is done with mild to normal force without vigorous or excessive force. When the user cannot sense the motion of the contents during shaking the foamable composition may be considered to be non-shakable. When the user can moderately sense the motion of the contents during the shaking, the foamable composition is considered moderately shakable. When the contents are flowable during shaking, the product is considered shakable.

As used herein, “RRT” refers to Relative Retention Time (RRT)—the ratio of the retention time of analyte peak relative to that of another used as a reference obtained under identical conditions. Each RRT represents a specific impurity

As used herein, “balling effect” refers to a granular feel or sensation experience upon rubbing a topical formulation onto the skin. In all examples where “balling” is mentioned the formulation is prepared without an active agent.

As used herein, “binary mixtures” refers to a mixture of two emollients or oils in addition to a base formulation. In one or more embodiments the base formulation comprises an elastomer. In one or more embodiments the base formulation comprises a polymeric gelling agent in oil. In one or more embodiments, the binary mixture refers to a combination of MCT oil and another oil/emollient in addition to the base formulation.

As used herein, “tertiary mixtures” refers to a mixture of three emollients or oils in addition to a base formulation. In one or more embodiments the base formulation comprises an elastomer. In one or more embodiments the base formulation comprises a polymeric gelling agent in oil. In one or more embodiments, the tertiary mixture refers to a combination of MCT oil and two other oil(s)/emollient(s) in addition to the base formulation.

As used herein, “transparent” refers to a formulation which allows light to pass through without being appreciably scattered so that under normal daylight conditions objects behind can be distinctly seen.

As used herein, “translucent” refers to a formulation which under normal daylight conditions lets some light pass through, but objects on the other side cannot be seen clearly. In contrast to transparent, a translucent material exhibits some appreciable light scattering and has a cloudy appearance.

As used herein, “opaque” or “hazy” refers to a formulation which under normal daylight conditions lets little or essentially no apparent light pass through.

As used herein, “Atopic Dermatitis Index (ADI)” refers to the sum of the scores of four factors consisting of: (1) the presence of erythema/haemorrhage on the skin, (2) edema/thickening of the skin, (3) excoriation/erosion of the skin and (4) dryness/peeling of the skin at the area of induction of atopic dermatitis.

The presence of erythema/haemorrhage is scored based on the following scoring grid:

0=no or no more erythema/haemorrhage on the skin,

1=weak erythema/haemorrhage on the skin,

2=moderate erythema/haemorrhage on the skin,

3=important erythema/haemorrhage on the skin.

The presence of edema/thickening is scored based on the following scoring grid:

0=no or no more edema/thickening of the skin,

1=weak edema/thickening of the skin,

2=moderate edema/thickening of the skin,

3=important edema/thickening of the skin.

The presence of excoriation/erosion is scored based on the following scoring grid:

0=no or no more excoriation/erosion of the skin,

1=weak excoriation/erosion of the skin,

2=moderate excoriation/erosion of the skin,

3=important excoriation/erosion of the skin.

The presence of dryness/peeling is scored based on the following scoring grid:

0=no or no more dryness/peeling of the skin,

1=weak dryness/peeling of the skin,

2=moderate dryness/peeling of the skin,

3=important dryness/peeling of the skin.

The sum of the scores of these factors provides a combined ADI, on a scale of 0 to 12. In one or more embodiments, the combined ADI score is below 3.

As used herein, “chemically stable” refers to a compound (active agent or excipient) or a composition where no significant decrease in assay and no significant increase in impurities and no significant appearance of breakdown products may be observed at the conditions and during the time period tested. A decrease in assay or increase in impurities may occur for example, when a compound or a composition is oxidized, degraded, and/or reacts upon exposure to air, light, skin, water, any pharmaceutical excipient, or any active agent under ambient conditions. By a significant decrease in assay is intended about or more than about 2% decrease of initial assay value. By a substantially significant decrease in assay is intended about or more than about 5% decrease of initial assay. By a significant increase in impurities or in breakdown products is intended about or more than about 2% decrease of the initial assay value. By a substantially significant increase in impurities or breakdown products is intended about or more than about 5% of the initial assay value. By no significant decrease in assay it is intended less than 1% decrease of initial assay value. By no significant increase in related compounds or breakdown impurities it is intended less than 1% increase of initial assay value. For clarity, if the initial assay value was 100% and the new assay value is 96% the decrease is 4%. Similarly, if the initial assay value for impurities or breakdown products is 0.1% and the new assay value is 1.1% the increase is 1%. In some embodiments, a significant or substantially significant decrease in assay and/or a significant or substantially significant increase in breakdown products/impurities, as described above, may occur in less than 24 hours, which could be e.g., less than 16 hours, less than 12 hours, less than 6 hours, less than 5 hours, less than 4 hours, less than 3 hours, less than 2 hours, or less than 1 hour, upon exposure under room temperature ambient conditions to, for example, air, light, skin, water, or pharmaceutical excipients or any active agent. The term “chemically unstable” refers to a compound or a composition that falls outside the above definition of chemically stable.

As used herein, the term “physically stable” refers to a compound or a composition where no significant change in its state is observed during the time period and conditions under which it is tested. For example, a physically stable formulation will allow for 5% or fewer agglomerates or will retain its same properties, allowing for a small change in balling effect overtime and conditions suitable for its use.

As used herein, the term “physically unstable” refers to a compound or a composition where a significant change in its state is observed during the time period and conditions under which it is tested. For example, a physically unstable formulation is one that results in apparent phase separation over time or conditions suitable for its use.

As used herein, “surface energy” refers to the amount of energy that exists in a unit area of interface (mJ/m2). The surface energy of a solid surface is conceptually the equivalent of the surface tension of a liquid. The surface energy of a solid is defined to be equal to the surface tension of the highest surface tension liquid that will completely wet the solid, with a contact angle of 0°. In one or more embodiments, the surface energy of a composition or a compound is about 15 mJ/m2 to about 25 mJ/m2, or about 18 mJ/m2 to about 22 mJ/m2, or about 19 mJ/m2 to about 22 mJ/m2, or about 21.1 mJ/m2 to about 21.5 mJ/m2, or about 21.2 mJ/m2 to about 21.5 mJ/m2, or about 21.3 mJ/m2 to about 21.5 mJ/m2, or about 21.4 mJ/m2 to about 21.5 mJ/m2, or any figure within these ranges.

As used herein, “surface tension” refers to the tension of the surface film of a liquid caused by the attraction of the particles in the surface layer by the bulk of the liquid, which tends to minimize surface area. It is equal to the amount of work necessary to create a unit area of air/liquid interface (mN/m=mJ/m2=dynes/cm). In one embodiment, the surface tension of a composition/compound is between about 25 mN/m to about 40 mN/m, or about 25 mN/m to about 35 mN/m, or about 27 mN/m to about 34 mN/m, or about 28 mN/m to about 34 mN/m, or about 29 mN/m to about 34 mN/m, or about 29 mN/m to about 32 mN/m, or about 29 mN/m to about 31 mN/m, or any figure within these ranges.

As used herein, “Interfacial Tension” refers to the amount of work necessary to create a unit area of liquid/liquid interface (mN/m=mJ/m2=dynes/cm). In one or more embodiments, the interfacial surface tension between an active agent and a surface/composition is between about 1 mN/m to about 3 mN/m, or about 1.2 mN/m to about 1.7 mN/m, or about 1.2 mN/m to about 1.5 mN/m, or about 1.9 mN/m to about 3 mN/m, or about 1.9 mN/m to about 2.2 mN/m, or about 2.5 mN/m to about 2.8 mN/m, or any figure within these ranges.

As used herein a “JAK receptor” in the context herein refers to a site in the skin or mucosal membrane to which a JAK inhibitor can bind, and amongst other things is capable of interfering with the JAK-STAT signalling pathway.

As used herein a “JAK inhibitor responsive disorder” and a “JAK associated disorder” can be used interchangeably depending on the context in which they are used as would be appreciated by a person skilled in the art and refer to a disorder, which is impacted by inhibiting the activity of one or more of the Janus kinase family of enzymes (such as JAK1, JAK2, JAK3, TYK2), thereby interfering with the JAK-STAT signalling pathway.

As used herein “homogenous” or “homogenous distribution” refers to the property of a composition, in which the particles and/or active agent and/or excipients are proportionally distributed throughout.

As used herein “micronized” refers to a substance reduced in size to a fine powder, the particles or crystals of which are measured in micrometers in diameter. A measurement of the particle or crystal size in suspension in a composition can be expressed as D90. If, for example, 90% of the particles or crystals in the suspension are less than 15 microns, then the D90 is 15 microns. In one or more embodiments, the D90 of the particles is less than about 50 μm. In one or more embodiments, it is less than 45 μm or less than 40 μm, or less than 35 μm, or less than 30 μm, or less than 25 μm, or less than 20 μm or less than 15 μm, or less than 10 μm. In one or more embodiments the D90 is between 30 μm and 20 μm, or is between 25 μm and 15 μm, or is between 20 μm and 10 μm, or is between 15 μm and 5 μm, or is between 10 μm and 3 μm. In one or more embodiments, micronization of tofacitinib results in broken crystals.

As used herein “non occlusive” refers to topical formulation or substance, which allows for significant trans-epidermal water loss initially when applied topically as an unbroken layer on healthy skin. By significant is intended in one or more embodiments of more than 30% water loss after 20 minutes following application.

As used herein “occlusive” refers to topical formulation or substance, which substantially retards or allows for no or negligible trans-epidermal water loss initially when applied topically as an unbroken layer on healthy skin.

As used herein “partially occlusive” refers to topical formulation or substance, which allows for moderate trans-epidermal water loss initially when applied topically as an unbroken layer on healthy skin.

As used herein a “penetration enhancer” refers to a compound or component of a topical formulation, which increases penetration of active ingredient through the skin barrier. In some embodiments, a penetration enhancer dissolves a significant proportion of active agent. In some embodiments, a penetration enhancer does not dissolve a significant proportion of active agent. In some embodiments a significant proportion is more than 0.1% by weight of composition.

As used herein, “hydrophilic” refer to a compound or a composition that is miscible with water. In one or more embodiments a composition may be “hydrophilic” in character even though it may comprise a compound that has some hydrophobic properties.

As used herein “not hydrophilic” refers to a compound or a composition that is not miscible with and/or repels water. In one or more embodiments a composition may be “not hydrophilic” in character even though it may comprise a compound that has some hydrophilic property.

As used herein a “quasi-layer” or “quasi-coating” refers to the general structure deposited on the surface of an area of skin or mucosa when a composition or carrier comprising a gel, or a flowable semi-solid, or a liquid carrier/composition, is spread on the surface of that area of skin or mucosa. The quasi-layer or coating is a potentially dynamic structure. In some embodiments, the quasi-layer or coating allows for evaporation of volatile components of the formulation at skin temperature such that the composition changes resulting in formation of a layer generally comprised of non-volatile residue on the skin. In some embodiments, the quasi-layer or coating allows for absorption of some components of the formulation into the skin or mucosa and thus results in formation of a layer comprised of non-absorbed residue, which covers the skin. In some embodiments the residue comprises both non-volatile and non-absorbed residue. Without being bound by any theory, the evaporation and or absorption of some components may assist penetration of the active agent or provide a higher local concentration of active agent on the skin or mucosa surface.

As used herein “free of preservatives” refers to compositions that comprise no or a negligible amount of preservatives. As used herein “essentially free of preservatives” refers to compositions that comprise less than 0.05% of preservatives by weight. In some embodiments, an essentially preservative-free composition comprises 0.04%, 0.03%, 0.020, or 0.01% preservatives by weight. The terms “substantially preservative-free” refer to compositions that comprise 3% or less than 3% of preservatives by weight. In some embodiments, the composition comprises less than 2% preservative by weight, or less than 1%, or less than 0.5%, or less than 0.4%, or less than 0.3%, or less than 0.2%, or less than 0.1%, or less than 0.09%, or less than 0.08%, or less than 0.07% or less than 0.06% preservative by weight.

As used herein free of “anti-oxidants” refers to compositions that comprises no or negligible amount of anti-oxidants. As used herein essentially free of “anti-oxidants” refers to compositions that comprises less than 0.05% of anti-oxidants. In some embodiments, an essentially anti-oxidant free composition comprises 0.04%, 0.03%, 0.02%, or 0.01% anti-oxidant by weight. In some embodiments, the composition comprises less than 0.04%, or less than 0.03%, or less than 0.02%, or less than 0.01%, or less than 0.005%, or less than 0.001% by weight of anti-oxidant. As used herein substantially free of “anti-oxidants” refers to compositions that comprises 2% or less than 2% by weight of anti-oxidant. In some embodiments, the composition comprises less than 1.5%, or less than 1%, or less than 0.5%, or less than 0.4%, or less than 0.3%, or less than 0.2%, or less than 0.1%, by weight of anti-oxidant.

As used herein free of “additional stabilizers” refers to compositions that comprises no or a negligible amount of additional stabilizers. As used herein essentially free of “additional stabilizers” refers to compositions that comprises less than 0.05% of additional stabilizers. In some embodiments, an essentially additional stabilizer-free composition comprises 0.04%, 0.03%, 0.02%, or 0.01% additional stabilizer by weight. In some embodiments, the composition comprises less than 0.04%, or less than 0.03%, or less than 0.02%, or less than 0.01%, or less than 0.005%, or less than 0.001% by weight of additional stabilizer. As used herein substantially free of “additional stabilizers” refers to compositions that comprises 2% or less than 2% by weight of additional stabilizers. In some embodiments, the composition comprises less than 1.5%, or less than 1%, or less than 0.5%, or less than 0.4%, or less than 0.3%, or less than 0.2%, or less than 0.1%, by weight of additional stabilizers.

As used herein the term ‘Washburn’ or‘Washburn method’ or ‘Washburn measurement’ refers to a method for measuring the contact angle and the surface free energy of porous substances such as bulk powder.

In a Washburn measurement, a glass tube with a filter base filled with powder, comes into contact with a test liquid. The liquid is drawn up as a result of capillary action. The increase in mass of the tube, which is suspended from a force sensor, is determined with respect to time during the measurement. This measurement allows to determine the contact angle for the powder and tested liquid. The Fowkes equation is used to calculate the surface free energy of a solid from the contact angle with liquids.

As used herein the term “suspended” refers to active agent particles being dispersed in a composition such that less than 0.1% by weight is dissolved within the composition. As used herein “substantially suspended” refers to active agent particles being dispersed in a composition such that less than 5% by weight is dissolved within the composition. As used herein “partly suspended” refers to a composition in which a proportion of the active ingredient is dissolved. In some embodiments, the proportion dissolved is at least about 0.1% by weight. In some embodiments, the proportion dissolved is at least about 0.2% by weight. In some embodiments, the proportion dissolved is at least about 0.3% by weight. In some embodiments, the proportion dissolved is at least about 0.4% by weight. In some embodiments, the proportion dissolved is at least about 0.5% by weight. In some embodiments, the proportion dissolved is at least about 0.6% by weight. In some embodiments, the proportion dissolved is at least about 0.7% by weight. In some embodiments, the proportion dissolved is at least about 1% by weight. In some embodiments, the proportion dissolved is at least about 5% by weight. In some embodiments, the proportion dissolved is at least about 10% by weight. In some embodiments, the proportion dissolved is at least about 15% by weight. For clarity, by way of example, the corollary of at least 0.6% dissolved is less than about 99.4% suspended. In one or more embodiments having a part dissolved may impact the rate and or the amount and or the depth/area of penetration.

As used herein, “a compound that dissolves the active agent” or “a compound that dissolves a proportion of the active agent” refers to a compound that facilitates active agent solubility of more than about 1 mg/g, i.e. more than about 0.1% by weight. Non limiting examples of compounds which dissolve tofacitinib citrate are dimethyl sulfoxide, polyethylene glycol, 400, polyethylene glycol 200, HCl (e.g. 0.02%), water, transcutol, propylene glycol dimethyl isosorbate and glycerin. In one or more embodiments for topical application a suitable solubility in a compound is about 5 mg/g (i.e. 0.5%) or higher. For example, compounds with such solubility for tofacitinib include dimethyl sulfoxide, polyethylene glycol 400, and polyethylene glycol 200. For clarity, if a compound with a solubility of 0.6% for tofacitinib represents 25% of a composition, and the total amount of tofacitinib is in excess of the amount that can be dissolved, then the amount of dissolved tofacitinib may be calculated as 0.15% (being one quarter of 0.6)

As used herein, “a compound that substantially dissolves the active agent” or “a compound that substantially dissolves a proportion of the active agent” refers to a compound that facilitates active agent solubility of between about 0.1 mg/g, to about 1 mg/g i.e. about 0.01% to about 0.1% by weight. Non limiting examples of compounds which substantially dissolve tofacitinib citrate are benzyl alcohol, ethanol, hexylene glycol, isopropyl alcohol and oleyl alcohol.

As used herein, “a compound that essentially dissolves the active agent” or “a compound that essentially dissolves a proportion of the active agent” refers to a compound that facilitates active agent solubility of between about 0.01 mg/g, to about 0.1 mg/g i.e. about 0.001% to about 0.01% by weight. Non limiting examples of compounds which essentially dissolve tofacitinib citrate are PPG-11 stearyl ether, diisopropyl adipate and isopropyl myristate.

As used herein, “a compound that does not dissolve the active agent” or “a compound that does not dissolve a proportion of the active agent” refers to a compound that allows for active agent solubility of less than about 0.01 mg/g, i.e. less than about 0.001% by weight. Non limiting examples of compound that do not dissolve tofacitinib citrate are MCT oil, isopropyl palmitate, cetearyl ethylhexanoate, squalane, isopropyl isostearate, dimethicone and cyclomethicone.

As used herein with respect to particle or crystal size, an “average uniform size” refers to average active agent size. The average can be expressed as a proportion of all the particles. Where 90% of the particles or crystals in the suspension are less than Y microns the D90 is Y microns. In other words, the great majority of particles are smaller than Y microns. In one or more embodiments, the D90 of the particles is in the range of about 5 μm to about 50 μm. In one or more embodiments the D90 is less than about 25 μm. In one or more embodiments the D90 is less than about 10 μm. In one or more embodiments the D90 is about 5 μm. In one or more embodiments the D90 is about 7.5 μm.

As used herein, “free of agglomerates” refers to a composition in which at least about 95% of the active agent is not present as agglomerates and/or does not form clusters. “substantially free of agglomerates” refers to a composition in which at least about 90% of the active agent is not present as agglomerates and/or does not form clusters.

As used herein, “adhesiveness” refers to the property of a physical attraction and interaction between different surfaces. It can refer to the attraction and interaction of a composition and a surface of an object, or it can refer to the attraction and interaction of a compound and a surface of an object and may compare it with the competing attraction and interaction of that compound and a composition in which it is suspended. In one or more embodiments adhesiveness can be expressed in terms of interfacial tension between, for example, an active agent and a surface or a composition. Adhesion of an active agent to a surface (for e.g. stainless steel) can occur when the interfacial tension between the active agent and the surface is lower than the interfacial tension between the active agent and the composition.

As used herein, “scavenger” refers to a compound that can capture molecules that promote product degradation. Scavengers can be, for example, but are not limited to free radical scavengers, or aldehyde scavengers. As used herein “impurity B” refers to a degradation product of tofacitinib namely N-methyl-N-[(3R4R)-4-methyl piperidin-3-yl]-7H-pyrrolo[2,3-d] pyrimidin-4-amine (C13H19N5). Non limiting examples of other impurities that can be identified with tofacitinib are: 3-{(3R,4R)-3-[(6-aminopyrimidin-4-yl)(methyl)amino]-4-methylpiperidin-1-yl}-3-oxopropanenitrile; and 3-[(3R,4R)-4-methyl-3-[methyl({7H-pyrrolo[2,3-d]pyrimidin-4-yl})amino]piperidin-1-yl]-3-oxopropanenitrile-N-oxide.

With respect to impurity B a negligible amount is less than about 0.13% w/w.

As used herein a “maintenance application” refers to a topical application of a composition in an amount that can help to sustain a steady-state level of a condition or disorder, or to reduce the possibility of a deterioration of a condition or a disorder, or to prevent a relapse, or return of a condition or a disorder.

As used herein an “elastomer” refers to an excipient or carrier that comprises a polymer with the property of elasticity and a solvent with which it is miscible. By property of elasticity is intended that it is a polymer that generally deforms under stress and generally returns to its original shape when the stress is removed. In one or more embodiments, the polymer has cross-links between flexible polymer chains. In general, elastomers may vary according to the amount and type of crosslinked polymer and also to the amount and type of solvent used. In one or more embodiments, elastomers with lower amounts of polymer and more solvent will, in general, have a lower viscosity and may provide a higher diffusion coefficient. Similarly, in one or more other embodiments, elastomers with higher amounts of polymer will, in general, have a higher viscosity and may provide a lower diffusion coefficient. In some embodiments, the elastomer used is thixotropic. In some embodiments, the elastomer is a mixture of crosslinked silicone and a silicone oil in which it is miscible. In some embodiments, the elastomer provides characteristics like pleasant and silky-smooth sensation as well as a non-tack and/or non-greasy feel. In some embodiments, the elastomer acts as a thickening agent. In some embodiments, the elastomer is non-occlusive and allows some amount of water loss in the applied area. In some embodiments, the elastomer is occlusive and physically prevents or retards water loss in the applied area. In some embodiments, the elastomer is partially occlusive and can allow an amount of water loss in the applied area that is reduced compared to when the elastomer is non-occlusive.

As used herein “not inert” refers to compounds that are chemically reactive with tofacitinib, e.g., causing tofacitinib to breakdown or form a new chemical entity, such as water, which can react with tofacitinib citrate at a high pH.

In one or more embodiments tofacitinib is considered chemically stable when not more than about 95% breaks down within a period of one month from manufacture in the formulation at room temperature, e.g., at 25° C. In some embodiments the period is two months, three months, four months or five months at 25° C. In one or more embodiments the period is six months at 25° C. In one or more other embodiments tofacitinib is considered chemically stable when not more than about 95% breaks down within a period of one month from manufacture in the formulation at 40° C. In some embodiments the period is two months, three months, four months, or five months at 40° C. In one or more embodiments the period is six months at 40° C.

### Topical Compositions

It should be noted that topical compositions disclosed herein can be applied to the target site as a gel, a flowable semi-solid or a liquid. In certain other embodiments, it can be applied as an emulsion, as an ointment, as a cream, as an oleogel, as an aerosol, as a spray or as a foam.

In one or more embodiment the composition is an emulsion. In one or more embodiment the composition is a water-in-oil emulsion. In one or more embodiment the composition is an oil-in-water emulsion. In one or more embodiment the composition is an emulsion comprising a hydrophobic agent, a fatty alcohol, surfactant, a gelling agent, a polyol and a preservative. See for example TOF013, example 12.

In one or more embodiment the composition is an oleogel. In one or more embodiments, the oleogel composition is a hydrophobic composition. In one or more embodiment the oleogel composition comprises a hydrophobic agent, an emollient, and a wax. See for example OT3.0005A, example 13.

In one or more embodiments, the composition can be applied as a transparent gel which allows light to pass through without being appreciably scattered so that under normal daylight conditions objects behind can be distinctly seen. In one or more embodiments, a transparent gel represents a composition with no or substantially no sediments, degradation products or phase separation. In one or more embodiments, the composition can be applied as a transparent gel without an active agent. In one or more embodiments, the composition can be applied as a transparent gel with an active agent.

In one or more embodiments, the composition can be applied as a translucent gel, which under normal daylight conditions lets some light pass through, but objects on the other side cannot be seen clearly. In one or more embodiments, the composition can be applied as a translucent gel without an active agent. In one or more embodiments, the composition can be applied as a translucent gel with an active agent.

In one or more embodiments, the composition can be applied as an opaque or hazy gel that blocks the passage of radiant energy and especially light. In one or more embodiments, the composition can be applied as an opaque or hazy gel without an active agent. In one or more embodiments, the composition can be applied as an opaque or hazy gel with an active agent. In one or more embodiments, the composition becomes opaque or hazy gel following the addition of an active agent. In one or more embodiments, the composition becomes opaque or hazy gel prior to the addition of an active agent.

Application of the claimed compositions can be, for example, hourly, twelve-hourly (e.g., twice daily), daily, alternate-day or intermittent, according to the condition of the patient. For reasons of compliance, less frequent applications, where possible, are preferable, e.g., daily single applications. In certain cases, where prolonged or long-term treatment is required, an initial dose is provided, followed by a gradual reduction to a lower maintenance dose, which can be increased if further outbreaks occur.

In one or more embodiments, the initial dose of a tofacitinib is about 0.1% to about 1.2% by weight of the composition. In one or more embodiments, the initial dose of a tofacitinib is about 0.5% to about 0.7% by weight of the composition. In one or more embodiments, the initial dose of a tofacitinib is about 0.5%, about 0.6%, or about 0.7% by weight of the composition. In one or more embodiments, the initial dose of a tofacitinib is about 0.6% tofacitinib by weight of the composition.

In one or more embodiments, the topical composition comprises a tofacitinib salt. The specific average particle size of the tofacitinib in one or more embodiments is less than or about 25 microns, or less than about 22 microns, or less than about 19 microns, or less than or about 16 microns, or less than or about 13 microns, or less than about 10 microns, or less than or about 9 microns, or less than or about 8 microns, or less than or about 7 microns, or less than or about 6 microns, or less than or about 5 microns. In one or more certain embodiments, 90% of the tofacitinib particles are less than or about one of the aforesaid amounts in size. In an embodiment, the average particle size ranges from about 6 microns to about 11 microns, or from about 7 microns to about 9 microns or from about 7.5 microns to about 8.5 microns. Skin penetration may be assisted or enhanced by having a smaller average particle size.

In one or more embodiments, the carrier is at a concentration of about 40% to about 95% by weight. In one or more embodiments, the carrier is at a concentration of about 42% to about 93% by weight. In one or more embodiments, the carrier is at a concentration of about 44% to about 91% by weight. In one or more embodiments, the carrier is at a concentration of about 50% to about 90% by weight. In one or more embodiments, the carrier is at a concentration of about 55% to about 90% by weight. In one or more embodiments, the carrier is at a concentration of about 60% to about 90% by weight. In one or more embodiments, the carrier is at a concentration of about 65% to about 90% by weight. In one or more embodiments, the carrier is at a concentration of about 70% to about 90% by weight. In one or more embodiments, the carrier is at a concentration of about 75% to about 90% by weight. In one or more embodiments, the carrier is at a concentration of at least about 40% by weight, or at least about 45% by weight, or at least about 50% by weight, or at least about 55% by weight, or at least about 60% by weight, or at least about 65% by weight, or at least about 70% by weight, or at least about 75% by weight, or at least about 80% by weight, or at least about 85% by weight, or at least about 90% by weight, or at least about 92% by weight, or at least about 94% by weight and any ranges between any two figures listed for example from about 55% to about 94%. In some embodiments, the carrier is at a concentration of less than about 95% by weight, i or s at a concentration of less than about 90% by weight, or is at a concentration of less than about 85% by weight, or less than about 80% by weight, or less than about 70% by weight, or less than about 60% by weight, or less than about 50% by weight. In one or more embodiments, the carrier is at a concentration of about 70% by weight, or about 72% by weight, or about 74% by weight, or about 76% by weight, or about 78% by weight, or about 80% by weight, or about 82% by weight, or about 84% by weight, or about 86% by weight, or about 88% by weight, or about 90% by weight, or about 92% by weight, or about 94% by weight, or about 96% by weight, or about 98% by weight. In one or more embodiments, the carrier is at a concentration of about 79.3% by weight, or about 82.4% by weight, or about 87% by weight, or about 87.4% by weight, or about 88% by weight, or about 88.24% by weight, or about 88.6% by weight.

In one or more embodiments, the carrier comprises at least one hydrophobic agent. In one or more embodiments the hydrophobic agent or carrier or at least one hydrophobic agent or carrier comprises or is selected from the group consisting of an oil, a mineral oil, a hydrocarbon oil, an ester oil, an ester of a dicarboxylic acid, a triglyceride oil, an oil of plant origin, an oil from animal origin, an unsaturated or polyunsaturated oil, a diglyceride, a PPG alkyl ether, an essential oil, a silicone oil, a liquid paraffin, an isoparaffin, a polyalphaolefin, a polyolefin, a polyisobutylene, a synthetic isoalkane, isohexadecane, isododecane, alkyl benzoate, alkyl octanoate, C12-C15 alkyl benzoate, C12-C15 alkyl octanoate, arachidyl behenate, arachidyl propionate, benzyl laurate, benzyl myristate, benzyl palmitate, bis(octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isostearyl tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, propyl myristate, propylene glycol myristyl ether acetate, propylene glycol dicaprate, propylene glycol dicaprylate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, alexandria laurel tree oil, an avocado oil, an apricot stone oil, a barley oil, a borage seed oil, a calendula oil, a canelle nut tree oil, a canola oil, a caprylic/capric a triglyceride castor oil, a coconut oil, a corn oil, a cotton oil, a cottonseed oil, an evening primrose oil, a flaxseed oil, a groundnut oil, a hazelnut oil, glycereth triacetate, glycerol triheptanoate, glyceryl trioctanoate, glyceryl triundecanoate, a hempseed oil, a jojoba oil, a lucerne oil, a maize germ oil, a marrow oil, a millet oil, a neopentylglycol dicaprylate/dicaprate, an olive oil, a palm oil, a passionflower oil, pentaerythrityl tetrastearate, a poppy oil, propylene glycol ricinoleate, a rapeseed oil, a rye oil, a safflower oil, a sesame oil, a shea butter, a soya oil, a soybean oil, a sweet almond oil, a sunflower oil, a sysymbrium oil, a syzigium aromaticum oil, a tea tree oil, a walnut oil, wheat germ glycerides, a wheat germ oil, PPG-2 butyl ether, PPG-4 butyl ether, PPG-5 butyl ether. PPG-9 butyl ether, PPG-12 butyl ether, PPG-14 butyl ether, PPG-15 butyl ether, PPG-15 stearyl ether, PPG-16 butyl ether, PPG-17 butyl ether, PPG-18 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether. PPG-10 cetyl ether, PPG-28 cetyl ether, PPG-30 cetyl ether, PPG-50 cetyl ether, PPG-30 isocetyl ether, PPG-4 lauryl ether, PPG-7 lauryl ether, PPG-2 methyl ether, PPG-3 methyl ether, PPG-3 myristyl ether, PPG-4 myristyl ether, PPG-10 oleyl ether, PPG-20 oleyl ether, PPG-23 oleyl ether, PPG-30 oleyl ether, PPG-37 oleyl ether, PPG-40 butyl ether, PPG-50 oleyl ether, PPG-11 stearyl ether, a herring oil, a cod-liver oil, a salmon oil, a cyclomethicone, a dimethyl polysiloxane, a dimethicone, an epoxy-modified silicone oil, a fatty acid-modified silicone oil, a fluoro group-modified silicone oil, a methylphenylpolysiloxane, phenyl trimethicone, a polyether group-modified silicone oil and mixtures of any two or more thereof. In some embodiments, the hydrophobic agent or carrier comprises or is selected from the group consisting of a soybean oil, a coconut oil, a cyclomethicone, a light mineral oil, a heavy mineral oil and mixtures thereof. In one or more embodiments, the solvent is tested individually for compatibility with an active agent, such as a tofacitinib or a fingolimod and is only used if it passes a compatibility test as described below in the Methods.

In one or more embodiments, the hydrophobic agent or carrier is at a concentration of about 75% to about 90% by weight. In one or more embodiments, the hydrophobic agent or carrier is at a concentration of about 55% to about 90% by weight. In one or more embodiments, the hydrophobic agent or carrier is at a concentration of about 50% to about 90% by weight. In one or more embodiments, the hydrophobic agent or carrier is at a concentration of at least about 40% by weight, at least about 45% by weight, at least about 50% by weight, at least about 55% by weight, at least about 60% by weight, at least about 65% by weight, at least about 70% by weight, at least about 75% by weight, at least about 80% by weight, at least about 85% by weight, at least about 90% by weight at least about 92% by weight, or at least about 94% by weight and any ranges between any two figures listed for example from about 55% to about 94%. In some embodiments, the hydrophobic agent or carrier is at a concentration of less than about 90% by weight, less than about 80% by weight, less than about 70% by weight, less than about 60% by weight, less than about 50% by weight. In one or more embodiments, the hydrophobic agent or carrier is at a concentration of about 70% by weight, or about 72% by weight, or about 74% by weight, or about 76% by weight, or about 78% by weight, or about 80% by weight, or about 82% by weight, or about 84% by weight, or about 86% by weight, or about 88% by weight, or about 90% by weight, or about 92% by weight, or about 94% by weight, or about 96% by weight, or about 98% by weight. In one or more embodiments, the hydrophobic agent or carrier is at a concentration of about 79.3% by weight, or about 82.4% by weight, or about 87% by weight, or about 87.4% by weight, or about 88% by weight, or about 88.24% by weight, or about 88.6% by weight. In one or more embodiments, the hydrophobic agent or carrier is about 10% to about 50% by weight, for example about 12% to about 45%, or about 12% to about 40%, or about 12% to about 35%, or about 12% to about 30%, or about 12% to about 25%, or about 12% to about 20%, or about 12% to about 15%, or about 11% to about 45%, or about 15% to about 40%, or about 18% to about 45%, or about 20% to about 40%, or about 20% to about 30%, or about 20% to about 25%, or about 10%, or about 12%, or about 14%, or about 15%, or about 16%, or about 17%, or about 18%, or about 20%, or about 22%, or about 24%, or about 26%, or about 28%, or about 30%, or about 32%, or about 34%, or about 36%, or about 38%, or about 40%, or about 42%, or about 44%, or about 46%, or about 48%, or about 50% by weight, or any other figure within these ranges.

In one or more other embodiments, the hydrophobic composition comprises a gelled oil. In one or more embodiments, the gelled oil is a gelled mineral oil. In one or more embodiments, the gelled mineral oil is a VERSAGEL®. VERSAGELs® are gelled oils or emollients that can come in different product forms including, for example, the VERSAGEL® m. VERSAGEL® p, VERSAGEL® r and VERSAGEL® s series, and provide various viscosity grades. There are also VERSAGELs® with isohexadecane, or with isododecane, or with hydrogenated polyisobutene, or with isopropylpalmitate. In an embodiment, it is VERSAGEL® 750 m. In an embodiment, it is VERSAGEL® 200 m. In an embodiment, it is VERSAGEL® 500 m. In an embodiment, it is VERSAGEL® 1600 m. VERSAGEL® m contains a mixture of mineral oil plus one or two or more of e.g., Ethylene/Propylene/Styrene Copolymer plus e.g., Butylene/Ethylene/Styrene Copolymer plus e.g., butylated hydroxyl toluene or similar gelling agents. In one or more embodiments, the gelled oil is at a concentration of about 55% to about 85% by weight. In one or more embodiments, the gelled oil is at a concentration of about 60% to about 80% by weight. In one or more embodiments, gelled oil is at a concentration of about 65% to about 75% by weight. In one or more embodiments, the gelled oil is at a concentration of about 55% to about 95% by weight. In one or more embodiments, the hydrophobic agent or carrier is at a concentration of about 75% to about 90% by weight. In one or more embodiments, the hydrophobic agent or carrier is at a concentration of about 21% to about 39% by weight. In one or more embodiments, the hydrophobic agent or carrier is at a concentration of about 26% to about 34% by weight. In one or more embodiments, the hydrophobic agent or carrier is at a concentration of about 9% to about 24% by weight. In one or more embodiments, the hydrophobic agent or carrier comprises a petrolatum at a concentration of about 9% to about 24% by weight, or about 26% to about 34% by weight or about 21% to about 39% by weight, or about 45% by weight, or about 50% by weight or about 55% by weight or about 60% by weight.

In one or more embodiments, the emollient comprises or is selected from the group consisting of glyceride oil, a branched chain ester, a branched hydrocarbon oil, an isopropyl ester, isostearic acid derivatives, isopropyl palmitate, isopropyl myristate, oleyl alcohol, PPG 15 Stearyl ether, cetearyl ethylhexanoate, MCT oil, cyclomethicone, dimethicone, cetearyl isononanoate, lanolin oil, diisopropyl dimerate, diisopropyl adipate, dimethyl isosorbide, soybean oil, glyceryl monooleate, glyceryl isostearate, glyceryl dicaprate, olive oil, maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, hydrogenated coco-glycerides, isononyl isononanoate, isotridecyl isononanoate, myristyl myristate, triisocetyl citrate, octyl dodecanol, octyl hydroxystearate, squalene, isopropyl isostearate, fish oil, tallow, palm oil, sunflower oil, rapeseed oil, soyabean oil, groundnut oil, coconut oil, peanut oil, corn oil, walnut oil, soya oil and mixtures thereof. Other examples of other suitable emollients can also be found in the Cosmetic Bench Reference, pp. 1.19-1.22 (1996), which is incorporated herein by reference for emollients.

In one or more embodiments, non-limiting examples of emollients alternative to squalane are squalene, pristane, mineral oil, hydrogenated polyisobutene, isohexadecane, isodecane, isododecane, branched alkanes and mixtures thereof.

In one or more embodiments, non-limiting examples of emollients alternative to isopropyl isostearate are: isostearyl isostearate, oleyl oleate, isocetyl stearate, hexyl laurate, isostearyl neopentanoate, ethylhexyl stearate, octyldodecyl neopentanoate, cetearyl octanoate, isodecyl neopentanoate, decyl oleate, isononyl Ethylhexanoate, isononyl isononanoate, hexyldecyl Ethylhexanoate, isotridecyl isononanoate, cetyl Ethylhexanoate, octyldodecyl neodecanoate, octyldodecyl myristate, hexyldecyl isostearate, ethylhexyl hydroxystearate, octyldodecyl stearoyloxystearate, diisopropyl dilinoleate, octyl isopalmitate, isodecyl oleate, octyl palmitate and mixtures thereof.

In one or more embodiments, the fatty alcohol and/or fatty acid have a melting point of at least about 40° C.

In one or more embodiments, the fatty alcohol comprises or is selected from the group consisting of lauryl alcohol, myristyl alcohol, cetyl alcohol, stearyl alcohol, arachidyl alcohol, behenyl alcohol, tetracosanol, hexacosanol, octacosanol, triacontanol, and tetratriacontanol. In one or more embodiments, the fatty acid comprises or is selected from the group consisting of dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid, and pentatriacontanoic acid.

In one or more embodiments, the fatty alcohol is about 3% to about 10% by weight. For example, about 3% by weight, or about 4% by weight, or about 5% by weight, or about 6% by weight, or about 7% by weight, or about 8% by weight, or about 9% by weight, or about 10% by weight. For example, about 4.1% by weight, or about 4.4% by weight, or about 4.5% by weight, or about 5% by weight, or about 5.6% by weight, or about 8.6% by weight.

In one or more embodiments, the fatty alcohol is less than about 8% by weight. For example, less than about 7% by weight or less than about 6% by weight, or less than about 5% by weight, or less than about 4% by weight.

In one or more embodiments, the carbon chain of the fatty alcohol or the fatty acid is substituted with a hydroxyl group.

In one or more embodiments, the fatty acid is 12-hydroxy stearic acid.

In one or more embodiments, the composition comprises a fatty acid. The fatty acid can be a straight chain fatty acid, a saturated fatty acid, an unsaturated fatty acid, a hydroxyl fatty acid or a branched fatty acid. In an embodiment the fatty acid is a therapeutically active fatty acid. In one or more embodiments the fatty acid is stearic acid. In an embodiment the fatty acid is a therapeutically active wax.

In one or more embodiments, the fatty acid acts as a foam adjuvant to evolve the foaming property of the composition and/or to stabilize the foam. In one or more embodiments the fatty acid can have 16 or more carbons in its carbon chain, such as hexadecanoic acid (C16) heptadecanoic acid, stearic acid (C18), arachidic acid (C20), behenic acid (C22), tetracosanoic acid (C24), hexacosanoic acid (C26), heptacosanoic acid (C27), octacosanoic acid (C28), triacontanoic acid, dotriacontanoic acid, tritriacontanoic acid, tetratriacontanoic acid and pentatriacontanoic acid, as well as fatty acids with longer carbon chains (up to C50), or mixtures thereof. In one or more other embodiments, the fatty acid is selected from the group consisting of fatty alcohols having 14 or less carbons in their carbon chain, such as dodecanoic acid myristic acid, myristoleic acid, and lauric acid.

Optionally, the carbon atom chain of the fatty acid may have at least one double bond; alternatively, the fatty acid can be a branched fatty acid. The carbon chain of the fatty acid also can be substituted with a hydroxyl group, such as 12-hydroxy stearic acid. In one or more preferred embodiments, the fatty acid is stearic acid.

In one or more embodiments, the composition comprises a “foam adjuvant”, comprising, e.g., a fatty alcohol, a fatty acid and/or a wax. In some embodiments the foam adjuvant is a fatty alcohol and a wax or a fatty acid and a wax. In some embodiments it is a wax. In some embodiments, the foam adjuvant comprises at least one of a fatty alcohol, a wax or a fatty acid. In some embodiments, the foam adjuvant is selected from a group consisting of a fatty alcohol, a wax and a fatty acid. In some embodiments, the foam adjuvant is a fatty alcohol. In some embodiments, the foam adjuvant is a fatty acid. In some embodiments, the foam adjuvant is a wax. In some embodiments, a wax has the properties of a foam adjuvant. In some embodiments a fatty alcohol, and/or a fatty acid and/or a wax is an adjuvant. In the context of the present disclosure fatty alcohols, fatty acids and waxes that are compatible with JAK inhibitors, and in particular with tofacitinib, are compatible adjuvants.

In one or more embodiments, foam adjuvants are amphipathic, and essentially hydrophobic with a minor hydrophilic region. For the purposes of forming an emulsion these foam adjuvants, unlike “standard” or “customary surfactants”, are not effective as stand-alone surfactants in emulsion compositions, because of their very weak emulsifying capacity on their own. Fatty alcohols and fatty acids have been loosely described as co-surfactants in foamable emulsion compositions because they assist customary surfactants to boost foam quality, help evolve the foaming properties and because they stabilize the foam in part because of their property as thickeners.

In one or more embodiments, the composition contains a polymeric agent. Exemplary polymeric agents are classified below in a non-limiting manner. In certain cases, a given polymer can belong to more than one of the classes provided below.

In one or more embodiments, the composition of the present invention includes a gelling agent. A gelling agent can control the residence of a therapeutic composition in the target site of treatment by increasing the viscosity of the composition, thereby limiting the rate of its clearance from the site. Many gelling agents are known in the art to possess mucoadhesive properties.

The gelling agent can be a natural gelling agent, a synthetic gelling agent and an inorganic gelling agent. Exemplary gelling agents that can be used in accordance with one or more embodiments of the present invention include, for example, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, starch, chemically modified starches and the like, semi-synthetic polymeric materials such as cellulose ethers (e.g. hydroxyethyl cellulose, hydroxypropyl cellulose, methyl cellulose, carboxymethyl cellulose, methylhydroxyethylcellulose, methylhydroxypropylcellulose, hydroxypropylmethyl cellulose, hydroxyethylcarboxymethylcellulose, carboxymethylcellulose and carboxymethylhydroxyethylcellulose), guar gum, hydroxypropyl guar gum, soluble starch, cationic celluloses, cationic guars, and the like, and synthetic polymeric materials, such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like. Mixtures of the above compounds are also contemplated.

Further exemplary gelling agents include the acrylic acid/ethyl acrylate copolymers and the carboxyvinyl polymers. Non-limiting examples include Carbopol® 934, Carbopol® 940, Carbopol® 950, Carbopol® 980, Carbopol® 951 and Carbopol® 981.

Yet, in other embodiments, the gelling agent includes inorganic gelling agents, such as silicone dioxide (fumed silica).

Mucoadhesive/bioadhesion has been defined as the attachment of synthetic or biological macromolecules to a biological tissue. Mucoadhesive agents are a class of polymeric biomaterials that exhibit the basic characteristic of a hydrogel, i.e. swell by absorbing water and interacting by means of adhesion with the mucous that covers epithelia. Compositions of the present invention may contain a mucoadhesive macromolecule or polymer in an amount sufficient to confer bioadhesive properties. The bioadhesive macromolecule enhances the delivery of biologically active agents on or through the target surface. The mucoadhesive macromolecule may be selected from acidic synthetic polymers, preferably having an acidic group per four repeating or monomeric subunit moieties, such as poly(acrylic)- and/or poly(methacrylic) acid (e.g., Carbopol®, Carbomer®), poly(methylvinyl ether/maleic anhydride) copolymer, and their mixtures and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts. Many mucoadhesive agents are known in the art to also possess gelling properties.

In one or more embodiments, the polymeric agent contains a film-forming component. The film-forming component may include a water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross-linking agent may be used to modify the polymer's characteristics. For example, esters such as dibutyl or diethyl phthalate, amides such as diethyldiphenyl urea, vegetable oils, fatty acids and alcohols such as oleic and myristyl acid may be used in combination with the cellulose derivative.

In one or more embodiments, the polymeric agent includes a phase change polymer, which alters the composition behavior from fluid-like prior to administration to solid-like upon contact with the target mucosal surface. Such phase change results from external stimuli, such as changes in temperature or pH and exposure to specific ions (e.g., Ca2+). Non-limiting examples of phase change polymers include poly(N-isopropylamide) and Poloxamer 407®.

In one or more embodiments, the composition comprises a silicone-based polymer. In one or more embodiments, non-limiting examples include dimethicone crosspolymer, polysilicone-11, polymethylsilsesquioxane and mixtures thereof.

In one or more embodiments, the composition comprises a polymer selected from the group including ethylene/propylene/styrene copolymer, butylene/ethylene/styrene copolymer, butylated hydroxyl toluene or similar gelling agents.

in one or more embodiments, the composition comprises stabilizers, in one or more embodiments, non-limiting examples are: benzalkonium chloride, benzyl alcohol, butylparaben, dehydroacetic acid/dehydroacetate, ethylparaben, imidazolindinyl urea, methylparaben, phenoxyethanol, phenylethyl alcohol, propylparaben, sorbic acid/sorbate, acetic acid/acetate, benzoic acid/benzoate, boric acid/borate, chlorocresol, lactic acid/lactate, benzethonium chloride, captan, cetylpyridinium chloride, chlorobutanol, chloroxylenol, m-cresol, diazodinyl urea, DMDM hydantoin, methylisothiazolinone/methylchloroisothiazolinone, phenol, propionic acid/propionate, quaternium-15, tragacanth gum, xylitol and mixtures thereof.

In one or more embodiments, the composition comprises an anti-oxidant. In one or more embodiments, non-limiting examples are: ascorbic acid/ascorbate, ascorbyl palmitate, butylated hydroxyanisole, butylated hydroxytoluene, citric acid/sodium citrate, disodium EDTA, propyl gallate, sodium metabisulfite sodium sulfite, sodium thiosulfate, tartaric acid/sod. Tartrate, tocopherol, tocophersolan and mixtures thereof.

In one or more embodiments, the modifying agent is a wax comprising or selected from the group consisting of a plant wax, carnauba wax, candelilla wax, ouricury wax, sugarcane wax, retamo wax, jojoba oil, an animal waxes, beeswax, a petroleum derived wax, a paraffin wax, polyethylene, and derivatives thereof.

In one or more embodiments, the modifying agent is a combination comprising (i) at least one fatty alcohol and at least one fatty acid; or (ii) at least one fatty alcohol and at least one wax; or (iii) at least one fatty acid and at least one wax; or (iv) at least one fatty alcohol, at least one fatty acid, and at least one wax.

In one or more embodiments, the at least one modifying agent comprises or is selected from the group consisting of a fatty alcohol, a fatty acid and a wax, wherein the fatty alcohols and/or fatty acids have at least 12 carbon atoms in their carbon backbone. In certain embodiments the modifying agent is a combination of a fatty alcohol and a fatty acid and/or a wax.

In some embodiments, the fatty alcohol and/or fatty acid and/or wax are solid at ambient temperature. In certain embodiments, the fatty alcohol and/or the fatty acid and/or the wax or the mixture of them have a melting point of more than about 40° C.

In one or more embodiments, the wax is about 0% to about 6% by weight. For example, about 1% by weight, or about 2% by weight, or about 3% by weight, or about 4% by weight, or about 5% by weight, or about 6% by weight. In one or more embodiments, the wax is about 0.2% by weight.

In one or more embodiments, the wax is less than about 4% by weight. For example, less than about 3% by weight, or less than about 2% by weight, or less than about 1% by weight, or less than about 0.5% by weight.

In one or more embodiments, the fatty acid is about 1% to about 10% by weight. For example, about 1% by weight or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

In one or more embodiments, the total amount of fatty acid fatty alcohol and wax, if present is about 1% to about 10% by weight. For example, about 1% by weight, or about 2% by weight, or about 3% by weight, or about 4% by weight, or about 5% by weight, or about 6% by weight, or about 7% by weight, or about 8% by weight, or about 9% by weight, or about 10% by weight. For example, about 2.4% by weight, or about 2.5% by weight, or about 3% by weight.

### Elastomers

In one or more embodiments, the elastomer is a cosmetic or pharmaceutical grade elastomer, known in the art. In one or more embodiments, the elastomer is a mixture of a silicone oil and a silicone crosspolymer. In one or more embodiments, the elastomer is a mixture of dimethicone and a silicone crosspolymer. In one or more embodiments, the elastomer is a mixture of cyclopentasiloxane and a silicone crosspolymer. In one or more embodiments, the elastomer is a mixture of silicone oil and a dimethicone/vinyl dimethicone crosspolymer. In one or more embodiments, the elastomer is a mixture of silicone oil and a petrolatum and dimethicone crosspolymer. In one or more embodiments, the elastomer is a mixture of silicone oil and a PEG-12 dimethicone crosspolymer. In one or more embodiments, the elastomer is a mixture of silicone oil and an EG-1 dimethicone/PPG-20 crosspolymer. In one or more embodiments the at least one elastomer comprises one or more of cyclopentasiloxane (and) polysilicone-11 (Grant MGS-Elastomer 1100), dimethicone (and) polysilicone-11 (Gransil DMG-3), a cyclopentasiloxane (and) petrolatum (and) polysilicone-11 (MGS-Elastomer I148P), cyclopentasiloxane and dimethicone cross polymer (ST-Elastomer 10) and dimethicone (and) dimethicone crosspolymer (DOWSIL™ 9041). In some embodiments the elastomer is ST elastomer 10. In one or more embodiments, non-limiting examples of elastomers are described in “Elastomers” Cosmetic Ingredients—SpecialChem Website (Dec. 30, 2019) (https://cosmetics.specialchem.com/selectors?indexpage=1&q=elastomer) and are incorporated by reference in their entirety. In one or more embodiments the elastomers are diluted by addition of a cylomethicone or a dimethicone.

### Silicone Thickening Agents

Silicone thickening agents comprise one or more polysiloxane-derived components. Such polysiloxanes are typically cross-linked and they have rubber-like characteristics, which require their solubilization in an oil, usually a silicone oil. An example of such a silicone thickening agent is an elastomer e.g., ST-Elastomer 10 (Dow Corning), which is a mixture of high molecular weight dimethicone crosspolymer (12%), in cyclopentasiloxane (cyclomethicone, silicone solvent). In one or more embodiments an elastomer is a main component of the carrier. In one or more embodiments, the silicone thickening agent can act as a base carrier. In one or more embodiments, the silicone thickening agent provides characteristics like pleasant and silky-smooth sensation as well as a non-tack and/or non-greasy feel. In one or more embodiments, the silicone thickening agent acts as a penetration enhancer.

In one or more embodiments, the carrier base comprises a silicone thickening agent. In one or more embodiments, the carrier base includes a silicone comprising a silicone thickening agent. In one or more embodiments, the carrier base includes a silicone comprising a silicone thickening agent and a silicone oil.

In one or more embodiments, the silicone is present in the composition in about 75% to about 95% by weight. For example about 78% to about 93%, or about 80% to about 92%, or about 82% to about 90%, or about 85% to about 88%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95% by weight or a range between any two of the aforesaid.

In one or more embodiments, the silicone is present in the composition in about 45% to about 75% by weight. For example about 48% to about 73%, or about 50% to about 70%, or about 52% to about 68%, or about 55% to about 65%, or about 58% to about 63%, or about 45%, or about 46%, or about 47%, or about 48%, or about 49%, or about 50%, or about 51%, or about 52%, or about 53%, or about 54%, or about 55%, or about 56%, or about 57%, or about 58%, or about 59%, or about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, by weight or a range between any two of the aforesaid.

In one or more embodiments, the silicone is present in the composition in about 45% or less by weight, or in about 44% or less, or in about 43% or less, or in about 42% or less, or in about 41% or less, or in about 40% or less, or in about 39% or less, or in about 38% or less, or in about 37% or less, or in about 36% or less, or in about 35% or less, or in about 34% or less, or in about 33% or less, or in about 32% or less, or in about 31% or less, or in about 30% or less, or in about 28% or less, or in about 26% or less, or in about 24% or less, or in about 22% or less, or in about 20% or less, or in about 18% or less, or in about 16% or less, or in about 15% or less, or in about 12% or less, or in about 10% or less by weight. In one or more other specific embodiments, the drug carrier is formulated with less than about 30% by weight of silicones, or less than about 25% by weight of silicones, or less than about 20% by weight of silicones, or less than about 15% by weight of silicones, or less than about 10% by weight of silicones, or less than about 7.5% by weight of silicones, or less than about 5% by weight of silicones or less than about 2% by weight of silicones; or less than about 1% by weight of silicones; or less than about 0.5% by weight of silicones; or about 1% to about 5% by weight of silicones or a range between any two of the aforesaid. In one or more other specific embodiments, the drug carrier does not comprise a silicone other than cyclomethicone or a dimethicone. In one or more other specific embodiments, the drug carrier does not comprise a silicone other than a cyclomethicone. In one or more other specific embodiments, the drug carrier does not comprise a silicone other than a dimethicone.

In one or more embodiments, semi-solid hydrophobic oils are a subsidiary component in the composition, for example being present at less than about 45%, at less than about 40%, at less than about 35%, at less than about 30%, at less than about 25%, less than about 20%, less than about 15%, less than about 10%, less than about 7.5%, less than about 5%, less than about 2.5%, less than about 1%, or less than about 0.5% by weight of the composition. In one or more alternative embodiments, semi solid oils are omitted.

In some embodiments, the composition can contain a hydrophobic oil and one or more modifying agents. In some embodiments, the compositions demonstrate increased viscosity of such oil, and to which when even small amounts of a suspended active ingredient are added, a substantial or synergistic increase in the viscosity of the composition can be observed.

### Viscosity

The viscosity of a composition is an important consideration when formulating semi-solid topical drug products. On the one hand, viscosity should be high enough to enable inter alia: (i) proper dispensing of the product on the patient's skin without having a runny liquid, (ii) an adequate skin feel to ensure patient compliance, (iii) if active ingredient is suspended, a uniform distribution of the active ingredient for avoidance of aggregates of API crystals. On the other hand, the viscosity of the drug product should be low enough to enable inter alia: (i) proper extrusion of the product from the container (e.g. tube or pump), (ii) good skin feel for improved patient compliance, (iii) an industrially applicable compounding and packaging manufacturing process.

The manufacture and scale-up of a semi-solid drug product comprising low concentration of active ingredient may also be challenging. When a low concentration of active ingredient is suspended, it may be difficult to obtain a uniform distribution of the active in the bulk product, especially on industrial scale. In addition, chemical stability issues may arise when the concentration of the active ingredient is decreased substantially due to the change in the active to excipient ratio. The viscosity of the formulation during manufacturing in one or more embodiments should facilitate homogenous mixing of the ingredients and uniform distribution of suspended matter.

In one or more embodiments, the viscosity is measured by an Anton Par Rheometer MCR302, plate/plate 50 mm geometry (“the Anton Par”). In one or more embodiments, shear force can be measured at different shear rates, e.g., 100 sec-1, 10

sec-1, 1 sec-1, or 0.1 sec-1. In one or more embodiments, the viscosity is measured using a DHR3 rheometer from TA instruments.

In one or more embodiments, when measured by the Anton Par, the compositions described herein have a viscosity range of about 3000 mPa·sec to about 9000 mPa·sec at a shear rate of 100 sec-1. For example, 3500 mPa·sec to about 8500 mPa·sec-1, or about 4000 mPa·sec to about 8000 mPa·sec-1, or about 4500 mPa·sec to about 7500 mPa·sec-1, or about 4700 mPa·sec to about 7500 mPa·sec-1, or about 4800 mPa·sec to about 7500 mPa·sec-1, or about 5000 mPa·sec to about 7200 mPa·sec-1, or about 5200 mPa·sec to about 7000 mPa·sec-1, or about 5400 mPa·sec to about 6900 mPa·sec-1, or about 5800 mPa·sec to about 6700 mPa·sec-1, or about 6000 mPa·sec to about 8000 mPa·sec-1, or about 6400 mPa·sec to about 8000 mPa·sec-1, or about 6800 mPa·sec to about 8000 mPa·sec-1 or any other figure within these ranges.

In one or more embodiments measured by the Anton Par, the compositions described herein have a viscosity range of about 15000 mPa·sec to about 35000 mPa·sec at a shear rate of 10 sec-1. For example, 15500 mPa·sec to about 32000 mPa·sec-1, or about 15800 mPa·sec to about 30000 mPa·sec-1, or about 16000 mPa·sec to about 29000 mPa·sec-1, or about 16500 mPa·sec to about 28500 mPa·sec-1, or about 16800 mPa·sec to about 28000 mPa·sec-1, or about 17000 mPa·sec to about 27800 mPa·sec-1, or about 17200 mPa·sec to about 27500 mPa·sec-1, or about 17400 mPa·sec to about 27000 mPa·sec-1, or about 17800 mPa·sec to about 26500 mPa·sec-1, or about 18000 mPa·sec to about 26000 mPa·sec-1, or about 18500 mPa·sec to about 25500 mPa·sec-1, or about 19000 mPa·sec to about 25000 mPa·sec-1, or about 19500 mPa·sec to about 24500 mPa·sec-1, or about 20000 mPa·sec to about 24000 mPa·sec-1, or about 20500 mPa·sec to about 23500 mPa·sec-1, or any other figure within these ranges. In one or more embodiments, the viscosity is measured by an Anton Par Rheometer MCR302, plate/plate 50 mm geometry.

In one or more other embodiments, the viscosity is measured by a Brookfield viscometer, such as a DV II CP. As will be appreciated by one skilled in the art viscosity measurements can vary according amongst other things according to the viscometer used, the shear rate used, the spindle and the container and the volume of composition.

In one or more embodiments, the viscosity increases when the temperature increases.

In one or more embodiments, the viscosity decreases when the temperature increases.

In one or more embodiments, in elastomer-based formulations (e.g., about 87% elastomer) the viscosity remains generally constant or constant when the temperature increases or upon temperature changes. This is unlike e.g., petrolatum-based formulations where the viscosity decreases with an increase in temperature. By “generally constant”, in one or more embodiments, is intended that fluctuations in viscosity of up to about 20% are acceptable. By constant in one or more embodiments allows for small fluctuations of up to about 10%. In some embodiments, the viscosity remains generally constant or constant between about 15° C. to about 37° C. In some embodiments, the viscosity remains generally constant or constant between about 16° C. to about 30° C., or between about 18° C. to about 27° C. or between about 20° C. to about 25° C. In one or more embodiments, in elastomer-based formulations the viscosity remains generally constant or constant when the temperature changes using oscillatory measurements.

In the context herein an elastomer-based formulation is one where the majority of the formulation comprises elastomer. In the context herein a petrolatum-based formulation is one where the majority of the formulation comprises petrolatum.

In terms of how much elastomer should be present in a formulation in order to form a gel rather than a liquid or runny formulation may vary on multiple factors including depending on the type of elastomer, the proportion of elastomer and the other components as will be appreciated by one skilled in the art. For example, elastomers with higher levels of polymers will generally be more viscous and more viscous elastomers should facilitate the presence of higher amounts of other ingredients and allow for a lower proportion of elastomer. Also, if one or more viscous oils (e.g., coconut oil) are provided in addition to elastomer a lower level of elastomer may be needed to achieve a gel, whereas if one or more liquid non or low viscous oils (e.g., light mineral oil) are provided in addition to elastomer a higher level of elastomer can be appropriate to form a gel. Similarly, adjusting the proportion of liquid silicone (e.g., cyclopentasiloxane) in the elastomer formulation downwards can lead to a lower level of elastomer being needed to achieve a gel whereas increasing the proportion of liquid silicone (e.g., cyclopentasiloxane) can lead to a higher level of elastomer being needed to achieve a gel.

### Polyol

The identification of a “polyol”, as used herein, is an organic substance that contains at least two hydroxy groups in its molecular structure. In one or more embodiments, the polyol is a diol (a compound that contains two hydroxy groups in its molecular structure). Examples of diols include propylene glycol (e.g., 1,2-propylene glycol and 1,3-propylene glycol), butanediol (e.g., 1,2-butanediol, 1,3-butanediol, 2,3-butanediol and 1,4-butanediol), butenediol (e.g., 1,3-butenediol and 1,4-butenediol), butynediol, pentanediol (e.g., pentane-1,2-diol, pentane-1,3-diol, pentane-1,4-diol, pentane-1,5-diol, pentane-2,3-diol and pentane-2,4-diol), hexanediol (e.g., hexane-1,6-diol hexane-2,3-diol and hexane-2,56-diol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the polyol is a triol (a compound that contains three hydroxy groups in its molecular structure), such as glycerin, butane-1,2,3-triol, butane-1,2,4-triol and hexane-1,2,6-triol.

In one or more embodiments, the polyol is a saccharide. Exemplary saccharides include, but are not limited to, monosaccharides, disaccharides, oligosaccharides, and sugar alcohols.

A monosaccharide is a simple sugar that cannot be hydrolyzed to smaller units. The empirical formula of a monosaccharide is (CH2O)n and can range in size from trioses (=3) to heptoses (n=7). Exemplary monosaccharide compounds are, e.g., ribose, glucose, fructose, and galactose.

Disaccharides are made up of two monosaccharides joined together, such as sucrose, maltose, and/or lactose.

In one or more embodiments, the polyol is a sugar alcohol (also known as a polyol, polyhydric alcohol, or polyalcohol) or a hydrogenated form of saccharide, whose carbonyl group (aldehyde or ketone, reducing sugar) has been reduced to a primary or secondary hydroxyl group. They are commonly used for replacing sucrose in foodstuffs, often in combination with high intensity artificial sweeteners to counter the low sweetness. Some exemplary sugar alcohols, which are suitable for use according to the present invention are mannitol, sorbitol, xylitol, maltitol, lactitol. (Maltitol and lactitol are not completely hydrogenated compounds—they are a monosaccharide combined with a polyhydric alcohol.) Mixtures of polyols, including (1) at least one polyol comprises or selected from a diol and a triol; and (2) a saccharide are contemplated within the scope of the present disclosure.

According to some embodiments, the composition is polyol free, i.e., the composition does not comprise any amount of polyols.

In other embodiments, the composition is substantially free of polyols and comprises less than about 5% by weight of the final concentration of polyols, or less than about 2% by weight, or less than about 1% by weight. In some embodiments the composition comprises de minimis amounts of polyols. Where a formulation includes insignificant or de minimis amounts of polyols, such as less than about 0.1%, or less than about 0.05% by weight, it is considered to be essentially free of them.

In one or more embodiments, the polyol is present in the composition to provide partial solubility. In one or more embodiments, the polyol is present in the composition at about 5% to about 30% by weight. For example, at about 7% to about 25%, or about 8% to about 20%, or about 8% to about 15%, or about 5%, or about 10%, or about 15%, or about 20%, or about 25%, or about 30% by weight, or a range between any two of the aforesaid.

In one or more embodiments, the polyol is linked to a hydrophobic moiety. In the context of the present disclosure, a polyol linked to a hydrophobic moiety is still defined as a “polyol” as long as it still contains two or more free hydroxyl groups.

In an embodiment, the polyol is linked to a hydrophilic moiety. In the context of the present disclosure, a polyol linked to a hydrophilic moiety is still defined as a “polyol” as long as it still contains two or more free hydroxyl groups.

In one or more embodiments, the composition is not hydrophilic or substantially not hydrophilic.

In one or more embodiments the composition is hydrophobic or substantially hydrophobic.

In one or more embodiments, the composition is free of or substantially free of one or more selected from the group consisting of surface-active agents, polymeric gelling agents, polyols, protic solvents, polar aprotic, solvents and short chain alcohols.

In one or more embodiments, the composition contains less than about 0.4% by weight of the composition, or less than about 0.2% by weight of the composition, or less than about 0.1%, or less than about 0.05% by weight of the composition of one or a combination of any two or more of surface-active agents, polymeric gelling agents, polyols, protic solvents, polar aprotic, solvents and short chain alcohols.

The ingredients as therapeutic agents

In one or more embodiments, the excipients in the composition can have a therapeutic effect that completes and/or enhances and/or complements the JAK inhibitor effect. In one or more embodiments, the excipients in the composition can have a therapeutic effect that completes and/or enhances and/or complements the S1PR modulator or agonist effect. In some embodiments, the excipient, when applied together with the active agent(s) can have a synergistic effect.

In certain embodiments, a hydrophobic agent or carrier can possess therapeutic properties. For example, some oils, e.g., some essential oils can kill microorganisms or impair their growth and can be effective or supportive in the treatment or prevention of conditions that involve microbial infection, such as bacterial, fungal and viral conditions. Additionally, hydrophobic agents can be useful for the treatment of conditions that involve damaged skin, such as psoriasis or atopic dermatitis. The combination of a hydrophobic agent or carrier and a therapeutically effective fatty alcohol or fatty acid may afford a beneficial effect in conditions characterized, for example, by infection and/or inflammation.

Fatty alcohols can also possess therapeutic properties. Long chain saturated and monounsaturated fatty alcohols, e.g., stearyl alcohol, erucyl alcohol, arachidyl alcohol and behenyl alcohol (docosanol) have been reported to possess antiviral, antiinfective, antiproliferative and anti-inflammatory properties (see, e.g., U.S. Pat. No. 4,874,794).

Longer chain fatty alcohols, e.g., tetracosanol, hexacosanol, heptacosanol, octacosanol, triacontanol, etc., are also known for their metabolism modifying properties, and tissue energizing properties.

In one or more embodiments, the active agent can be a placebo or a cosmetic agent. In one or more embodiments the composition is suitable for use in the manufacture of a medicament including a placebo or active agent.

### Combination of Active Agents

Several disorders involve a combination of more than one etiological factor; and therefore, the use of more than one active agent is advantageous. For example, psoriasis involves excessive cell proliferation and inadequate cell differentiation as well as inflammation. Atopic dermatitis involves keratinocyte growth abnormality, skin dryness and inflammation. Bacterial, fungal and viral infections involve pathogen colonization at the affected site and inflammation. Hence, in many cases, the inclusion of a combination of active agents in the pharmaceutical composition can be desirable. Thus, in one or more embodiments, the composition includes at least two active agents, in a therapeutically effective concentration.

In one or more embodiments, there is provided a composition in which the composition comprises at least two active agents including, or selected from the group consisting of a JAK inhibitor (e.g., a tofacitinib), an S1PR modulator or agonist (e.g., a fingolimod), an antibiotic agent, a steroidal anti-inflammatory agent, an immunosuppressive agent, an immunomodulator, an immunoregulating agent, a hormonal agent, an androgen, an estrogen, a prostaglandin, an antiandrogen agent, a testosterone inhibitor, a dihydrotestosterone inhibitor, a serine protease inhibitor, a cysteine protease inhibitor, antibacterial agent, an antifungal agent, an antiviral agent, an antiparasitic agent, antimicrobial, an anti-itching agent, an anti-scarring agent, an antipruritic, an antihistamine, a retinoid, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, an anesthetic, an antiallergic agent, a keratolytic agent, urea, a urea derivative, a peptide, a neuropeptide, an allergen, an immunogenic substance, a dicarboxylic acid, azelaic acid, sebacic acid, adipic acid, fumaric acid, a retinoid, an antiproliferative agent, an anticancer agent, a photodynamic therapy agent, a metal, silver, a metal oxide, titanium dioxide, zinc oxide, zirconium oxide, iron oxide, silicone oxide, an organo-metallic compound, and organo-boron compound, an organo-beryllium compound, a tellurium compound, an anti-wart agent and a coal tar. In some embodiments there are provided a combination of two or more agents from any of the aforesaid categories, e.g, a combination of two or more JAK inhibitors or a combination of a JAK inhibitor e.g., tofacitinib or a pharmaceutically acceptable salt thereof and a S1PR modulator or agonist e.g., fingolimod or a pharmaceutically acceptable salt thereof.

In one or more embodiments the composition comprises a JAK inhibitor and one or more other active agents. In some embodiments the composition comprises two or more JAK inhibitors. In some embodiments the composition comprises two or more JAK inhibitors and one or more other active agents.

In one or more embodiments, the composition comprises a combination of JAK inhibitor and an anti-itching agent. In one or more embodiments, the composition comprises a combination of JAK inhibitor and an anti-pruritic agent. In one or more embodiments, the composition comprises a combination of JAK inhibitor and a retinoid. In one or more embodiments, the composition comprises a combination of JAK inhibitor and an anesthetic agent. In one or more embodiments, the composition comprises a combination of JAK inhibitor and an antibiotic. In one or more embodiments, the composition comprises a combination of JAK inhibitor and a steroid. In one or more embodiments, the composition comprises a combination of JAK inhibitor and an antihistamine.

In one or more embodiments, the antihistamine is present at about 0.5% to about 2%. For example, diphenhydramine hydrochloride at about 1% by weight.

In one or more embodiments, the steroid is present at about 0.001% to about 5%. For example, triamcinolone acetonide at about 0.025% by weight.

In one or more embodiments, the retinoid is present at about 0.01% to about 3%. For example, adapalene at about 0.25% or about 0.3%, or about 0.35% by weight.

In one or more embodiments, the antibiotic is present at about 0.5% to about 10%. For example, doxycycline at about 1.5%, or about 2.25%, or about 3% by weight.

In one or more embodiments, the composition comprises a combination of JAK inhibitor and a fingolimod.

There are four known mammalian JAKs: JAK1 (Janus kinase-1), JAK2, JAK3 (also known as Janus kinase, leukocyte; JAKL; and L-JAK), and TYK2 (protein-tyrosine kinase 2).

Non limiting examples of JAK inhibitors are: pyrrolopyridine and pyrrolopyrimidines, cyclobutene, tyrphostin AG490, tofacitinib, decemotinib (VX-509), ruxolitinib, baricitinib, CYT387, GLPG0634, AC-430, fibotinib (GLPG0634), peficitinib (ASP015K), ABT-494, cerdulatinib, fedratinib, filgotinib and pacritinib.

In one or more embodiments, a combination of any two or more of an antibacterial, an anti-inflammatory, an antifungal, and an antiviral agent is contemplated.

In one or more embodiments the hydrophobic composition further comprises an anti-infective agent, selected from the group of an antibiotic agent, an antibacterial agent, an antifungal agent, an agent that controls yeast, an antiviral agent, and an antiparasitic agent. In one embodiment, the anti-infective agent comprises a tricyclic antibiotic. Not only can combining the anti-infective effect of a hydrophobic composition with an anti-infective agent result in an improved or in some embodiments a synergistic effect and consequently higher success rate of the treatment, but the combination e.g., with a modifying agent can achieve a formulation in which the active pharmaceutical ingredient is chemically stable and the formulation is physically stable. Moreover, the use of hydrophobic-based, water-free formulations can maximize the antimicrobial and antiviral potentials of the formulations.

Topical delivery can, in one or more embodiments, be improved by using a hydrophobic carrier with a hydrophobic API.

In one or more embodiments, the storage in scaled, light, and airtight containers or canisters can assist in preserving the formulations.

In one or more embodiments the addition of at least one additional active agent is optional.

In some embodiments the topical composition comprising a JAK inhibitor (e.g., a tofacitinib) is co-administered with an oral drug (e.g., an antibiotic, an antifungal, an antiviral, an antipruritic, an antihistamine or a steroid). In some embodiments the topical composition comprising a JAK inhibitor (e.g., a tofacitinib) and a fingolimod is co administered with an oral drug (e.g., an antibiotic, an antifungal, an antiviral, an antipruritic, an antihistamine or a steroid) in a therapeutically effective amount. In some embodiments the antipruritic is serlopitant. For example, in some embodiments about 5 gm to 15 mg of serlopitant is given as an initial oral dose. In some embodiments the daily oral dose is about 1 mg to about 25 mg. In some embodiments the daily oral dose is about 3 mg to about 12 mg. In some embodiments the daily dose of serlopitant is about 3 mg, about 4 mg, about 5 mg, about 6 mg, about 7 mg, about 8 mg, about 9 mg, about 10 mg, about 11 mg, or about 12 mg.

Wherever a specific active agent is used herein, it can be substituted by another form of the same active agent. For example, in one or more embodiments, tofacitinib citrate can be substituted by another form of tofacitinib or in one or more embodiments, fingolimod hydrochloride can be substituted by another form of fingolimod. The term “form” can include, for example, salts, hydrates, crystals, polymorphs, enantiomers, isomers, ions, complexes, and the like. In one or more embodiments, the active agent can be in the form of a salt, a hydrate, a crystal, one or more polymorphs, one or more enantiomers, an isomer, an ion, a complex, or any other pharmaceutically acceptable form. In one or more embodiments the form is a base, for example tofacitinib base or fingolimod base. Whenever the term “a tofacitinib” or “a fingolimod” is used it is inclusive or all the various forms.

In one or more embodiments, the concentration of the additional active agent is in a range between about 0.1% to about 10% by weight of the composition (e.g., about 0.1% to about 8% by weight, or about 0.1% to about 5% by weight, or about 0.1% to about 3% by weight, or about 0.1% to about 2% by weight, or about 0.1% to about 1% by weight, or about 0.1% to about 0.75% by weight, or about 0.1% to about 0.5% by weight, or about 0.1% to about 0.25% by weight, or about 0.25% to about 10% by weight, or about 0.5% to about 10% by weight, or about 1% to about 10% by weight, or about 2% to about 10% by weight, or about 4% to about 10% by weight, or about 6% to about 10% by weight, or about 7% to about 10% by weight, or about 8% to about 10% by weight, or about 0.5% to about 2.0% by weight, or about 0.75% to about 1.5% by weight, or about 1% to about 3% by weight, or about 1% to about 4% by weight, or about 2% to about 6% by weight). In some embodiments, the concentration of the additional active agent is at least about 0.05% by weight, or is at least about 0.1% by weight, or at least about 0.5% by weight, or at least about 1% by weight, or at least about 2% by weight, or at least about 4% by weight, or at least about 6% by weight, or at least about 8% by weight or at least about 10% by weight or is between any two aforesaid amounts. In one or more embodiments, additional active agent is therapeutically effective in low amounts and the concentration of the additional active agent is in a range between about 0.0001% and about 0.1% by weight of the composition (e.g., about 0.0005% to about 0.05% by weight, or about 0.001% to about 0.01% by weight)

In one embodiment, the composition is useful for treating atopic dermatitis.

In another embodiment, the composition is useful for treating psoriasis.

In yet another embodiment, the composition is useful for treating an eczema.

In some embodiments, patients treated with the compositions disclosed herein are diagnosed with atopic dermatitis. The diagnosis of AD is made clinically and is based on historical features, morphology and distribution of skin lesions, and related clinical signs. Formal sets of criteria have been developed by various groups to assist in classification. For example, patients may be diagnosed according to Eichenfield et al. (“Guidelines of care for the management of atopic dermatitis,” J. Am. Acad. Dermatol., 70(2):338-346) using the following criteria: (i) essential features (must be present); (ii) important features (adding support to diagnosis); (iii) associated features; and (iv) exclusionary conditions. From time to time, skin biopsy specimens or other tests (such as serum immunoglobulin E, potassium hydroxide preparation, patch testing, and/or genetic testing) may be beneficial to rule out other or associated skin conditions.

For example, essential features for diagnosing a subject with atopic dermatitis may include pruritus and eczema (acute, subacute, chronic). The eczema may consist of (i) typical morphology and age-specific patterns and (ii) chronic or relapsing history. Age-specific patterns may include (1) facial, neck, and extensor involvement in infants and children, (2) current or previous flexural lesions in any age group, and (3) sparing of the groin and axillary regions.

Some key features which are seen in most atopic dermatitis cases and which add support to the diagnosis include (1) early age of onset. (2) atopy which include personal and/or family history and immunoglobulin E reactivity, and (3) xerosis.

There are other clinical features associated with atopic dermatitis which may help with the diagnosis of atopic dermatitis but are too broad to be used for defining or identifying atopic dermatitis for research and epidemiologic studies. These include (1) atypical vascular responses (e.g., facial pallor, white dermographism, delayed blanch response), (2) keratosis pilaris/pityriasis alba/hyperlinear palms/ichthyosis, (3) ocular/periorbital changes, (4) other regional findings (e.g., perioral changes/periauricular lesions), and (5) perifollicular accentuation/lichenification/prurigo lesions.

A diagnosis of atopic dermatitis also depends on excluding other conditions, such as: scabies, seborrheic dermatitis, contact dermatitis (irritant or allergic), ichthyoses, cutaneous T-cell lymphoma, psoriasis, photosensitivity dermatoses, immune deficiency diseases, and Erythroderma of other causes.

For patients with assumed atopic dermatitis there are no specific biomarkers that can be recommended for diagnosis and/or assessment of disease severity. Monitoring of immunoglobulin E levels is not recommended for the routine assessment of disease severity. It is recommended that clinicians ask general questions about itch, sleep, impact on daily activity, and persistence of disease, and that presently available scales be used mainly when practical.

Physicians should be conscious of and assess for conditions associated with atopic dermatitis, such as rhinitis/rhinoconjunctivitis, asthma, food allergy, sleep disturbance, depression, and other neuropsychiatric conditions, and it is recommended that physicians discuss them with the patient as part of the treatment/management plan, when suitable. An integrated, multidisciplinary approach to care may be valuable and is suggested for atopic dermatitis patients who present with common associations.

In some embodiments, patients treated with the compositions disclosed herein are diagnosed with psoriasis. Psoriasis is a chronic inflammatory multi organ disease with well characterized pathology appearing in the skin and often the joints. Although the disease has many characteristic and even pathognomonic features, no confirmed diagnostic criteria exist for cutaneous psoriasis and there is no unified classification for the clinical spectrum of the disease. Earlier approaches that have been taken to classify psoriasis include age of onset, severity of the disease, and morphologic evaluation. The latter has produced plaque, guttate, pustular, and erythrodermic as subtypes of psoriasis. Unlike other autoimmune diseases, histopathological examination and blood tests are generally not valuable tools in making the diagnosis of psoriasis. However, occasionally, dermatopathologic evaluation may assist in confirming the diagnosis of psoriasis. Thus, in most cases the diagnosis of psoriasis is dependent mostly on pattern recognition that is morphologic evaluation of skin lesions and joints. K. Smriti et al., “Diagnosis and classification of psoriasis,” Autoimmunity Reviews, 13(4-5):490-495.

One diagnostic criterion of cutaneous psoriasis is based on clinical appearance (see www.dermnetnz.org for representative images). The most frequent presentation is chronic plaque psoriasis (psoriasis vulgaris) and is characterized by well demarcated bright red plaques covered by adherent silvery white scales. These may affect any body site, often symmetrically, especially the scalp and extensor surfaces of limbs. The differential diagnosis includes eczema, tinea, lichen planus and lupus erythematosus. The appearance of the plaques may be modified by emollients and topical treatments, which readily remove the scale. Scaling is reduced at flexural sites, on genital skin and in palmoplantar disease.

Guttate psoriasis describes the rapid development of multiple small papules of psoriasis over wide areas of the body. The differential diagnosis includes pityriasis rosea, viral exanthems and drug eruptions.

Generalized pustular psoriasis is rare and is characterized by the development of multiple sterile non-follicular pustules within plaques of psoriasis or on red tender skin. This may occur acutely and be associated with fever. The differential diagnosis includes pyogenic infection, vasculitis and drug eruptions. See Diagnosis and Management of Psoriasis and Psoriatic Arthritis in Adults, A national clinical guideline Scottish Intercollegiate Guidelines Network, 2010, page 8.

The stability of compositions containing an active agent e.g., a tofacitinib or a fingolimod can be monitored at about e.g. 5° C., 25° C., 30° C. and 40° C. and satisfactory stability results are obtained.

In one or more embodiments, there is provided a composition in which the composition comprises an additional agent including one or more of a disinfectant, an alpha hydroxyl acid, lactic acid, glycolic acid, a beta-hydroxy acid, a protein, a haptene, an oxidizing agent, an antioxidant, benzoyl chloride, calcium hypochlorite, magnesium hypochlorite, an anti-wrinkle agent, a radical scavenger, talc, carbon, a skin whitening agent, a skin protective agent, a masking agent, a refatting agent, and a lubricating agent.

In one or more embodiments, the concentration of the additional agent is about any of the amounts or between about one or more of any of the aforesaid ranges for the additional active agent.

### ADDITIONAL EMBODIMENTS

Additional embodiments of the disclosure include a two-part formulation comprising a first component formulation and a second component formulation, which requires mixing of two components prior to administration by the patient. This is cumbersome and has no or little practical or viable value. Although mixing of the two component formulations results in substantial solubilization of the active agent, thus rendering it “suitable for topical delivery” a further disadvantage of a two part formulation is if the active agent stabilized in the first component undergoes degradation in the presence of the second component when combined upon expulsion or left on the skin for a while following expulsion.

In one or more embodiments, the topical composition is a two-part composition comprising a first component formulation and a second component formulation. Where the first component formulation and second component formulation are mixed prior to use is in one or more embodiments disadvantageous. In one or more embodiments, the first component of the two-part composition is intended for active agent stabilization and second component is intended for active agent solubilization. In one or more embodiments, the active agent stabilized in the first component formulation of the two-part composition is incompatible with the second component and forms degradation products upon mixing of the two components or shortly thereafter.

In one or more embodiments, a composition, which is made up of at least two components or parts and wherein the at least two components or parts are stored separately prior to use and combined or mixed or intended to be combined or mixed upon administration or shortly prior to administration is disadvantageous. For example, in one or more embodiments a composition comprising a first component or part formulation comprising an active agent with a hydrophobic agent or carrier and elastomer and a second component or part formulation comprises a penetration enhancer that is incompatible with the active agent or a substance in the first component or part formulation or vice versa is disadvantageous. In one or more embodiments where the penetration enhancer comprises water, ethanol, a short chain alcohol or a protic solvent is disadvantageous.

In one or more embodiments, a composition containing skin irritants, such as surfactants and short chain alcohols is disadvantageous. In one or more embodiments, a composition is free of skin irritants, such as surfactants and short chain alcohols. In one or more embodiments a composition is essentially free of skin irritants such as surfactants and short chain alcohols. In one or more embodiments a composition is substantially free of skin irritants such as surfactants and short chain alcohols.

In one or more embodiments an ointment base vehicle is greasy and thus reduces patient compliance and is disadvantageous. In one or more embodiments an ointment base vehicle comprises petrolatum and is disadvantageous.

In some embodiments the composition comprises about or less than 15% occlusive agent e.g., petrolatum. In some embodiments the composition comprises about or less than 10%, or 7.5%, or 5%, or 2.5% or 1% occlusive agent. In one or more embodiments the composition is free or substantially free of occlusive agents.

The nature of a formulation in general terms is determined by the content of the formulation and for foamable compositions also by the inclusion of propellant the type of propellant and the amount of propellant. If no propellant or less than 3% propellant is included the formulation is a liquid, or semi-solid, or a gel. If the content includes propellant say about 3% to about 50% it can emerge as a foam. If the content includes more than 50% of propellant say even up 95% it can emerge as a spray. In one or more embodiments, e.g., where the propellant is separate from the content, the content may be expelled as a mousse, cream, gel, lotion or any other flowable substance. In one or more embodiments a spray is disadvantageous. In one or more embodiments the carrier or composition is not a spray. In one or more embodiments the propellant is less than 55%, or less than 50%, or less than 45%, or less than 40%, or less than 35% or less than 30%, or less than 20% or less than 10% or less than 5% less than 3% or less than 2% or less than 1%. In one or more embodiments the formulation is not a foam. In one or more embodiments the carrier or composition is not a liquid. In one or more embodiments the carrier or composition is a semi-solid. In one or more embodiments the carrier or composition is a gel.

In one or more embodiments the carrier or composition is not hydrophilic or substantially not hydrophilic.

In one or more embodiments, the carrier or composition is a hydrophobic carrier. In one or more embodiments the hydrophobic carrier is free of or substantially free of hydrophilic compounds.

In one or more embodiments, the carrier or composition is free or substantially free of at least of one or more of water, hydrophilic solvents, surface-active agents, protic solvents, polar protic solvents, aprotic solvents, polyols, short chain alcohols, propellant and aldehyde scavengers. In one or more embodiments, the carrier is essentially free of one or more of the aforesaid. In one or more embodiments, the carrier comprises less than about 0.4%, or less than about 0.3%, or less than about 0.2%, or less than about 0.1%, or less than about 0.05% of one or more of the aforesaid.

In one or more embodiments, the carrier or composition is free, essentially free or substantially free of aldehyde scavengers comprising glycerine and anti-oxidants. In one or more embodiments, the vehicle is free, essentially free or substantially free, of anti-oxidant e.g., comprising one or more of alpha-tocopherol, butyl hydroxy anisol (BHA), butyl hydroxy toluene (BHT) and propyl gallate.

In one or more other embodiments, the carrier or composition is free, essentially free, or substantially free of one or more of a liquid fatty alcohol, isopropyl myristate, a minocycline, a tetracycline, adapalene, a retinoid, and an aldehyde scavenger.

In one or more embodiments, the carrier or composition is free, essentially free or substantially free of one or more of dimethyl isosorbide, glycerin, ethanol, propylene glycol, butylene glycol, hexylene glycol, PEG 200, PEG 400, PEG 600, PEG 3350 and diethylene glycol monoethyl ether.

In one or more embodiments, the carrier or composition is free, essentially free, or substantially free of a solvent which can dissolve tofacitinib, wherein said solvent includes one or more of or is selected from the group consisting of dimethyl sulfoxide, propylene glycol, glycerin, polyethylene glycol, isopropyl alcohol, methanol, sodium pyrrolidone carboxylate, 2-hydroxypropyl-γ-cyclodextrin, acetone, purified water, ethanol, 1-propanol, butanediol, 2-(2-ethoxyethoxy)ethanol (transcutol) and mixtures thereof.

In one or more embodiments, the composition comprises a hydrophobic carrier and a JAK kinase inhibitor as the sole active agent. In one or more embodiments, the JAK inhibitor is suspended or partly suspended in the composition. In one or more embodiments, the composition comprises a therapeutically effective amount of a first active agent consisting of a JAK kinase inhibitor and wherein the vehicle does not comprise a second active agent. In one or more embodiments, the JAK inhibitor is a JAK 3 and or a JAK 1 inhibitor. In some embodiments, the JAK inhibitor is a tofacitinib, e.g., a tofacitinib salt, e.g. tofacitinib citrate. In one or more embodiments, the composition comprises a therapeutically effective amount of a first active agent consisting of a S1PR modulator or agonist e.g., fingolimod or a pharmaceutically acceptable salt thereof (e.g., fingolimod hydrochloride or fingolimod phosphate) and wherein the vehicle does not comprise a second active agent.

In one or more embodiments, the carrier or composition comprises less than about 45%, or less than about 40%, or less than about 35%, or less than about 30%, or less than about 25%, or less than about 20%, or less than about 15%, or less than about 10%, or less than about 5%, or less than about 4%, or less than about 3%, or less than about 2%, or less than about 1%, or less than about 0.9%, or less than about 0.8%, or less than about 0.7%, or less than about 0.6%, or less than about 0.5%, or less than about 0.4%, or less than about 0.3%, less than about 0.2%, or less than about 0.1%, or less than about 0.05%, aprotic polar solvents.

In one or more embodiments, the carrier or composition comprises less than about 80%, or less than about 75%, or less than about 70%, or less than about 65%, or less than about 60%, or less than about 55%, or less than about 50% aprotic polar solvents.

In one or more embodiments, the carrier or composition comprises less than about 45%, or less than about 40%, or less than about 35%, or less than about 30%, or less than about 25%, or less than about 20%, or less than about 15%, or less than about 10%, or less than about 5%, or less than about 4%, or less than about 3%, or less than about 2%, or less than about 1%, or less than about 0.9%, or less than about 0.8%, or less than about 0.7%, or less than about 0.6%, or less than about 0.5%, or less than about 0.4%, or less than about 0.3%, or less than about 0.2%, or less than about 0.1% or less than about 0.05% dimethyl sulfoxide or propylene glycol.

In one or more embodiments, the carrier or composition comprises less than about 80%; or less than about 75%, or less than about 70%; or less than about 65% or less than about 60% or less than about 55% or less than about 50% dimethyl sulfoxide or propylene glycol.

In one or more embodiments, the solvent comprises or is a combination of dimethyl sulfoxide with at least one of propylene glycol, ethanol, and water and wherein the solvent less than about 80%, or less than about 75%, or less than about 70%, or less than about 65%, or less than about 60%, or less than about 55%, or less than about 50%, or less than about 45%, or less than about 40%, or less than about 35%, or less than about 30%, or less than about 25%, or less than about 20%, or less than about 15%, or less than about 10% or less than about 7.5% or less than about 5% or less than about 2 or less than about 1% of the composition.

In one or more other embodiments the carrier and composition is free, essentially free, or substantially free of a polymeric agent or a gelling agent other than the cross polymers which are part of elastomers or polymers which are part of Versogel®, when the carrier or composition comprises an elastomer or a Versogel®.

In one or more embodiments, the JAK inhibitor is solvated, substantially solvated, or partially solvated by the hydrophobic agent. In one or more embodiments, the JAK inhibitor is not solvated by the hydrophobic agent.

In some embodiments the composition comprises fingolimod. Fingolomod or 2-amino-2-[2-(4-octylphenyl)ethyl]propane-1,3-diol is an aminodiol that consists of propane-1,3-diol having amino and 2-(4-octylphenyl)ethyl substituents at the 2-position. It is a sphingosine 1-phosphate receptor modulator (S1PR1, S1P1) used for the treatment of relapsing-remitting multiple sclerosis. A prodrug, fingolimod is phosphorylated by sphingosine kinase to active metabolite fingolimod-phosphate, a structural analogue of sphingosine 1-phosphate. It has a role as an immunosuppressive agent, a prodrug, an antineoplastic agent, a sphingosine-1-phosphate receptor agonist and a CB1 receptor antagonist. It is an aminodiol and a primary amino compound. Fingolimod hydrochloride is the hydrochloride salt form of fingolimod.

When fingolimod binds to S1PR1 on lymphocytes and causes transient receptor activation followed by S1PR1 internalization and degradation it results in the sequestration of lymphocytes in lymph nodes and in turn can reduce the amount of circulating peripheral lymphocytes and the infiltration of lymphocytes into target tissues. Fingolimod can modulate macrophage proliferation, and cytokine release.

In one or more embodiments a sphingosine-1-phosphate receptor 1 (S1PR1, S1P1) modulator (e.g., a fingolimod), may be effective in treating dermatological disorders involving inflammation and or lymphocyte action. In some embodiments the dermatological disorder can include one or more of psoriasis, a dermatomyositis, eczema, dermatitis, atopic dermatitis, acne, rosacea, a disorder of the pilosebaceous unit, scarring, alopecia and vitiligo.

In one or more embodiments the effectiveness of the sphingosine-1-phosphate receptor 1 (S1PR1, S1P1) modulator, in treating dermatological disorders may be improved by combining it with a JAK inhibitor (e.g., a tofacitinib). In one or more embodiments the sphingosine-1-phosphate receptor 1 (S1PR1, S1P1) modulator and or the JAK inhibitor are given orally. In one or more embodiments the sphingosine-1-phosphate receptor 1 (S1PR1, S1P1) modulator and or the JAK inhibitor are applied topically at and around the site of the disorder. In some embodiments they are given both orally and topically. By giving the active agents topically it is possible to reduce the potential systemic side effects. This can be particularly beneficial with both classes of drugs since lower doses and lower systemic levels can result in reduced side effects. By developing a carrier which has no or low penetration enhancers and or a low effect on penetration such that intradermal penetration is limited or minimal there is provided in one or more embodiments a product that will result in a significantly reduced number adverse events and lead to events that are transitory and classified a low or moderate rather than severe.

In one or more embodiments the active agent modulates lysophospholipid (LP) receptors as therapeutic targets through the LP receptor branch containing sphingosine 1-phosphate (S1P) receptors. In one or more embodiments lysophospholoipid modulators can treat or ameliorate a dermatological disorder. In one or more embodiments the dermatological disorder involves inflammation.

In some embodiments a therapeutically effective effect amount of a fingolimod is applied topically (either as a salt (e.g. fingolimod hydrochloride) or base) either alone or in combination with a JAK inhibitor (e.g., tofacitinib (either as a salt (e.g., tofacitinib citrate) or base) to treat or ameliorate a dermatological disorder, such as atopic dermatitis, ichthyosis vulgaris, psoriasis, dermatitis, eczema, vitiligo, alopecia, alopecia totalis, alopecia universalis, autoimmune bullous skin disorder such as pemphigus vulgaris (PV) or bullous pemphigoid (BP), skin rash, skin irritation, skin sensitization (e.g., contact dermatitis or allergic contact dermatitis), chronic atypical neutrophilic dermatosis with lipodystrophy and elevated temperature (CANDLE), pustulosis palmoplantaris, ichtyosis, actinic keratosis, pruritus, rosacea, lupus erythematosus, skin inflammation, skin itch, skin infection, acne, and acne vulgaris. In one or more other embodiments the disorder which can be treated or ameliorated by a fingolimod (either as a salt (e.g. fingolimod hydrochloride) alone or in combination with a JAK inhibitor (e.g., tofacitinib (either as a salt (e.g., tofacitinib citrate) or base) can also be folliculitis, furunculosis, keratosis pilaris, hidradentitis suppurativa, pyoderma gangrenosum, a lichenification disorder e.g., lichen planus, sclerosus, lichen simplex chronicus, neurodermatitis, primary cicatricial alopecias, such as lichen planopilaris and frontal fibrosing alopecia, and cellulitis. The term lichenification is classed as a secondary skin lesion wherein the characteristic features of skin thickening, hyperpigmentation, and exaggerated skin lines are noted. Lichenification can be further divided into primary and secondary types. Primary lichenification signifies lichen simplex chronicus, also known as neurodermatitis circumscripta. Secondary lichenification occurs in atopic dermatitis, infective eczematous dermatoses, psoriasis, psoriasiform dermatosis, xerosis, pityriasis rubra pilaris, porokeratosis, vegetative growths, anxiety, and obsessive-compulsive disorders.

In some embodiments fingolimod is a salt (e.g., fingolimod hydrochloride) and in some it is fingolimod base. In some embodiments a fingolimod salt is combined with a JAK inhibitor as a base and in some embodiments with a JAK inhibitor as a salt. In some embodiments fingolimod base is combined with a JAK inhibitor as a base and in some embodiments with a JAK inhibitor as a salt. In some embodiments the JAK inhibitor is a pan JAK inhibitor. In some embodiments the JAK inhibitor is a JAK 1 inhibitor, in some embodiments a JAK 2 inhibitor, in some embodiments a JAK 3 inhibitor and in some embodiments a combination of two (e.g., JAK 1 inhibitor and JAK 3 inhibitor) or more.

In some embodiments a therapeutically effective effect amount of a fingolimod applied topically in combination with a JAK inhibitor (e.g., a tofacitinib salt or base) is capable of restoring skin barrier. In some embodiments a therapeutically effective effect amount of a fingolimod applied topically in combination with a JAK inhibitor (e.g., a tofacitinib salt or base) is capable of increasing the level of filaggrin in the skin. In some embodiments a therapeutically effective effect amount of a fingolimod applied topically in combination with a JAK inhibitor (e.g., T) can reduce the level of infection (e.g., Staphylococcus aureus). In some embodiments the reduction in infection is bacterial, in some embodiments fungal, in some embodiments viral and in some embodiments two or more thereof. In some embodiments a therapeutically effective effect amount of a fingolimod applied topically in combination with a JAK inhibitor (e.g., a tofacitinib salt or base) can reduce the level of allergic response. In some embodiments a therapeutically effective effect amount of a fingolimod applied topically in combination with a JAK inhibitor (e.g., a tofacitinib salt or base) can reduce the level of inflammation. In some embodiments a therapeutically effective effect amount of a fingolimod applied topically in combination with a JAK inhibitor (e.g., a tofacitinib salt or base) can reduce the level (e.g., of infection, or allergic response, or inflammation) or increase the level (e.g., of skin restoration or filaggrin) by a level more than by each of the individual drugs. In some embodiments a therapeutically effective effect amount of a fingolimod applied topically in combination with a JAK inhibitor (e.g., a tofacitinib salt or base) can reduce the level of one or more of dendritic cell migration, cytokine production, recruitment of cells involved in inflammation, such as lymphocytes, macrophages, and monocytes. In some embodiments a therapeutically effective effect amount of a fingolimod applied topically in combination with a JAK inhibitor (e.g., a tofacitinib salt or base) can reduce the level of itching. In some embodiments a therapeutically effective effect amount of a fingolimod applied topically in combination with a JAK inhibitor (e.g., a tofacitinib salt or base) can reduce the level of mast cell infiltration in the dermis. In some embodiments a therapeutically effective effect amount of a fingolimod applied topically in combination with a JAK inhibitor (e.g., a tofacitinib salt or base) can reduce the level of risk of a dermatological disorder (e.g., atopic dermatitis, ichthyosis vulgaris, psoriasis and scarring). In one or more embodiments there is a synergistic effect when a therapeutically effective effect amount of a fingolimod is applied topically in combination with a JAK inhibitor (e.g., a tofacitinib salt or base).

Skin wound healing is a process that consists of three sequential phases: inflammation, proliferation, and regeneration. During the activation of the inflammatory cascade numerous inflammatory cells infiltrate the damaged area and release cytokines. The cytokines stimulate the migration of keratinocytes and fibroblasts to the wound site and subsequent proliferation of these cells begins (e.g., 4-5 days) later. Fibroblasts secrete extracellular matrix (ECM) proteins such as fibronectin, collagen and hyaluronic acid, resulting in the formation of granulation tissue. During the proliferation phase, abundant vascularization and angiogenesis play a key role in supplying the inflammatory cells and fibroblasts for the formation of an occasional granulation matrix.

Silicone gels comprising polysiloxanes are applied as a treatment for reducing scars. The initial action of a silicone is to occlude or seal the scar and restore the barrier function of the stratum corneum by reducing transepidermal water loss (TEWL). Dehydration causes cytokine-mediated signalling from keratinocytes to dermal fibroblasts which increase production of collagen. However, over production of collagen can result in thick raised unsightly scars. Occlusion keeps the scar hydrated, so fewer signals are sent to the wound creating less scar tissue. Despite its occlusive properties, silicone provides optimal permeability to maximize oxygen transfer across the surface of the skin which enhances wound healing. Silicone also helps transfer tension from the edges of the wound to the silicone. These tension forces that normally widen scars are reduced due to absorption of tension by the silicone. Silicone also reduces the redness of the scar by preventing the creation of new blood vessels. Silicone also creates a negative static field which aligns and organizes the collagen fibers in a more uniform pattern. The negative field also tends to pull in or cause the involution of raised scars. In summary occlusion potential resulting in less production of collagen, tension transfer, decreased capillary activity and collagen alignment from silicone treatment may result in enhanced scar healing.

Elastomer based formulations may provide an alternative lesser occlusive or non-occlusive platform for the treatment and reduction of scars. In one or more embodiments the elastomer-based technology and formulations described herein can be applied to improve the appearance of scars and to prevents abnormal or excessive scar formation. In one or more embodiments elastomer-based formulations can diminish the appearance of hypertrophic scars and keloids. In some embodiments, elastomer-based formulations can diminish scars and keloids with a raised and/or discolored appearance. In some embodiments, elastomer-based formulations can soften and flatten raised scars. In some embodiments, elastomer-based formulations can reduce the redness associated with scars. In some embodiments, elastomer-based formulations can be effective for both old and new scars. In one or more embodiments elastomer-based formulations are suitable for use in adults, teenagers, adolescents, and children. In some embodiments, elastomer-based formulations can be suitable for use on people with sensitive skin. In some embodiments elastomer-based formulations can be used on scars that result from surgery, injury, burns, acne, rosacea, psoriasis, dermatitis, cuts, insect bites, and others. Various elastomers (such as elastomers listed in Table 1 herein may be used as the basis. The elastomer may be suspended/dispersed in a volatile silicone fluid (e.g., a cyclomethicone or a dimethicone) in various proportions. Higher levels of silicone fluid in proportion to the elastomer can ease the viscosity of the elastomer whilst lower levels can produce more viscous gels. In one or more embodiments elastomer based formulation are formulated with ST-Elastomer 10. This is a blend of a unique silicone elastomer and a volatile silicone fluid which acts as a film forming and thickening agent for water-in-oil and water-in-silicone formulations and silicone fluid. In contrast to currently commercially available silicone products, it is a non-occlusive mixture of high molecular weight crosslinked silicone (e.g., about 12%) in decamethylcyclopentasiloxane (cyclomethicone). Instead ST-Elastomer 10 offers, for example, slight sebum absorption, dry & silky smoothness and non-greasy & non-tacky feel on the skin.

In one or more embodiments the introduction of emollients in elastomer based formulations may further help the treatment and reduction of scars. In some embodiments they may also help reduce itch. In some embodiments they may help unclog skin pores. Unblocking the pilosebaceous units may be beneficial and especially in the case of facial scars. In one or more embodiments beneficial emollients comprise an isopropyl ester, e.g., isopropyl isostearate and or a saturated or branched hydrocarbon oil e.g., squalane. In one or more embodiments beneficial emollients comprise one or more of an MCT oil, mineral oil, or IPP. In one or more embodiments beneficial emollients comprise a plant-based oil such as soybean oil or coconut oil. Such oils may have antibacterial properties. In one or more embodiments one or more of the adhesiveness, surface energy, surface tension, or interfacial tension of the composition is reduced e.g., to discourage or reduce adhesion. Without being bound by any theory this approach is contrary to the general approach of silicone gels described above where upon application the gel is to form an adhesive film over the area of the scar.

In one or more embodiments compositions that provide the scar treatment potential of low-occlusive elastomer-based formulations or non-occlusive elastomer-based formulations alone or together with active agents that can modulate the inflammatory response to improve the treatment, reduction and healing of scars may be beneficial and provide advantages over the prior art occlusive siloxanes.

In one or more embodiments agents that can modulate the inflammatory response are immunosuppressive agent and a sphingosine-1-phosphate receptor agonist. In one or more embodiments the active agent is an immunosuppressive agent and or a sphingosine-1-phosphate receptor agonist (e.g., a fingolimod, such as the free base, salt, hydrochloride, or phosphate). In one or more embodiments the active agent is a JAK inhibitor (e.g., such as the free base, salt, citrate). In some embodiments the active agent is a combination of an immunosuppressive agent and or a sphingosine-1-phosphate receptor agonist and a JAK inhibitor. In some embodiments the combination is a fingolimod and a tofacitinib.

Keloids and hypertrophic scars are excessive scar formations with chronic inflammation and capillary vasculogenesis. Hypertrophic scar formation is considered a result of the imbalance between extracellular matrix synthesis and degradation during wound healing. Fingolimod is an analogue of sphingosine-1-phosphate (S1P). S1P is a lipid mediator, which is involved in inflammatory cell recruitment and angiogenesis. Fingolimod is a functional agonist of S1P receptor 1 (S1PR1), and inhibits sphingosine kinase 1 (SphK1), which produces S1P. Tofacitinib is a small-molecule JAK inhibitor and has been shown to inhibit cytokines directly and leads to rapid attenuation of JAK-STAT signalling in keratinocytes.

Without being bound by any theory, a topical silicone elastomer composition comprising fingolimod alone or in combination with a tofacitinib can provide improved anti-scarring and healing properties as both the carrier and active agents have an effect on accelerated and improved scar treatment and healing. In one or more embodiments the elastomer-based carrier and one or both active agents have a synergistic effect on scar treatment and reduction and may lead to an accelerated treatment and healing. All this can be an ancillary to the benefits of the one or both active agents in ameliorating and treating dermatological disorders involving inflammation. The fingolimod is directed to the inflammatory response involved in wound healing by decreasing recruitment of inflammatory cells to the local region, and further inhibiting angiogenesis and tofacitinib is involved in attenuation of JAK-STAT signalling in keratinocytes. Whereas, silicone is involved inter alia with the mechanical aspects of wound healing. In addition, as ST-elastomer offers slight sebum absorption it may assist targeted penetration of the active agent or provide a higher local concentration of active agent in the sebum.

In some embodiments the amount of a fingolimod applied topically is about 0.0001% to about 0.1% by weight of the composition. In some embodiments the amount of a fingolimod applied topically is about 0.0002% to about 0.1% by weight of the composition. In some embodiments the amount of a fingolimod applied topically is about 0.0005% to about 0.05% by weight of the composition. In some embodiments the amount of a fingolimod applied topically is about 0.001% to about 0.01% by weight of the composition. In some embodiments the amount of a fingolimod applied topically is about 0.001% to about 1% by weight of the composition. In some embodiments the amount of a fingolimod applied topically is about 0.002% to about 0.1% by weight of the composition. In some embodiments the amount of a fingolimod applied topically is about 0.005% to about 0.01% by weight of the composition. In some embodiments the amount of a fingolimod applied topically is about 0.001% to about 0.05% by weight of the composition. In some embodiments the amount of a fingolimod applied topically is about 0.0001% to about 10% by weight of the composition. In some embodiments the amount of a fingolimod applied topically is above about 0.001% by weight of the composition. In some embodiments the amount of a fingolimod applied topically is above about 0.005% by weight of the composition. In some embodiments the amount of a fingolimod applied topically is above about 0.01% by weight of the composition. In some embodiments the amount of a fingolimod applied topically is about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1% by weight of the composition. In some embodiments, the amount of a fingolimod applied topically is about 0.0015%, about 0.0025%, about 0.0035%, about 0.0045%, about 0.0055%, about 0.0065%, about 0.0075%, about 0.0085%, about 0.0095%, about 0.015%, about 0.025%, about 0.035%, about 0.045%, about 0.055%, about 0.065%, about 0.075%, about 0.085%, about 0.095%, about 0.1%, about 0.11%, 0.12%, 0.13%, 0.14%, 0.15%, 0.16%, 0.17%, 0.18%, 0.19% and 0.2% by weight of the composition. In some embodiments a fingolimod is applied topically in any of the aforesaid amounts together with at least one additional active agent e.g., a JAK inhibitor (e.g., a tofacitinib). In one or more embodiments any of the aforesaid amounts of a fingolimod when used in combination with a e.g., a JAK inhibitor (e.g., a tofacitinib) may be reduced by about 0.1%, by 0.25%, by 0.3%, by 0.4%, by 0.5%, by 0.6%, by 0.7%, 0.8%, 0.9%, by 1%, by 2%, by 3%, by 4%, by 5%, by 6%, by 7%, by 8%, by 9%, by 10%, by 15%, by 20%, by 25%, by 30%, by 35%, by 40%, by 45%, by 50%, by 55%, by 60%, or by 75%.

In some embodiments, the amount of a fingolimod applied topically is about 0.001%, about 0.002%, about 0.003%, about 0.004%, about 0.005% about 0.006%, about 0.007%, about 0.008%, about 0.009%, about 0.01% about 0.02%, about 0.03%, about 0.04%, about 0.05%, about 0.06%, about 0.07%, about 0.08%, about 0.09%, or about 0.1%. In some embodiments, the amount of a fingolimod applied topically is about 0.1%, about 0.11%, about 0.12%, about 0.13%, about 0.14%, about 0.15%, about 0.16%, about 0.17%, about 0.18%, about 0.19% or about 0.2% by weight of the composition. In some embodiments, the amount of a fingolimod applied topically is about 0.0015%, to about 0.02% or about 0.004% to about 0.01% by weight of the composition. In some embodiments, the amount of a fingolimod applied topically is between about 0.001% and about 0.03% by weight of the composition. In some embodiments, the amount of a fingolimod applied topically is between about 0.005%, and about 0.02% by weight of the composition. In some embodiments a fingolimod is applied topically in any of the aforesaid amounts together with at least one additional active agent e.g., a JAK inhibitor (e.g., a tofacitinib).

In some embodiments, the amount of a fingolimod applied topically is about 0.002% to about 0.1% by weight of the composition and the amount of a tofacitinib is about 0.1% to about 1% by weight of the composition. In some embodiments, the amount of a fingolimod applied topically is about 0.005% to about 0.01% by weight of the composition and the amount of a tofacitinib is about 0.3% to about 0.6% by weight of the composition.

In some embodiments, the amount of a fingolimod applied topically is about 0.005% to about 0.2% by weight of the composition and the amount of a tofacitinib is about 0.1% to about 0.6% by weight of the composition. In some embodiments, the amount of a fingolimod applied topically is about 0.005% by weight of the composition and the amount of a tofacitinib is about 0.6% by weight of the composition. In some embodiments, the amount of a fingolimod applied topically is about 0.01% by weight of the composition and the amount of a tofacitinib is about 0.6% by weight of the composition. In some embodiments, the amount of a fingolimod applied topically is about 0.01% by weight of the composition and the amount of a tofacitinib is about 0.3% by weight of the composition. In some embodiments, the amount of a fingolimod applied topically is about 0.01% by weight of the composition and the amount of a tofacitinib is about 0.1% by weight of the composition.

In some embodiments a therapeutically effective effect amount of a fingolimod applied topically in combination with a JAK inhibitor (e.g., a tofacitinib salt or base) can normalize skin pH. In some embodiments a therapeutically effective effect amount of a fingolimod applied topically in combination with a JAK inhibitor (e.g., a tofacitinib salt or base) can increase the concentration of NMF (Natural Moisturizing Factor) in skin. In some embodiments a therapeutically effective effect amount of a fingolimod applied topically in combination with a JAK inhibitor (e.g., a tofacitinib salt or base) can increase the concentration of PCA (pyrrolidone carboxylic acid) and UCA (urocanic acid) in skin. In some embodiments, the increased concentration of PCA and/or UCA has inhibitory effects on Staphylococcus aureus and is beneficial in the treatment of a skin disorder, e.g., atopic dermatitis. In some embodiments a therapeutically effective effect amount of a fingolimod applied topically in combination with a JAK inhibitor (e.g., a tofacitinib salt or base) can reduce trans epidermal water loss (TEWL). In some embodiments a therapeutically effective effect amount of a fingolimod applied topically in combination with a JAK inhibitor (e.g., a tofacitinib salt or base) can reduce the antigen-capture by Langerhans cells, thereby reducing skin inflammatory response.

In some embodiments the amount of a fingolimod applied topically is about 0.0001% to about 10% by weight of the composition and the amount of a tofacitinib is about 0.01% to about 10% by weight of the composition. In some embodiments the amount of a fingolimod applied topically is about 0.001% to about 1% by weight of the composition and the amount of a tofacitinib is about 0.05% to about 3.05% by weight of the composition. In some embodiments the amount of a fingolimod applied topically is about 0.002% to about 0.1% by weight of the composition and the amount of a tofacitinib is about 0.1% to about 1% by weight of the composition. In some embodiments the amount of a fingolimod applied topically is about 0.005% to about 0.01% by weight of the composition and the amount of a tofacitinib is about 0.3% to about 0.6% by weight of the composition. In some embodiments the amounts of a fingolimod and a tofacitinib are as described elsewhere in the specification and or in the Examples.

In one or more embodiments having a combination of active agents (e.g., a fingolimod and a tofacitinib) allows for the amount used of one or both active agents to be reduced whilst providing a therapeutic effect in treating or ameliorating a disorder. In some embodiments the amount of an active agent (e.g., a fingolimod or a tofacitinib or both) can be reduced by about 5%, or about 10%, or about 15%, or about 20%, or about 25%, or about 30% by weight in the composition when used in combination with at least one other active agent. In some embodiments the amount of an active agent can be reduced by about 35%, or about 40%, or about 45%, or about 50%, or about 55%, or about 60%, or about 75% by weight in the composition when used in combination with at least one other active agent. In some embodiments each active agent can be reduced in similar proportions. In some embodiments two or more active agents in combination may be reduced in different proportions, for example for every 1% reduction by weight of one active agent the amount of another active agent may be reduced by 0.1%, by 0.25, by 0.3%, by 0.4%, by 0.5%, by 0.6%, by 0.7%, 0.8%, 0.9%, by 1%, by 2%, by 3%, by 4%, by 5%, by 6%, by 7%, by 8%, by 9%, or by 10% whilst achieving a therapeutic effect. The advantages of a combination to treat or ameliorate a disorder include being able to provide an improved therapeutic effect, and or a therapeutic effect at a lower dosage or frequency, and or reduced or minimized potential side effects and adverse events.

In one or more embodiments there is provided a composition for use in the manufacture of a medicament comprising a JAK inhibitor (e.g., a tofacitinib) and or a S1PR modulator or agonist (e.g., a fingolimod) having an effect of ameliorating or treating a dermatological disorder. In one or more embodiments there is provided the use of a composition in the manufacture of a medicament comprising a JAK inhibitor (e.g., a tofacitinib) and or a S1PR modulator or agonist (e.g., a fingolimod) having an effect of ameliorating or treating a dermatological disorder. In one or more embodiments the dermatological disorder one or more of the disorders or conditions described elsewhere herein.

In one or more embodiments compositions comprising tofacitinib or a pharmaceutically active salt thereof may also be read as including fingolimod or a pharmaceutically active salt thereof. In one or more embodiments compositions comprising fingolimod or a pharmaceutically active salt thereof may also be read as including tofacitinib or a pharmaceutically active salt thereof.

### EXAMPLES

### Materials

Exemplary ingredients suitable for the production of compositions disclosed herein are listed in Table 1. Each ingredient may in some embodiments have two or more functions, as will be appreciated by one skilled in the art of which one by way of a non-limiting example is indicated in the Table.

TABLE 1
Exemplary Ingredients Suitable to Produce Compositions

| Chemical Name | Commercial Name | Supplier | Exemplary Function |
|---|---|---|---|
| Isopropyl Palmitate | Crodamol IPP | Croda | Diluent |
| Isopropyl Myristate | Crodamol IPM | Croda | Diluent |
| Oleyl Alcohol | Kollicream OA | BASF | Diluent |
| Diisopropyl adipate | Schercemol DIA | Lubrizol | Diluent |
| PPG 15 Stearyl ether | Arlamol E | Croda | Diluent |
| Cetearyl Ethylhexanoate | Lanol 1688 | SEPPIC | Diluent |
| Squalane | Neossance squalane | Centerchem | Diluent |
| Isopropyl Isostearate | Crodamol IPIS | Croda | Diluent |
| Cyclomethicone & | ST-Elastomer 10 | DuPont | Carrier |
| dimethicone crosspolymer | | | |
| MCT oil | Miglyol 812N | IOI Oleo | Diluent |
| Cyclomethicone | ST-Cyclomethicone 5NF | DuPont | Diluent |
| Dimethicone | Q7-9120 Silicon fluid | DuPont | Diluent |
| PEG 400 | PEG400 Super Refined | Croda | Solvent |
| PEG 3350 | Pluriol E 3350 | BASF | Thickener |
| Propylene glycol | Kollisolv PG | BASF | Solvent |
| Glycerin | Glycerin | KLK Oleo | Solvent |
| Dimethyl sulfoxide | Dimethyl sulfoxide | Bio-Lab | Solvent |
| (DMSO) | | | |
| Butylated hydroxyl | Butylated | Sigma Aldrich | Anti-oxidant |
| anisole (BHA) | hydroxyanisole | | |
| Stearyl alcohol | Kolliwax SA | BASF | Thickener |
| Stearic acid | Kolliwax SA | BASF | Thickener |
| Ceteareth 20 | Kolliphor CS20 | BASF | Surfactant |
| Glyceryl monostearate | Kolliwax GMS | BASF | Surfactant |
| Glyceryl isostearate | Glyceryl isostearate | CONNOK | Surfactant |
| Hypromellose K100M | Methocel K100M | Colorcon Dow | Gelling agent |
| Citric acid | Citric acid anyhdrous | Merck | pH adjusting agent |
| Benzyl alcohol | Benzyl alcohol | Merck | Preservative |
| Glyceryl Behenate | Compritol 888 | Gattefossé | Thickener |
| Hydrogenated Castor Oil | Kolliwax HCO | BASF | Thickener |
| mineral oil | Blandol | Sonnebom | Diluent |
| Zea Mays Starch | Amidon De Mais Extra | ROQUETTE | Sensory agent |
| | Blank | | |
| Cetearyl Isononanoate | Cetiol SN | BASF | Diluent |
| Glyceryl behenate | Compritol 888ATO | Gattefosse | Thickener |
| Transcatol | Ethoxydiglicol | Gattefosse | Solvent |
| Dimethyl Isosorbide | Gransolve | Sriken/ZGrant | Solvent |
| Ethanol | Ethanol dehydrated | Bio Lab | Solvent |
| Hexylene glycol | Hexylene glycol | Sigma Aldrich | Solvent |
| Isopropyl alcohol | Propan-2-ol | Gadot | Solvent |
| PPG-15 Stearyl ether | CETIOL E | BASF | Diluent |
| Propyl paraben | Propyl 4- | Sharon | Preservative |
| | Hydroxibenzoate | | |
| Methyl paraben | Methyl 4- | Sigma Aldrich | Preservative |
| | hydroxybenzoate | | |
| Carboxymethylcellulose | Sodium carboxymethyl | CP KELCO | Thickener |
| | cellulose(Cekol | | |
| | 30000, Cekol 50000) | | |
| Xanthan gum | Xantural 11k | CP KELCO | Thickener |
| Petrolatum | Pioner 3476 | Hansen & | Carrier |
| | | Rosenthal | |
| Gelled mineral oil | Versagel M750 | Calumet | Carrier |
| (Mineral Oil (and) | | Lubricants | |
| Ethylene/Propylene/ | | | |
| Styrene Copolymer (and) | | | |
| Butylene/Ethylene/ | | | |
| Styrene Copolymer) | | | |
| Cyclopentasiloxane (and) | MGS-Elastomer 1100 | Grant | Gelling agent |
| Polysilicone-11 | | | |
| Cyclopentasilicone (and) | ST Elastomer 1148P | Grant | Gelling agent |
| Petrolatum (and) | | | |
| Polysilicone-11 | | | |
| Polysilicone 11 in | Gransil DMG-3 | Grant | Gelling agent |
| Dimethicone (3 cSt) | Elastomer | | |
| Polysilicone 11 in | Gransil DM-5 Elastomer | Grant | Gelling agent |
| Dimethicone (5 cSt) | | | |
| Poly silicone 11 in | Gransil DMG-6 Elastomer | Grant | Gelling agent |
| Dimethicone (6 cSt) | | | |
| Soybean oil | Soybean oil | Henry & Lamotte | Diluent |
| Tofacitinib citrate | Tofacitinib Citrate | Apotex | Active agent |
| Tofacitinib free base | | Synthesized by | Active agent |
| | | Nuvisan for | |
| | | Foamix | |
| Cyclopentasiloxane (and) | MGS-Elastomer 1100 | Grant | Carrier |
| Polysilicone-11 | | | |
| Dimethicone (and) | Gransil DMG-3 | Grant | Carrier |
| Polysilicone-11 | | | |
| Minocycline | Minocycline | Hovione | Active agent |
| hydrochloride | hydrochloride | | |
| Triamcinolone acetonide | Triamcinolone acetonide | NewChem | Active agent |
| Mometasone furoate | Mometasone furoate | Sicor | Active agent |
| Nicotinamide | Niacinamide | DSM Nutritional | Active agent |
| Adapalene | Adapalene | Excella | Active agent |
| Fingolimod hydrochloride | Fingolimod | Teva | Active agent |
| | hydrochloride | | |
| Fingolimod base | Fingolimod base | | Active agent |

### Methods

### Experimental Method A: Skin Penetration (IVPT)

Human skin from cosmetic reduction surgery, dermatomed to 500 μm, is mounted between the donor and receptor compartments of a vertical diffusion cell with an exposed dosing surface area of ˜0.6 cm2.

The skin is dosed with approx. 6 mg of formulation to achieve a dose of ˜10 mg/cm2. Receptor solution is collected at pre-determined intervals over the course of 24 hours and analyzed using a liquid chromatography with tandem mass spectrometry (LC-MS/MS) analytical method.

Following 24 hours, the residual formulation is removed from the surface of the skin. The skin surface is tape-stripped up to five times to remove residual formulation and the top of the skin surface layers (Stratum Corneum). The epidermis is then heat-separated from the dermis. The active agent is extracted from the skin layers and the amount of active agent delivered to the epidermis and dermis is then determined by LC-MS/MS.

### Experimental Method B: In-Vitro Release Testing (IVRT)

Release test is performed using a Franz-cell apparatus. The tested formulation is placed on a suitable membrane, and a suitable receptor fluid is placed in the receptor chamber. The concentration of the active agent in the receptor fluid is measured over time, and the release rate is calculated.

### Experimental Method C: AD Murine Model

Atopic Dermatitis is induced by once daily application for 38 days of 100 μl of 0.5% 2,4-dinitrochlorobenzene (DNCB) solution onto the shaved back skin of mice. Starting on day 32, test formulations are administered once daily onto the shaved back skin of mice in addition to DNCB. Several measurements are taken at different time points:

Daily from Day 32: Body weight, mortality, behaviour (scratching), general condition, AD index.

Day 39: Histological analysis of skin samples, microscopic scoring of AD, blood assays, assay for pro-inflammatory cytokines IL-1, IL-6, IL-18, TNF-α, IgE, and histamine assays.

### Experimental Method D: Solubility Test

Saturated solutions of tofacitinib citrate in various solvents are generated by agitating (stirring) an excess amount of solid crystalline tofacitinib citrate in the corresponding solvent at ambient conditions. The resulting solution, which is in equilibrium with the solid phase, is filtered and analyzed by high-performance liquid chromatography (HPLC) after at least 24 hours of agitation to determine concentration of dissolved tofacitinib citrate. An additional analysis is performed after 48 hours of agitation to confirm the determined saturated concentration.

### Experimental Method E: Compatibility Test

Solid tofacitinib citrate is added to (1) MCT oil, (2) water, and (3) to the mixtures of water with each of the excipients. The mixtures are tightly closed and exposed to 60° C. protected from light. After exposure to elevated temperature, the mixtures are equilibrated with ambient conditions and the tofacitinib and its degradation products are analyzed by HPLC.

### Experimental Method F: MTT Test and Interleukin—La Release Test

The test consists of a topical exposure of the reconstructed human epidermis (RhE) model to the test items followed by a cell viability test. The reduction of cell viability following exposure to chemicals is used to predict skin irritation potential.

The cell viability is measured by dehydrogenase conversion of MIT [(3-4,5-dimethyl thiazole 2-yl) 2,5-diphenyltetrazoliumbromide] into a blue formazan salt that is quantitatively measured after extraction from tissues by absorbance at 570 nm.

Irritant chemicals are identified by their ability to decrease cell viability below a defined threshold level. A test sample is considered an irritant if viability is ≤50% as compared to control sample.

The concentration of Human IL-1α released into the culture media during the sample exposure period is measured in EpiDerm culture medium samples using a Human IL-α immunoassay Quantikine® ELISA kit. The signal is measured at 450 nm and the IL-1a concentrations in samples is calculated based upon generation of linear standard curve. A two-fold increase or greater in IL-1α concentration, compared to the negative control, is considered a positive induction response.

### Experimental Method G: Hen's Egg Test Chorioallantoic Membrane (Het-Cam-2)

Fertilized hen's eggs are rotated in an incubator for 9 days, after which any defective eggs are discarded. On day 10, the shell around the air cell is removed and the inner membranes are extracted to reveal the chorioallantoic membrane (CAM). Test samples are added to the membrane, ensuring that at least 50% of the CAM surface area is covered. Each sample is applied on three eggs and left in contact for up to 5 minutes. The membrane is examined for vascular damage and the time taken for injury to occur is recorded. Irritancy is scored according to the speed at which damage occurs.

### Experimental Method H: Chemical Stability

Topical formulations comprising tofacitinib are packaged into glass jars or laminated aluminum tubes and exposed to 25° C. or 40° C. for 1, 3 or 6 months or longer e.g. 12 months, and to 50° C. for 1 month. The samples are analyzed for tofacitinib and its degradation products by HPLC. Where another active agent such as fingolimod is present in the formulations the samples are analysed for that active agent and its degradation products by HPLC. Stability of the formulation containing tofacitinib (e.g. 1.0% tofacitinib citrate corresponding to 0.6% tofacitinib or e.g., 0.5% w/y of tofacitinib citrate corresponding to 0.3% tofacitinib and Fingolimod (e.g., 0.0112% w/w of fingolimod hydrochloride corresponding to Fingolimod 0.01% or e.g., 0.00112% w/w of fingolimod hydrochloride corresponding to Fingolimod 0.001%) fingolimod are examined at 5° C., 40° C. and 50° C. for 3 weeks, 2 months or longer. Samples are also examined at 25° C. Tofacitinib and fingolimod are analyzed by high-performance liquid chromatography utilizing Acquity H-Class Waters HPLC (or equivalent), equipped with LUNA Omega PS C18 column (or equivalent) and photo-diode array detector. The gradient elution is employed. The detection oftofacitinib is performed at 280 nm and the detection of fingolimod is performed at 215 nm.

### Experimental Method I: Physical Properties

Topical formulations without an active agent are packaged into glass vials and classified into a transparent, translucent or an opaque appearance. In addition, formulation fluidity is evaluated and the formulation is then classified as a gel, a flowable semi solid or a liquid. Some formulations are rubbed into the skin to evaluate balling effect (i.e., the presence of small beads causing a slightly granular/grainy feel on the skin).

### Experimental Method J: Interfacial Tension Predictions

Characterization of tofacitinib citrate samples for surface energy with polar and dispersive components is performed using the Washburn method for contact angles and the Fowkes theory to calculate surface energies.

The contact angle values for tofacitinib citrate are obtained for diisomethane and for water. Based on the contact angle data, the surface energy is calculated and polar and dispersive components of surface energy as well as surface polarity are estimated.

Surface tension components and contact angles on polytetrafluoroethylene (PTFE) surface are determined for different oils and oil mixtures used for tofacitinib formulations. Based on this data the surface tension and the surface polarity are calculated for these oils and oil mixtures and the interfacial tension for tofacitinib citrate with these oils and oil mixtures is determined.

The interfacial tension between stainless steel and a tofacitinib salt, e.g., tofacitinib citrate is also determined and compared with the same values for oil mixtures to assess potential adhesion of tofacitinib crystals to stainless steel, when the dispersion is performed in different oil mixtures in contact with stainless steel surfaces.

### Experimental Method K: Foam Quality

Foam quality is graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam; upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

### Experimental Method L: Collapse Time

Collapse Time, which is a measure of thermal stability, is measured by dispensing a given quantity of foam and recording e.g., photographing sequentially its appearance over time while incubating at 36° C. The collapse time is defined as the time when the foam height reaches 50% of its initial height. However, if the foam takes longer than a threshold time, e.g., 180 s, to collapse to 50% of its initial height, then the collapse time may be recorded as >180 s. By way of illustration one foam may remain at 100% of its initial height for three minutes, a second foam may collapse to 90% of its initial height after three minutes, a third foam may collapse to 70% of its initial height after three minutes, and a fourth foam may collapse to 51% of its initial height after three minutes. Nevertheless, in each of these four cases the collapse time is recorded as >180 seconds. For practical purposes a foam is more easily applied to a target area if most of the foam remains intact for a reasonable period of time at 36° C. e.g., for more than 100 seconds, or more than 180 seconds. If, for example, the foam is reduced to 50% of its original height after 100 s, it would be recorded as having a collapse time of 100 s.

### Experimental Method M: Viscosity/Rheology: Alternative Methods (A, B and C)

A—Determination of viscosity is performed utilizing a MCR302 rheometer (or equivalent) equipped with 50 mm sandblasted plate-plate geometry at 25° C. The measurement is performed at a constant shear rate, of 5 sec-1 for 60 seconds and the mean value is reported.

B—Determination of viscosity is made using a DHR3 rheometer (TA instruments), or equivalent. The geometry used is a 40 mm plate-plate with 1000 μm gap and with temperature controlled by a Peltier bottom plate. Rotational measurements are made to obtain the viscosity at 36 s-1.

C—Oscillatory measurements are performed to obtain the viscoelastic parameters. All measurements are made within the linear viscoelastic region, where the storage modulus G′ and the loss modulus G″ are frequency independent. The complex viscosity is determined based on G′ and G″ during temperature sweep from 25° C. to 90° C. with the heating rate of 5° C./minute.

### Experimental Method N: Adhesion

Adhesion or adhesiveness is measured. Adhesiveness is defined as the force (g) needed to overcome attraction between two surfaces which are in contact. Measurements may be made, for example, using the LFRA Brookfield (DV II CP) Texture Analyzer. The two surfaces can be sections of artificial, actual tissue, or skin, and measure about 2×2 cm. During the measurement, one surface is positioned in the middle of a Petri dish and the other surface is attached to the base of texture analyzer probe. A sample of is spread uniformly on the surface that is on the Petri dish. The probe is moved down and up, first bringing the two sections into contact, then separating them. The Texture Analyzer measures the force for separating the surfaces, wherein the adhesive force is expressed as a negative force with the force to bring the two sections in contact as a positive force.

### Experimental Method O: pKa Studies

Minipigs are treated topically on 10% body surface area, once daily for 14 days with the tested formulation. On day 14, blood samples are collected: pre-dose, 1, 2, 4, 8, and 24 hours post-dose, and plasma samples are analyzed for their tofacitinib content by LC-MS/MS.

### Experimental Method P: Product Homogeneity

Formulation batches are tested for active agent content in the top, middle and bottom portions of compounding vessel during manufacturing or in the top middle and bottom of the package during stability testing, each time in duplicates (E1, E2).

### Experimental Method O: In-Vivo Psoriasis Animal Model

Psoriasis is induced by once daily application for 6 days (from day 1 to day 6) of 65 mg of Aldara™ cream onto the shaved back skin of mice. Starting on day 7 and until day 13, 100 mg of test formulations are administered once daily onto the shaved back skin of mice in addition to 65 mg of Aldara™ cream. Several measurements are taken during treatment period from day 7 to day 14: psoriasis index (PASI), body weight, mortality, behaviour, histological analysis of skin samples, blood assays for biomarkers.

PASI scoring takes into account the following 3 parameters:

The presence of erythema on the skin at the area of induction of psoriasis with the following scoring grid:
0=no or no more erythema on the skin,
1=weak erythema on the skin,
2=moderate erythema on the skin,
3=severe erythema on the skin.

The presence of induration of the skin at the area of induction of psoriasis with the following scoring grid:
0=no or no more induration of the skin,
1=weak induration of the skin,
2=moderate induration of the skin,
3=severe induration of the skin.

The presence of peeling of the skin at the area of induction of psoriasis with the following scoring grid:
0=no or no more dryness/peeling of the skin,
1=weak dryness/peeling of the skin,
2=moderate dryness/peeling of the skin,
3=severe dryness/peeling of the skin.

The sum of the scores of these 3 parameters gives the PASI.

### Experimental Method R: Process of Manufacturing a Gel or a Foam

The following procedures are used to produce gel or foam samples, in which only the steps relevant to each formulation are performed depending on the type and nature of ingredients used. All the steps are conducted at room temperature unless otherwise stated.

The oil components of the formulation are combined with active agent such as tofacitinib citrate in a suitable container and thoroughly mixed and homogenized until active agent crystals are well dispersed and no aggregation is observed under microscope. ST-Elastomer 10 is added to the oil fraction containing the active agent in three equal portions, while mixing until homogeneous mixture is generated.

For preparation of a combination product the oil components of the formulation are combined with a combination of active agents such as tofacitinib or a salt thereof (e.g., tofacitinib citrate) and fingolimod or a salt thereof (e.g., fingolimod HCl) in a suitable container and thoroughly mixed and homogenized until the active agents are well dispersed and no aggregation is observed under microscope. ST-Elastomer 10 is added to the oil fraction containing the active agents in three equal portions, while mixing until homogeneous mixture is generated.

The preparation of a combination formulation may include the steps provided below.
Step 1—API's (e.g., Fingolimod HCl and then Tofacitinib Citrate) are mixed sequentially with the one or more oil components such as MCT oil, Squalene and IPIS until they are wetted. In some embodiments, the API's may be added separately, or together. In some embodiments, each is added to an oil e.g., fingolimod to oil component A and mixed; tofacitinib to oil component B and mixed; and A and B are then added together and mixed. In some embodiments, oil components A and B can be the same (such as a combination of MCT oil, Squalene and IPS in the same proportions) or different (A could be MCT oil and B squalene and IPIS or vica versa). In one embodiment, Fingolimod HCl is mixed with the oil mixture of MCT oil (5%), Squalene (2%) and IPIS (2%) in a 100 mL beaker, stirred with a spatula until the active ingredient is wetted. The resulting pre-mix is placed into sonicator (40 Mhz) for 10 min, while continuing stirring with the spatula. Absence of agglomerates is confirmed by microscopic examination. Tofacitinib is added to the oil mixture, with dispersed Fingolimod HCl, stirred with a spatula until the active ingredient is wetted. The resulting pre-mix is placed into a sonicator (40 Mhz) for 10 min, while stirring with the spatula. The result of steps 1 and 2 is referred to as the “active phase.” Absence of agglomerates is confirmed by microscopic examination.
Step 2—ST Elastomer 10 is placed into a beaker and the active phase is added thereto under stirring.
Step 3—The container which contained the active phase is rinsed with 3% MCT oil and the rinse is added to the beaker of step 3.
Step 4- Stirring is continued for approximately 10 min until the active phase is fully integrated to form a homogenous gel composition utilizing a Rayneri defloculator blade at the speed of 400 rpm.
Step 5: For gel compositions, the formulation is packaged in suitable containers.
Step 6: For foamable compositions,
   a) surfactant(s) and optionally fatty alcohol(s) and/or fatty acid(s) are added prior to step 1 the surfactant(s), fatty alcohol(s) and fatty acid(s) (if any) are added to the oil components and heated (e.g., to slightly above the melting temperature of the fatty alcohol(s) and acid(s) with stirring until they are homogenously dispersed. The mixture is allowed to cool/cooled to room temperature with stirring. The APIs can then be added to the oil component comprising surfactant etc., as indicated in step 1: and
   b) the compositions are packaged in aerosol canisters which are crimped with a valve, pressurized with propellant and equipped with an actuator suitable for foam dispensing. Optionally, a metered dosage unit can is utilized, to achieved delivery of desirable and/or repeatable measured doses of foam.
   c) pressurizing is carried out using a hydrocarbon gas or gas mixture. Canisters are filled and then warmed for 30 seconds in a warm bath at 50° C. and well shaken immediately thereafter.
Step 7: The canisters or containers are labelled.

### Experimental Method S: Reconstructed Human Epidermis Model for AD Efficacy

Reconstructed Human Epidermis (RHE) tissues are produced and grown by StratiCELL. To generate the morphological and functional aspects of AD, RHE samples are exposed to Th2 cytokines IL-4, IL-13, IL-25. Th-2 cytokines are applied for 48 h in the culture medium of epidermis. Test formulations are applied simultaneously with the cytokines on the stratum corneum of the epidermis during the same 48 h. The tissue morphology is assessed by histology and hemalum/eosin (H/E) staining—quantification of a fluorescent dye (biotin) diffusion through the epidermis in order to evaluate the barrier function.

### Example 1. Solubility of Tofacitinib Citrate in Different Excipients and Solvents

Tofacitinib citrate solubility was tested in different excipients and solvents as described in the Methods section.

As can be seen in Table 2, the dissolution of significant amounts of the drug was challenging, and only a few solvents solubilized tofacitinib citrate to a concentration typically considered sufficient for topical application (about 0.5% (5 mg/g) or higher). Among these solvents were DMSO (>15%), polyethylene glycol 400 (about 0.7%) and polyethylene glycol 200 (about 0.5%), which typically are not preferred for treating irritated or sensitive skin, such as in the case of atopic dermatitis or psoriasis.

Tofacitinib citrate's solubility was also tested in pure excipients. When preparing a full formulation, excipients with a marginal solubilization capacity, such as PEGs, were diluted and may not have provided sufficient tofacitinib solubility. Additional excipients tested included: benzyl alcohol, cyclomethicone, dimethyl isosorbate, ethanol, glycerin, isopropyl alcohol, propylene glycol, hexylene glycol, transcutol, water, 0.02% HCl, oleyl alcohol. PPG-11 Stearyl ether, diisopropyl adipate, isopropyl myristate, MCT oil, isopropyl palmitate, cetearyl ethylhexanoate, squalane, isopropyl isostearate, dimethicone presented low solubility of tofacitinib citrate. Tofacitinib citrate was not detected in 0.05% NaOH due to degradation of Tofacitinib.

TABLE 2

| | Solubility of |
|---|---|
| | TOF Citrate, |
| Solvent | mg/g |
| DMSO | >150 |
| Polyethylene Glycol 400 | 6.96 |
| Polyethylene Glycol 200 | 5.16 |
| 0.02% HCl | 4.06 |
| Water | 2.77 |
| Transcutol | 2.01 |
| Propylene Glycol | 1.70 |
| Dimethyl isosorbate | 1.27 |
| Glycerin | 0.97 |
| Benzyl Alcohol | 0.81 |
| Ethanol | 0.61 |
| Hexylene Glycol | 0.25 |
| Isopropyl Alcohol | 0.24 |
| Oleyl alcohol | 0.09 |
| PPG-11 Stearyl ether | 0.02 |
| Diisopropyl adipate | 0.02 |
| Isopropyl myristate | <0.02 |
| MCT oil (Caprylic/capric triglycerides) | <0.01 |
| Isopropyl palmitate | ND |
| Cetearyl ethylhexanoate | ND |
| Squalane | ND |
| Isopropyl isostearate | ND |
| Dimethicone (Xiameter PMX 200 Silicone Fluid 200 CST) | ND |
| Dimethicone (Xiameter PMX 200 Silicone Fluid 5 CST) | ND |
| Cyclomethicone | ND |
| 0.05% NaOH | ND |
| | (degraded) |

ND = Non-detected

### Example 2. Tofacitinib Citrate Compatibility with Different Excipients and Stability Evaluation in Aqueous Solutions at Different pH

Tofacitinib citrate mixtures with different excipients were incubated for 10 weeks at 60° C. and evaluated by tofacitinib assay and degradation products as described in the Methods section. As can be seen in Table 3a, tofacitinib citrate was not compatible with a broad range of topically acceptable excipients including surfactants, polymers, polar solvents in the presence of water. However, tofacitinib was unexpectedly found to be compatible with MCT oil and to be degraded only to a relatively small extent by water (non-buffered).

TABLE 3a

| | TOF | Deg. |
|---|---|---|
| Sample name | assay | products |
| TOF in MCT oil | 108.4 | 0.09 |
| TOF in Water + Hypermellose K100M | 14.2 | 4.64 |
| TOF in Water + Xanthan Gum | 7.7 | 28.23 |
| TOF in Water + Carboxymethylcellulose | 48.1 | 9.33 |
| TOF in Water + Methyl Paraben | 81.6 | 4.14 |
| TOF in Water + Propyl Paraben | 93.0 | 3.01 |
| TOF in Water + Glyceryl Monosteatate | 75.6 | 2.62 |
| TOF in Water + Ceteareth 20 | 67.7 | 4.57 |
| TOF in Water + Glycerin | 63.9 | 10.16 |
| TOF in Water + Stearic Acid | 79.4 | 3.64 |
| TOF in Water + Stearyl Alcohol | 73.1 | 2.21 |
| TOF in Water | 106.7 | 1.90 |

TABLE 3b

| | RRT | RRT | RRT | RRT | | Imp | RRT | RRT |
|---|---|---|---|---|---|---|---|---|
| Sample | 0.40 | 0.48 | 0.80 | 0.89 | TOF | B | 1.29 | 1.42 |
| pH 3, 1 w | | 0.10 | | 0.10 | 93.92 | 0.20 | | |
| pH 3, 2 w | 0.09 | 0.11 | | 0.09 | 100.09 | 0.30 | | 0.09 |
| pH 4, 1 w | | | | | 102.57 | 0.09 | | |
| pH 4, 2 w | | | | | 106.31 | 0.13 | | |
| pH 4.5, 1 w | | | | | 108.64 | | | |
| pH 4.5, 2 w | | | | | 99.84 | 0.10 | | |
| pH 5, 1 w | | | | | 105.06 | 0.12 | 0.09 | 0.14 |
| pH 5, 2 w | | | 0.08 | | 101.02 | 0.16 | 0.08 | 0.14 |
| pH 6, 1 w | | | | | 99.67 | 0.34 | | |
| pH 6, 2 w | | | | 0.09 | 100.09 | 0.52 | | |

RRT—Relative retention time (RRT) is the ratio of the retention time of analyte peak relative to the retention time of Tofacitinib obtained under identical conditions. Each RRT represents a specific impurity.

Tofacitinib Citrate solution in 50 mM citrate buffer of different pH, in the range of 3 to 6, was exposed to 60° C. for one or two weeks and evaluated for tofacitinib assay and degradation products. As can be seen in table 3b, a pH of 3 resulted in several impurities. A pH of 4 to 4.5 resulted in negligible amount of impurity B. To the contrary. pH 5 and 6 as well as pH 3 resulted in increased level of impurity B and appearance of additional degradation products. These results indicated only a narrow range of pH, about 4 to about 4.5 is compatible with tofacitinib citrate, which makes the formulation of the drug in topical aqueous products challenging. In one or more embodiments a pH between about 4 to about 5 is compatible with tofacitinib citrate. In one or more embodiments a pH, between about 4 to about 4.5 is compatible with tofacitinib citrate. In one or more embodiments the pH, is about 4, about 4.1, about 4.2, about 4.3, about 4.4, about 4.5, about 4.6, about 4.7, about 4.8, about 4.9 or about 5.

### Example 3. Skin Penetration of an Active Agent in Emulsion-Based and Ointment-Based Formulations

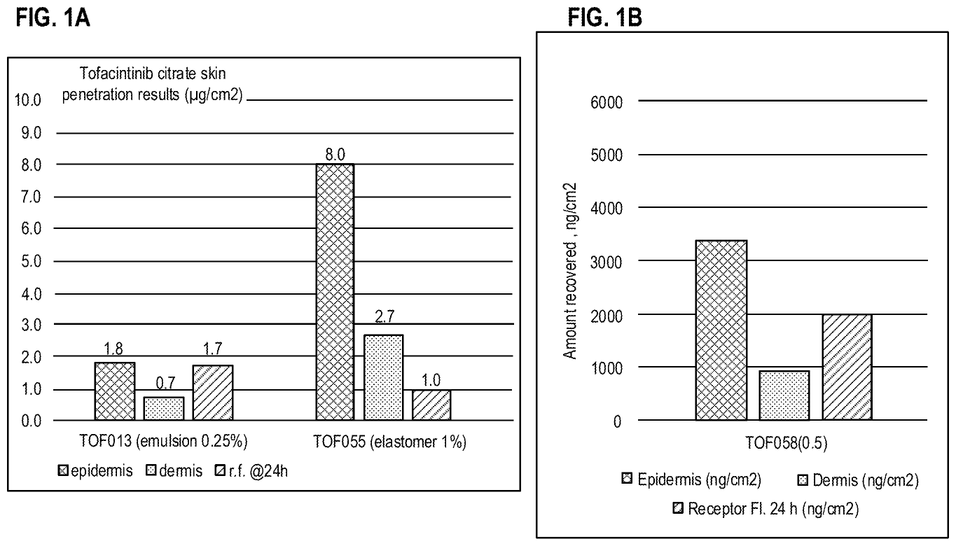

An emulsion-based formulation and an ointment-based (petrolatum) formulation (Table 4a) were tested for skin penetration of tofacitinib. Skin penetration was measured in a vertical diffusion system as described in the Methods section. As can be seen in Tables 4a, 4b and FIG. 1A when formulated in an emulsion carrier, skin penetration profile of tofacitinib showed higher delivery to receptor fluid (representing higher systemic delivery). The ratio of total skin delivery to systemic delivery in an emulsion-based carrier was only about 1.5. The ratio of dermis delivery to systemic delivery in an emulsion-based carrier was about 0.4, indicating higher delivery of tofacitinib through the skin compared to delivery to the dermis.

As can be seen in Tables 4a, 4b and FIG. 1B, when formulated in an ointment (petrolatum)-based carrier, skin penetration profile of tofacitinib showed higher systemic delivery. The ratio of total skin delivery to systemic delivery in an ointment (petrolatum)-based carrier was only about 2.2. Ratio of dermis delivery to systemic delivery in an ointment (petrolatum)-based carrier was about 0.5, indicating higher delivery of tofacitinib through the skin compared to delivery to the dermis. Without being bound by any theory it may be possible that the increased penetration through the skin is due to increased hydration of the skin, either by the water phase of the emulsion carrier or by the skin occlusive properties of petrolatum in the ointment carrier. Such increased hydration may have caused partial solubilization of the active agent resulting in its increased penetration through the skin.

Accordingly, it appeared challenging to achieve focused or targeted delivery of the drug to the epidermis and dermis, with low amounts of systemic delivery, in the case of an emulsion or an ointment formulation.

TABLE 4a

| | | | Ointment-based |
|---|---|---|---|
| | | Emulsion-based | formulation |
| | | formulation | (TOF058) |
| | | (TOF013) (0.15*) | (0.3**) |
| Ingredients | | % w/w | % w/w |
| MCT oil | | 10.00 | 13.00 |
| Stearyl alcohol | | 2.00 | — |
| Ceteareth 20 | | 3.50 | — |
| Glyceryl monostearate | | 1.50 | — |
| Hypromellose K100M | | 0.50 | — |
| Citrate buffer, 50 mM, pH 4.5 | | 76.25 | — |
| Glycerin | | 5.00 | — |
| Benzyl alcohol | | 1.00 | — |
| Petrolatum | | — | 86.50 |
| Tofacitinib citrate*** | | 0.25 | 0.50 |
| Total | | 100.00 | 100.00 |
| Results | | | |
| Amount recovered | Epidermis | 1.8 | 3.4 |
| (tofacitinib) μg/cm² | Dermis | 0.7 | 0.96 |
| | Receptor fluid | 1.7 | 2.0 |
| | Epidermis | 7.3 | 6.8 |
| | Dermis | 2.7 | 1.9 |
| Amount recovered | Receptor fluid | 6.8 | 4.0 |
| (tofacitinib) % | | | |
| applied dose | | | |

*0.15% tofacitinib corresponds to 0.25% tofactinib citrate.
**0.3% tofacitinib corresponds to 0.50% tofactinib citrate.
***non-micronized tofacitinib citrate is used for this study

TABLE 4b

| | Ratio skin to | Ratio dermis to |
|---|---|---|
| Formulation | systemic delivery | systemic delivery |
| Emulsion-based form al ation | 1.5 | 0.4 |
| (TOF013) | | |
| Ointment-based formulation | ca. 2.2 | ca. 0.5 |
| (TOF 058) | | |

### Example 4. Physical Properties of Elastomer-Based Carrier Formulations with Different Amounts of MCT Oil

Formulations with various proportions of medium chain triglycerides (MCT oil) in ST-elastomer 10 were evaluated for their physical properties. Viscosity was measured according to experimental method M Part A. As can be seen in table 5, as MCT oil content increased the viscosity of the formulation decreased resulting in liquid and/or flowable formulations. Low viscosity and/or liquid formulations are less desirable in the case of suspensions of drugs, due the higher risk of drug aggregation or sedimentation. In addition, increased amounts of MCT oil resulted in a translucent appearance. Without being bound by any theory, such translucent appearance may predict future phase separation. To the contrary, a formulation with a relatively low amount of MCT oil (about 10%) and high amount of ST-elastomer 10 (about 90%) resulted in a clear transparent gel. This last gel however, as indicated below showed a balling effect when rubbed into the skin and presented a slightly granular feel.

TABLE 5

| | OT1.0002P | OT1.0003P | OT1.0004P | OT1.0001P | OT1.0016P | OT1.0012P |
|---|---|---|---|---|---|---|
| Ingredient | % w/w | | | | | |
| ST-Elastomer 10 | 50 | 70 | 80 | 86 | 88 | 90 |
| MCT oil | 50 | 30 | 20 | 14 | 12 | 10 |
| Total | 100 | 100 | 100 | 100 | 100 | 100 |
| Results | | | | | | |
| Visual | TL | TL | TL | Slightly | TP gel | TP gel |
| appearance in a | liquid | liquid | flowable | TL | | |
| glass bottle | | | semi | gel | | |
| | | | solid | | | |
| Balling | NO | NO | NO | NO | NO | YES |

TP = Transparent; TL = Translucent

### Example 5. Physical Properties of Elastomer-Based Carrier Formulations with Different Amounts of Alternative Oils, Such as Isopropyl Palmitate and Isopropyl Myristate

Formulations with various proportions of either isopropyl palmitate or isopropyl myristate in ST-elastomer 10 were evaluated for their physical properties. Viscosity was measured according to experimental method M Part A. As can be seen in tables 6a and 6b, as isopropyl palmitate or isopropyl myristate amounts increased, the viscosity of the formulations decreased resulting in liquid and/or flowable formulations. Low viscosity and/or liquid formulations are less desirable in the case of suspensions of drugs, due the higher risk of drug aggregation or sedimentation. Formulations comprising isopropyl palmitate and isopropyl myristate exhibited greater clarity and broader compatibility with ST-elastomer 10 than formulations with MCT oil. Formulations with relatively low amounts of isopropyl palmitate and isopropyl myristate (about 10%) and high amounts of ST-elastomer 10 (about 90%) resulted in clear transparent gels. These gels however, similar to 10% MCT oil, 90% elastomer-based formulation (See example 5; OT1.0012P), showed a balling effect when rubbed into the skin and presented a slightly granular feel. To the contrary, formulations OT1.0009P (14% isopropyl myristate), OT1.0005P (14% isopropyl palmitate) and OT1.0016P (Example 5, 12% MCT oil) resulted in transparent gels with no balling effect. Accordingly, formulations comprising a mixture of ST-Elastomer and various oils can be formulated to obtain a gel without balling effect when rubbed into the skin.

TABLE 6a
Isopropyl Palmitate

| | OT1. | OT1. | OT1. | OT1. | OT1. |
|---|---|---|---|---|---|
| | 0006P | 0008P | 0007P | 0005P | 0013P |
| Ingredient | % w/w | | | | |
| ST-Elastomer | 50 | 70 | 80 | 86 | 90 |
| 10 | | | | | |
| Isopropyl | 50 | 30 | 20 | 14 | 10 |
| Palmitate | | | | | |
| Total | 100 | 100 | 100 | 100 | 100 |
| Results | | | | | |
| Visual | TL liquid | TL | TP gel | TP gel | TP gel |
| appearance | | flowable | | | |
| in a glass | | semi-solid | | | |
| bottle | | | | | |
| Balling | NO | NO | NO | NO | YES |

TP = Transparent;
TL = Translucent

TABLE 6b
Isopropyl myristate

| | OT1.0011P | OT1.0010P | OT1.0009P | OT1.0014P |
|---|---|---|---|---|
| Ingredient | % w/w | | | |
| ST-Elastomer | 70 | 80 | 86 | 90 |
| 10 | | | | |
| Isopropyl | 30 | 20 | 14 | 10 |
| myristate | | | | |
| Total | 100 | 100 | 100 | 100 |
| Results | | | | |
| Visual | TP | TP gel | TP | TP gel |
| appearance in | flowable | | | |
| a glass bottle | semi solid | | gel | |
| Balling | NO | NO | NO | YES |

TP = Transparent;
TL = Translucent

### Example 6. Binary Mixtures of MCT Oil and Alternative Oils at Different Ratios Combined with a Fixed Amount of ST-Elastomer 10

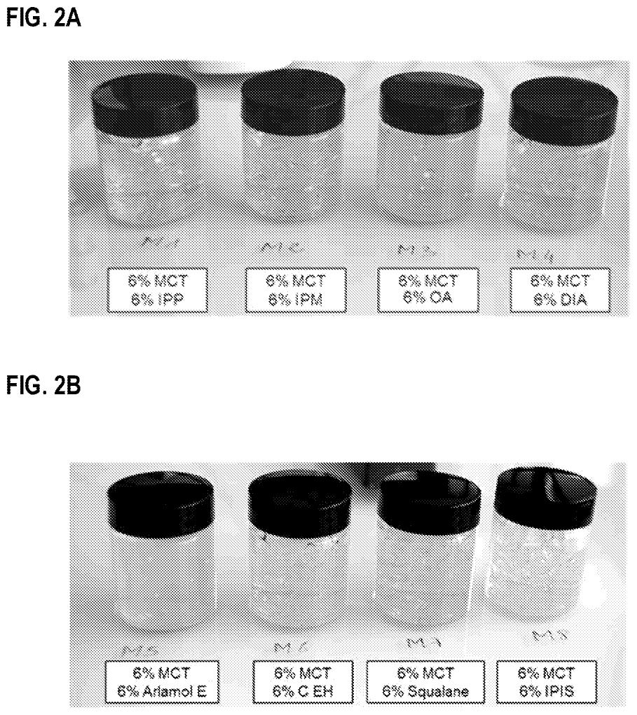

Formulations with different proportions of MCT oil and alternative oils comprising together 12% of total in ST-elastomer 10 were evaluated for their physical properties. Viscosity was measured according to experimental method M Part A. As can be seen in table 7a and FIGS. 2A and 2B, mixtures of MCT oil and isopropyl palmitate, isopropyl myristate, diisopropyl adipate, cetearyl ethylhexanoate, squalane and isopropyl isostearate at 1:1 ratio were compatible with elastomer and resulted in transparent gels. MCT oil-Oleyl alcohol mixture resulted in slightly translucent gel whereas MCT oil-PPG-15 stearyl ether mixture showed significant reduction in viscosity and translucent appearance.

As can be seen in table 7b, all mixtures containing MCT and alternative oils at 5:1 ratio, comprising together 12% of total, were compatible with ST-Elastomer 10 and resulted in transparent gels. Reduction of oleyl alcohol from 6% w/w (M3: table 7a) to 2% w/w (M15; table 7b) created a blend that was compatible with 10% w/w MCT and 88% w/w ST-elastomer 10.

TABLE 7a
Binary MCT oil—alternative oil mixtures at 1:1 ratio, combined with
ST-elastomer 10

| | M1 | M2 | M3 | M4 | M5 | M6 | M7 | M8 |
|---|---|---|---|---|---|---|---|---|
| Ingredient | % w/w | | | | | | | |
| MCT oil | 6 | 6 | 6 | 6 | 6 | 6 | 6 | 6 |
| Isopropyl | 6 | | | | | | | |
| Palmitate | | | | | | | | |
| Isopropyl | | 6 | | | | | | |
| Myristate | | | | | | | | |
| Oleyl Alcohol | | | 6 | | | | | |
| Diisopropyl | | | | 6 | | | | |
| adipate | | | | | | | | |
| PPG 15 Stearyl | | | | | 6 | | | |
| ether | | | | | | | | |
| Cetearyl | | | | | | 6 | | |
| Ethylhexanoate | | | | | | | | |
| Squalane | | | | | | | 6 | |
| Isopropyl | | | | | | | | 6 |
| Isostearate | | | | | | | | |
| ST-Elastomer 10 | 88 | 88 | 88 | 88 | 88 | 88 | 88 | 88 |
| Total | 100 | 100 | 100 | 100 | 100 | 100 | 100 | 100 |
| Results | | | | | | | | |
| Visual | TP gel | TP gel | TL gel | TP gel | TL | TP gel | TP gel | TP gel |
| appearance in a | | | | | flowable | | | |
| glass bottle | | | | | semi | | | |
| | | | | | solid | | | |

TP = Transparent; TL = Translucent

TABLE 7b
Binary MCT oil—alternative oil mixtures at 5:1 ratio, combined with
ST-elastomer 10

| | M13 | M14 | M15 | M16 | M17 | M18 | M19 |
|---|---|---|---|---|---|---|---|
| Ingredient | % w/w | | | | | | |
| MCT oil | 10 | 10 | 10 | 10 | 10 | 10 | 10 |
| Isopropyl Palmitate | 2 | | | | | | |
| Isopropyl Myristate | | 2 | | | | | |
| Oleyl Alcohol | | | 2 | | | | |
| Diisopropyl adipate | | | | 2 | | | |
| Cetearyl Ethylhexanoate | | | | | 2 | | |
| Squalane | | | | | | 2 | |
| Isopropyl Isostearate | | | | | | | 2 |
| ST-Elastomer 10 | 88 | 88 | 88 | 88 | 88 | 88 | 88 |
| Total | 100 | 100 | 100 | 100 | 100 | 100 | 100 |
| Results | | | | | | | |
| Visual appearance in a | TP gel | TP gel | TP gel | TP gel | TP gel | TP gel | TP gel |
| glass bottle | | | | | | | |

### Example 7. Tertiary Oil Blends Such as (4:1:1; MCT Oil:Oil A:Oil B) Combined with ST-Elastomer 10

Formulations with tertiary oil blends of MCT oil and alternative oils, comprising together 12% of total, in ST-elastomer 10 were evaluated for their physical properties. As can be seen in Table 8, tertiary oil blends in ST-elastomer 10 provided clear gels.

TABLE 8

| | | | | | M24 |
|---|---|---|---|---|---|
| | M20 | M21 | M22 | M23 | (OT1.021A) |
| Ingredients | % w/w | | | | |
| MCT oil | 8 | 8 | 8 | 8 | 8 |
| Isopropyl | 2 | 2 | | | |
| Myristate | | | | | |
| Oleyl Alcohol | 2 | | 2 | 2 | |
| Squalane | | 2 | 2 | | 9 |
| Isopropyl | | | | 2 | 2 |
| Isostearate | | | | | |
| ST-Elastomer 10 | 88 | 88 | 88 | 88 | 88 |
| Total | 100 | 100 | 100 | 100 | 100 |
| Results | | | | | |
| Visual appearance | TP gel | TP gel | TP gel | TP gel | TP gel |
| in a glass bottle | | | | | |

TP = Transparent;
TL = Translucent

### Example 8. Skin Penetration Study for Formulations Based on ST-Elastomer 10 and MCT Oil

Formulations comprising MCT oil, ST-Elastomer 10 and different amounts of tofacitinib citrate (non-micronized) were tested for skin penetration in a vertical diffusion system as described in the Methods section.

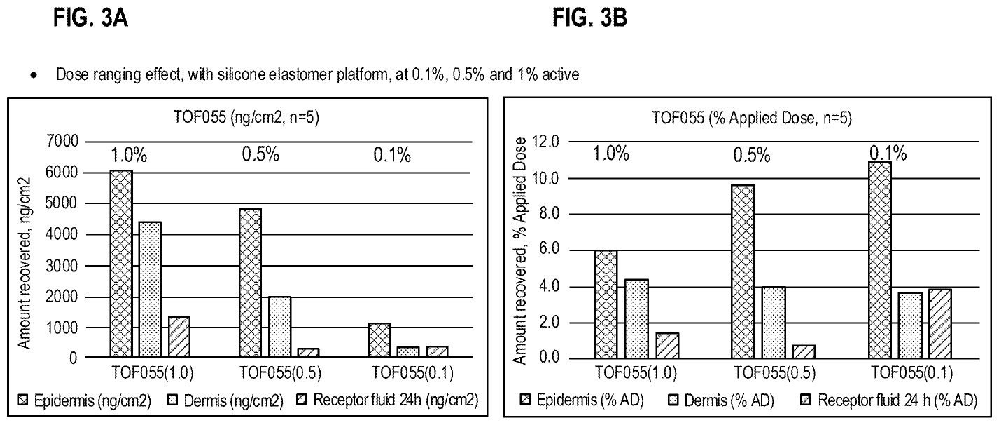

As indicated in Table 9 and FIGS. 3A and 3B tofacitinib delivery to the skin was higher than delivery through the skin (into the receptor fluid). Moreover, Elastomer-MCT oil-based formulation showed a dose-dependent delivery of tofacitinib to skin layers.

TABLE 9

| | | TOF 055 | TOF 055 | TOF 055 |
|---|---|---|---|---|
| | | (0.6*) | (0.3**) | (0.06***) |
| | | 190131S | 190210S | 190210S |
| Ingredient | | % w/w | % w/w | % w/w |
| MCT oil | | 13.00 | 13.00 | 13.00 |
| ST-Elastomer 10 | | 86.00 | 86.50 | 86.90 |
| Tofacitinib citrate | | 1.00 | 0.50 | 0.10 |
| Total | | 100.00 | 100.00 | 100.00 |
| Results | | | | |
| Amount | Epidermis | 6100 | 4850 | 1100 |
| recovered | Dermis | 4400 | 1980 | 370 |
| μg/cm² | Receptor | 1400 | 340 | 380 |
| | fluid | | | |
| Amount | Epidermis | 6.1 | 9 7 | 10.9 |
| recovered | Dermis | 4.4 | 4.0 | 3.7 |
| % applied | Receptor | 1.4 | 0.7 | 3.8 |
| dose | fluid | | | |

*0.6% tofacitinib corresponds to 1.0% tofacitinib citrate
**0.3% tofacitinib corresponds to 0.5% tofacitinib citrate
***0.06% tofacitinib corresponds to 0.1% tofacitinib citrate

### Example 9. Skin Penetration Study for Elastomer-Based Formulations with and without MCT Oil and for an Alternative Petrolatum-Based Ointment Formulation

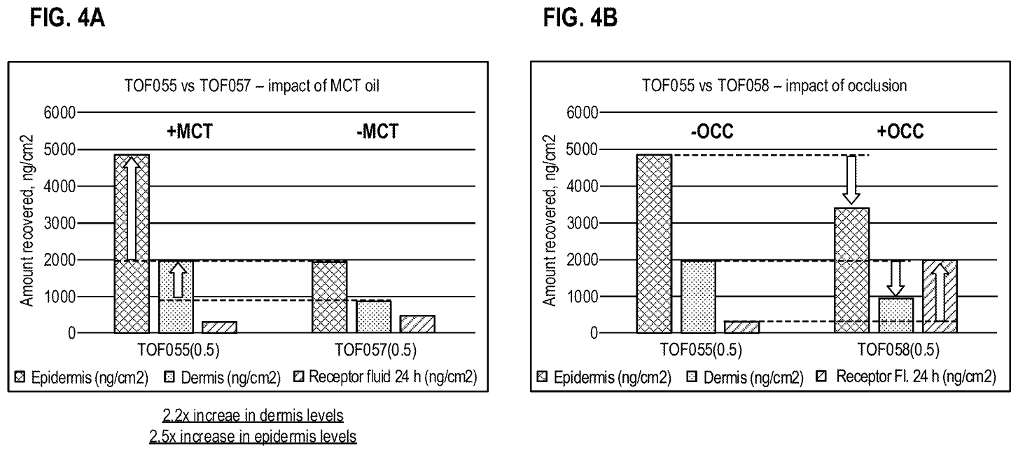

Formulations comprising ST-Elastomer 10 with and without MCT oil (TOF 055 and TOF 057, respectively) and a formulation with an occlusive agent (e.g., petrolatum) instead of elastomer (TOF 058) were tested for tofacitinib skin penetration in a vertical diffusion system as described in the Methods section. As can be seen in table 10a and FIG. 4A, a formulation based on elastomer (with cyclomethicone) resulted in tofacitinib penetration into the dermis and epidermis and to a reduced extent through the skin. Such results may indicate that elastomer itself could serve as a skin penetration enhancer. Elastomer-based-formulation with MCT oil showed increased penetration of tofacitinib to the dermis and epidermis compared to a formulation without MCT oil. Therefore, it follows that MCT oil in combination with elastomer increased penetration of tofacitinib to the dermis and epidermis.

Formulation with an occlusive agent (petrolatum) instead of elastomer resulted in a decreased penetration of tofacitinib to the skin and an increased penetration through the skin (into the receptor fluid) compared to an elastomer-based formulation with MCT oil (Table 10a and FIG. 4B). Without being bound by any theory, it may be that the occlusive agent created a barrier that prevented or reduced trans-epidermal water loss and led to increased water content in the skin, which in turn assisted solubilizing tofacitinib, leading to its increased penetration through the skin.

As can be seen from the results, MCT oil seems to help drive the active agent into the skin in the presence of elastomer but not in the presence of petrolatum (where the penetration is rather through the skin). These results indicate the combination of elastomer and MCT oil provide a unique synergistic favorable effect on penetration. Such penetration results are even more surprising when considering the fact that the active agent is suspended and not dissolved, which is known not to favor skin penetration.

TABLE 10a

| | | TOF055 (0.3*) | TOF057 (0.3*) | TOF058 (0.3*) |
|---|---|---|---|---|
| Ingredients | | % w/w | | |
| ST-Elastomer 10 | | 86.50 | 86.50 | |
| MCT oil | | 13.00 | | 13.00 |
| Cyclomethicone | | | 13.00 | |
| Petrolatum | | | | 86.50 |
| Tofacitinib citrate ** | | 0.5 | 0.5 | 0.5 |
| Total | | 100.0 | 100.0 | 100.0 |
| Results | | | | |
| Amount | Epidermis | 4.85 | 1.97 | 3.40 |
| recovered | Dermis | 1.98 | 0.90 | 0.96 |
| μg/cm² | Receptor | 0.34 | 0.48 | 2.00 |
| | fluid | | | |
| Amount | Epidermis | 9.7 | 3.9 | 6.8 |
| recovered | Dermis | 4.0 | 1.8 | 1.9 |
| % applied | Receptor | 0.7 | 1.0 | 4.0 |
| dose | fluid | | | |

*0.3% tofacitinib corresponds to 0.5% tofacitinib citrate.
**non-micronized tofacitinib citrate is used for this study

Table 10b summarizes the differences in skin delivery to systemic delivery ratio of tofacitinib for elastomer-based formulations with and without MCT oil (TOF 055 and TOF 057, respectively), for a petrolatum-based ointment formulation (TOF058) and for an emulsion formulation (TOF 013). As can be seen in table 10b, the presence of MCT oil resulted in an increase in tofacitinib penetration to the skin in about >200% (235%), >800% (814%) and >1200% (1240%) compared to elastomer-based formulation without MCT oil (TOF057), a petrolatum-based formulation (TOF058) and an emulsion-based formulation (TOF013), respectively. Similarly, the presence MCT oil resulted in an increase in tofacitinib penetration to the dermis in about >200% (205%), >1000% (1060)% and >1300% (1350)% compared to elastomer-based formulation without MCT oil (TOF057), a petrolatum-based formulation (TOF058) and an emulsion-based formulation (TOF013), respectively.

TABLE 10b

| | Skin to systemic | | Dermis to systemic | |
|---|---|---|---|---|
| | delivery | | delivery | |
| | | % Increase with | | % Increase with |
| | | Elastomer + | | Elastomer + |
| | Ratio | MCT (about) | Ratio | MCT (about) |
| Oil in water Emulsion | 1.5 | +1240% | 0.4 | +1350% |
| (TOF 013) | | | | |
| Petrolatum with MCT | 2.2 | +814% | 0.5 | +1060% |
| (TOF058) | | | | |
| Elastomer without | 6 | +235% | 1.9 | +205% |
| MCT (TOF057) | | | | |
| Elastomer with MCT | 20.1 | — | 5.8 | — |
| (TOF055) | | | | |

### Example 10. Skin Penetration Study for Elastomer-Based Formulations Comprising MCT Oil or a Combination of MCT Oil and Alternative Emollients Compared to a Comparative PEG-Ointment Formulation

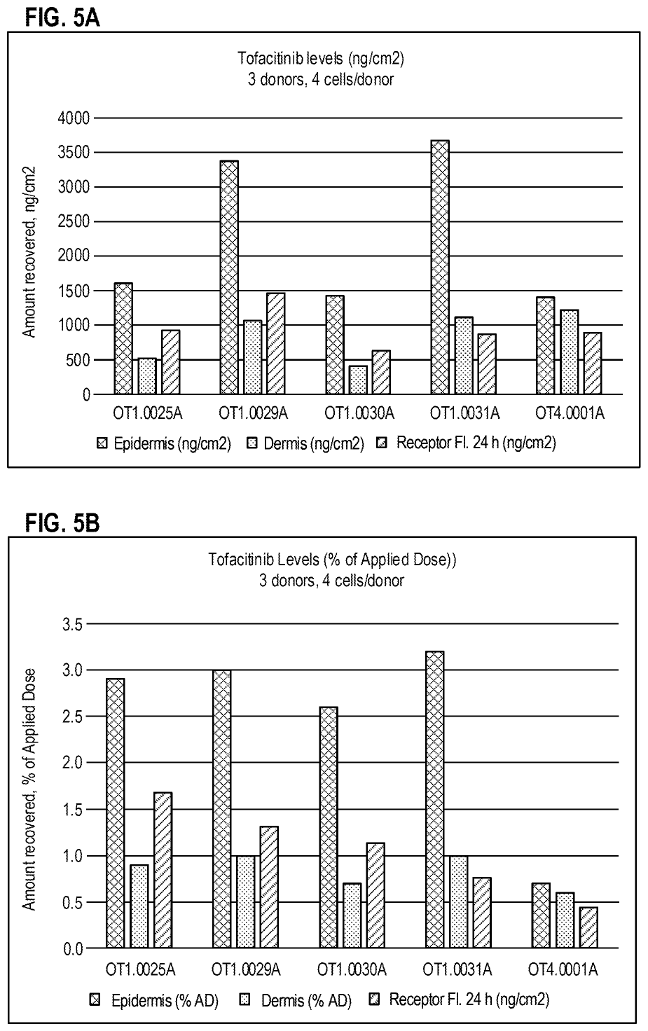

Elastomer-based tofacitinib citrate (micronized) formulations comprising MCT oil or MCT oil in combination with alternative emollients (e.g., squalane and isopropyl isostearate), and a tofacitinib base PEG-ointment were tested for skin penetration. As can be seen in table 11a and FIGS. 5A and 5B there were no significant differences in skin penetration between elastomer-based formulations with MCT oil (OT1.0025A and OT1.0029A) and elastomer-based formulations with a combination of MCT oil and alternative emollients such as squalane and isopropyl isostearate (OT1.0030A and OT1.0031A) at a comparable strength. Elastomer-based formulations at a strength of 1.2% tofacitinib (2% tofacitinib citrate; OT1.0029A and OT1.0031A) showed significantly superior penetration to the epidermis and similar penetration to the dermis as compared to 2% tofacitinib PEG ointment (OT4.0001A). Such superior penetration results are unexpected as the PEG ointment comprises a higher strength of tofacitinib compared to the elastomer-based formulations (2% vs. 1.2%). Moreover, the PEG ointment-based formulation comprised a dissolved active agent, which is expected to yield an improved skin penetration compared to a suspended active agent (as in the elastomer-based formulations). The results are further surprising as the PEG ointment comprises a free base tofacitinib that is hydrophobic and not charged and is thus more likely to penetrate the skin compared to tofacitinib citrate salt, a charged compound. Despite the lower strength, the suspended active agent and the sat form of the active agent, the elastomer-based formulations present a superior penetration profile as compared to a PEG ointment-based formulation.

TABLE 11a

| | | | | | | OT4.0001A |
|---|---|---|---|---|---|---|
| | | OT1.0025A | OT1.0029A | OT1.0030A | OT1.0031A | (2.0; PEG |
| | | (0.6-m*) | (1.2-m**) | (0.6-m*) | (1.2-m**) | ointment) |
| | | % | % | % | % | % |
| Ingredients | | w/w | w/w | w/w | w/w | w/w |
| Tofacitinib citrate | | 1 | 2 | 1 | 2 | |
| Tofacitinib free base | | | | | | 2 |
| ST-Elastomer 10 | | 87 | 86 | 87 | 86 | |
| MCT oil | | 12 | 12 | 8 | 8 | |
| Squalane | | | | 2 | 2 | |
| Isopropyl isostearate | | | | 2 | 2 | |
| PEG 400 | | | | | | 30 |
| PEG 3350 | | | | | | 30 |
| Propylene glycol | | | | | | 18 |
| Glycerin | | | | | | 17.9 |
| Oleyl alcohol | | | | | | 2 |
| BHA | | | | | | 0.1 |
| Total | | 100 | 100 | 100 | 100 | 100 |
| Results | | | | | | |
| Amount recovered | Epidermis | 1603 | 3377 | 1430 | 3673 | 1402 |
| ng/cm² | Dermis | 517 | 1070 | 408 | 1110 | 1218 |
| | Receptor | 924 | 1469 | 629 | 868 | 886 |
| | fluid | | | | | |
| Amount recovered | Epidermis | 2.9 | 3.0 | 2.6 | 3.2 | 0.7 |
| % applied dose | Dermis | 0.9 | 1.0 | 0.7 | 1.0 | 0.6 |
| | Receptor | 1.7 | 1.3 | 1.1 | 0.8 | 0.4 |
| | fluid | | | | | |

*0.6% Tofacitinib corresponds to 1% tofacitinib citrate
**1.2% Tofacitinib corresponds to 2% tofacitinib citrate
m = micronized

As can be seen in Table 11b and FIG. 5B the fraction of applied active pharmaceutical ingredient (API) delivered to the skin (out of the total applied API) in elastomer-based formulations was significantly higher than that of a PEG-based ointment.

TABLE 11b

| | Fraction of applied API |
|---|---|
| Formulation | delivered to skin |
| Elastomer based formulation | 3.6% |
| containing 0.6% tofacitinib (eq. to | |
| 1.0% tofacitinib citrate) average of | |
| formulations OT1.0025A and | |
| OT1.0030A | |
| Elastomer based formulation | 4.1% |
| containing 1.2% tofacitinib (eq. to | |
| 2.0% tofacitinib citrate) average of | |
| formulations OT1.0029A and | |
| OT1.0031A | |
| PEG Ointment based formulation | 1.3% |
| containing 2.0% tofacitinib (as | |
| tofacitinib Base) | |

### Example 11. In-Vivo Atopic Dermatitis Animal Model Study

### Part A

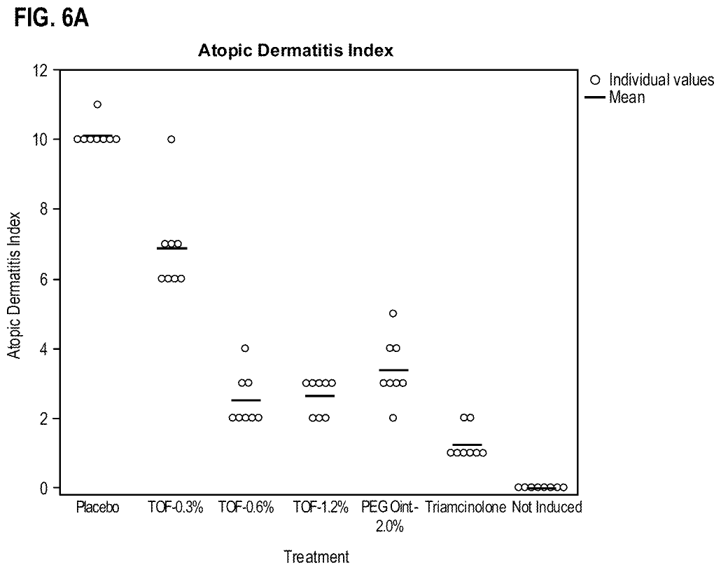

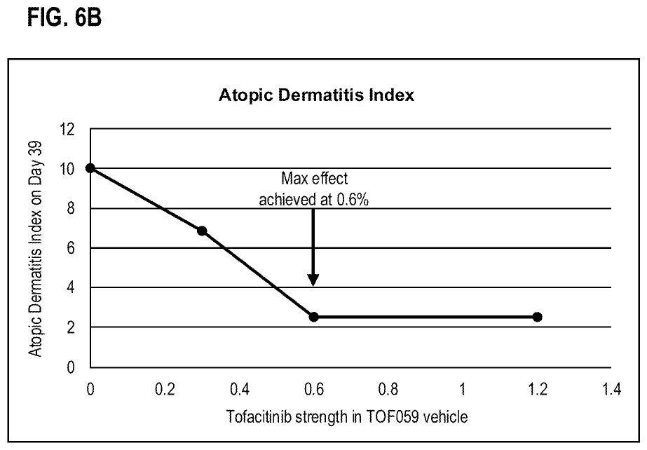

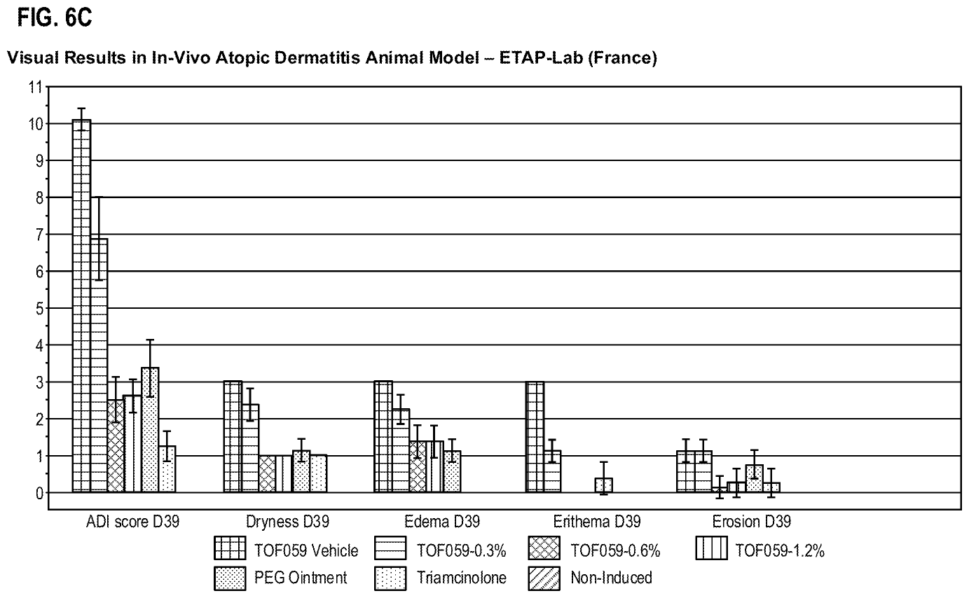

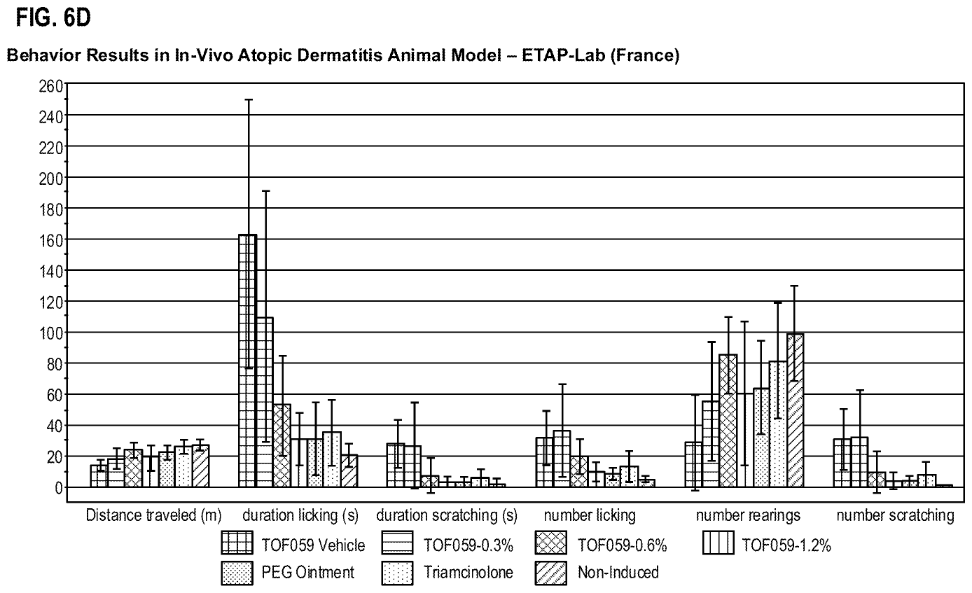

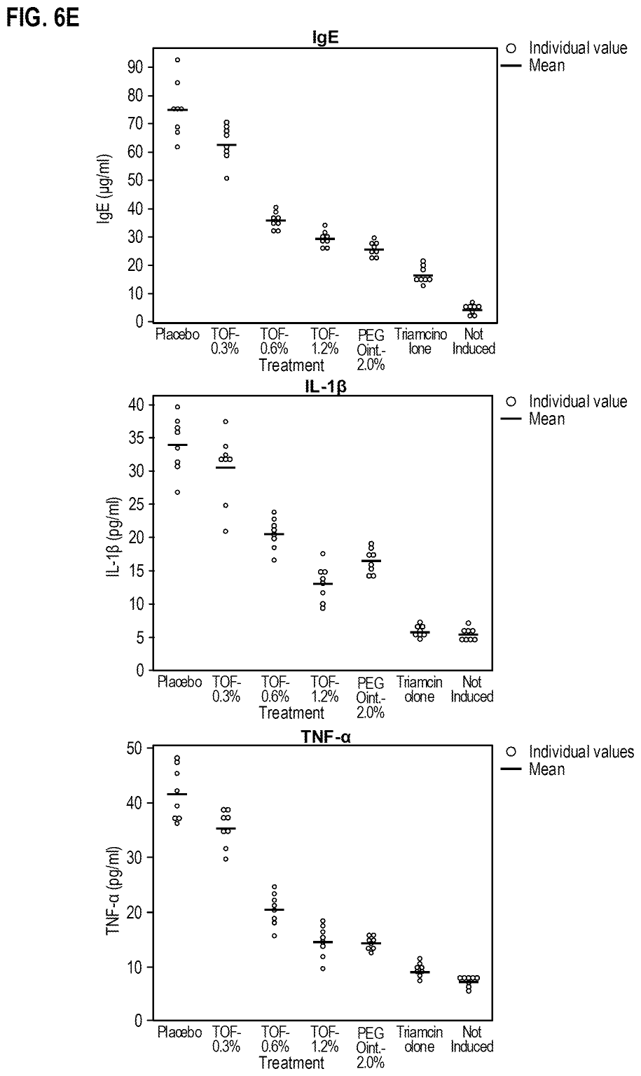

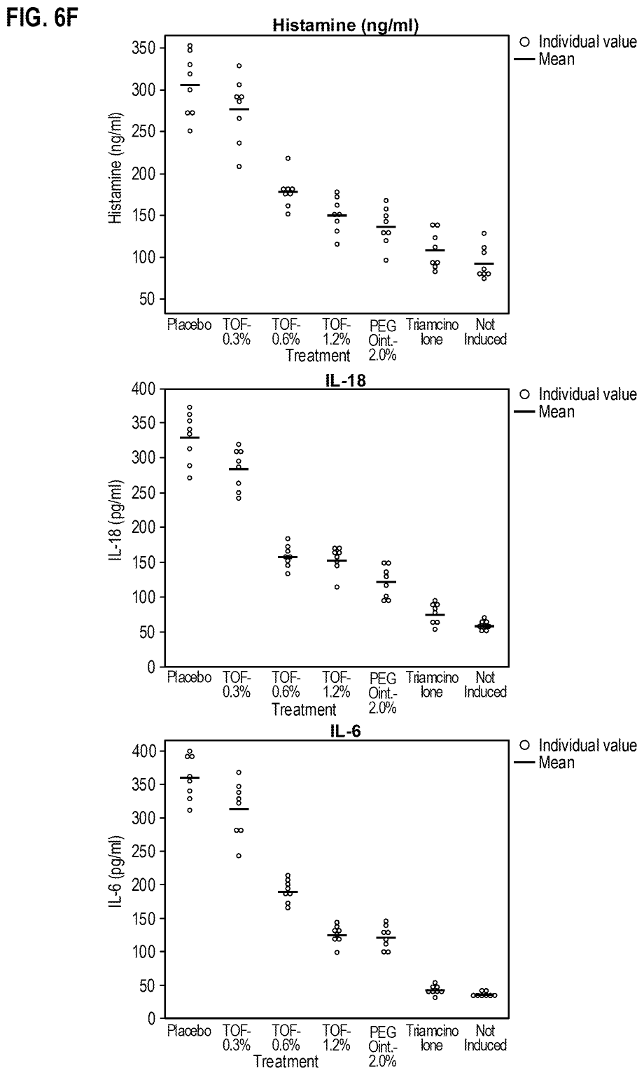

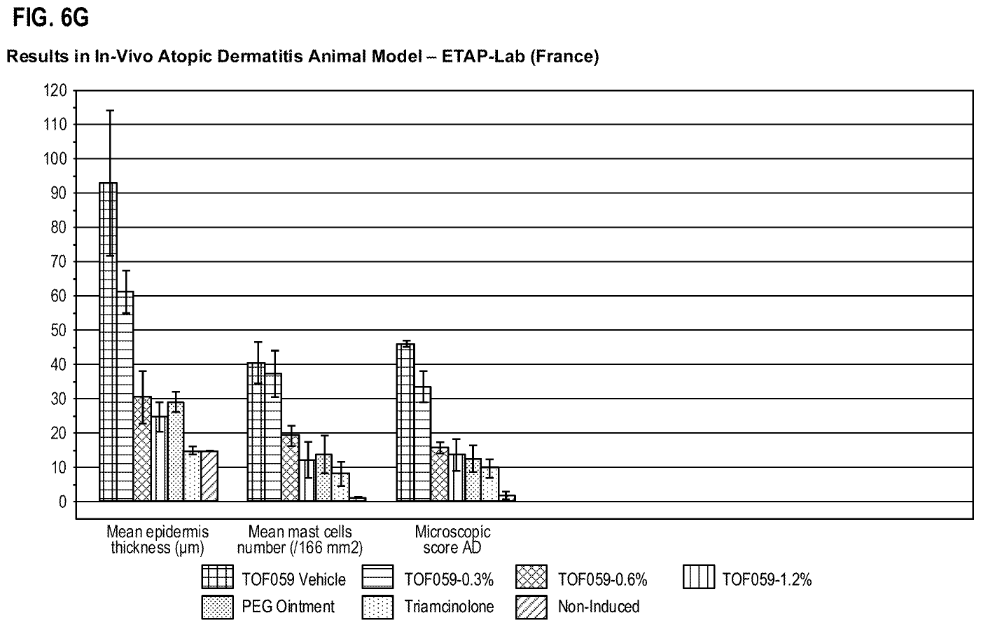

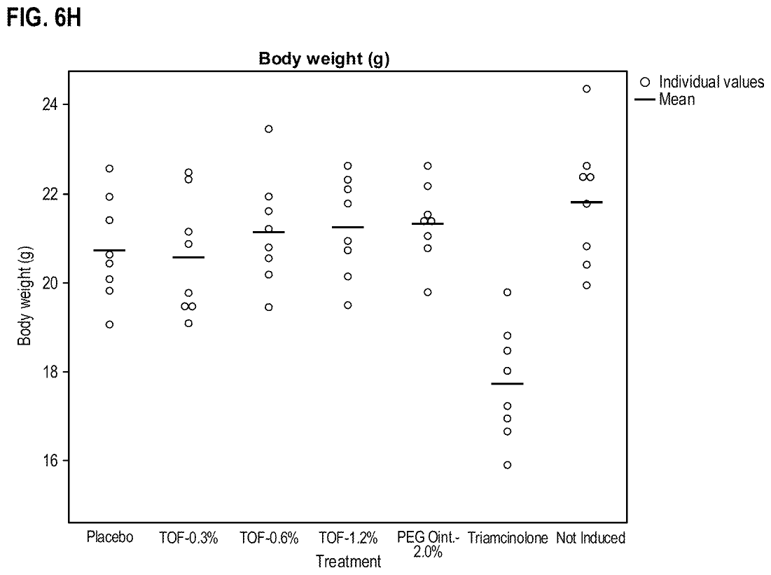

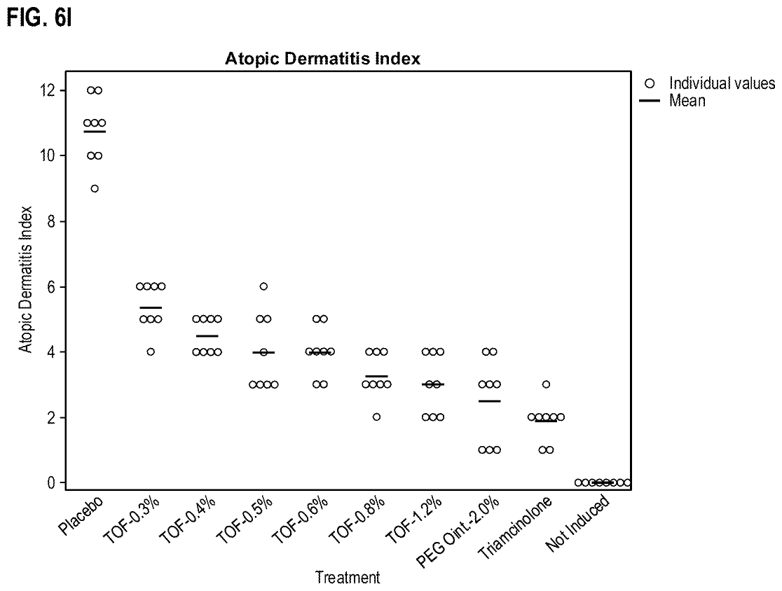

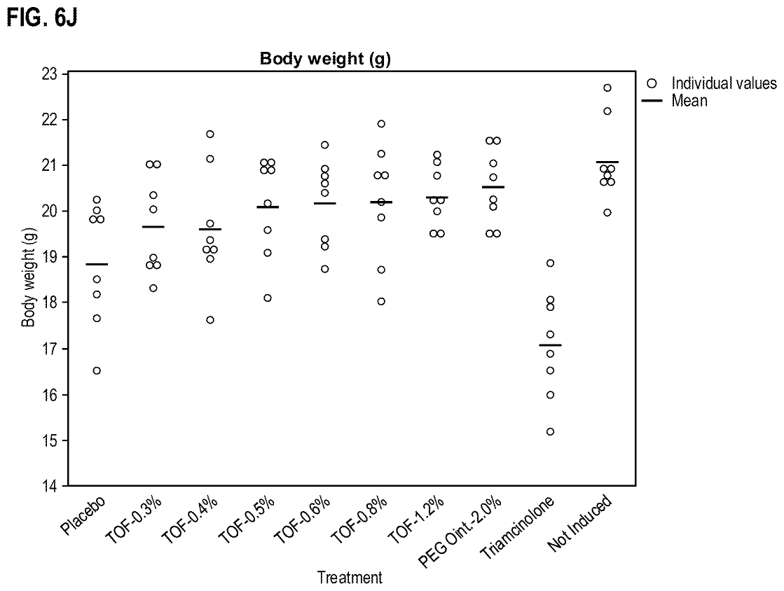

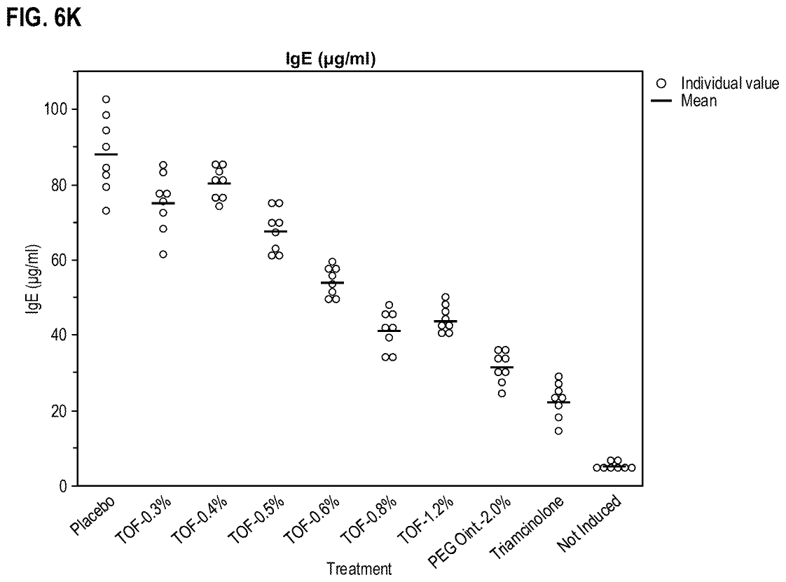

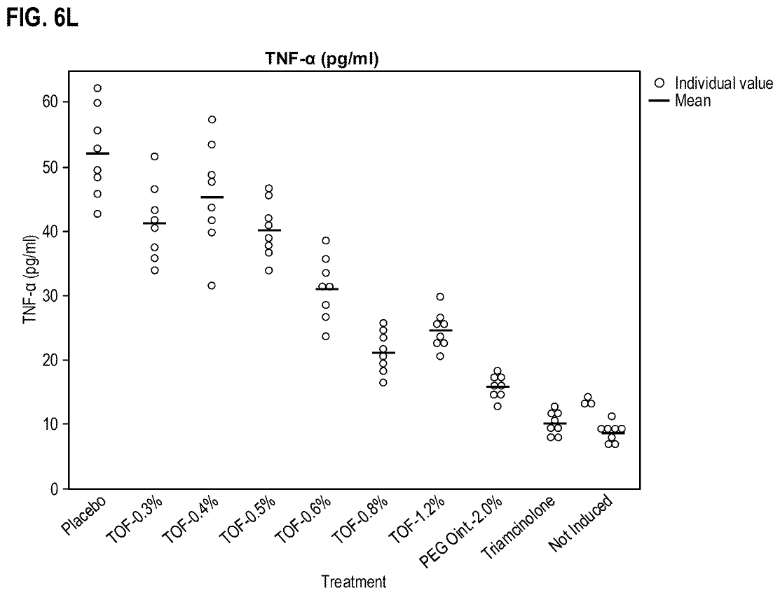

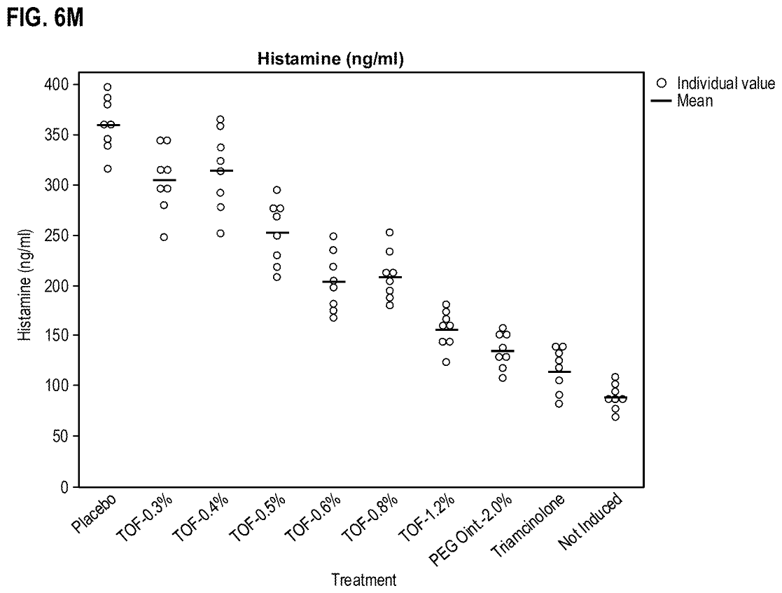

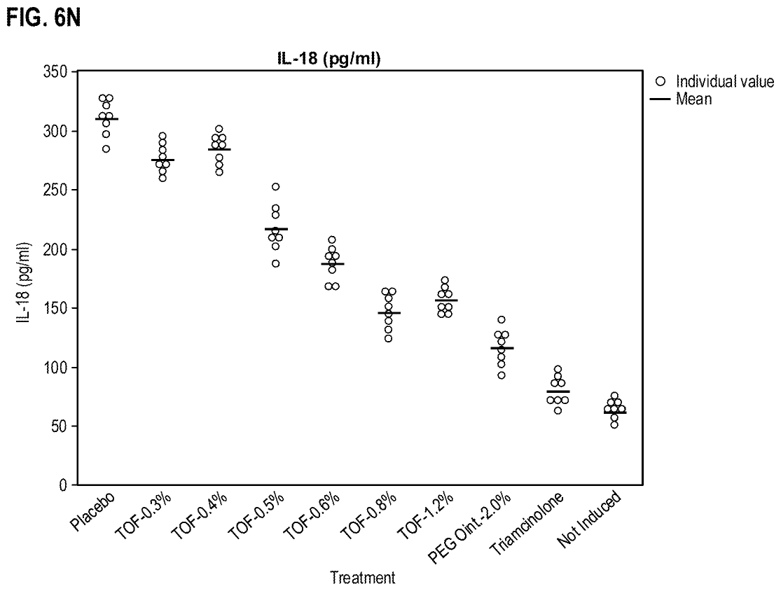

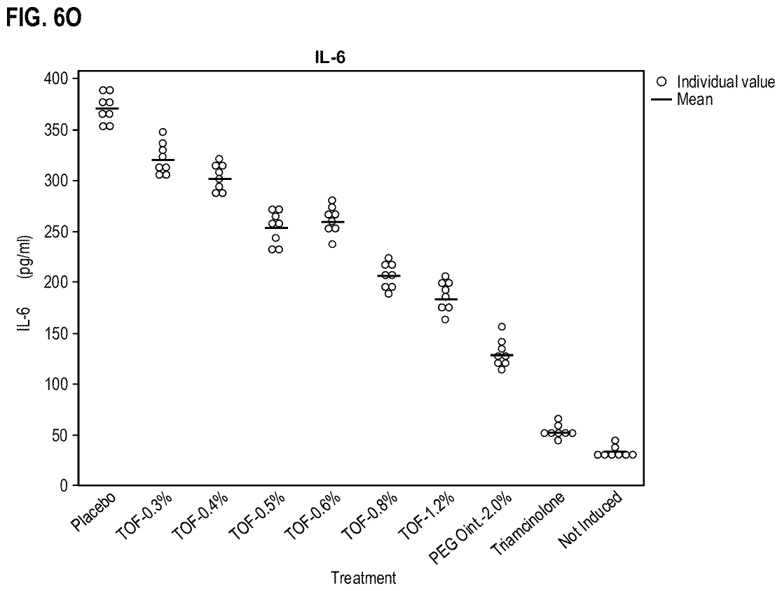

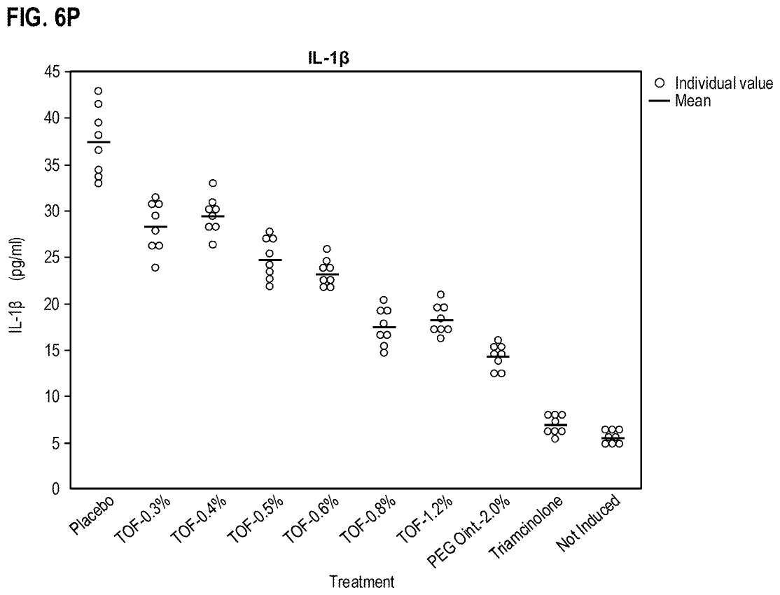

Elastomer-based formulations with MCT oil at different tofacitinib strengths were tested in an in-vivo atopic dermatitis animal model and compared to PEG ointment-based formulation and a steroid commercial product (triamcinolone acetonide 0.1% cream). As can be seen from Table 12a and FIG. 6A, elastomer-based formulations were effective in reducing Atopic Dermatitis Index (ADI). As indicated in FIG. 6B, maximum efficacy was achieved in a 0.6% tofacitinib elastomer-based formulation. Increase in tofacitinib strength to 1.2% did not result in significant reduction in ADI as compared to 0.6% tofacitinib elastomer-based formulation. This result is surprising as such increase in tofacitinib strength resulted in an increased skin penetration, see example 9. Without being bound by any theory, it may be that the JAK receptors in the dermis and epidermis become effectively saturated or almost so with tofacitinib at 0.6% concentration and therefore although skin penetration increased with 1.2% tofacitinib there was no significant observable effect on ADI. ADI for animals treated with 0.6% and 1.2% tofacitinib elastomer-based formulation was of similar compared to ADI for animals treated with 2% tofacitinib PEG ointment-based formulation. The similar effect of the elastomer-based formulations compared to the PEG ointment-based formulation was surprising as the PEG ointment-based formulation included a higher tofacitinib dose, with the drug in a dissolved state, and as a free base of tofacitinib, which was expected to provide an improved treatment for atopic dermatitis. ADI for animals treated with elastomer-based formulations was significantly higher than the index for animals treated with triamcinolone acetonide cream (2.5 and 2.6 vs. 1.3, respectively. <0.001). However, as can be seen in table 12b and FIG. 6H, Triamcinolone acetonide treatment significantly reduced animal body weight. Elastomer-based tofacitinib formulation presented beneficial effects in several of the parameters that constitute the Atopic Dermatitis Index. Such parameters include but are not limited to visual parameters (FIG. 6C) such as skin dryness, edema, erythema and erosion, behavior parameters (FIG. 6D) such as duration of licking, duration of scratching, number of licking, number of rearing and number of scratching; reduction in biomarkers related to inflammation such as IgE, IL-1β and TNF-α (FIG. 6E), histamine, IL-18 and IL-6 (FIG. 6F) and epidermis thickness, mast cell numbers and microscopic atopic dermatitis score (FIG. 6G).

TABLE 12a

| | | | TOF059 | | | | | |
|---|---|---|---|---|---|---|---|---|
| | | TOF059 | OT1.0016A | TOF059 | TOFO59 | PEG | | Not |
| | | Placebo | *0.3% m | **0.6% m | ***1.2% m | ointment | Triamcinolone | Induced |
| Ingredients | | | | | | | | |
| Tofacitinib | | | | | | 2 | | |
| Tofacitinib Citrate | | 0 | 0.5 | 1 | 2 | | | |
| ST-Elastomer 10 | | 88 | 87.5 | 87 | 86 | | | |
| MCT oil (Migliol 812N) | | 12 | 12 | 12 | 12 | | | |
| PEG 400 | | | | | | 30 | | |
| PEG 3350 | | | | | | 30 | | |
| Propylene glycol | | | | | | 18 | | |
| Glycerin | | | | | | 17.9 | | |
| Oleyl alcohol | | | | | | 2 | | |
| BHA | | | | | | 0.1 | | |
| Total | | 100 | 100 | 100 | 100 | 100 | | |
| IL-1β (pg/ml) | Mean | 33.9 | 30.5 | 20.5 | 13.1 | 16.5 | 5.7 | 5.4 |
| | S.D | 4.2 | 5.2 | 2.3 | 2.7 | 1.8 | 0.8 | 1 |
| IL-6 (pg/ml) | Mean | 359.7 | 313.1 | 189.9 | 125.2 | 120.5 | 42.5 | 35.8 |
| | S.D | 32.3 | 41.2 | 15.4 | 13.3 | 16.7 | 6.6 | 2.9 |
| IL-18 (pg/ml) | Mean | 328.9 | 284 | 157.5 | 152.2 | 121.5 | 75.3 | 58.2 |
| | S.D | 35.6 | 29.2 | 16 | 17.3 | 22.7 | 13.4 | 5.2 |
| TNF-α (pg/ml) | Mean | 41.6 | 35.4 | 20.4 | 14.5 | 14.3 | 9.1 | 7.3 |
| | S.D | 4.9 | 3.3 | 3 | 2.9 | 1.1 | 1.1 | 1 |
| IgE (μg/ml) | Mean | 75 | 62.6 | 35.8 | 29.4 | 25.5 | 16.5 | 4.3 |
| | S.D | 9.8 | 6.3 | 2.9 | 2.7 | 2.5 | 2.8 | 1.5 |
| Histamine (ng/ml) | Mean | 305.8 | 277.1 | 178 | 150.1 | 136.6 | 108.5 | 92.6 |
| | S.D | 37.7 | 38.8 | 19.3 | 20.2 | 22.7 | 22.7 | 19.3 |
| Body weight D39 (g) | Mean | 20.7 | 20.6 | 21.1 | 21.3 | 21.3 | 17.7 | 21.8 |
| | S.D | 1.2 | 1.3 | 1.2 | 1.1 | 0.9 | 1.3 | 1.4 |
| ADI D39 | Mean | 10.1 | 6.9 | 2.5 | 2.6 | 3.4 | 1.3 | 0 |
| | S.D | 0.4 | 1.4 | 0.8 | 0.5 | 0.9 | 0.5 | 0 |

*0.3% Tofacitinib corresponds to 0.5% tofacitinib citrate
**0.6% Tofacitinib corresponds to 1% tofacitinib citrate
***1.2% Tofacitinib corresponds to 2% tofacitinib citrate
m = micronized

TABLE 12b

| | Mean Body Weight |
|---|---|
| | Change |
| Treatment | d39-d32 |
| TOF059 Placebo | +0.20 |
| TOF059-1.2% | +0.90 |
| TOF059-0.3% | +0.08 |
| TOF059-0.6% | +0.74 |
| PEG Ointment | +0.89 |
| Triamcinolone | −2.80 |

(+) weight gain
(−) weight loss

### Part B—

A second study was conducted to evaluate elastomer-based formulations containing MCT oil, squalane and iso-propyl iso-stearate (IPIS) at different tofacitinib strengths (Table 12c) compared to PEG ointment formulation and a steroid commercial product (triamcinolone acetonide 0.1% cream) in an Atopic Dermatitis animal model and in accordance with the protocol set out in Methods section and the same parameters were evaluated as in the first study. The second study examined, amongst other things, the effect of addition of squalane and IPIS when compared with the first study results shown in Table 12b and provided a more comprehensive study including additional active agent concentration points.

Elastomer-based tofacitinib formulation presented beneficial effects in several parameters that constitute the Atopic Dermatitis Index, such as skin dryness, edema, erythema and erosion. Blood samples were collected on day 39 (last day of treatment) and analysed for biomarkers. A reduction in the level of inflammation biomarkers, such as IgE, IL-10 and TNF-α; histamine, IL-18 and IL-6 (Table 12C) and FIGS. 6I-6P was observed. The reduction trend of biomarkers as a function of tofacitinib concentration of the second study was consistent with that of the first study. Histological evaluation of the animal skin on day 39 (last day of treatment), which examined epidermis thickness, mast cell numbers and microscopic atopic dermatitis score showed similar trends as well (Table 12F).

As can be seen from FIG. 6I and Table 12C, elastomer-based formulations containing squalane and IPIS demonstrated reduction in Atopic Dermatitis Index (ADI D39). The efficacy of treatment, expressed in reduction of ADI versus treatment with placebo, was dose dependent, exhibiting significant efficacy (reduction of ADI) even at the lowest tested dose of 0.3% of Tofacitinib, exhibiting the highest reduction in ADI at tofacitinib concentration of 1.2%. The reduction of ADI of elastomer-based formulations of Tofacitinib in the range of strengths from 0.5% to 1.2% were within experimental variation (standard deviation) from each other and from reduction of ADI demonstrated by PEG ointment formulation, containing 2% of tofacitinib free base (Table 12C). As can be seen in Table 12D, the results of this study were consistent with observations for the first study of elastomer and MCT oil-based formulations of tofacitinib presented in Example 11 Part A (Table 12A and FIG. 6A).

As in the study presented in Example 11 Part A, the ADI reduction for animals treated with elastomer-based formulations was smaller than for animals treated with triamcinolone acetonide cream (FIG. 6I and Table 12C). However, as in the study in Example 11 Part A, treatment with Triamcinolone acetonide resulted in significant reduction in animal body weights between and Day 32 (the first day of treatment) and Day 39 (the last day of treatment), while for animals treated with tofacitinib formulations no reduction of body weight was observed (Table 12E).

In conclusion, elastomer based topical composition comprising tofacitinib demonstrated efficacy and tolerability in an AD mice animal model. A dose-dependent relationship was noted, with a significant reduction in ADI at 0.3% tofacitinib, and dose dependent efficacy noted with higher efficacy in the range 0.5-1.2% tofacitinib. The results also indicate that increasing the dose further may result in a lower index. In contrast, a steroid commercial product did not show good tolerability, as evidenced by a significant loss of animal body weight.

TABLE 12C

| | | | | | | | | 2% | | |
|---|---|---|---|---|---|---|---|---|---|---|
| | | TOF | TOF | TOF | TOF | TOF | TOF | PEG | | Not |
| | Placebo | 0.3* | 0.4* | 0.5* | 0.6* | 0.8* | 1.2* | ointment | Triamcinolone | induced |
| Ingredients | % w/w | | | | | | | | | |
| Tofacitinib | 0 | 0.5 | 0.67 | 0.83 | 1.0 | 1.33 | 2 | | | |
| Citrate** | | | | | | | | | | |
| Tofacitinib free | | | | | | | | 2 | | |
| base | | | | | | | | | | |
| ST-Elastomer 10 | 88 | 87.5 | 87.33 | 87.17 | 87 | 86.67 | 86 | | | |
| MCT oil | 8 | 8 | 8 | 8 | 8 | 8 | 8 | | | |
| Squalane | 2 | 2 | 2 | 2 | 2 | 2 | 2 | | | |
| Isopropyl | 2 | 2 | 2 | 2 | 2 | 2 | 2 | | | |
| isostearate | | | | | | | | | | |
| PEG 400 | | | | | | | | 30 | | |
| PEG 3350 | | | | | | | | 30 | | |
| Propylene glycol | | | | | | | | 18 | | |
| Glycerin | | | | | | | | 17.9 | | |
| Oleyl alcohol | | | | | | | | 2 | | |
| BHA | | | | | | | | 0.1 | | |
| Total | 100 | 100 | 100 | 100 | 100 | 100 | 100 | 100 | | |
| IL-1β (pg/ml) mean | 37.4 | 28.3 | 29.5 | 24.7 | 23.3 | 17.5 | 18.3 | 14.3 | 6.9 | 5.6 |
| S.D | 3.7 | 2.7 | 1.9 | 2.3 | 1.5 | 2 | 1.6 | 1.3 | 1 | 0.7 |
| IL-6 (pg/ml) mean | 370.9 | 320.5 | 301.5 | 253.4 | 259.8 | 206 | 183.5 | 128.7 | 52.4 | 32.3 |
| S.D | 14 | 15.2 | 12.4 | 16.1 | 12.2 | 12.5 | 12.2 | 14.2 | 6 | 4.3 |
| IL-18 (pg/ml) mean | 310.3 | 275.1 | 283.8 | 216.9 | 186.9 | 145.7 | 156.6 | 116.1 | 80.1 | 62.3 |
| S.D | 15 | 11.6 | 12.5 | 20.3 | 14 | 13.8 | 10.5 | 15.1 | 12 | 6.5 |
| TNF-α (pg/ml) | 52 | 41.3 | 45.4 | 40.1 | 31.1 | 21.2 | 24.6 | 15.8 | 10.1 | 8.7 |
| mean | | | | | | | | | | |
| S.D | 6.8 | 5.8 | 8.1 | 4.3 | 4.8 | 3.2 | 2.9 | 1.8 | 1.7 | 1.5 |
| IgE (μg/ml) mean | 87.9 | 75 | 80.3 | 67.6 | 53.9 | 41.2 | 43.7 | 31.4 | 22.3 | 5 |
| S.D | 10.1 | 7.7 | 4.2 | 5.8 | 3.6 | 5.2 | 3.1 | 4.1 | 4.4 | 0.9 |
| Histamine (ng/ml) | 360.2 | 304.5 | 314.9 | 252.6 | 203 | 208.7 | 155.3 | 134.5 | 114.1 | 87.8 |
| mean | | | | | | | | | | |
| S.D | 27 | 32.3 | 39.6 | 31.4 | 29.2 | 24.6 | 17.6 | 17.2 | 20.2 | 12.4 |
| Body weight D39 | 18.8 | 19.6 | 19.6 | 20.1 | 20.2 | 20.2 | 20.3 | 20.5 | 17.1 | 21.1 |
| (g) mean | | | | | | | | | | |
| S.D | 1.3 | 1.1 | 1.3 | 1.1 | 0.9 | 1.3 | 0.6 | 0.8 | 1.2 | 0.9 |
| ADI D39 mean | 10.75 | 5.38 | 4.5 | 4 | 4 | 3.25 | 3 | 2.5 | 1.88 | 0 |
| S.D | 1.04 | 0.74 | 0.53 | 1.2 | 0.76 | 0.71 | 0.93 | 1.31 | 0.64 | 0 |

*TOF0.3 contains 0.5% of tofacitinib citrate corresponding to 0.3% of tofacitinib base
*TOF0.4 contains 0.67% of tofacitinib citrate corresponding to 0.4% of tofacitinib base
*TOF0.5 contains 0.83% of tofacitinib citrate corresponding to 0.5% of tofacitinib base
*TOF0.6 contains 1% of tofacitinib citrate corresponding to 0.6% of tofacitinib base
*TOF0.8 contains 1.33% of tofacitinib citrate corresponding to 0.8% of tofacitinib base
*TOF1.2 contains 2% of tofacitinib citrate corresponding to 1.2% of tofacitinib base
**Tofacitinib citrate micronized is used for this study

TABLE 12D
Comparison of ADI at Day 39 between first
and second in-vivo AD study

| | ADI 039 | | | |
|---|---|---|---|---|
| | Study 2 | | Study 1 | |
| Treatment | Mean | Std Dev | Mean | Std Dev |
| Placebo | 10.75 | 1.04 | 10.1 | 0.4 |
| TOF-0.3% | 5.38 | 0.74 | 6.9 | 1 4 |
| TOF-0.4% | 4.50 | 0.53 | | |
| TOF-0.5% | 4.00 | 1.20 | | |
| TOF-0.6% | 4.00 | 0.76 | 2.5 | 0.8 |
| TOF-0.8% | 3.25 | 0.71 | | |
| TOF-1.2% | 3.00 | 0.93 | 2.6 | 0.5 |
| PEG Oint.-2.0% | 2.50 | 1.31 | 3.4 | 0.9 |
| Triamcinolone | 1.90 | 0.64 | 1.3 | 0.5 |
| Not Induced | 0 | 0 | 0 | 0 |

TABLE 12E
Mean body weight change between Day 32 and Day 39

| | Mean Body Weight Change | |
|---|---|---|
| | Day 39-Day 32 | |
| Treatment | Study 1 | Study 2 |
| Placebo | 0.20 | −0.22 |
| TOF-0.3% | 0.08 | 0.40 |
| TOF-0.4% | | 0.64 |
| TOF-0.5% | | 0.85 |
| TOF-0.6% | 0.74 | 0.96 |
| TOF-0.8% | | 1.12 |
| TOF-1.2% | 0.90 | 1.03 |
| PEG Oint.-2.0% | 0.89 | 1.31 |
| Triamcinolone | −2.80 | −2.06 |

TABLE 12F
Histological evaluation of the animal skin on day 39

| | Microscopic | Mean mast cells | |
|---|---|---|---|
| | score | number | Mean epidermis |
| Treatment | AD | (/166 mm²) | thickness (μm) |
| Placebo | 44.25 | 47.19 | 102.50 |
| TOF-0.3% | 30.88 | 36.25 | 65.31 |
| TOF-0.4% | 27.00 | 40.31 | 67.81 |
| TOF-0.5% | 26.75 | 30.00 | 55.00 |
| TOF-0.6% | 17.38 | 25.00 | 40.00 |
| TOF-0.8% | 18.00 | 27.50 | 44.38 |
| TOF-1.2% | 15.25 | 17.50 | 30.94 |
| PEG Oint.-2.0% | 15.00 | 16.56 | 34.06 |
| Triamcinolone | 11.50 | 10.94 | 14.69 |
| Not induced | 1.38 | 1.69 | 15.00 |

### Example 12. Elastomer—MCT Oil-Based Formulation and Emulsion-Based Formulation Tested in an MTT Test and an IL1-α Release Test for Cell Viability and Skin Irritation

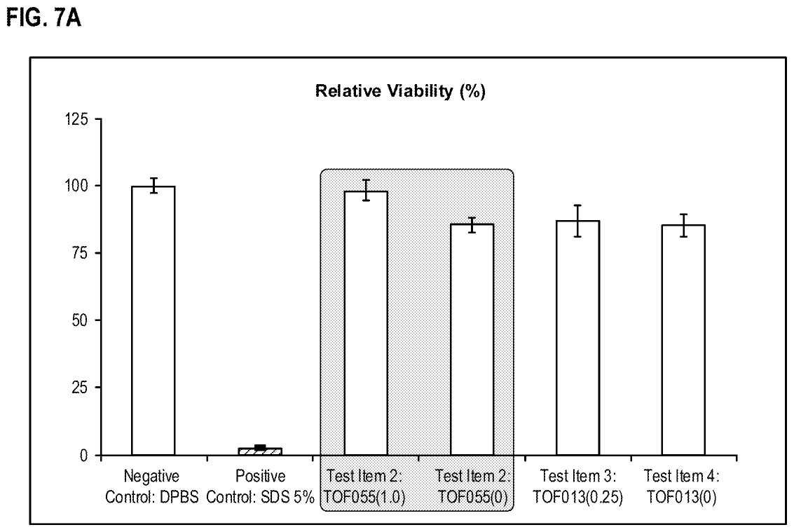

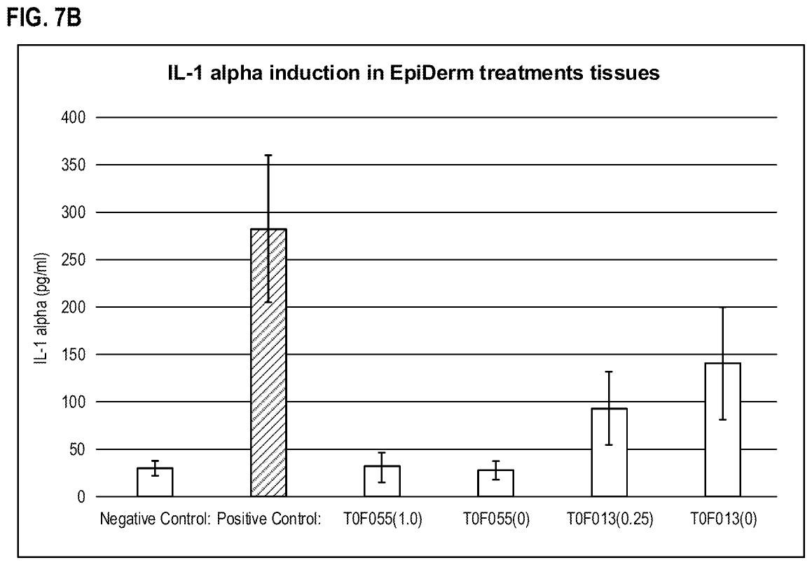

Placebo and active elastomer-based formulations comprising MCT oil and placebo and active emulsion-based formulations were tested in an MTT test and IL-1α release test (see Table 13). Formulations were compared with a concentrated soap (SDS 5%) as a positive control and a buffer (DPBS) as a negative control. As can be seen in FIG. 7A, results indicated no effect on cell viability in all formulation tested. Test articles were comparable to the negative control. In addition, as can be seen in FIG. 7B, results indicated no effect on IL1-α release, a marker for irritation, in the elastomer-based formulations that were comparable to the negative control. To the contrary, emulsion-based formulations showed increased IL1-α release compared to the negative control. Thus, these results indicated the elastomer-based formulations may have a better tolerability profile than the emulsion-based formulations.

TABLE 13

| | TOF055(0) | TOF055(0.6)* | TOF013(0.15)** | TOF013(0} |
|---|---|---|---|---|
| Ingredients | % w/w | | | |
| MCT oil | 13.00 | 13.00 | 10.00 | 10.00 |
| ST-Elastomer 10 | 87.00 | 86.00 | | |
| Tofacitinib citrate | 0 | 1.00 | 0.25 | |
| Stearyl alcohol | | | 2.00 | 2.00 |
| Ceteareth 20 | | | 3.50 | 3.50 |
| Glyceryl monostearate | | | 1.50 | 1.50 |
| Hypromellose K100M | | | 0.50 | 0.50 |
| Citrate buffer, 50 mM, pH | | | 76.25 | 76.50 |
| 4.5 | | | | |
| Glycerin | | | 5.00 | 5.00 |
| Benzyl alcohol | | | 1.00 | 1.00 |
| Total | 100.00 | 100.00 | 100.00 | 100.00 |

*0.6% Tofacitinib corresponds to 1% Tofacitinib citrate (non-micronized)
**0.15% Tofacitinib corresponds to 0.25% Tofacitinib citrate (non-micronized)

### Example 13. Elastomer Based Formulations Comprising MCT Oil in Combination with Alternative Emollients and an Oleogel-Based Formulation Tested in an MTT Test and an IL1-α Release Test for Cell Viability and Skin Irritation

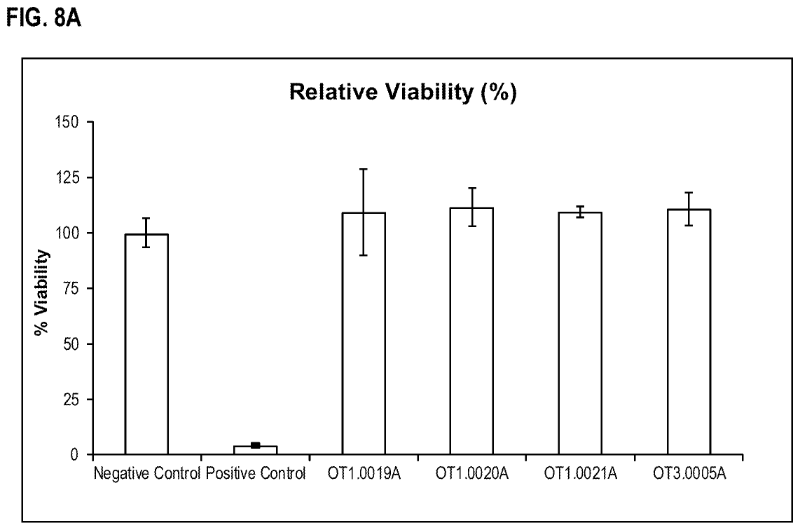

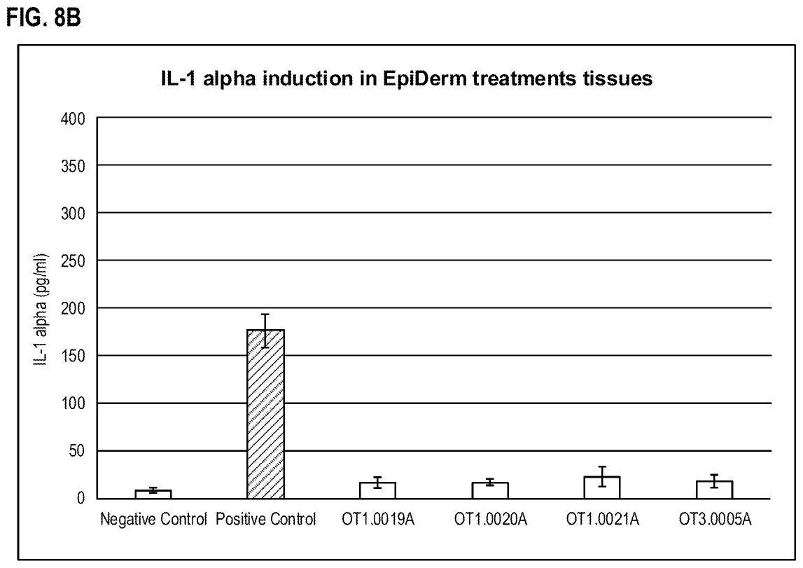

Elastomer-based formulations comprising MCT oil and alternative emollients (e.g. isopropyl palmitate, isopropyl myristate, or a combination of squalane and isopropyl isostearate) and an oleogel-based formulation were tested in an MTT test and IL-1α release test (see Table 14). Formulations were compared with a concentrated soap (SDS 5%) as a positive control and a buffer (DPBS) as a negative control. As can be seen in FIG. 8A, results indicated no effect on cell viability in all formulation tested. Test articles were comparable to the negative control. In addition, as can be seen in FIG. 8B, results indicated no effect on IL1-α release, a marker for irritation, in all formulations tested. Test articles were comparable to the negative control. Thus, results indicated the elastomer-based formulations and the oleogel-based formulation have similar tolerability profile.

TABLE 14

| | OT1.0019A | OT1.0020A | OT1.0021A | OT3.0005A |
|---|---|---|---|---|
| | (0.3*) | (0.3*) | (0.3*) | (0.3*) |
| Ingredients | % w/w | | | |
| Tofacitinib | 0.5 | 0.5 | 0.5 | 0.5 |
| Citrate** | | | | |
| ST-Elastomer 10 | 87.5 | 87.5 | 87.5 | |
| MC T oil | 10 | 10 | 8 | 19.5 |
| Isopropyl | 2 | | | 5 |
| Palmitate | | | | |
| Isopropyl | | 2 | | |
| myristate | | | | |
| Squalane | | | 2 | |
| Isopropyl | | | 7 | |
| isostearate | | | | |
| Glyceryl | | | | 16 |
| Behenate | | | | |
| Hydrogenated | | | | 2 |
| Castor Oil | | | | |
| Cyclomethicone | | | | 13 |
| White | | | | 37 |
| mineral oil | | | | |
| Zea Mays | | | | |
| Starch | | | | 7 |
| Total | 100 | 100 | 100 | 100 |

*0.3% Tofacitinib corresponds to 0.5% Tofacitinib citrate
**Non-micronized Tofacitinib citrate was used for this study

### Example 14. HET-CAM Assay (Hen's Egg-Chorioallantoic Membrane Test) for Elastomer-Based Formulations and Emulsion-Based Formulations

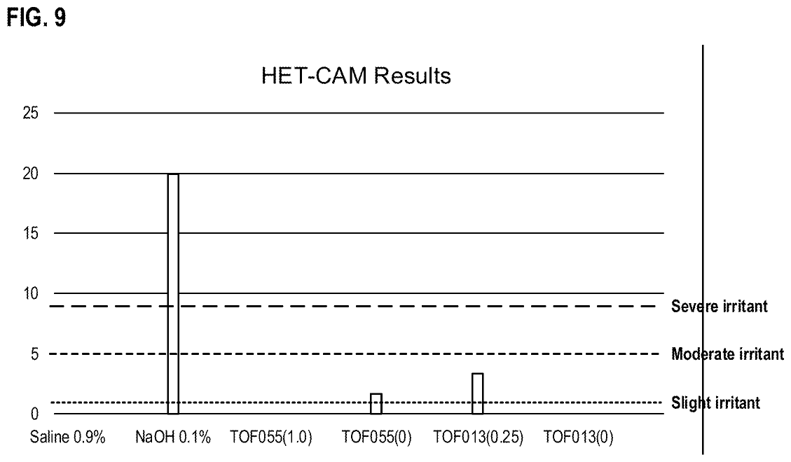

Placebo and active elastomer-based formulations comprising MCT oil and placebo and active emulsion-based formulations (see Table 13 above) were tested in a HET-CAM assay. Formulations were compared with NaOH 0.1% solution as a positive control and saline 0.9% as a negative control. As can be seen in FIG. 9, the elastomer-based active formulation showed no irritation and was classified as a non-irritant. To the contrary, the emulsion-based active formulation was classified as more than slightly irritating. Thus, these results indicated the elastomer-based formulations have a better tolerability potential than the emulsion-based formulations.

### Example 15. Day 14 Tofacitinib Plasma Levels in Minipigs Applied an Elastomer-Based Formulation

Two minipigs were treated topically once-daily for 14 days with formulation TOF055 comprising 0.3% of tofacitinib (as citrate) (Table 16a). On day 14, blood samples were collected, and plasmas were analyzed for their tofacitinib content as described in the Methods section. As can be seen in Table 16b, the highest tofacitinib concentration in the minipigs' plasma was about 2.71 ng/mL (2710 μg/mL).

TABLE 16a

| | TOF055(0.3*) |
|---|---|
| Ingredients | % w/w |
| MCT oil | 13.00 |
| ST-Elastomer 10 | 86.50 |
| Tofacitinib citrate | 0.50 |
| Total | 100.00 |

*0.3% Tofacitinib corresponds to 0.5% Tofacitinib citrate (non-micronized)

TABLE 16b

| | | Nominal | Tofacitinib |
|---|---|---|---|
| | Sampling | sampling | concentration |
| Subject no. | day | time [h] | [pg/mL] |
| PQ2001 | 14 | 0 | 675 |
| | 14 | 1 | 1050 |
| | 14 | 2 | 1080 |
| | 14 | 4 | 1720 |
| | 14 | 8 | 1320 |
| | 14 | 24 | 688 |
| PQ2101 | 14 | 0 | 2710 |
| | 14 | 1 | 2380 |
| | 14 | n | 2480 |
| | 14 | 4 | 1480 |
| | 14 | 8 | 916 |
| | 14 | 24 | 521 |

### Example 16. Stability Data of Elastomer-Based Formulations

Elastomer-based formulations were tested for active agent chemical stability for 1-3 months at 25° C., 40° C. and 50° C. As can be seen in Tables 17b-g the formulations were chemically stable for at least 1 month at 50° C. and at least 3 months at 25° C. and 40° C. These results are surprising as tofacitinib citrate was found to be incompatible with many excipients such as surfactants, polymers, polar solvent and water at acidic or basic pH. (See Example 3). For example, PEG-ointment comparative formulation (See Examples 11 and 12) requires anti-oxidants and aldehyde scavengers to stabilize the formulation. To the contrary, elastomer-based formulations do not require such stabilizers to provide chemical stability. In addition, formulations were tested for active agent distribution in different portions of the vial (product homogeneity) as described in the Methods section. Batches of the product containing 0.6% of Tofacitinib and 0.5% of Fingolimod were tested at 10 kg scale. Sampling was conducted at the end of 10 min of final mixing from the top, middle and bottom of the tank, respectively.

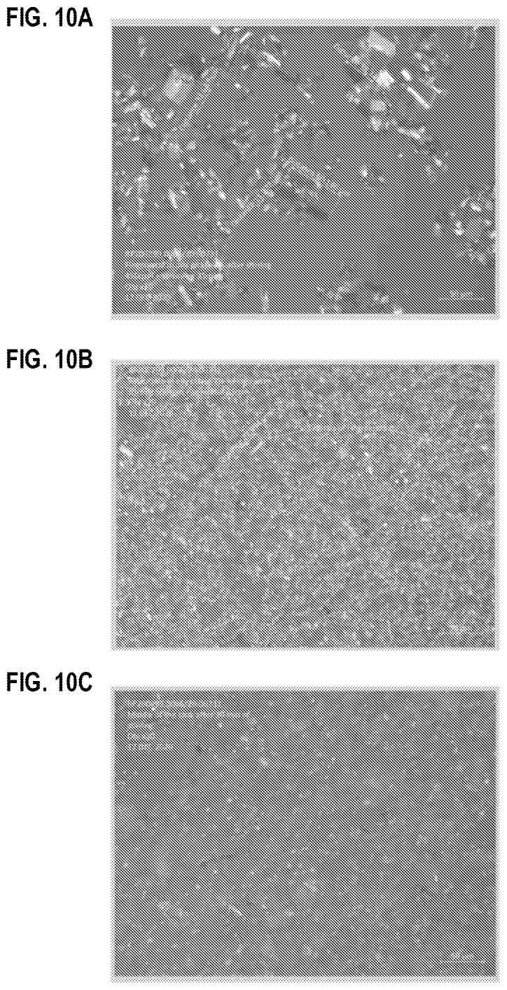

As can be seen in Table 17i, elastomer-based formulation presented a homogeneous distribution of tofacitinib throughout the packaging container. As can be seen in Table 17 h, elastomer-based formulation presented a homogeneous distribution of tofacitinib and fingolimod throughout the packaging container. This is also demonstrated in FIG. 10A-C. FIG. 10A fingolimod is seen homogenously dispersed in the oil phase alone. FIG. 10B fingolimod and tofacitinib are both homogenously dispersed in the oil phase. FIG. 10C shows tofacitinib and fingolimod are both homogenously dispersed in the final formulation. A sample taken from the middle of the tank after oil phase was added to ST Elastomer-10 and mixed for 10 minutes. These experiments confirm that both of the active pharmaceutical ingredients are distributed uniformly in the carrier.

TABLE 17a

| | OT1.0016A | OT1.0016A | OT1.0018A | OT1.0019A | OT1.0020A | OT1.0021A | OT1.0022A |
|---|---|---|---|---|---|---|---|
| | (0.3) | (0.3-m) | (0.3) | (0.3) | (0.3) | (0.3) | (0.3) |
| Ingredients | % w/w | | | | | | |
| Tofacitinib | 0.5 | 0.5 | 0.5 | 0.5 | 0.5 | 0.5 | 0.5 |
| Citrate** | | | | | | | |
| ST-Elastomer | 87.5 | 87.5 | 87.5 | 87.5 | 87.5 | 87.5 | 87.5 |
| 10 | | | | | | | |
| MCT oil | 12 | 12 | 6 | 10 | 10 | 8 | 8 |
| Isopropyl | | | 6 | 2 | | | |
| Palmitate | | | | | | | |
| Isopropyl | | | | | 2 | | |
| myristate | | | | | | | |
| Oleyl alcohol | | | | | | | 2 |
| Squalane | | | | | | 2 | 2 |
| Isopropyl | | | | | | 2 | |
| isostearate | | | | | | | |
| Total | 100 | 100 | 100 | 100 | 100 | 100 | 100 |

*0.3% Tofacitinib corresponds to 0.5% tofacitinib citrate
**Tofacitinib Citrate non-micronized was used for this study

TABLE 17b
0T1.0016A (0.3)

| | Tofacitinib content (%/T = 0) | |
|---|---|---|
| Temp | 1M | 3M |
| 25° C. | 101.3 | 100.3 |
| 40° C. | 100.3 | 103.0 |
| 50° C. | 100.7 | N/A |

TABLE 17c
OT1.0018A (0.3)

| | Tofacitinib content (%/T = 0) | |
|---|---|---|
| Temp | 1M | 3M |
| 25° C. | 102.7 | 101.3 |
| 40° C. | 103.3 | 100.3 |
| 50° C. | 102.3 | N/A |

TABLE 17d
OT1.0019A (0.3)

| | Tofacitinib content (%/T = 0) | |
|---|---|---|
| Temp | 1M | 3M |
| 25° C. | 99.0 | 100.7 |
| 40° C. | 99.7 | 100.3 |
| 50° C. | 98.0 | N/A |

TABLE 17e

| | OT1.0020A (0.3) | |
|---|---|---|
| | Tofacitinib content (%/T = 0) | |
| Temp | 1M | 3M |
| 25° C. | 102.0 | 100.7 |
| 40° C. | 100.7 | 101.0 |
| 50° C. | 101.0 | N/A |

TABLE 17f
OT1.0021A (0.3)

| | Tofacitinib content (%/T = 0) | |
|---|---|---|
| Temp | 1M | 3M |
| 25° C. | 101.6 | 98.4 |
| 40° C. | 100.6 | 100.3 |
| 50° C. | 101.0 | N/A |

TABLE 17g
OT1.0022A (0.3)

| | Tofacitinib content (%/T = 0) | |
|---|---|---|
| Temp | 1 M | 3 M |
| 25° C. | 100.0 | 100.0 |
| 40° C. | 101.0 | 99.3 |
| 50° C. | 101.6 | N/A |

TABLE 17h
OT1.0022A (0.3)

| | Tofacitinib, % | Fingolimod, % |
|---|---|---|
| Sample | LC | LC |
| Top of the tank | 100.6 | 99.3 |
| Middle of the tank | 100.5 | 98.0 |
| Bottom of the tank | 98.5 | 99.0 |

TABLE 17i
OT1.0021A (0.3) product homogeneity

| | | Tofacitinib, | |
|---|---|---|---|
| | Duplicates | % LC | % (w/w) |
| Top | E1 | 101.3% | 0.30 |
| | E2 | 102.3% | 0.31 |
| Middle | E1 | 100.9% | 0.30 |
| | E2 | 100.3% | 0.30 |
| Bottom | E1 | 101.1% | 0.30 |
| | E2 | 100.4% | 0.30 |

### Example 17. Active Agent Interfacial Tension with Stainless Steel

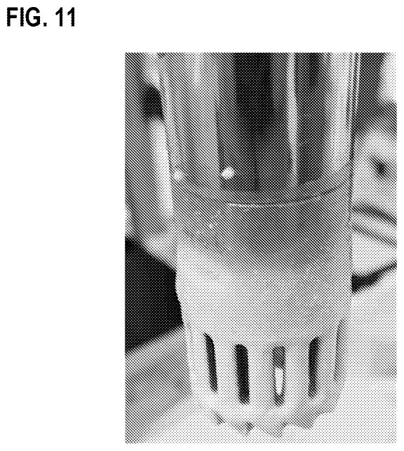

Different elastomer-based formulations (see Table 18a) were manufactured and observed. Specific formulations (OT1.0016A, OT1.0018A, and OT1.0019A) showed some accumulation of the active agent (tofacitinib citrate) on the stainless mixing propellers displayed as a white film on stainless steel propeller blades (See FIG. 11, formulation OT1.0016A, micronized API). The white film is a mixture containing formulation excipients and active agent crystals. In that film, the concentration of active agent crystals is higher than in the rest of the formulation.

Other formulations showed no or substantially less material adherence to stainless steel mixing propellers (OT1.0021A, OT1.0022A and OT1.0020A). Elastomer based formulations comprising squalane did not present active agent accumulation on the stainless-steel propellers.

TABLE 18a

| | OT1.0016A | OT1.0018A | OT1.0019A | OT1.0020A | OT1.0021A | OT1.0022A |
|---|---|---|---|---|---|---|
| | (0.3*) | (0.3*) | (0.3*) | (0.3*) | (0.3*) | (0.3*) |
| Ingredients | % w/w | | | | | |
| Tofacitinib | 0.5 | 0.5 | 0.5 | 0.5 | 0.5 | 0.5 |
| citrate | | | | | | |
| ST-Elastomer 10 | 87.5 | 87.5 | 87.5 | 87.5 | 87.5 | 87.5 |
| MCT oil | 12 | 6 | 10 | 10 | 8 | 8 |
| Isopropyl | | 6 | 9 | | | |
| Palmitate | | | | | | |
| Oleyl alcohol | | | | 2 | | 2 |
| Squalane | | | | | 2 | 2 |
| Isopropyl | | | | | 2 | |
| isostearate | | | | | | |
| Total | 100 | 100 | 100 | 100 | 100 | 100 |
| Results | | | | | | |
| Process | White film | White film | White film | Minimal | No white | No white |
| observations | on | on | on | white film | film on | film on |
| | propeller | propeller | propeller | on | propeller | propeller |
| | blade | blade | blade | propeller | blade | blade |
| | | | | blade | | |

*0.3% Tofacitinib corresponds to 0.5% tofacitinib citrate (non-micronized)

TABLE 18b

| | | | Tofacitinib | |
|---|---|---|---|---|
| | | | Citrate non- | Tofacitinib |
| | | | micronized | Citrate |
| | | | Lot | micronized Lot |
| | Overall | | TOF0000004 | TOF0000004-04 |
| | Surface | Surface | Interfacial | Interfacial |
| | Tension | Polarity | Tension | Tension |
| | (mN/m) | (%) | (mN/m) | (mN/m) |
| Stainless | 38.76 | 21.52 | 1.62 | 2.50 |
| Steel | | | | |
| OT1.0016P | 28.61 | 28.43 | 1.88 | 2.72 |
| OT1.0021P | 29.66 | 31.20 | 1.45 | 2.17 |
| OT1.0022P | 30.01 | 32.71 | 1.29 | 1.95 |

A surface tension study was performed to characterize active agent samples, various oils and specific formulations for Surface energy with polar and dispersive components. The Washburn method was used to determine contact angles and the Fowkes theory was used to calculate surface energies.

Two Tofacitinib citrate samples were tested: Tofacitinib citrate non-micronized and Tofacitinib citrate micronized. Surface tension measurements were performed on several oils and the following formulations: OT1.0016P (elastomer-MCT oil-based formulation), OT1.0021P (elastomer-based formulation comprising a combination of MCT oil, squalane and isopropyl isostearate) and OT1.0022P (elastomer-based formulation comprising a combination of MCT oil, squalane and oleyl alcohol). As can be seen in table 18b, the interfacial tensions between both tofacitinib citrate samples and the stainless steel were lower than the interfacial tension with OT1.0016P. Therefore, without being bound by any theory, this difference in interfacial tension may explain the active agent sticking to stainless steel observed with formulation OT1.0016P and not with formulations OT1.0021P and OT1.0022P (where their interfacial tensions with tofacitinib citrate samples were lower than those of the stainless steel).

Thus, the results indicated that oleyl alcohol, isopropyl isostearate and squalane may help lowering interfacial tensions with tofacitinib citrate samples and reduce stickiness/adhesion to stainless steel. Without being bound by any theory, it may be that those oils raise the overall surface tension of the oil mix and make it (in the oleyl alcohol and isopropyl isostearate cases) more polar and thereby closer in overall surface energy and surface polarity to the tofacitinib citrate samples. Accordingly, it was surprisingly found that the addition of small amounts of certain oils to the elastomer/MCT oil-based formulation, have the ability to modify the surface tension properties of tofacitinib with regards to stainless steel, to such an extent that it can effectively reduce or suppress sticking to metal surfaces.

Several preparations were made and measured. Whilst a variation in interfacial tension values of different micronization preparations was observed s, the interfacial tension between the active agent and the composition including squalane and isopropyl isostearate was consistently below that of the interfacial tension between the active agent and the stainless steel. Likewise, in the absence of squalane and isopropyl isostearate the interfacial tension between the active agent and the composition was consistently above that of the interfacial tension between the active agent and the stainless steel. One of the preparations and its measurements is presented above by way of illustration.

### Example 18. Alternative Formulations with a Reduced Amount of Elastomer

Formulations comprising a reduced amount of an elastomer component without active agents, were prepared as shown in Tables 19.

TABLE 19

| | OT2. | OT2. | OT2. | OT2. | OT2. |
|---|---|---|---|---|---|
| | 0002P | 0003P | 0004P | 0005P | 0007P |
| ST-Elastomer 10 | 15 | 15.5 | 17 | 17 | 17 |
| MCT oil | 36 | 36 | 36 | 20 | 20 |
| Isopropyl Palmitate | 21 | 21 | 21 | 7 | 7 |
| Isopropyl myristate | | | | 15 | 10 |
| Glyceryl Behenate | 16 | 16 | 14 | 16 | 16 |
| Cetearyl Isosonanoate | 10 | 10 | 10 | | |
| Hydrogenated Castor Oil | 2 | 1.5 | 2 | 2 | 2 |
| Cyclomethicone | | | | 13 | 13 |
| Oleyl alcohol | | | | 10 | 5 |
| White mineral oil | | | | | 10 |
| Total | 100 | 100 | 100 | 100 | 100 |

### Example 19. Alternative Oleogel-Based Carrier and Active Formulations

Formulations comprising an oleogel-based carrier with or without tofacitinib were prepared as shown in Table 20. In one or more embodiments the formulations illustrated in Table 20 are prepared with the addition of 0.01% fingolimod.

TABLE 20

| | | | | OT3.0005A |
|---|---|---|---|---|
| | OT3.0001P | OT3.0002P | OT3.0004P | (0.3) |
| Tofacitinib* | 0 | 0 | 0 | 0.5 |
| Citrate | | | | |
| Fingolimod** | | | | |
| HCl | | | | |
| MCT oil (Migliol | 30 | 20 | 20 | 19.5 |
| 812N) | | | | |
| Isopropyl | | | | 5 |
| Palmitate | | | | |
| (Crodamol IFF) | | | | |
| Isopropyl | | 10 | 5 | |
| myristate | | | | |
| (Crodamol IPM) | | | | |
| Glyceryl | 16 | 16 | 16 | 16 |
| Behenate | | | | |
| (Compritol 888) | | | | |
| Hydrogenated | 2 | 2 | 2 | 9 |
| Castor Oil | | | | |
| (Kaliwax HCO) | | | | |
| Cyclomethicone | 22 | 13 | 13 | 13 |
| (ST- | | | | |
| Cyclomethicone | | | | |
| 5NF) | | | | |
| Oleyl alcohol | | | | |
| (Kallicream OA) | | | | |
| White mineral | 23 | 32 | 37 | 37 |
| oil (Blandol) | | | | |
| Zea Mays Starch | 7 | 7 | 7 | 7 |
| (Amidon De | | | | |
| Mais Extra | | | | |
| Blank) | | | | |
| Total | 100 | 100 | 100 | 100 |

*0.3% Tofacitinib corresponds to 0.5% Tofacitinib citrate
**0.01% fingolimod corresponds to 0.0112% of fingolimod HCl

### Example 20. Alternative Emulsion-Based Carrier Formulations

Formulations comprising an emulsion-based carrier without active agents were prepared as shown in Tables 21a-d.

TABLE 21a

| | CR1. | CR1. | CR1. | CR1. | CR1. |
|---|---|---|---|---|---|
| | 0006P | 0009P | 0010P | 0011P | 0012P |
| Gelot 64 | 5 | 5 | 5 | 5 | 5 |
| Xantan gum | 0.3 | 0.3 | 0.3 | 0.3 | 0.3 |
| Cetyl alcohol | 2 | 2 | 2 | 2 | 2 |
| MCT oil | 6 | 6 | 6 | 6 | 6 |
| White Mineral Oil | 10 | 10 | 10 | 10 | 10 |
| Propyl paraben | 0.1 | 0.1 | 0.1 | 0.1 | 0.1 |
| Methyl paraben | 0.2 | 0.2 | 0.2 | 0.2 | 0.2 |
| Water. | 76.4 | 71.4 | 66.4 | | |
| Glycerin | | 5 | 5 | 5 | 5 |
| Transcutol | | | 5 | 5 | |
| Acetate buffer 15 mM | | | | 66.4 | 71.4 |
| Total | 100 | 100 | 100 | 100 | 100 |

TABLE 21b

| | CR1.0013P | CR1.0014P | CR1.0020P | CR1.0021P | CR1.0022P | CR1.0023P |
|---|---|---|---|---|---|---|
| Gelet 64 | 5 | 5 | 5 | 5 | 5 | 5 |
| Xantan gam | 0.3 | 0.3 | 0.3 | 0.3 | 0.3 | 0.3 |
| Cetyl alcohol | 2 | 2 | 2 | 2 | 2 | 2 |
| MCT oil | 6 | 6 | 6 | 6 | 6 | 6 |
| White Mineral Oil | 10 | 10 | 10 | 10 | 10 | 10 |
| Propyl paraben | 0.1 | 0.1 | 0.1 | 0.1 | 0.1 | 0.1 |
| Methyl paraben | 0.2 | 0.2 | 0.2 | 0.2 | 0.2 | 0.2 |
| Water | 66.4 | | 68.9 | | | |
| Glycerin | 5 | 5 | 5 | 5 | 5 | 5 |
| Transcutol | | | | 5 | | |
| Propylene glycol | 5 | 5 | | | | 5 |
| Acetate buffer 15 mM | | 66.4 | | 68.9 | 63.9 | 63.9 |
| Cyclodextrines | | | 2.5 | 2.5 | 2.5 | 2.5 |
| (1% β + 1.5% HP-Y) | | | | | | |
| Total | 100 | 100 | 100 | 100 | 100 | 100 |

TABLE 21c

| | CR2.0006P | CR2.0009P | CR2.0012 | CR2.00113P | CR2.0014P | CR2.0015P | CR2.0016P |
|---|---|---|---|---|---|---|---|
| Brij S2 | 3 | 3 | 3 | 3 | 3 | 3 | 3 |
| Brij S721 | 2 | 2 | 2 | 2 | 2 | 2 | 2 |
| Stearyl alcohol | 1 | 1 | 1 | 1 | 1 | 1 | 1 |
| Stearic acid | 1.5 | 1.5 | 1.5 | 1.5 | 1.5 | 1.5 | 1.5 |
| Isopropyl palmitate | 5 | 5 | 5 | 5 | 5 | 5 | 5 |
| Arlamol PS11E | 4 | 4 | 4 | 4 | 4 | 4 | 4 |
| Dimethicone 350 cst | 1 | 1 | 1 | 1 | 1 | 1 | 1 |
| Propyl paraben | 0.1 | 0.1 | 0.1 | 0.1 | 0.1 | 0.1 | 0.1 |
| Methyl paraben | 0.2 | 0.2 | 0.2 | 0.2 | 0.2 | 0.2 | 0.2 |
| Water | 72.2 | 77.2 | 72.2 | | 74.7 | | |
| Glycerin | 5 | 5 | 5 | 5 | 5 | 5 | 5 |
| Transcutol | | | 5 | | | 5 | |
| Propylene glycol | 5 | | | | | | 5 |
| Cyclodextrines | | | | | 2.5 | 2.5 | 2.5 |
| (1% β + 1.5% HP-Y) | | | | | | | |
| Acetate buffer 15 mM | | | | 77.2 | | 69.7 | 69.7 |
| Total | 100 | 100 | 100 | 100 | 100 | 100 | 100 |

TABLE 21d

| | TG1.0003P | TG1.0004P | TG1.0005P | TG1.0006P | TG1.0007P | TG1.0008P | TG1.0009P |
|---|---|---|---|---|---|---|---|
| Octyldodecanol | 1 | 1 | 1 | 1 | 1 | 1 | 1 |
| Isopropyl | 4 | 4 | 4 | 4 | 4 | 4 | 4 |
| palmitate | | | | | | | |
| MCT oil | 5 | 5 | 5 | 5 | 5 | 5 | 5 |
| (Migliol 812) | | | | | | | |
| Beeswax | 0.5 | 0.5 | 0.5 | 0.5 | 0.5 | 0.5 | 0.5 |
| Sorbitan oleate | 4 | 4 | 4 | 4 | 4 | 4 | 4 |
| Cithrol DPHS | 1 | 1 | 1 | 1 | 1 | 1 | 1 |
| Sepineo P600 | 4 | 4 | 4 | 4 | 4 | 4 | 4 |
| Phenoxyethanol | 1 | 1 | 1 | 1 | 1 | 1 | \| |
| Propyl paraben | 0.1 | 0.1 | 0.1 | 0.1 | 0.1 | 0.1 | 0.1 |
| Methyl | 0.2 | 0.2 | 0.2 | 0.2 | 0.2 | 0.2 | 0.2 |
| paraben | | | | | | | |
| Water | 74.2 | 69.2 | 69.2 | | | | |
| Glycerin | 5 | 5 | 5 | 5 | 5 | 5 | 5 |
| Transeutol | | 5 | | 5 | | 5 | |
| Propylene | | | 5 | | 5 | | 5 |
| glycol | | | | | | | |
| Cyclodextrines | | | | | | 2.5 | 2.5 |
| (1% B + 1.5% | | | | | | | |
| HP-Y) | | | | | | | |
| Acetate buffer | | | | 69.2 | 69.2 | 66.7 | 66.7 |
| 15 mM | | | | | | | |
| Total | 100 | 100 | 100 | 100 | 100 | 100 | 100 |

### Example 21. Microscopic Evaluation of an Elastomer-Based Formulation Comprising MCT Oil and Additional Emollients with a Micronized Tofacitinib Citrate

An elastomer-based formulation comprising a micronized tofacitinib citrate (Table 22) was prepared and examined under a microscope. As can be seen in FIG. 12, no or low agglomeration was observed. The majority of tofacitinib particles (>95%) were in a diameter of less than 25 μm. This result is surprising as decreasing particle size often results in increased Van der Waals' interactions and electrostatic attraction between particles leading to particles agglomeration. Hence, micronized particles are often considered prone to agglomeration, compromising the increase in surface area gained by micronization. The result of this experiment showed that an elastomer-based formulation with a micronized tofacitinib citrate could be formulated without significant agglomeration of the active agent.

TABLE 22

| | OT1.0031A |
|---|---|
| | (1.2-m**) |
| Ingredients | % w/w |
| Tofacitinib citrate | 2 |
| Tofacitinib free base | |
| ST-Elastomer 10 | 86 |
| MCT oil | 8 |
| Squalane | 2 |
| Isopropyl isostearate | 2 |
| Total | 100 |

**1.2% Tofacitinib corresponds to 2% Tofacitinib citrate
m = micronized

### Example 22. In-Vivo Psoriasis Animal Model for Testing Elastomer-Based Formulations with Different Strengths of Active Agent

Investigation of the effect of the different formulations (Table 23) in a Psoriasis animal model was undertaken in accordance with the protocol set out in Methods section. The investigation addressed, amongst other things, the effect of the different active agent concentrations on the various parameters of the Psoriasis animal model. Elastomer-based formulations with MCT oil, isopropyl isostearate and squalane, at different tofacitinib strengths were tested in an in-vivo psoriasis animal model and compared to three control arms (i) PEG ointment-based formulation (ii) a calcipotriol commercial ointment (Daivonex) and (iii) a calcipotriol+betamethasone dipropionate commercial ointment (Daivobet).

TABLE 23

| | | OT1.0021 | | | PEG | | | |
|---|---|---|---|---|---|---|---|---|
| | | TOF- | TOF- | TOF- | oint | | | |
| | Placebo | 0.3%* | 0.6%* | 1.2%* | 2% | | | Not |
| Ingredients | % w/w | | | | | Daivonex | Daivobet | Induced |
| Tofacitinib Citrate | 0 | 0.5 | 1 | 2 | — | | | |
| Tofacitinib free base | — | — | — | — | 2 | | | |
| ST-Elastomer 10 | 88 | 87.5 | 87 | 86 | — | | | |
| MCT oil | 8 | 8 | 8 | 8 | — | | | |
| Squalane | 2 | 2 | 2 | 2 | — | | | |
| Isopropyl isostearate | 2 | 2 | 2 | 2 | — | | | |
| PEG 400 | — | — | — | — | 30 | | | |
| PEG 3350 | — | — | — | — | 30 | | | |
| Propylene glycol | — | — | — | — | 18 | | | |
| Glycerin | — | — | — | — | 17.9 | | | |
| Oleyl alcohol | — | — | — | — | 2 | | | |
| BHA | — | — | — | — | 0.1 | | | |
| Total | 100 | 100 | 100 | 100 | 100 | | | |
| PASI (mean) | 11 | 10.4 | 7.6 | 3.4 | 2.1 | 1.3 | 0.3 | 0 |
| Std Dev | 0.5 | 0.7 | 1.3 | 0.5 | 0.4 | 0.7 | 0.5 | 0 |
| Body weight mean)D14 (g) | 17 | 16.8 | 16.4 | 17.3 | 17.0 | 15.0 | 14.7 | 19.5 |
| Std Dev | 1.4 | 0.9 | 0.6 | 1.2 | 1.5 | 0.9 | 0.8 | 0.8 |
| Body weight difference | −0.2 | −0.2 | −0.6 | 0.3 | −0.1 | −2 | −2.8 | 2 |
| mean)D14 − D1 (g) | | | | | | | | |
| Std Dev | 0.8 | 0.6 | 0.8 | 0.7 | 0.6 | 0.7 | 0.6 | 0.6 |

*0.3% tofacitinib corresponds to 0.5% tofacitinib citrate
*0.6% tofacitinib corresponds to 1% tofacitinib citrate
*1.2% tofacitinib corresponds to 2% tofacitinib citrate

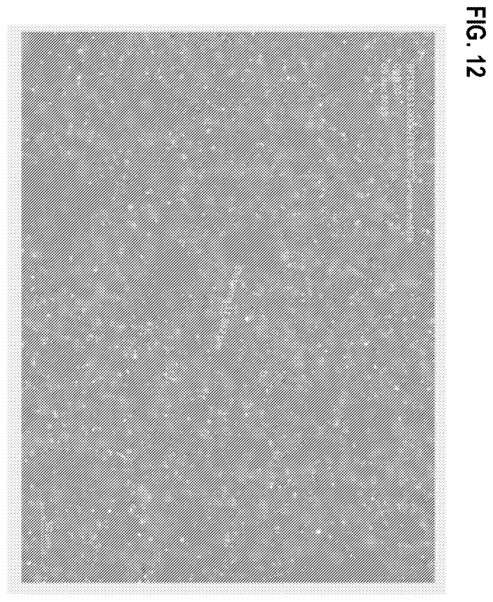

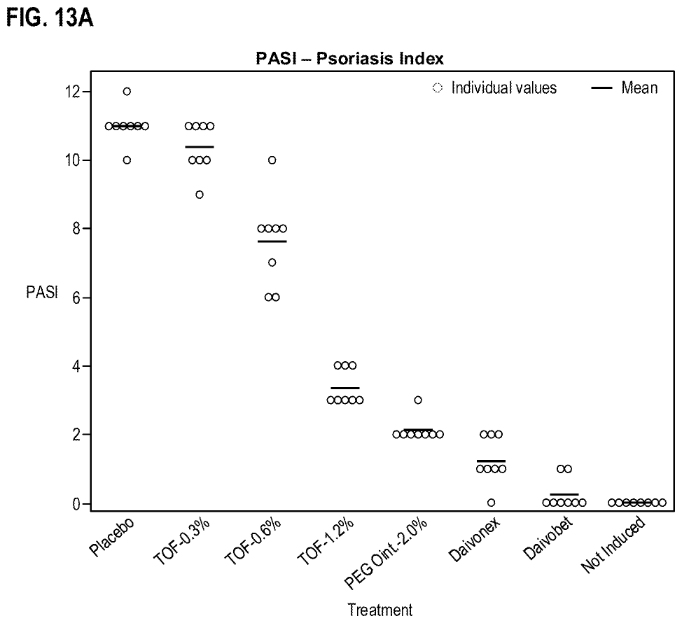

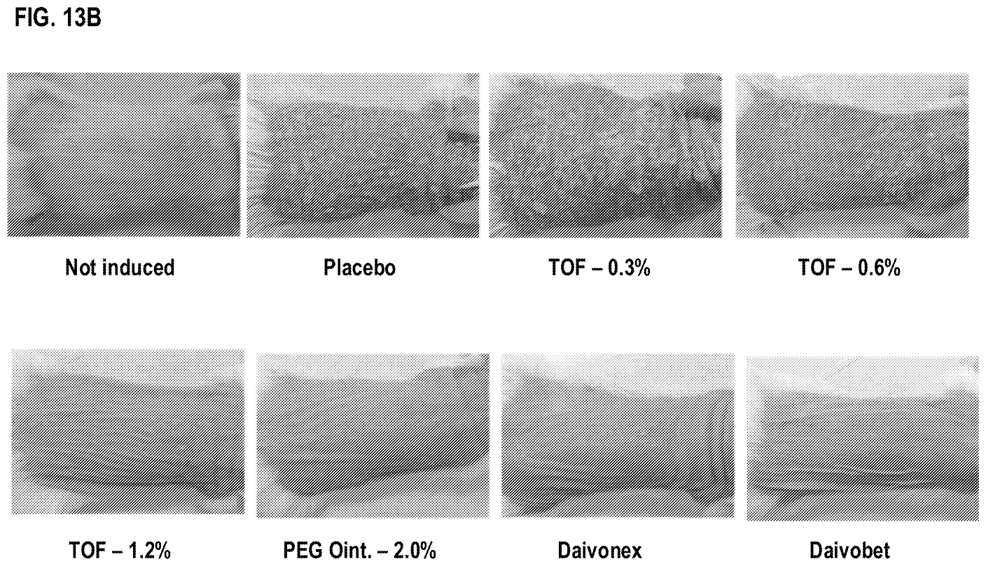

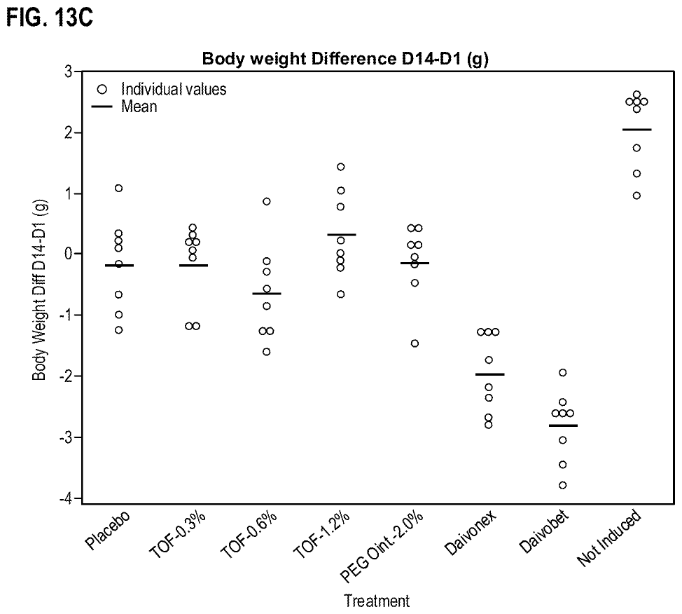

Measurements were taken during treatment period from day 7 to day 14: A progressive reduction in PASI score was observed from day 7 to day 14 (not shown) and results are presented as of day 14. As can be seen from FIGS. 13A and 131B, elastomer-based formulations were effective in reducing Psoriasis Index (PASI). A dose-response was observed for the tofacitinib elastomer-based formulations, with a higher efficacy achieved at 1.2% strength. The 1.2% tofacitinib elastomer-based formulation had a PASI score similar to the 2.0% PEG ointment. PASI for animals treated with elastomer-based formulations was higher than the index for animals treated with Daivonex or Daivobet. However, as can be seen in FIG. 13C, Daivonex and Daivobet treatments reduced animal body weight compared to the tofacitinib treatments at day 14 compared to day one, which indicates that the tofacitinib treatments were better tolerated. In addition, as can be seen in FIG. 13B, skin thinning was observed for the mice treated with Daivobet, which is a known side-effect of topically applied steroids.

### Example 23. Skin Penetration Study for Elastomer-Based Formulations Comprising Different Oils

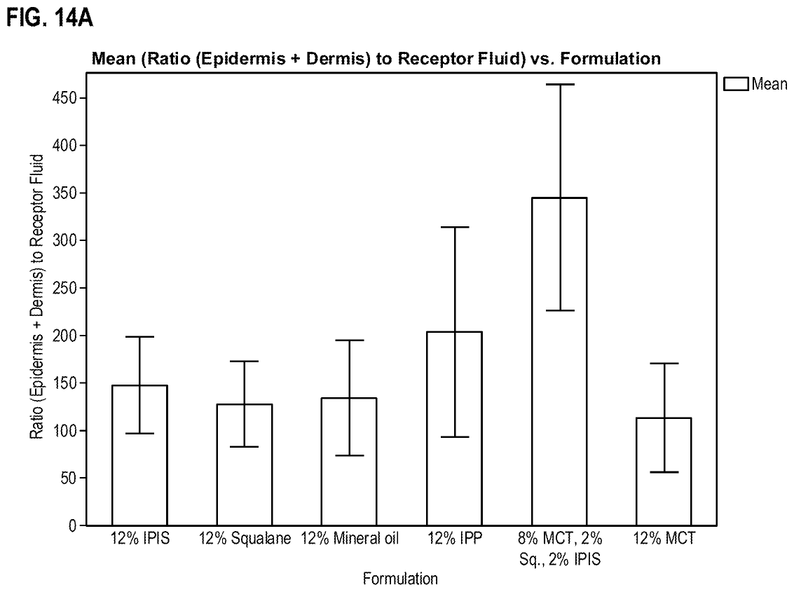

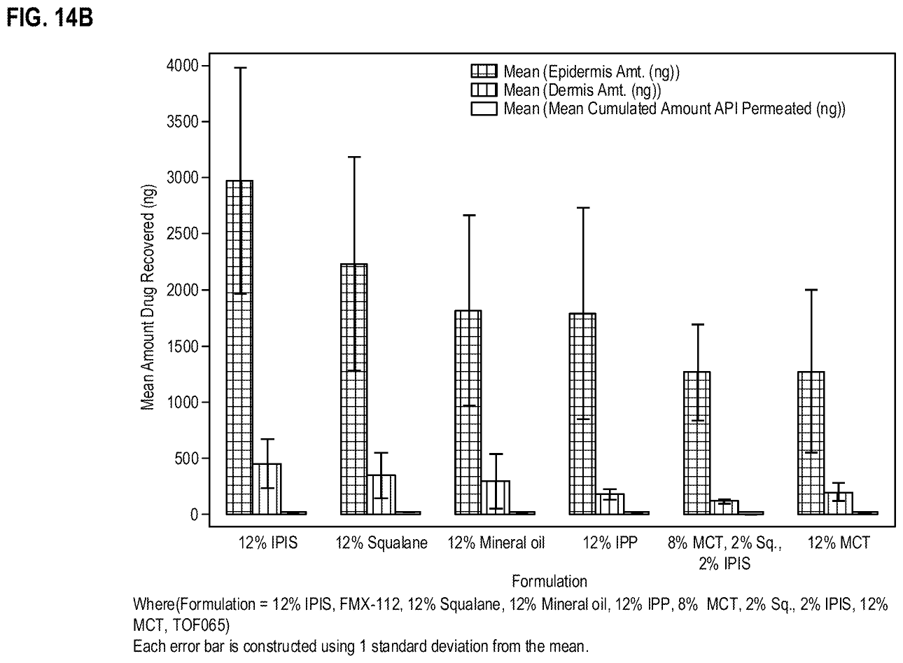

Investigation of the effect of the different formulations (Tables 24a-c) on skin penetration was undertaken in accordance with the protocol set out in Methods section. The investigation examined, amongst other things, the effect of the different oils on skin penetration. As can be seen in Tables 24a-c and FIG. 14A-B, addition of squalane and/or isopropyl isostearate improved delivery oftofacitinib citrate into the dermis and epidermis and improved the skin to systemic delivery ratio.

TABLE 24a

| | | | | TOF063 | | | TOF074 |
|---|---|---|---|---|---|---|---|
| | | | | 12% | TOF082 | TOF083 | 8% MCT, |
| | | TOF059 | TOF061 | Mineral | 12% | 12% | 2% Sq, |
| | | 12% MCT | 12% IPP | oil | Squalaze | IPIS | 2% IPIS |
| | | 0.6 m* | | | | | |
| Ingredients | | w/w | | | | | |
| Tofacitinib Citrate | | 1 | 1 | 1 | 1 | 1 | 1 |
| ST-Elastomer 10 | | 87 | 87 | 87 | 87 | 87 | 87 |
| MCT oil | | 12 | | | | | 8 |
| Isopropyl Palmitate | | | 12 | | | | |
| Mineral oil | | | | 12 | | | |
| Squalane | | | | | 12 | | 2 |
| Isopropyl isostearate | | | | | | 12 | 2 |
| Total | | 100 | 100 | 100 | 100 | 100 | 100 |
| Results | | | | | | | |
| Amount | Epidermis | 2.13 | 2.99 | 3.04 | 3.73 | 4.96 | 2.11 |
| recovered | Dermis | 0.34 | 0.31 | 0.50 | 0.59 | 0.76 | 0.21 |
| μg/cm² | Receptor | 0.019 | 0.013 | 0.009 | 0.016 | 0.034 | 0.006 |
| | fluid | | | | | | |
| Amount | Epidermis | 3.39 | 4.76 | 4.98 | 6.54 | 8.50 | 3.60 |
| recovered | Dermis | 0.53 | 0.52 | 0.80 | 0.97 | 1.27 | 0.34 |
| % applied | Receptor | 0.02 | 0.02 | 0.02 | 0.03 | 0.07 | 0.02 |
| dose | fluid | | | | | | |

*0.6% tofacitinib corresponds to 1% tofacitinib citrate
m = micronized API

TABLE 24b

| | | BP190133.0017 | TOF074(0.6)* |
|---|---|---|---|
| ST-Elastomer 10 | | 87.4 | 87 |
| MCT oil | | 8 | 8 |
| Squalane | | 2 | 2 |
| IPIS | | 2 | 2 |
| Tofacitinib free base | | 0.6 | |
| Tofacitinib citrate | | | 1.0 |
| Total | | 100 | 100 |
| Results | | | |
| Amount recovered | Epidermis | 4.21 | 2.11 |
| μg/cm² | Dermis | 0.35 | 0.21 |
| | Receptor fluid | 0.009 | 0.006 |
| Amount recovered | Epidermis | 7.34 | 3.60 |
| % applied dose | Dermis | 0.61 | 0.34 |
| | Receptor fluid | 0.03 | 0.02 |

*formulation TOF074(0.6) is equivalent to formulation 021A, containing 1.0% of tofacitinib citrate, corresponding to 0.6% of tofacitinib

TABLE 24c

| | | | | | | Receptor | Skin to systemic | |
|---|---|---|---|---|---|---|---|---|
| | | Epidermis Amt. | | Dermis Amt. | | Fluid | delivery ratio | |
| | | (ng) | | (ng) | | Amt. | (Epidermis + Bermis | |
| | | | Std | | Std | (ng) | to Receptor Fluid) | |
| Formulation | | Mean | Dev | Mean | Dev | Mean | Mean | Std Dev |
| TOF083 | 12% IPIS | 2976.00 | 1009.79 | 452.60 | 217.25 | 23.17 | 147.98 | 50.41 |
| TOF082 | 12% | 2240.33 | 949.28 | 350.83 | 201.69 | 20.23 | 128.09 | 44.30 |
| | Squalane | | | | | | | |
| TOF063 | 12% | 1825.33 | 848.57 | 300.87 | 241.11 | 15.79 | 134.65 | 60.14 |
| | Mineral | | | | | | | |
| | oil | | | | | | | |
| TOF061 | 12% IPP | 1672.00 | 997.74 | 185.20 | 43.27 | 9.12 | 203.64 | 110.28 |
| TOF074 | 8% MCT, | 1290.20 | 469.55 | 123.20 | 17.01 | 4.09 | 345.57 | 118.84 |
| | 2% Sq, | | | | | | | |
| | 2% IPIS | | | | | | | |
| TOF059 | 12% MCT | 1280.17 | 721.77 | 205.83 | 80.70 | 13.08 | 113.61 | 57.28 |

### Example 24. Skin Penetration Study of a Formulation Based on Tofacitinib Citrate and Equivalent Formulation Based on Free Base Tofacitinib

Formulations comprising either tofacitinib citrate or free base tofacitinib were compared for skin penetration, in accordance with the protocol set out in Methods section.

As can be seen in Table 25, a formulation comprising free base tofacitinib resulted in a higher penetration of the active agent into the epidermis as compared to a formulation comprising tofacitinib citrate.

TABLE 25

| | | BP190133.0017 | TOF074(0.6)* |
|---|---|---|---|
| ST-Elastomer 10 | | 87.4 | 87 |
| MCT oil | | 8 | 8 |
| Squalane | | 2 | 2 |
| IPIS | | 2 | 2 |
| Tofacitinib free base | | 0.6 | |
| Tofacitinib citrate | | | 1.0 |
| Total | | 100 | 100 |
| Results | | | |
| Amount recovered | Epidermis | 4.21 | 2.11 |
| μg/cm² | Dermis | 0.35 | 0.21 |
| | Receptor fluid | 0.009 | 0.006 |
| Amount | Epidermis | 7.34 | 3.60 |
| recovered % | Dermis | 0.61 | 0.34 |
| applied dose | Receptor fluid | 0.03 | 0.02 |

*formalation TOF074(0.6) is equivalent to formulation 021A, containing 1.0% of tofacitinib citrate, corresponding to 0.6% of tofacitinib

### Example 25. Elastomer-Based Formulations Comprising Different Emollients Tested for Physical Properties

Investigation of the effect of the different formulations (Table 26) on physical properties was undertaken in accordance with the protocol set out in Methods section. The investigation examined, amongst other things, the effect of the different oils on the physical properties of the formulations. Viscosity was measured according to experimental method M Part A.

Formulations including MCT oil, isopropyl palmitate, mineral oil, squalane or isopropyl isostearate resulted in transparent gels with no balling effect. Oleyl alcohol or soybean oil were less compatible with elastomer, resulting in translucent flowable semi-solid formulations. Low viscosity and/or flowable formulations are less desirable for o suspending drugs, due to a potentially higher risk of drug aggregation and or sedimentation. In addition, translucent appearance is an indication of a multi-phase system and may be an indicator to predict a potential phase separation. Thus, in one or more embodiments, the formulations provided herein comprises a MCT oil, isopropyl palmitate, a mineral oil, squalane, isopropyl isostearate or mixtures of two or more thereof. In some embodiments unsaturated fatty alcohols like oleyl alcohol are present in low amounts, e.g., about 0.1% to 5%, or about 0.1% to 2%, or less than about 1%, or less than about 0.4%. In some embodiments highly unsaturated vegetable oils like soybean oil are present in low amounts, e.g., about 0.1% to 5%, or about 0.1% to 2%, or less than about 1%, or less than about 0.4%. In one or more embodiments, the formulations provided herein are free or substantially free of oleyl alcohol and/or a soybean oil.

TABLE 26

| | | OT1.00 | | | OT1.00 | | OT1.00 |
|---|---|---|---|---|---|---|---|
| | OT1.0016P | IPP | OT1.00OA | OT1.00MO | SO | OT1.00SQ | IPIS |
| Ingredients | % w/w | | | | | | |
| ST-Elastomer 10 | 88 | 88 | 88 | 88 | 88 | 88 | 88 |
| MCT oil | 12 | | | | | | |
| Isopropyl Palmitate | | 12 | | | | | |
| Oleyl alcohol | | | 12 | | | | |
| Mineral oil | | | | 12 | | | |
| Soybean oil | | | | | 12 | | |
| Squalane | | | | | | 12 | |
| Isopropyl isostearate | | | | | | | 12 |
| Total | 100 | 100 | 100 | 100 | 100 | 100 | 100 |
| Results | | | | | | | |
| Visual appearance | TP gel | TP gel | TL | TP gel | TL | TP gel | TP gel |
| in a glass bottle | | | flowable | | flowable | | |
| | | | semi- | | semi- | | |
| | | | solid | | solid | | |
| Balling | NO | NO | NO | NO | NO | NO | NO |

TP = Transparent;
TL = Translucent

### Example 26. Elastomer-Based Formulations Comprising a Combination of MCT Oil and Additional Emollients at Different Concentrations Tested for Physical Properties

Investigation of the effect of the different formulations (Table 27) on physical properties was undertaken in accordance with the protocol set out in Methods section. The investigation examined, amongst other things, the effect of oil combinations at different concentrations on the physical properties of the formulations. The amount of the oil fraction and elastomer varied, while the ratio between the oils remained constant in this experiment, i.e. the ratio MCT oil:Squalane:IPIS remained 4:1:1 in all the formulations tested. Formulations comprising about 82% to about 94% elastomer were tested. Formulations comprising about 85% to about 91% ST-Elastomer resulted in transparent gels and exhibited no balling effect. Formulations comprising less than about 85% of ST-Elastomer resulted in flowable semi-solids with no balling effect. Formulations comprising more than about 91% of ST-Elastomer resulted in transparent gels that exhibited a balling effect. A formulation comprising 90% ST-Elastomer, MCT oil (6.67%), squalane (1.67%) and isopropyl isostearate (1.67%) resulted in a transparent gel with no balling effect (Table 27) whereas a formulation comprising 90% ST-Elastomer with 10% MCT oil resulted in transparent gel with a balling effect. Thus, without being bound by any theory it could be suggested that squalane and isopropyl isostearate may assist in expanding the range of elastomer that can be used and still result in an acceptable formulation. i.e. a transparent gel formulation with no balling effect. In one or more embodiments, the formulations provided herein comprise about 85% to about 91% ST-Elastomer by weight. For example, about 85%, about 86%, about 87%, about 88%, about 89%, about 90%, or about 91% by weight of ST-Elastomer.

TABLE 27

| | 085(0)- | 078(0)- | 079(0)- | 071(0)- | 072(0)- | 073(0)- | 074(0)- | 075(0)- | 076(0)- | 077(0)- | 080(0)- | 081(0)- | 084(0)- |
|---|---|---|---|---|---|---|---|---|---|---|---|---|---|
| | 200108S | 200108S | 200108S | 200105S | 200105S | 200105S | 200105S | 200105S | 200105S | 200105S | 200108S | 200108S | 200108S |
| Ingredient | % w/w | | | | | | | | | | | | |
| ST-Elastomer 10 | 94.0 | 93.0 | 92.0 | 91.0 | 90.0 | 89.0 | 88.0 | 87.0 | 86.0 | 85.0 | 84.0 | 83.0 | 82.0 |
| MCT oil | 4.0 | 4.67 | 5.33 | 6.0 | 6.67 | 7.33 | 8.0 | 8.67 | 9.33 | 10.0 | 10.67 | 11.33 | 12.0 |
| Squalane | 1.0 | 1.17 | 1.33 | 1.5 | 167 | 1.83 | 2.0 | 2.17 | 2.33 | 2.5 | 2.67 | 2.83 | 3.0 |
| Isopropyl isostearate | 1.0 | 1.17 | 1.33 | 1.5 | 1.67 | 1.83 | 2.0 | 2.17 | 2.33 | 2.5 | 2.67 | 2.83 | 3.0 |
| Total | 100 | 100 | 100 | 100 | 100 | 100 | 100 | 100 | 100 | 100 | 100 | 100 | 100 |
| ST-Elastomer:Oils | 94:6 | 93:7 | 92:8 | 91:9 | 90:10 | 89:11 | 88:8 | 87:13 | 86:14 | 85:15 | 84:16 | 83:17 | 82:18 |
| ratio | | | | | | | | | | | | | |
| Results | | | | | | | | | | | | | |
| Visual appearance | TP | TP | TP | TP | TP | TP | TP | TP | TP | TP | TP | TP | TL |
| in a glass bottle | gel | gel | gel | gel | gel | gel | gel | gel | gel | gel | flowable | flowable | flowable |
| | | | | | | | | | | | semi- | semi- | semi- |
| | | | | | | | | | | | solid | solid | solid |
| Balling | Balling | Balling | Balling | No | No | No | No | No | No | No | No | No | No |
| | | | | balling | balling | balling | balling | balling | balling | balling | balling | balling | balling |

TP = Transparent;
TL = Translucent

### Example 27. Formulations with Alternative Elastomers

Investigation of the effect of the different formulations (Table 28) on various physical parameters was undertaken in accordance with the protocol set out in Methods section. The investigation examined, amongst other things, the effect of the different elastomers on the physical properties of the formulation. As can be seen in Table 28, all formulations tested resulted in gels. It should be noted that all gels were white and not transparent, since the active agent was homogeneously dispersed within the formulation. In one or more embodiments, the formulations provided herein comprise ST-Elastomer 10, ST-Elastomer 1148, Gransil DMG-6, Gransil DM-5 Elastomer or mixtures thereof.

TABLE 28

| | OT1.0030A | OT1.00ELA | 0T1.0030ELB | OT1.0030ELC |
|---|---|---|---|---|
| | (0.6 *m) | (0.6*m) | (0.6*m) | (0.6*m) |
| | 086(0.6)- | 102(0.6)- | 103(0.6)- | 092(0.6)- |
| | 200109S | 200206S | 200206S | 200115S |
| Ingredients | % w/w | | | |
| Tofacitinib Citrate | 1 | 1 | 1 | 1 |
| ST-Elastomer 10 | 87 | | | |
| ST-Elastomer 1148 | | 87 | | |
| Gransil DMG-6 | | | 87 | |
| Gransil DM-5 | | | | 87 |
| Elastomer | | | | |
| Squalane | 2 | 2 | 2 | 2 |
| Isopropyl isostearate | 2 | 2 | 2 | 2 |
| MCT oil | 8 | 8 | 8 | 8 |
| Total | 100 | 100 | 100 | 100 |
| Results | | | | |
| Visual appearance | White gel | White gel | White gel | White gel |
| in a glass bottle | | | | |
| Physical stability 3M | Homogenous | Homogenous | Homogenous | Homogenous |
| 40 C. | distribution of | distribution of | distribution of | distribution of |
| | API crystals | API crystals | API crystals | API crystals |

*0.6% tofacitinib corresponds to 1% tofacitinib citrate
m = micronized API

### Example 28. Elastomer-Based Formulation with Surfactant

Investigation of the chemical and physical parameters of a formulation (Table 29) was undertaken in accordance with the protocol set out in Methods section. The investigation examined, amongst other things, the effect of addition of surfactant on the chemical and physical properties of the formulation. As can be seen in Table 29, an elastomer-based Tofacitinib Citrate formulation comprising surfactant resulted in a white gel which is chemically stable after 3 months at 40° C. In one or more embodiments the formulations provided herein comprise surfactant. In one or more embodiments the formulations provided herein comprise surfactant are chemically stable after 3 months at 40° C.

TABLE 29

| | OT1.00GMS |
|---|---|
| | (0.6*m) |
| Ingredients | w/w |
| Tofacitinib Citrate | 1 |
| ST-Elastomer 10 | 86 |
| Glyceryl monostearate | 1 |
| Squalane | 2 |
| IPIS | 2 |
| MCT Oil | 8 |
| Total | 100 |
| Results | |
| Visual appearance | White gel |
| in a glass bottle | |
| Chemical stability | 102.9 |
| after 3 Mo at 40° C. | |
| (Tofacitinib assay, %/T0) | |

*0.6% tofacitinib corresponds to 1% tofacitinib citrate
m = micronized API

### Example 29. Elastomer-Based Formulations

Evaluation of elastomer-based formulations as foamable formulations (Table 30) for various foam properties was undertaken in accordance with the protocol set out in Methods section. As can be seen in Table 30, addition of propellant to these elastomer-based formulations—without a surfactant and or saturated fatty alcohols/saturated fatty acids resulted in poor foams. See Example 1 for an illustration of a foamable composition adding e.g., a non-ionic surfactant with a HLB lower than 7 and a saturated fatty alcohol.

TABLE 30

| | 0T1.0025A | OT1.L1 | OT1.0030A |
|---|---|---|---|
| | (0.6*m) | (0.6*m) | (0.6*m) |
| Ingredients | % w/w | | |
| Tofacitinib Citrate | 1 | 1 | 1 |
| ST-Elastomer 10 | 87 | 87 | 87 |
| Squalane | | | 2 |
| Isopropyl isostearate | | | 2 |
| MCT oil | 12 | 6 | 8 |
| Oleyl alcohol | | 6 | |
| Total | 100 | 100 | 100 |
| AP-70 (butane, | 14 | 14 | 14 |
| isobutane, propane | | | |
| mixture) | | | |
| Results | | | |
| Foam quality | poor | poor | poor |

*0.6% tofacitinib corresponds to 1% tofacitinib citrate
m = micronized API

### Example 30. Elastomer-Based Formulations with Different Active Agent Combinations

Investigation of the effect of the different formulations (Table 31) on various physical parameters was undertaken in accordance with the protocol set out in Methods section. The investigation examined, amongst other things, the effect of different active agent combinations on the physical properties of the formulation. As can be seen in Table 31, an elastomer-based formulation comprising a combination of tofacitinib citrate and doxycycline hyclate resulted in a yellow gel, due to the yellow color of doxycycline. An elastomer-based formulation comprising tofacitinib citrate and nicotinamide resulted in white gel. However, nicotinamide particles were visible in this formulation, since this active ingredient was non-micronized. In one or more embodiments, an elastomer-based formulation as provided herein comprises a combination of two or more active agents. In one or more embodiments, the compositions described herein are suitable for inclusion of insoluble active agents. In one or more embodiments the insoluble or suspended active agents are micronized.

TABLE 31

| | OT1. | OT1. | OT1. | OT1. |
|---|---|---|---|---|
| | 00TOMH | 00TOTA | 00TOMF | 00TONA |
| | (0.6 m*) | (0.6 m*) | (0.6 m*) | (0.6 m*) |
| Ingredients | % w/w | | | |
| Tofacitinib | 1 | 1 | 1 | 1 |
| Citrate | | | | |
| Doxycycline | 3.5 | | | |
| hyclate** | | | | |
| Triamcinolone | | 0.1 | | |
| acetonide | | | | |
| Mometasone | | | 0.05 | |
| furoate | | | | |
| Nicotinamide | | | | 10 |
| ST-Elastomer 10 | 84.5 | 86.9 | 86.95 | 77 |
| Squalane | 2 | 2 | 2 | 2 |
| Isopropyl | 2 | 2 | 9 | 2 |
| isostearate | | | | |
| MCT oil | 8 | 8 | 8 | 8 |
| Total | 100 | 100 | 100 | 100 |
| Results | | | | |
| Appearance | Yellow | White | White | White gel with |
| | gel | gel | gel | particles of NCA |
| | | | | (non-micronized) |
| | | | | visible by eye |

*0.6% tofacitinib corresponds to 1% tofacitinib citrate
**The amount of doxycycline hyclate in the formulation is adjusted by the potency of the doxycycline hyclate.
m = micronized API

### Example 31. Solubility of Tofacitinib in Formulation Based on ST Elastomer 10, MCT Oil, Squalene and IPIS

The solubility of tofacitinib citrate was tested in a formulation based on OT1.0021 without dimethicone crosspolymer. (i.e., ST-Elastomer 10 is substituted by cyclomethicone (see Table 32 below) in accordance with the protocol set out in Methods section.) This test provided an estimate of the solubility of tofacitinib citrate in formulation OT1.0021. Results indicated that the solubility oftofacitinib citrate in formulation P was below the detection limit (<0.01 ppm). Thus, it was estimated that tofacitinib citrate is not soluble and present as dispersed solid particles in formulation OT1.0021. In one or more embodiments the elastomer-based formulation comprises a non-soluble active agent. In one or more embodiments, the elastomer-based formulation comprises a suspended active agent.

TABLE 32

| | FP(0.6*m) |
|---|---|
| Ingredients | w/w |
| Tofacitinib Citrate | 1.0 |
| Cyclomethicone | 87 |
| MCT oil | 8 |
| Squalane | 2 |
| Isopropyl isostearate | 2 |
| Total | 100 |

*0.6% tofacitinib corresponds to 1% tofacitinib citrate
m = micronized API

### Example 32. Water Activity

The water activity of formulation OT1.0021 at 1.2% of Tofacitinib and for corresponding placebo (see Table 33 below) was measured in accordance with the protocol set out in USP<1112>. Water activity was found to be low in both active and placebo formulations.

TABLE 33

| | OT1.0021(12 *m) | OT1.0021(0.0) |
|---|---|---|
| Ingredients | % w/w | |
| Tofacitinib Citrate | 2.0 | 0,0 |
| ST-Elastomer 10 | 86 | 88 |
| MCT oil | 8 | 8 |
| Squalane | 2 | 2 |
| Isopropyl isostearate | 2 | 2 |
| Total | 100 | 100 |
| Water activity | 0.23 | 0.28 |

*1.2% tofacitinib corresponds to 2% tofacitinib citrate
m = micronized API

### Example 33. Formulations Comprising Tofacitinib Dissolved (Partially or Entirely)

Formulations comprising tofacitinib citrate in a dissolved state (partially or entirely) were prepared. A formulation comprising 20% DMSO resulted in phase separation. A formulation comprising 10% DMSO resulted in almost transparent (i.e. tofacitinib was soluble in the formulation) gel. A formulation comprising propylene glycol resulted in a white gel (i.e. part of the tofacitinib was soluble in the formulation and the insoluble part caused the white color appearance). In one or more embodiments, the active agent is soluble in the formulation. In one or more embodiments, the active agent is partially soluble in the formulation. In one or more embodiments, the active agent is non soluble in the formulation.

In some embodiments there is provided a formulation in which tofacitinib has some solubility. In some embodiments the tofacitinib salt is solubilized in DMSO or another known solvent for tofacitinib salt. In some embodiments the DMSO is about 5% to about 15%, e.g., about 55, about 7.5% about 10% about 12.5% or about 15% by weight of the composition.

TABLE 34

| | FR1(0.6 * m) | FR2(0.6 * m) | FR3(0.6 * m) |
|---|---|---|---|
| Ingredients | % w/w | | |
| Tofacitinib Citrate | 1.0 | 1.0 | 1.0 |
| ST-Elastomer 10 | 77 | 67 | 77 |
| MCT oil | 8 | 8 | 8 |
| Squalane | 2 | 2 | 2 |
| Isopropyl isostearate | 2 | 2 | 2 |
| Dimethyl Sulfoxide | 10 | 20 | |
| Propylene Glycol | | | 10 |
| Total | 100 | 100 | 100 |
| Results | | | |
| Visual appearance | TP gel | Phase | White gel |
| | | separation | |

*0.6% tofacitinib corresponds to 1% tofacitinib citrate
m = micronized API
TP Transparent;
TL = Translucent

### Example 34. Elastomer-Based Formulations Comprising Alternative Emollients

Evaluation of elastomer-based formulations (Table 35) for an additional emollient was undertaken in accordance with the protocol set out in Methods section. A PPG 15 stearyl ether elastomer-based formulations was prepared and resulted in white viscous liquid.

TABLE 35

| | OT1.00PPG |
|---|---|
| | (0.6-m*) |
| Ingredients | |
| Tofacitinib Citrate* | 1 |
| ST-Elastomer 10 | 87 |
| PPG 15 Stearyl ether | 12 |
| Total | 100 |
| Results | |
| Visual appearance | White |
| | liquid |

*0.6% tofacitinib corresponds to 1% tofacitinib citrate
m = micronized API

### Example 35. Formulations Comprising Gelled Oil

Versagel-based formulations with different oil combinations were prepared and evaluated for visual appearance. As can be seen in Table 36, formulations comprising versagel in combination with MCT oil, squalane and isopropyl isostearate or in combination with cyclomethicone resulted in white gels. A formulation comprising 99% versagel and 1% active agent resulted in a white gel. In one or more embodiments, the formulations provided herein comprise a gelled mineral oil. In one or more embodiments, the formulations provided herein comprise a versagel.

TABLE 36

| | OT1.00VG | OT1.00VMSI | OT1.00VC | OT1.00VCSI |
|---|---|---|---|---|
| | (0.6-m*) | (0.6-m*) | (0.6-m*) | (0.6-m*) |
| Ingredients | % w/w | | | |
| Tofacitinib titrate | 1 | 1 | 1 | 1 |
| Versagel | 99 | 87 | 87 | 87 |
| MCT oil | | 8 | | |
| Cyclomethicone | | | 12 | 8 |
| Squalane | | 2 | | 2 |
| Isopropyl isostearate | | 2 | | 2 |
| Total | 100 | 100 | 100 | 100 |
| Results | | | | |
| Visual appearance | White gel | White gel | White gel | |

*0.6% tofacitinib corresponds to 1% tofacitinib citrate
m = micronized API

### Example 36. Elastomer-Based Formulations with Different Active Agents (without Tofacitinib)

Elastomer-based formulations comprising different active agents were prepared and evaluated for visual appearance. As can be seen in Table 37, an elastomer-based formulation comprising minocycline hydrochloride resulted in a yellow gel (due to the color of the active agent). An elastomer-based formulation comprising adapalene resulted in a white gel. An elastomer-based formulation comprising doxycycline hyclate resulted in a yellow gel (due to the color of the active agent). An elastomer-based formulation comprising non-micronized nicotinamide resulted in a gel with visible particles of nicotinamide. In one or more embodiments, the formulations provided herein comprise elastomer-based formulation comprising minocycline hydrochloride. In one or more embodiments, the formulations provided herein comprise elastomer-based formulation comprising adapalene. In one or more embodiments, the formulations provided herein comprise elastomer-based formulation comprising doxycycline hyclate. In one or more embodiments the active agents are micronized. In some embodiments they are provided as nanoparticles.

TABLE 37

| | OT1.00MC | OT1.00AD | OT1.00DOX | OT1.00NA |
|---|---|---|---|---|
| Ingredients | % w/w | | | |
| minocycline | 4 | | | |
| hydrochloride* | | | | |
| Adapalene | | 0.3 | | |
| Doxycycline | | | 3.5 | |
| Hyclate** | | | | |
| Nicotinamide | | | | 10 |
| ST-Elastomer 10 | 84 | 87.7 | 84.5 | 78 |
| Squalane | 2 | 2 | 2 | 2 |
| Isopropyl | 2 | 2 | 2 | 2 |
| isostearate | | | | |
| MCT oil | 8 | 8 | 8 | 8 |
| Total | 100 | 100 | 100 | 100 |
| Results | | | | |
| Appearance | Yellow gel | Light | Yellow gel | Gel with |
| | | white gel | | particles |
| | | | | of API |
| | | | | visible |

*The amount of minocycline hydrochloride in the formulation is adjusted by the potency of the minocycline hydrochloride.
*The amount of doxycycline byclate in the formulation is adjusted by the potency of the doxycycline hyclate.

### Example 37. Chemical Stability of Elastomer-based formulation with Tofacitinib and Fingolimod

Investigation of the chemical stability of a formulation containing 0.6% of tofacitinib (1% of tofacitinib citrate) and 0.01% of fingolimod hydrochloride (Table 38) was undertaken in accordance with the protocol set out in Methods section. At initiation of investigation the formulation was physically stable and exhibited homogeneous distribution of the crystals and no aggregates were observed. As can be seen in Table 38, the elastomer-based tofacitinib citrate and fingolimod hydrochloride combination formulation was found to be chemically stable for up to 2 months at 5° C. and as high as at 50° C. Fingolimod hydrochloride was observed to be stable at 5° C., 40° C. and 50° C. Although chemical stability for tofacitinib was not determined at initial time point and at 3 weeks at 40° C., the measurements taken at three weeks at 5° C. and at 50° C. suggest that it is stable. This is further supported in view of chemical stability observed with a tofacitinib formulation at 1-3 months at 25° C. and 40° C. (see Example 16 Table 17f OT1.0.021A (0.3)). In one or more embodiments the formulations provided herein comprising tofacitinib (e.g., tofacitinib citrate) and fingolimod (e.g., fingolimod hydrochloride) are compatible. In one or more embodiments the formulations comprising tofacitinib citrate and fingolimod hydrochloride are chemically stable e.g., after 3 weeks at 5° C., 40° C. or 50° C. In one or more embodiments the formulation is physically stable e.g., after 2 months at 5° C., 40° C. or 50° C.

TABLE 38

| Ingredients | | | % w/w | | | |
|---|---|---|---|---|---|---|
| Tofacitinib Citrate | | | 1.0 | | | |
| Fingolimod HCl | | | 0.01 | | | |
| ST Elastomer-10 | | | 86.99 | | | |
| MCT oil | | | 8.0 | | | |
| IPIS | | | 2.0 | | | |
| Squalene | | | 2.0 | | | |
| Total | | | 100 | | | |
| | | 3 | 3 | 3 | 2 | 2 |
| | | weeks | weeks | weeks | months | months |
| | | at | at | at | at | at |
| Results: | Initial | 5° C. | 40° C. | 50° C. | 40° C. | 50° C. |
| Assay of | 99.9 | 100.3 | 99.9 | 94.1 | 97.2 | 95.0 |
| Fingolimod, | | | | | | |
| % LC | | | | | | |
| Assay of | not | 99.3 | not | 99.1 | 99.2 | 98.6 |
| Tofacitinib, | tested | | tested | | | |
| % LC | | | | | | |

### Example 38. In-Vivo Atopic Dermatitis Animal Model Study

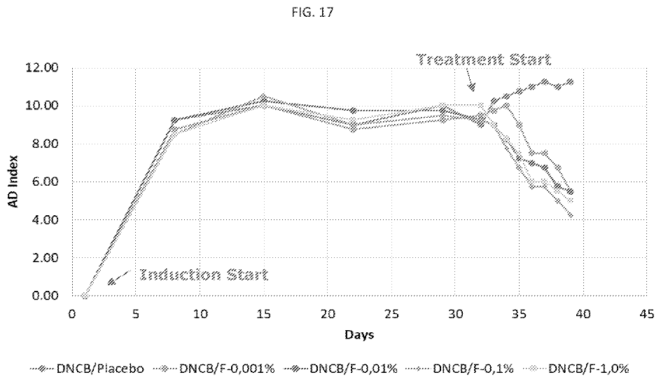

A study was conducted to evaluate elastomer-based formulations containing fingolimod hydrochloride at different strengths in an Atopic Dermatitis animal model in accordance with the protocol set out in Experimental Method C. Formulations containing fingolimod hydrochloride were compared to a placebo formulation, as negative control. The evaluated formulations are presented in Table 39. The treatments evaluated in the study are presented in Table 39a and the results evaluated in the study are provided in Table 39b and are shown in FIG. 17.

The study results evaluated in the study are presented in Table 39c. According to Table 39b, treatment with the compound at 0.001% w/w and 0.01% w/w resulted in body weight increase, while the body weight was lower and similar to placebo group in the treatment group with the compound at 0.1% w/w and 1% w/w. This body weight decrease shows that 0.1% w/w and 1% w/w were not well tolerated. In addition, all fingolimod arms reduced the AD Index compared to the placebo arm. A dose-effect relationship was observed. After 6 days of treatment, the AD Index was still in a decrease trend for all fingolimod arms. The 0.01% arm had a quicker onset of action compared to the 0.001% arm. On its own fingolimod hydrochloride at 0.01% appears to be effective and increasing the concentration to say 0.1% may not provide additional benefit in atopic dermatitis. In combination with a second active agent though increasing fingolimod hydrochloride strengths above 0.01% may still allow for an additional decrease in the concentration of the second active agent e.g., a tofacitinib. On the other hand, the presence of the second active agent e.g., tofacitinib citrate may facilitate a further reduction of the fingolimod hydrochloride. From a benefit/risk ratio perspective, a dose in the region of 0.01% strength appears to be a good starting point to establish an appropriate dose for fingolimod citrate when used alone for treatment of AD on a human subject. The optimum strength ranges may differ and be reduces when used in a combination product. Without being bound by any theory, a synergistic effect between the carrier and the two active ingredients can allow for lower dosages and or higher efficacy of each active agent respectively in treating or ameliorating the disorder. In one or more embodiments the fingolimod hydrochloride concentration may be lower than 0.01% when combined with say tofacitinib citrate at about 0.6%. In one or more embodiments the tofacitinib citrate concentration may be lowered by combining it with fingolimod hydrochloride, for example, say a 1.2% concentration of tofacitinib citrate may be lowered to 0.6% when combined with say fingolimod hydrochloride at about 0.01%. In one or more embodiments tofacitinib citrate may be lowered below 0.6%, such as to 0.5%, 0.4% or 0.3% when combined with say fingolimod hydrochloride at higher amount above about 0.01%, e.g., 0.02%, 0.03%, 0.04%, or 0.05%. Note that in this connection all the amounts of fingolimod tested other than the highest amount of 1% did not result in a weight loss (See Table 39b).

TABLE 39

| | FMX114 | FMX114 | FMX114 | FMX114 | FMX114 |
|---|---|---|---|---|---|
| | (0/0) | (0/0.001) | (0/0.01) | (0/0.1) | (0/1.0) |
| Ingredient | % w/w | | | | |
| Fingolimod HC1* | 0.0 | 0.00112 | 0.0112 | 0.112 | 1.12 |
| MCT oil | 8.0 | 8.0 | 8.0 | 8.0 | 8.0 |
| Squalene | 2.0 | 2.0 | 2.0 | 2.0 | 2.0 |
| Isopropylisosterate (IPIS) | 2.0 | 2.0 | 2.0 | 2.0 | 2.0 |
| ST Elastomer-10 | 88.0 | 87.99888 | 87.9888 | 87.888 | 86.88 |
| Total | 100.0 | 100.0 | 100.0 | 100.0 | 100.0 |

TABLE 39a

| | Treatment group | Formulation |
|---|---|---|
| 1 | Negative control (induced, placebo treatment) | FMX114 (0/0) |
| 2 | Fingolimod 0.001% | FMX114 (0/0.001) |
| 3 | Fingolimod 0.01% | FMX114 (0/0.01) |
| 4 | Fingolimod 0.1% | FMX114 (0/0.1) |
| 5 | Fingolimod 1% | FMX114 (0/1.0)1 |

TABLE 39b

| Treatment | Body Weight | Body weight change | AD Index (ADI) |
|---|---|---|---|
| Ann | D39 (g) | d39−d32 | D39 |
| Placebo | 19.46 | −0.60 | 11.25 |
| 0.001% | 21.08 | +1.07 | 5.50 |
| Fingolimod | | | |
| 0.01% | 21.14 | +1.30 | 5.50 |
| Fingolimod | | | |
| 0.1% | 19.74 | +0.68 | 4.25 |
| Fingolimod | | | |
| 1% | 18.76 | −0.61 | 5.00 |
| Fingolimod | | | |

(−) weight loss
(+) weight gain

### Example 39. Skin Penetration Study for a Formulation Comprising Gelled-Oil

Investigation of skin penetration of a formulation (Table 40) was undertaken in accordance with the protocol set out in Methods section. The investigation examined the effect of gelled oil and its combination with MCT oil on skin penetration. As can be seen in Table 40, and in comparison to formulation TOF059, addition of gelled oil (e.g., versagel) reduced skin penetration into the dermis and epidermis.

TABLE 40

| | | TOF059 | |
|---|---|---|---|
| | | 16A (0.6m*) | TOF065(0.6 m*) |
| Ingredients | | % w/w | % w/w |
| Tofacitinib Citrate | | 1 | 1 |
| ST-Elastomer 10 | | 87 | |
| Versagel | | | 87 |
| MCT oil | | 12 | 12 |
| Total | | 100 | 100 |
| Results | | | |
| Epidermis | | 2.13 | 0.65 |
| Derails | | 0.34 | 0.14 |
| Amount | Receptor fluid | 0.019 | |
| recovered % | | | |
| μg/cm² | | | |
| Amount | Epidermis | 3.39 | 1.15 |
| recovered % | Dermis | 0.53 | 0.24 |
| applied dose | Receptor fluid | 0.02 | 0.01 |

*0.6% tofacitinib corresponds to 1% tofacitinib citrate
m = micronized API

### Example 40. In-Vitro Human Skin Model for Atopic Dermatitis

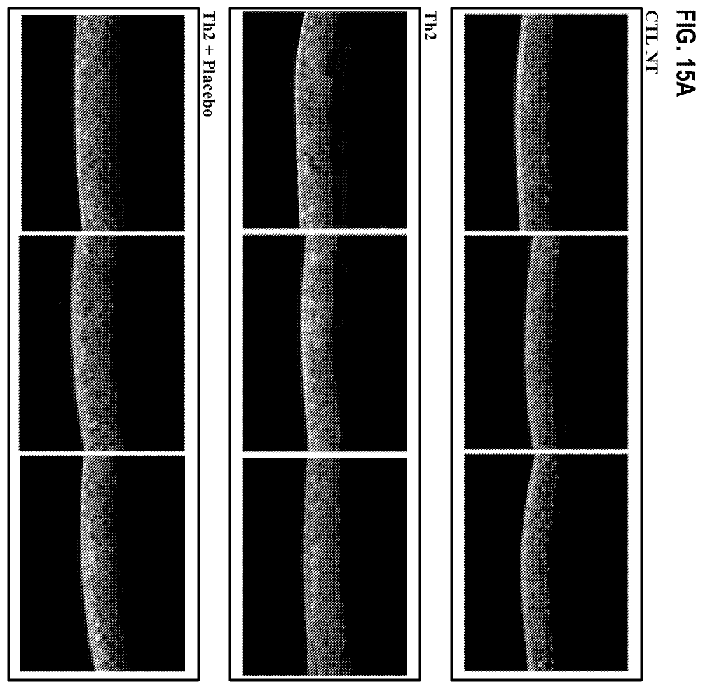

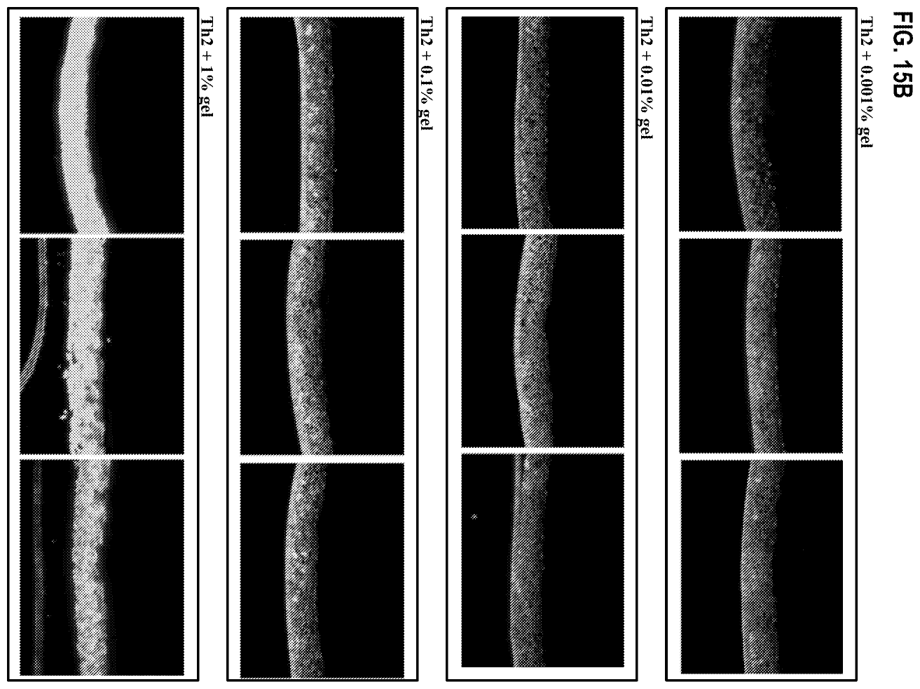

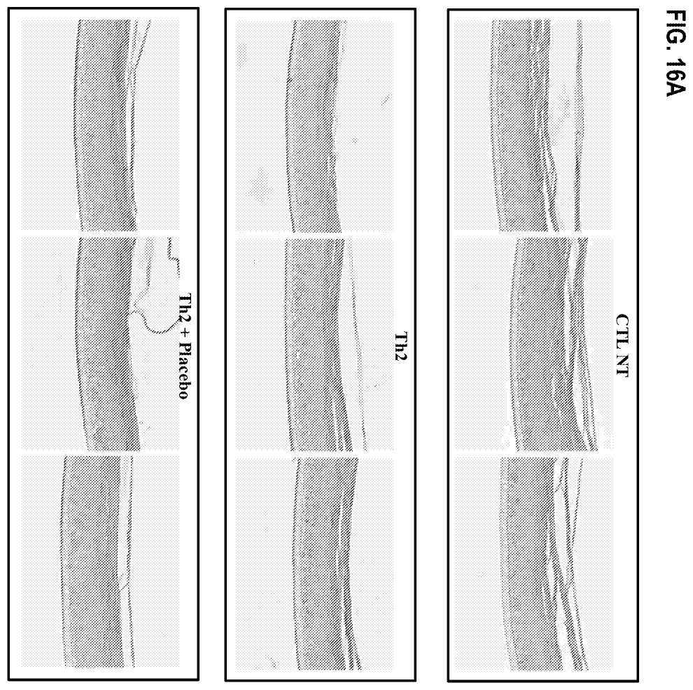

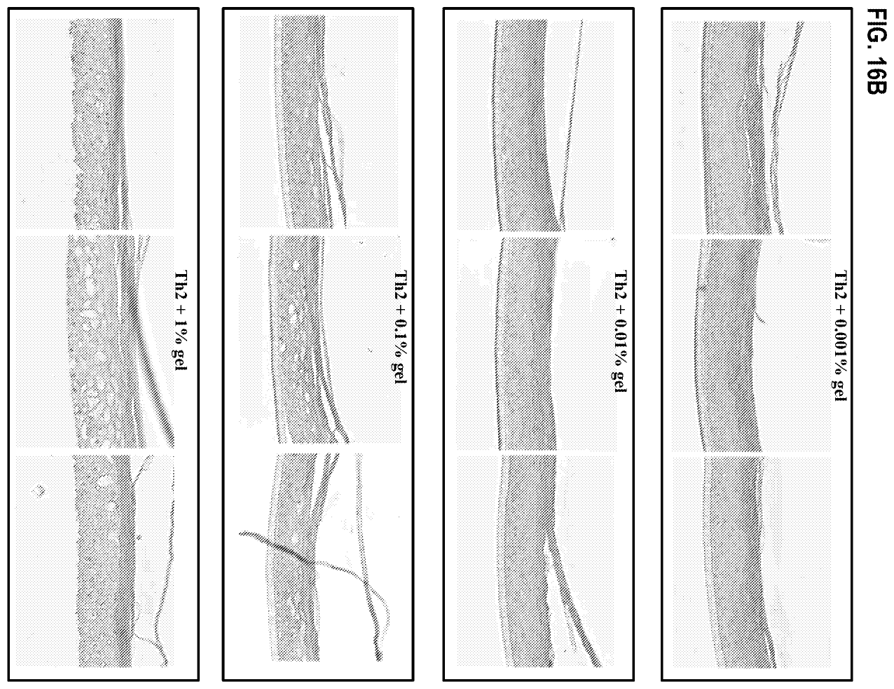

Fingolimod containing formulations (see Table 39 above) were evaluated in Reconstructed Human Epidermis (RHE) Th2 AD model by StratiCELL, Isnes, Belgium as described Experimental section Method S. As shown in FIGS. 15A and 15B, Th2 interleukins stimulation of RHE generated a significant increase in biotin immunofluorescence detected in RHE induced but untreated and RHE induced and treated with placebo (see FIG. 15A middle and bottom row respectively) as compared to control uninduced skin (top row FIG. 15A). Treatment with Fingolimod at the concentration of 0.001% w/w and 0.01% w/w reduced the Th2-induced barrier alteration (FIG. 15B top and middle row respectively). Increased barrier alteration was observed for Fingolimod at a concentration of 0.1% and 1% (FIG. 15B two bottom rows. As shown in FIGS. 16A and 16B that placebo had no observable affect on the morphology. RHEs treated with Th2 interleukins and untreated and placebo showed morphological changes similar to atopic skin such as epidermal hyperplasia (acanthosis), intercellular oedema (spongiosis) and impaired barrier properties. (FIG. 16A middle and bottom row respectively) as opposed to normal uninduced skin (FIG. 16A top row) Fingolimod at a concentration of 0.001% w/w and 0.01% w/w and reduced the Th2-induced barrier alteration (FIG. 16B top and middle row respectively) while increased fragilization was observed with junction/adhesion loss and disorganization of cell layers at Fingolimod concentrations of 0.1% w/w and 1% w/w (FIG. 16B bottom two rows respectively).

Without being bound by any theory it is postulated when higher doses are used there may be transient side effects which pass over time. In one or more embodiments, over short term periods it seems the higher concentrations can damage skin surface although over longer time periods the skin surface may recover.

### Example A. In-Vivo Psoriasis Animal Model for Testing Elastomer-Based Formulations with Different Strengths of Active Agent and Different Combinations of Active Agents

Investigation of the effect of the different formulations containing tofacitinib and/or fingolimod (Table A and A1) in Psoriasis animal model is undertaken in accordance with the protocol set out in Methods section. The investigation provides a more comprehensive study PGP-88 T2 including different active agent concentration points.

TABLE A

| | | | | | | | | | 2% |
|---|---|---|---|---|---|---|---|---|---|
| | | | | | | | | | PEG |
| | placebo | 0.3* | 0.4* | 0.5* | 0.6* | 0.8* | 1.2* | 1.5* | ointment |
| Ingredients | % w/w | | | | | | | | |
| Tofacitinib Citrate | 0 | 0.5 | 0.67 | 0.83 | 1.0 | 1.33 | 2.0 | 2.5 | |
| Tofacitinib free base | | | | | | | | | 2.0 |
| ST-Elastomer 10 | 88 | 87.5 | 87.33 | 87.17 | 87 | 86.67 | 86 | 85.5 | |
| MCT oil | 8 | 8 | 8 | 8 | 8 | 8 | 8 | 8 | |
| Squalane | 2 | 2 | 2 | 2 | 2 | 2 | 2 | 2 | |
| Isopropyl isostearate | 2 | 2 | 2 | 2 | 2 | 2 | 2 | 2 | |
| PEG 400 | | | | | | | | | 30 |
| PEG 3350 | | | | | | | | | 30 |
| Propylene glycol | | | | | | | | | 18 |
| Glycerin | | | | | | | | | 17.9 |
| Oleyl alcohol | | | | | | | | | 2 |
| BHA | | | | | | | | | 0.1 |
| Total | 100 | 100 | 100 | 100 | 100 | 100 | 100 | 100 | 100 |
| PASI (mean) | | | | | | | | | |
| Std Dev | | | | | | | | | |
| Body weight | | | | | | | | | |
| mean)D14 (g) | | | | | | | | | |
| Std Dev | | | | | | | | | |

*0.3% tofacitinib corresponds to 0.5% tofacitinib citrate
*0.4% tofacitinib corresponds to 0.67 tofacitinib citrate
*0.5% tofacitinib corresponds to 0.83% tofacitinib citrate
*0.6% tofacitinib corresponds to 1% tofacitinib citrate
*0.8% tofacitinib corresponds to 1.33% tofacitinib citrate
*1.2% tofacitinib corresponds to 2.0% tofacitinib citrate
*1.5% tofacitinib corresponds to 2.5% tofacitinib citrate

TABLE A1

| | 021 | 021 | 021 | 021 |
|---|---|---|---|---|
| | (0.6*/0.001**) | (0.6*/0.01***) | (0.0/0.001**) | (0.0/0.01***) |
| Ingredients | % w/w | | | |
| Tofacitinib Citrate | 1.0 | 1.0 | 0.0 | 0.0 |
| Fingolimod | 0.0011.2 | 0.0112 | 0.00112 | 0.0112 |
| Hydrochloride | | | | |
| ST-Elastomer 10 | 86.99888 | 86.9888 | 87.99888 | 87.9888 |
| MCT oil | 8 | 8 | 8 | 8 |
| Squalane | 2 | 7 | 2 | 2 |
| Isopropyl isostearate | 2 | 2 | 2 | 2 |
| PEG 400 | | | | |
| PEG 3350 | | | | |
| Propylene glycol | | | | |
| Glycerin | | | | |
| Oleyl alcohol | | | | |
| BHA | | | | |
| Total | 100 | 100 | 100 | 100 |
| PASI (mean) | | | | |
| Std Dev | | | | |
| Bodyweight mean)D14 | | | | |
| (g) | | | | |
| Std Dev | | | | |

*0.6% tofacitinib corresponds to 1% of tofacitinib citrate
**0.001% fingolimod corresponds to 0.00112% of fingolimod HCl
***0.01% fingolimod corresponds to 0.0112% of fingolimod HCl

### Example B. Skin Penetration Study of Fingolimod Hydrochloride Alone or in Combination with Tofacitinib Citrate for a Formulation Comprising Gelled-Oil

Investigation of skin penetration of a formulation comprising Fingolimod Hydrochloride alone or in combination Tofacitinib Citrate (Table B) is undertaken in accordance with the protocol set out in Methods section. The investigation examines the effect of gelled oil and its combination with MCT oil on skin penetration of Fingolimod Hydrochloride alone or in combination Tofacitinib Citrate. Inter alia the following is measured, amount recovered μg/cm2, and amount recovered % applied dose in the epidermis, dermis, and receptor fluid. The amounts in the pilosebaccous appendages may also be determined.

TABLE B

| | 021 (0.6*/ | VSG (0,6*/ | 021(0.0/ | VSG (0.0/ |
|---|---|---|---|---|
| | 0.01**) | 0.01**) | 0.01**) | 0.01 **) |
| Ingredients | % w/w | % w/w | % w/w | % w/w |
| Tofacitinib Citrate | 1 | 1 | 0.0 | 0.0 |
| Fingolimod Hydrochloride | 0.0112 | 0.0112 | 0.0112 | 0.0112 |
| ST-Elastomer 10 | 86.9888 | | 87.9888 | |
| Versagel | | 86.9888 | | 87.9888 |
| MCT oil | 12 | 12 | 12 | 12 |
| Total | 100 | 100 | 100 | 100 |
| Results | | | | |

*0.6% tofacitinib corresponds to 1% of tofacitinib citrate
**0.01% fingolimod corresponds to 0.0112% of fingolimod HCl

### Example C. Skin Penetration Study for Formulations Comprising Active Agent at Different Particle Sizes

Investigation of the effect of the different formulations (Tables C-C2) on skin penetration is undertaken in accordance with the protocol set out in Methods section. The investigation examines, amongst other things, the effect of the different tofacitinib and/or fingolimod particle sizes on skin penetration.

TABLE C

| | OT1.0030Anm | OT1.0030Am | OT1.0030Ana |
|---|---|---|---|
| | (0.6*) | (0.6*) | (0.6*) |
| Ingredients | % w/w | | |
| Tofacitinib Citrate | 1 | | |
| (non-micronized) | | | |
| Tofacitinib Citrate | | 1 | |
| (micronized) | | | |
| Tofacitinib Citrate | | | 1 |
| (nanosized) | | | |
| ST-Elastomer 10 | 87 | 87 | 87 |
| Squalane | 2 | 2 | 9 |
| Isopropyl isostearate | 2 | 2 | 2 |
| MCT oil | 8 | 8 | 8 |
| Total | 100 | 100 | 100 |

*0.6% tofacitinib corresponds to 1% tofacitinib citrate

TABLE C1

| | 0.001*(non- | | |
|---|---|---|---|
| | micro) | 0.001*(micro) | 0.001*(nano) |
| Ingredients | % w/w | | |
| Fingolimod HCl | 0.0112 | | |
| (non-micronized) | | | |
| Fingolimod HCl | | 0.0112 | |
| (micronized) | | | |
| Fingolimod HCl | | | 0.0112 |
| (nanosized) | | | |
| ST-Elastomer 10 | 87.9888 | 87.9888 | 87.9888 |
| Squalane | 2 | 2 | 2 |
| Isopropyl isostearate | 2 | 2 | 2 |
| MCT oil | 8 | 8 | 8 |
| Total | 100 | 100 | 100 |

*0.01% fingolimod corresponds to 0.0112% of fingolimod HCl

TABLE C2

| | 0.6*/ | 0.6*/ | 0.6*/ |
|---|---|---|---|
| | 0.001** | 0.001** | 0.001** |
| | (non-micro) | (micro) | (nano) |
| Ingredients | % w/w | | |
| Fingolimod HCl (non-micronized) | 0.0112 | | |
| Fingolimod HCl (micronized) | | 0.0112 | |
| Fingolimod HCl (nanosized) | | | 0.0112 |
| Tofacitinib Citrate (non- | 1.0 | | |
| micronized) | | | |
| Tofacitinib Citrate (micronized) | | 1.0 | |
| Tofacitinib Citrate (nanosized) | | | 1.0 |
| ST-Elastomer 10 | 86.9888 | 86.9888 | 86.9888 |
| Squalane | 2 | 2 | 2 |
| Isopropyl isostearate | 2 | n | 2 |
| MCT oil | 8 | 8 | 8 |
| Total | 100 | 100 | 100 |

*0.6% tofacitinib corresponds to 1% of tofacitinib citrate
**0.01% fingolimod corresponds to 0.0112% of fingolimod HCl

### Example D. Active Agent Interfacial Tensions

Measurement of surface energy are performed for tofacitinib free base and tofacitinib salts (adipate, myristate, laurate) in accordance with the protocol set out in Methods section to predict interaction of tofacitinib base and salts with stainless steel, copper and polytetrafluoroethylene (PTFE).

### Example E. IVRT—Release of Tofacitinib from Topical Compositions

In-vitro release testing (IVRT) of tofacitinib and/or fingolimod from different formulations (as described in Table E and Table E1 respectively) is performed in accordance with the protocol set out in Methods section.

TABLE E

| | | | PEG |
|---|---|---|---|
| | 021(0.6*m) | 058B(0.6*m) | ointment |
| Ingredients | % w/w | | |
| Tofacitinib free base | | | 2 |
| Tofacitinib Citrate | 1.0 | 1.0 | |
| ST-Elastomer 10 | 87 | | |
| MCT oil | 8 | 8 | |
| Squalane | o | o | |
| Isopropyl isostearate | 2 | 2 | |
| Petrolatum | | 87 | |
| PEG 400 | | | 30 |
| PEG 3350 | | | 30 |
| Propylene glycol | | | 18 |
| Glycerin | | | 17.9 |
| Oleyl alcohol | | | 2 |
| BHA | | | 0.1 |
| Total | 100 | 100 | 100 |

*0.6% tofacitinib corresponds to 1% tofacitinib citrate
m = micronized API

TABLE E1

| | 0.0/0.01** | 0.0/0.01** | 0.6*/0.01** | 0.6*/0.01** |
|---|---|---|---|---|
| Ingredients | % w/w | | | |
| Tofacitinib Citrate | 0.0 | 0.0 | 1.0 | 1.0 |
| Fingolimod HCl | 0.0112 | 0.0112 | 0.0112 | 0.0112 |
| ST-Elastomer 10 | 87.9888 | | 86.9888 | |
| MCT oil | 8 | 8 | 8 | 8 |
| Squalane | 2 | 2 | 2 | 2 |
| Isopropyl isostearate | 2 | 2 | 2 | 2 |
| Petrolatum | | 87.9888 | | 86.9888 |
| PEG 400 | | | | |
| PEG 3350 | | | | |
| Propylene glycol | | | | |
| Glycerin | | | | |
| Oleyl alcohol | | | | |
| BHA | | | | |
| Total | 100 | 100 | 100 | 100 |

### Example F. Water-In-Elastomer and Polar Solvent-In-Elastomer Formulations Comprising Tofacitinib

Water-in-Elastomer and Polar Solvent-in-Elastomer Formulations are prepared comprising tofacitinib citrate and/or Fingolimod HCl in a dissolved state (partially or entirely).

TABLE F

| | FS1(0.6* | FS2(0.6* | FS1(0.0/ | FS2(0.0/ | FS1(0.6*/ | FS2(0.6*/ |
|---|---|---|---|---|---|---|
| | m) | m) | 0.01**) | 0.01** | 0.01**) | 0.01**) |
| Ingredients | % w/w | | | | | |
| Tofacitinib Citrate | 1.0 | 1.0 | 0.0 | 0.0 | 1.0 | 1.0 |
| Fingolimod HCl | | | 0.0112 | 0.0112 | 0.0112 | 0.0112 |
| ST-Elastomer 10 | 64 | 64 | 64.9888 | 64.9888 | 63.9888 | 63.9888 |
| MCT oil | 8 | 8 | 8 | 8 | 8 | 8 |
| Squalane | 2 | 7 | 4 | 2 | 2 | 2 |
| Isopropyl isostearate | 2 | 7 | 4 | 2 | 2 | 2 |
| Water | 10 | | 10 | | 10 | |
| Hexylene Glycol | | 10 | | 10 | | 10 |
| Polysorbate 20 | 5 | 5 | 5 | 5 | 5 | 5 |
| Dow Corning ® 3225C | 8 | 8 | 8 | 8 | 8 | 8 |
| Formulation Aid | | | | | | |
| Total | 100 | 100 | 100 | 100 | 100 | 100 |

*0.6% tofacitinib corresponds to 1% tofacitinib citrate
m = micronized API
**0.01% fingolimod corresponds to 0.0112% of fingolimod HCl

In one or more embodiments there is provided an elastomer-based emulsion. In some embodiments the emulsion is a water in oil (elastomer-based) emulsion.

### Example G. Elastomer-Based Formulations Comprising a Combination of MCT Oil and Alternative Emollients

Elastomer formulations comprising tofacitinib citrate and/or fingolimod HCl with MCT oil and various emollients are prepared as shown in Tables G-G2 according the protocol described in the Methods.

TABLE G

| | OT1.00MOM | OT1.00OLM | OT1.00DISPAM | OT1.00GMM | OT1.00GIM | OT1.00GDM |
|---|---|---|---|---|---|---|
| | (0.6-m*) | (0.6-m*) | (0.6-m*) | (0.6-m*) | (0.6-m*) | (0.6-m*) |
| Ingredients | % w/w | | | | | |
| Tofacitinib Citrate | 1 | 1 | 1 | 1 | 1 | 1 |
| ST-Elastomer 10 | 87 | 87 | 87 | 87 | 87 | 87 |
| MCT oil | 6 | 6 | 6 | 6 | 6 | 6 |
| Mineral oil | 6 | | | | | |
| Olive oil | | 6 | | | | |
| Diisopropyl adipate | | | 6 | | | |
| Glyceryl monooleate | | | | 6 | | |
| Glyceryl isostearate | | | | | 6 | |
| Glyceryl dicaprate | | | | | | 6 |
| Total | 100 | 100 | 100 | 100 | 100 | 100 |

*0.6% tofacitinib corresponds to 1% tofacitinib citrate
m = micronized API

TABLE G1

| | OT1.00MOM | OT1.00OLM | OT1.00DISPAM | OT1.00GMM | OT1.00GIM | OT1.00GDM |
|---|---|---|---|---|---|---|
| | (0.01**) | (0.01**) | (0.01**) | (0.01**) | (0.01**) | (0.01**) |
| Ingredients | % w/w | | | | | |
| Fingolimod HCl* | 0.0112 | 0.0112 | 0.0112 | 0.0112 | 0.0112 | 0.0112 |
| ST-Elastomer 10 | 87.9888 | 87.9888 | 87.9888 | 87.9888 | 87.9888 | 87.9888 |
| MC T oil | 6 | 6 | 6 | 6 | 6 | 6 |
| Mineral oil | 6 | | | | | |
| Olive oil | | 6 | | | | |
| Diisopropyl adipate | | | 6 | | | |
| Glyceryl monooleate | | | | 6 | | |
| Glyceryl isostearate | | | | | 6 | |
| Glyceryl dicaprate | | | | | | 6 |
| Total | 100 | 100 | 100 | 100 | 100 | 100 |

**0.01% fingolimod corresponds to 0.0112% of fingolimod HCl

TABLE G2

| | OT1.00MOM | OT1.00OLM | OT1.00DISPAM | OT1.00GMM | OT1.00GIM | OT1.00GDM |
|---|---|---|---|---|---|---|
| | (0.6-m*0.01**) | (0.6-m*0.01**) | (0.6-m*0.01**) | (0.6-m*0.01**) | (0.6-m*0.01**) | (0.6-m*0.01**) |
| Ingredients | % w/w | | | | | |
| Fingolimod HCl* | 0.0112 | 0.0112 | 0.0112 | 0.0112 | 0.0112 | 0.0112 |
| Tofacitinib Citrate** | 1.0 | 1.0 | 1.0 | 1.0 | 1.0 | 1.0 |
| ST-Elastomer 10 | 86.9888 | 86.9888 | 86.9888 | 86.9888 | 86.9888 | 86.9888 |
| MCT oil | 6 | 6 | 6 | 6 | 6 | 6 |
| Mineral oil | 6 | | | | | |
| Olive oil | | 6 | | | | |
| Diisopropyl adipate | | | 6 | | | |
| Glyceryl monooleate | | | | 6 | | |
| Glyceryl isostearate | | | | | 6 | |
| Glyceryl dicaprate | | | | | | 6 |
| Total | 100 | 100 | 100 | 100 | 100 | 100 |

*0.6% tofacitinib corresponds to 1% tofacitinib citrate
**0.01% fingolimod corresponds to 0.0112% of fingolimod HCl

### Example H. Skin Penetration Study for Formulations Comprising Different Tofacitinib Salts

Investigation of the effect of the different formulations (Table H) on skin penetration is undertaken in accordance with the protocol set out in Methods section. The investigation examines, amongst other things, the effect of the different tofacitinib salts/free base alone or in combination with fingolimod HCl on skin penetration.

TABLE H

| | | | | | | OT1.0030AF | OT1.00TAF | OT1.00TLF | 0T100TMF |
|---|---|---|---|---|---|---|---|---|---|
| | 0T1.0030A | OT1.00TA | OT1.00TL | OT1.00TM | OT10030 | (0.6- | (0.6-m**/ | (0.6-m**/ | (0.6-m**/ |
| Ingredients | (0.6-m*) | (0.6-m*) | (0.6-m*) | (0.6-m*) | (0.6-m*) | m*0.01***) | 0.01***) | 0.01***) | 0.01***) |
| Tofacitinib | 1 | | | | | 1 | | | |
| Citrate | | | | | | | | | |
| Tofacitinib | | 1 | | | | | 1 | | |
| Adipate | | | | | | | | | |
| Tofacitinib | | | 1 | | | | | 1 | |
| Laurate | | | | | | | | | |
| Tofacitinib | | | | 1 | | | | | 1 |
| Myristate | | | | | | | | | |
| Tofacitinib | | | | | 1 | | | | |
| free base | | | | | | | | | |
| Fingolimod | | | | | | 0.0112 | 0.0112 | 0.0112 | 0.0112 |
| HCl*** | | | | | | | | | |
| ST-Elastomer | 87 | 87 | 87 | 87 | 87 | 86.9888 | 86.9888 | 86.9888 | 86.9888 |
| 10 | | | | | | | | | |
| Squalane | 2 | 2 | 2 | 2 | 2 | 2 | 2 | 2 | 2 |
| Isopropyl | 2 | 2 | 2 | 2 | 2 | 2 | 2 | 2 | 2 |
| isostearate | | | | | | | | | |
| MCT oil | 8 | 8 | 8 | 8 | 8 | 8 | 8 | 8 | 8 |
| Total | 100 | 100 | 100 | 100 | 100 | 100 | 100 | 100 | 100 |

*0.6% tofacitinib corresponds to 1% tofacitinib citrate
**Equivalent to 1% salt
m = micronized API
***0.01% fingolimod corresponds to 0.0112% of fingolimod HCl

### Example I. Elastomer-Based Foamable Formulations PGP-100 T2

Silicone oils are known defoamers so preparation of foams comprising high amounts of elastomers dispersed in silicone oils may be challenging. Adding high amounts of surfactants and/or fatty alcohols may overcome the defoaming effect of elastomers but may lead to an overly viscous formulation resulting in a valve block. In some embodiments, the composition comprises less than 5% by weight of a surfactant; about or less than 4.5% by weight; about or less than 4% by weight; about or less than 3.5% by weight; or about or less than 3% by weight or about or less than 2.5%, or about or less than 2% by weight or about or less than 1% by weight. In some embodiments the composition comprises about or more than 5% by weight of a surfactant when a dilutent is added. In some embodiments the composition comprises reduced amounts of elastomer as described in Example 18 and surfactant and/or fatty alcohols are added thereto. In some embodiments a combination of surfactant with a low HLB (lipophilic surfactant) and surfactant with a high HLB (hydrophilic surfactants) are used to generate a foam. In some embodiments the surfactant comprises a silicone surfactant. Because elastomers and silicone oils are defoamers then in one or more embodiments in order to generate a foam higher levels of foam adjuvants and surfactants may be needed, and or it may be appropriate at the same time to lower the proportion of elastomer and or silicone oils in the formulation (e.g., by increasing the level of emollient or other solvents gin order to generate a foam. In formulating a foamable composition, in one or more embodiments, the amounts of solids suspended in the formulation should be adjusted so as not to potentially cause a block in the canister valve. The formulation should also be adjusted so that after addition of propellant it is shakable, or moderately so in the canister.

Without being bound by any theory it is postulated that a silicone surfactant comprising oil- and silicone soluble groups would lower the surface tension of the oil thereby enable foam generation. In one or more embodiments the foamable elastomer composition comprises a silicone surfactant. In one or more embodiments a suitable silicone surfactant, by way of example, includes DOWSIL™ ES-5600 Silicone Glycerol Emulsifier (INCI Name: Cetyl Diglyceryl Tris(Trimethylsiloxy)silylethyl Dimethicone), DOWSIL™ BY 25-337 Silicone Emulsifier (INCI Name: PEG/PPG-19/19 Dimethicone (and) C13-16 Isoparaffin (and) C10-13 Isoparaffin), and DOWSIL BY 22-008M (INCI Name: Cyclopentasiloxane (and) PEG/PPG-19/19 Dimethicone).

Evaluation of elastomer-based foamable formulations (Table 1) for various foam properties is undertaken in accordance with the protocol set out in Methods section. The compositions comprise tofacitinib citrate and/or fingolimod HCl and a surfactant with or without a fatty alcohol, which may improve foam properties.

TABLE I

| | | Composition | | Composition | | Composition |
|---|---|---|---|---|---|---|
| | | with | | with | | with |
| | Composition | surfactant | Composition | surfactant | Composition | surfactant |
| | with | and fatty | with | and fatty | with | and fatty |
| | surfactant | alcohol | surfactant | alcohol | surfactant | alcohol |
| Ingredients | % w/w | | | | | |
| Tofacitinib Citrate | 1 | 1 | | | 1 | 1 |
| Fingolimod HCl** | | | 0.0112 | 0.0112 | 0.0112 | 0.0112 |
| ST-Elastomer 10 | 823 | 81 | 83.9888 | 81.9888 | 82.9888 | 80.9888 |
| Squalane | 2 | 2 | 2 | 2 | 2 | 2 |
| Isopropyl isostearate | 2 | 2 | 2 | 2 | 2 | 2 |
| MCT oil | 8 | 8 | 8 | 8 | 8 | 8 |
| Glyceryl monostearate | 4 | 4 | 4 | 4 | 4 | 4 |
| Cetostearyl alcohol | — | 2 | — | 2 | — | 2 |
| Total | 100 | 100 | 100 | 100 | 100 | 100 |
| AP-70 (butane, isobutane, | 12 | 12 | 12 | 12 | 12 | 12 |
| propane mixture) | | | | | | |

*0.6% tofacitinib corresponds to 1% tofacitinib citrate
m = micronized API
*0.01% fingolimod corresponds to 0.0112% of fingolimod HCl

TABLE I-1

| | | Composition | | Composition | | Composition |
|---|---|---|---|---|---|---|
| | | with | | with | | with |
| | Composition | surfactant | Composition | surfactant | Composition | surfactant |
| | with | and fatty | with | and fatty | with | and fatty |
| | surfactant | alcohol | surfactant | alcohol | surfactant | alcohol |
| Ingredients | % w/w | | | | | |
| Tofacitinib Citrate | 1 | 1 | | | 1 | 1 |
| Fingolimod HCl** | | | 0.0112 | 0.0112 | 0.0112 | 0.0112 |
| ST-Elastomer 10 | 15 | 15.5 | 17 | 17 | 17 | 15 |
| White mineral oil | 68 | 65.5 | 66.9888 | 64.9888 | 65.9888 | 65.9888 |
| Squalane | 2 | 2 | 2 | 2 | 2 | 2 |
| Isopropyl isostearate | 2 | 2 | 2 | 2 | 2 | 2 |
| MCT oil | 8 | 8 | 8 | 8 | 8 | 8 |
| Glyceryl monostearate | 4 | 4 | 4 | 4 | 4 | 4 |
| Cetostearyl alcohol | — | 2 | — | 2 | — | 2 |
| Total | 100 | 100 | 100 | 100 | 100 | 100 |
| AP-70 (butane, isobutane, | 12 | 12 | 12 | 12 | 12 | 12 |
| propane mixture) | | | | | | |

### Example J. Elastomer-Based Formulations Comprising Alternative Emollients

Evaluation of physical properties of Elastomer-based foamable formulations comprising tofacitinib citrate and/or fingolimod HCl (Tables J-J2) with various emollients is undertaken in accordance with the protocol set out in the Methods section. In one or more embodiments the effect of replacing PPG stearyl ether with one or more of glyceryl monooleate, glyceryl isostearate, glyceryl dicaprate is investigated. See Table J-J2.

TABLE J

| | OT1.00GM | OT1.00GI | OT1.00GD | OT1.00OL | OT1.00DISPA |
|---|---|---|---|---|---|
| | (0.6-m*) | (0.6-m*) | (0.6-m*) | (0.6-m*) | (0.6-m*) |
| Ingredients | | | | | |
| Tofacitinib Citrate | 1 | 1 | 1 | 1 | 1 |
| ST-Elastomer 10 | 87 | 87 | 87 | 87 | 87 |
| Glyceryl monooleate | 12 | | | | |
| Glyceryl isostearate | | 12 | | | |
| Glyceryl dicaprate | | | 12 | | |
| PPG 15 Stearyl ether | | | | | |
| Olive oil | | | | 12 | |
| Diisopropyl adipate | | | | | 12 |
| Total | 100 | 100 | 100 | 100 | 100 |
| Results | | | | | |

*0.6% tofacitinib corresponds to 1% tofacitinib citrate
m = micronized API

TABLE J1

| | OT1.00GM | OT1.00GI | OT1.00GD | OT1.00OL | OT1.00DISPA |
|---|---|---|---|---|---|
| | (0.0-0.01) | (0.0-0.01) | (0.0-0.01) | (0.0-0.01) | (0.0-0.01) |
| Ingredients | | | | | |
| Fingolimod HCl** | 0.0112 | 0.0112 | 0.0112 | 0.0112 | 0.0112 |
| ST-Elastomer 10 | 87.9888 | 87.9888 | 87.9888 | 87.9888 | 87.9888 |
| Glyceryl monooleate | 12 | | | | |
| Glyceryl isostearate | | 12 | | | |
| Glyceryl dicaprate | | | 12 | | |
| PPG 15 Stearyl ether | | | | | |
| Olive oil | | | | 12 | |
| Diisopropyl adipate | | | | | 12 |
| Total | 100 | 100 | 100 | 100 | 100 |
| Results | | | | | |

*0.6% tofacitinib corresponds to 1% tofacitinib citrate
**0.01% fingolimod corresponds to 0.0112% of fingolimod HCl

TABLE J-2

| | OT1.00GM | OT1.00GI | OT1.00GD | OT1.00OL | OT1.00DISPA |
|---|---|---|---|---|---|
| | (0.6-m/0.01) | (0.6-m/0.01) | (0.6-m/0.01) | (0.6-m/0.01) | (0.6-m/0.01) |
| Ingredients | | | | | |
| Tofacitinib Citrate* | 1 | 1 | 1 | 1 | 1 |
| Fingolimod HCl** | 0.0112 | 0.0112 | 0.0112 | 0.0112 | 0.0112 |
| ST-Elastomer 10 | 86.9888 | 86.9888 | 86.9888 | 86.9888 | 86.9888 |
| Glyceryl monooleate | 12 | | | | |
| Glyceryl isostearate | | 12 | | | |
| Glyceryl dicaprate | | | 12 | | |
| PPG 15 Stearyl ether | | | | | |
| Olive oil | | | | 12 | |
| Diisopropyl adipate | | | | | 12 |
| Total | 100 | 100 | 100 | 100 | 100 |
| Results | | | | | |

*0.6% tofacitinib corresponds to 1% tofacitinib citrate
**0.01% fingolimod corresponds to 0.0112% of fingolimod HCl

### Example K. Elastomer-Based Formulations with Different Concentrations of Nicotinamide and/or Fingolimod HCl and/or Tofacitinib Citrate Combinations

Investigation of the effect of the different formulations (Table K) containing different concentrations of Nicotinamide and/or tofacitinib citrate and/or Fingolimod HCl on various physical parameters is undertaken in accordance with the protocol set out in Methods section.

TABLE K

| | | | | | | 5% NA | | 3% NA |
|---|---|---|---|---|---|---|---|---|
| | 5% NA | 5% NA | 3% NA | 3% NA | 5% NA | with tof | 3% NA | with tof |
| | with tor | w/o tof | with tof | w/o tof | with fing | and fing | with fing | and fing |
| ST-Elastomer 10 | 82 | 83 | 84 | 85 | 82.9888 | 82.9888 | 84.9888 | 84.9888 |
| MCT oil | 8 | 8 | 8 | 8 | 8 | 8 | 8 | 8 |
| Squalane | 2 | 2 | 2 | 2 | 2 | 7 | 2 | 2 |
| IPIS | 2 | 2 | 2 | 2 | 2 | 2 | 2 | 2 |
| tofacitinib citrate | 1 | | 1 | | | 1 | | 1 |
| fingolimod HCl** | | | | | 0.0112 | 0.0112 | 0.0112 | 0.0112. |
| Nicotinamide | 5 | 5 | 3 | 3 | 5 | 5 | 2 | 3 |
| Total | 100 | 100 | 100 | 100 | 100 | 100 | 100 | 100 |

*0.6% tofacitinib corresponds to 1% tofacitinib citrate
**0.01% fingolimod corresponds to 0.0112% of fingolimod HCl

### Example L. In-Vivo Atopic Dermatitis Animal Model Study

A study is conducted to evaluate elastomer-based formulations containing tofacitinib citrate and fingolimod hydrochloride at different strengths in an Atopic Dermatitis animal model in accordance with the protocol set out in Experimental Method C. These dual active formulations (formulations 3, 4, 5, 6) are compared to fingolimod hydrochloride in elastomer based formulations (formulations 1, 2). The results described in Examples 11 for individual formulations of tofacitinib citrate in elastomer-based formulation (formulations 1, 2) when applied individually are compared to the fingolimod hydrochloride formulations (formulations 1 and 2) and dual active formulations (formulations 3, 4, 5, 6). The evaluated formulations are presented in Table L1. The treatments evaluated in the study are presented in Table L2.

Note that methodology is also described in Experimental Method “C” and the results of a single active such as tofacitinib or fingolimod elastomer-based formulations are described in Example 11 above (see Table 12c) and Example 38 above. In some embodiments, elastomer-based formulations comprising fingolimod hydrochloride and/or tofacitinib citrate are administered.

In some embodiments, successive application of tofacitinib citrate only in elastomer-based formulations followed by fingolimod hydrochloride in petrolatum-based formulations or elastomer based formulations is administered.

In one or more embodiments the study may be repeated with a broader and more wide-ranging dose variations. In one or more embodiments, for example, the amount of tofacitinib citrate is from 0.1% to 1.1% increasing in 0.2% increments and the amount of fingolimod hydrochloride is from 0.001 to 0.03 increasing in 0.005% increments.

TABLE L1

| | | | 3 | 4 | 5 | 6 |
|---|---|---|---|---|---|---|
| | 1 | 2 | TOF 0.6 + | TOF 0.6 + | TOF 0.6 + | TOF 0.3 + |
| | Fingolimod | Fingolimod | Fingolimod | Fingolimod | Fingolimod | Fingolimod |
| Formulation # | 0.005 | 0.02 | 0.005 | 0.01 | 0.02 | 0.02 |
| Ingredients | | | | | | |
| Fingolimod HCl | 0.0056 | 0.0224 | 0.0056 | 0.0112 | 0.0224 | 0.0224 |
| Tofacitinib Citrate | | | 1 | 1 | 1 | 0.5 |
| ST-Elastomer 10 | 87.9944 | 87.9776 | 86.9944 | 86.9888 | 86.9776 | 87.4776 |
| Squalene | 2 | 2 | 2 | 2 | 2 | 2 |
| Isopropyl | 2 | 2 | 2 | 2 | 2 | 2 |
| Isostearate | | | | | | |
| MCT oil | 8 | 8 | 8 | 8 | 8 | 8 |
| Total | 100 | 100 | 100 | 100 | 100 | 100 |

TABLE L2

| | Treatment group | Formulation |
|---|---|---|
| 1 | Negative control (induced, no treatment) | |
| 2 | fingolomid 0.005% | Formulation 1 |
| 3 | fingolomid 0.02% | Formulation 2 |
| 4 | Tofacitinib 0.6% + Fingolimod 0.005% | Formulation 3 |
| 5 | Tofacitinib 0.6% + Fingolimod 0.01% | Formulation 4 |
| 6 | Tofacitinib 0.6% + Fingolimod 0.02% | Formulation 5 |
| 7 | Tofacitinib 0.3% + Fingolimod 0.02% | Formulation 6 |
| 8 | Positive control − triamcinolone acetonide | |
| 9 | Naïve (not induced) | |

### Example M. Elastomer-Based Formulations Comprised of Tofacitinib Citrate and Fingolimod Hydrochloride

Evaluation of elastomer-based formulations comprising of fingolimod hydrochloride alone or a combination of tofacitinib Citrate and fingolimod hydrochloride at different ratios (Table M) is undertaken to evaluate their physical properties, stability, and skin penetration.

TABLE M

| T-F | T-F | T-F | T-F | T-F | T-F | | | |
|---|---|---|---|---|---|---|---|---|
| 0.3- | 0.3- | 0.3- | 0.6- | 0.6- | 0.6- | F 0.0- | F 0.0- | F 0.0- |
| 0.001 | 0.005 | 0.01 | 0.001 | 0.005 | 0.001 | 0.001 | 0.005 | 0.01 |

% w/w

| | | | | | | | | | |
|---|---|---|---|---|---|---|---|---|---|
| Tofacitinib Citrate | 0.5 | 0.5 | 0.5 | 1 | 1 | 1 | 0 | 0 | 0 |
| Fingolimod | 0.001 | 0.05 | 0.01 | 0.001 | 0.005 | 0.01 | 0.001 | 0.05 | 0.01 |
| Hydrochloride | | | | | | | | | |
| ST-Elastomer 10 | 87.499 | 87.45 | 87.49 | 86.999 | 86.995 | 86.99 | 87.999 | 87.95 | 87.99 |
| Squalane | 2 | 2 | 2 | 2 | 2 | 2 | 2 | 2 | 2 |
| Isopropyl | 2 | 2 | 2 | 2 | 2 | 2 | 2 | 2 | 2 |
| isostearate | | | | | | | | | |
| MCT oil | 8 | 8 | 8 | 8 | 8 | 8 | 8 | 8 | 8 |
| Total | 100 | 100 | 100 | 100 | 100 | 100 | 100 | 100 | 100 |

Note:
0.3% tofacitinib corresponds to 0.5% tofacitinib citrate;
0.6% tofacitinib corresponds to 1% tofacitinib citrate
0.01% fingolimod corresponds to 0.0112% of fingolimod HCl

### Example N. Elastomer-Based Formulations Comprised of Tofacitinib Citrate and Fingolimod Hydrochloride or Fingolimod Hydrochloride Alone

Evaluation of elastomer-based formulations comprising of MCT oil, isopropyl isostearate and squalene at different ratios and combination of tofacitinib citrate and fingolimod hydrochloride or fingolimod hydrochloride alone (Table N and N1 respectively) is undertaken to evaluate their physical properties including homogenous/phase separation, aggregation, sedimentation, viscosity and visual appearance (e.g. transparent or translucent or cloudy solid or flowing), homogenous distribution of API crystals), chemical stability and skin penetration.

TABLE N

| | T-F | T-F | T-F | T-F | T-F | T-F |
|---|---|---|---|---|---|---|
| | 0.3- | 0.3- | 0.3- | 0.3- | 0.3- | 0.3- |
| | 0.01-A | 0.01-B | 0.01-C | 0.01-D | 0.01-E | 0.01-F |
| | % w/w | | | | | |
| Tofacitinib | 0.5 | 0.5 | 0.5 | 0.5 | 0.5 | 0.5 |
| Citrate | | | | | | |
| Fingolimod | 0.01 | 0.01 | 0.01 | 0.01 | 0.01 | 0.01 |
| Hydrochloride | | | | | | |
| ST-Elastomer | 87.49 | 87.49 | 8783.49 | 87.49 | 87.49 | 87.49 |
| 10 | | | | | | |
| Squalane | 2 | 4 | 2 | 6 | 4 | 6 |
| Isopropyl | 4 | 2 | 6 | 2 | 6 | 4 |
| isostearate | | | | | | |
| MCT oil | 6 | 6 | 4 | 4 | 2 | 2 |
| Total | 100 | 100 | 100 | 100 | 100 | 100 |

Note:
0.3% tofacitinib corresponds to 0.5% tofacitinib citrate;
0.6% tofacitinib corresponds to 1% tofacitinib citrate
0.01% fingolimod corresponds to 0.0112% of fingolimod HCl

TABLE N-1

| | T-F | T-F | T-F | T-F | T-F | T-F |
|---|---|---|---|---|---|---|
| | 0.0- | 0.0- | 0.0- | 0.0- | 0.0- | 0.0- |
| | 0.01-A | 0.01-B | 0.01-C | 0.01-D | 0.01-E | 0.01-F |
| | % w/w | | | | | |
| Fingolimod | 0.01 | 0.01 | 0.01 | 0.01 | 0.01 | 0.01 |
| Hydrochloride | | | | | | |
| ST-Elastomer | 87.99 | 87.99 | 87.99 | 87.99 | 87.99 | 87.99 |
| 10 | | | | | | |
| Squalane | 2 | 4 | 2 | 6 | 4 | 6 |
| Isopropyl | 4 | 2 | 6 | 2 | 6 | 4 |
| isostearate | | | | | | |
| MCT oil | 6 | 6 | 4 | 4 | 2 | 2 |
| Total | 100 | 100 | 100 | 100 | 100 | 100 |

0.01% fingolimod corresponds to 0.0112% of fingolimod HCl

### Example O. Elastomer-Based Formulations Comprised of Tofacitinib Citrate and Fingolimod Free Base or Fingolimod Free Base Alone

Alternative to fingolimod hydrochloride, the free base form of fingolimod is also evaluated alone or in combination with tofacitinib citrate.

Evaluation of elastomer-based formulations comprising of combination of Tofacitinib Citrate and Fingolimod base or fingolimod hydrochloride alone at different ratios (Table O) is undertaken to evaluate their physical properties, stability, and skin penetration.

TABLE O

| | T-F | T-F | T-F | T-F | T-F | T-F | T-F | T-F |
|---|---|---|---|---|---|---|---|---|
| | 0.3- | 0.3- | 0.3- | 0.6- | 0.6- | 0.6- | 0.0- | 0.0- |
| | 0.001 | 0.005 | 0.01 | 0.001 | 0.005 | 0.001 | 0.001 | 0.01 |
| | % w/w | | | | | | | |
| Tofacitinib Citrate | 0.5 | 0.5 | 0.5 | 1 | 1 | 1 | 0 | 0 |
| Fingolimod base | 0.001 | 0.05 | 0.01 | 0.001 | 0.005 | 0.01 | 0.001 | 0.01 |
| ST-Elastomer 10 | 87.499 | 87.45 | 87.49 | 86.999 | 86.995 | 86.99 | 87.999 | 87.99 |
| Squalane | 2 | 2 | 2 | 2 | 2 | 2 | 2 | 2 |
| Isopropyl isostearate | 2 | 2 | 2 | 2 | 2 | 2 | 2 | 2 |
| MCT oil | 8 | 8 | 8 | 8 | 8 | 8 | 8 | 8 |
| Total | 100 | 100 | 100 | 100 | 100 | 100 | 100 | 100 |

Note:
0.3% tofacitinib corresponds to 0.5% tofacitinib citrate;
0.6% tofacitinib corresponds to 1% tofacitinib citrate
0.01% fingolimod corresponds to 0.0112% of fingolimod HCl

### Example P. Solubility of Fingolimod Hydrochloride and Fingolimod Base in Different Solvents

Fingolimod HCl and fingolimod base solubility is tested in different excipients (Table P) as described in the Experimental Method D.

TABLE P

| | Solubility of | Solubility of |
|---|---|---|
| | Fingolimod | Fingolimod |
| Solvent | HCl, mg/g | base, mg/g |
| DMSO | | |
| Polyethylene Glycol 400 | | |
| Water | | |
| Transcutol | | |
| Propylene Glycol | | |
| Glycerin | | |
| Benzyl Alcohol | | |
| Ethanol | | |
| Hexylene Glycol | | |
| Isopropyl Alcohol | | |
| Oleyl alcohol | | |
| Diisopropyl adipate | | |
| Isopropyl myristate | | |
| MCT oil (Caprylic/capric | | |
| triglycerides) | | |
| Isopropyl palmitate | | |
| Squalane | | |
| Isopropyl isostearate | | |
| Cyclomethicone | | |

### Example Q. Solubility of Fingolimod Hydrochloride or Fingolimod Base and Tofacitinib Citrate or Tofacitinib Base Alone or in One or More Combinations in Formulations Based on ST Elastomer 10, MCT Oil, Squalene and IPIS are Evaluated

The solubility of fingolimod HCl or fingolimod base and tofacitinib citrate or tofacitinib base are evaluated for formulations comprised of MCT oil, squalene, isopropyl isostearate and ST-Elastomer 10. To evaluate solubility, the mixture of MCT oil, isopropyl isostearate and squalene is combined with cyclomethicone, which is utilized as liquid substitution ST-Elastomer, is used (see Table Q below). The mixture is then equilibrated with excess amount of either tofacitinib citrate or fingolimod hydrochloride or fingolimod base to achieve saturated solution in accordance with the protocol set out in Experimental Method D.

TABLE Q

| Ingredients | % w/w | | | | | | |
|---|---|---|---|---|---|---|---|
| Cyclomethicone | 88 | 88 | 88 | 88 | 88 | 88 | 88 |
| Isopropyl isostearate | 2 | 2 | 4 | 2 | 6 | 4 | 6 |
| Squalane | 2 | 4 | 2 | 6 | 2 | 6 | 4 |
| MCT oil | 8 | 6 | 6 | 4 | 4 | 2 | 2 |
| Total | 100 | 100 | 100 | 100 | 100 | 100 | 100 |

### Example R. Evaluation of Compatibility of Tofacitinib Citrate with Fingolimod Hydrochloride in Different Solvents for Topical Delivery

Combinations of tofacitinib citrate (“TOFc”) with fingolimod hydrochloride (“Fingh”) (Table R),—dispersed or partially dissolved or fully dissolved—in different solvents are evaluated for chemical stability of Tofacitinib and Fingolimod. The mixtures are incubated for 5 weeks at 60° C. and evaluated by HPLC for tofacitinib assay and degradation products as well as for fingolimod assay and degradation products as described in Experimental Method E.

TABLE R

| | TOF | TOF Deg. | Fing | Fing Deg. |
|---|---|---|---|---|
| Sample name | assay | products | assay | products |
| TOFc + Fingh in MCT oil | | | | |
| TOFc + Fingh in Squalane | | | | |
| TOFc + Fingh in Isopropyl Isosterate | | | | |
| TOFc + Fingh in Cyclomethicone | | | | |
| TOFc + Fingh in Propylene glycol | | | | |
| TOFc + Fingh in Glycerin | | | | |
| TOFc + Fingh in Oleyl alcohol | | | | |
| TOFc + Fingh in Oleic acid | | | | |
| TOFc + Fingh in Mineral oil | | | | |
| TOFc + Fingh in Petrolatum | | | | |
| TOFc + Fingh in Octisalate | | | | |
| TOFc + Fingh in Diisopropyl adipate | | | | |
| TOFc + Fingh in Isopropyl myristate | | | | |

Fingolimod hydrochloride alone (Table R1) (dispersed or partially dissolved or fully dissolved) in different solvents is evaluated for chemical stability. The mixtures are incubated for 5 weeks at 60° C. and evaluated by HPLC for fingolimod assay and degradation products as described in Experimental Method E.

TABLE R1

| | | Fing Deg. |
|---|---|---|
| Sample name | Fing assay | products |
| Fing in MCT oil | | |
| Fing in Squalane | | |
| Fing in Isopropyl Isosterate | | |
| Fing in Cyclomethicone | | |
| Fing in Propylene glycol | | |
| Fing in Glycerin | | |
| Fing in Oleyl alcohol | | |
| Fing in Oleic acid | | |
| Fing in Mineral oil | | |
| Fing in Petrolatum | | |
| Fing in Octisalate | | |
| Fing in Diisopropyl adipate | | |
| Fing in Isopropyl myristate | | |

Note that methodology is described in Experimental Methods “E” and “H” and the chemical stability results of Tofacitinib citrate alone (Table 17b-g above) (dispersed or partially dissolved or fully dissolved) in different solvents are described in Example 16.

### Example Ra. Evaluation of Compatibility of Tofacitinib Base with Fingolimod Base in Different Solvents for Topical Delivery

Example R above is repeated with tofacitinib free base and fingolimod free base in combination and also separately instead of their salts.

### Example S. Elastomer-Based Formulations Tofacitinib Citrate and Fingolimod HCl in Combination with Different Anti-Scar Agents

Silicone elastomer-based formulations, comprising tofacitinib citrate and fingolimod hydrochloride are prepared for the prevention or treatment of scars (such as skin scars, burns, hypertrophic scars, keloid scars, post-surgery scars). Optionally, additional agents known for their anti-scarring activity, such as hyaluronic acid, pentamidine, nicotinic acid, caffeine are added to the formulation as shown in Table S.

TABLE S

| Ingredients | % w/w | % w/w | % w/w | % w/w | % w/w |
|---|---|---|---|---|---|
| Tofacitinib Citrate | 1 | 1 | 1 | 1 | 1 |
| Fingolimod HCl | 0.01 | 0.01 | 0.01 | 0.01 | 0.01 |
| ST-Elastomer 10 | 86.99 | 85.99 | 85.99 | 85.99 | 86.99 |
| Squalane | 2 | 2 | 2 | 2 | 2 |
| Isopropyl isostearate | 2 | 2 | 2 | 2 | 2 |
| MCT oil | 8 | 8 | 8 | 8 | 8 |
| Hyaluronic acid | | 1 | | | |
| Pentamidine | | | 1 | | |
| Nicotinic acid | | | | 1 | |
| Caffeine | | | | | 1 |
| Total | 100 | 100 | 100 | 100 | 100 |

### Example T. Formulations Comprising of Tofacitinib Citrate and Fingolimod HCl in Different Platforms

Formulations comprising tofacitinib citrate and fingolimod hydrochloride are prepared in various alternative delivery platforms, such as emulsions, ointments and oil gels as illustrated in Table T.

TABLE T

| | Ointment | Oilgel | Emulsion |
|---|---|---|---|
| Ingredients | % w/w | % w/w | % w/w |
| Tofacitinib Citrate | 1 | 1 | 1 |
| Fingolimod HCl | 0.01 | 0.01 | 0.01 |
| Petrolatum | 86.99 | — | — |
| Squalane | 2 | — | — |
| Isopropyl isostearate | 2 | — | — |
| MCT oil | 8 | 78.99 | 10 |
| Cetostearyl alcohol | — | 20 | 2 |
| Ceteareth 20 | — | — | 3.5 |
| Glyceryl monostearate | — | — | 1.5 |
| Hypromellose K100M | — | — | 0.5 |
| Citrate buffer, 50 mM, pH 4.5 | — | — | 75.49 |
| Glycerin | — | — | 5 |
| Benzyl alcohol | — | — | 1 |
| Total | 100 | 100 | 100 |

### Example U. Physical Properties of Elastomer-Based Carrier Formulations with Different Amounts of MCT Oil

Formulations with various proportions of medium chain triglycerides (MCT oil) in ST-elastomer 10 comprising tofacitinib citrate and/or fingolimod hydrochloride are prepared as shown in Tables U-U2 and are evaluated for their physical properties, such as homogenous/phase separation, aggregation, sedimentation, viscosity and visual appearance (e.g., transparent or translucent or cloudy solid or flowing), homogenous distribution of API crystals).

TABLE U

| | OT1. | OT1. | OT1. | OT1. | OT1. | OT1. |
|---|---|---|---|---|---|---|
| | 0002 | 0003 | 0004 | 0001 | 0016 | 0012 |
| | (0.6/0.0) | (0.6/0.0) | (0.6/0.0) | (0.6/0.0) | (0.6/0.0) | (0.6/0.0) |
| Ingredient | % w/w | | | | | |
| Tofacitinib | 1.0 | 1.0 | 1.0 | 1.0 | 1.0 | 1.0 |
| Citrate | | | | | | |
| ST- | 49 | 69 | 79 | 85 | 87 | 89 |
| Elastomer | | | | | | |
| 10 | | | | | | |
| MCT oil | 50 | 30 | 20 | 14 | 12 | 10 |
| Total | 100 | 100 | 100 | 100 | 100 | 100 |

TABLE U-1

| | OT1.0002 | OT1.0003 | OT1.0004 | OT1.0001 | OT1.0016 | OT1.0012 |
|---|---|---|---|---|---|---|
| | (0.0/0.01) | (0.0/0.01) | (0.0/0.01) | (0.0/0.01) | (0.0/0.01) | (0.0/0.01) |
| Ingredient | % w/w | | | | | |
| Fingolimod HCl | 0.01 | 0.01 | 0.01 | 0.01 | 0.01 | 0.01 |
| ST-Elastomer 10 | 49.99 | 69.99 | 79.99 | 85.99 | 87.99 | 89.99 |
| MCT oil | 50 | 30 | 20 | 14 | 12 | 10 |
| Total | 100 | 100 | 100 | 100 | 100 | 100 |

TABLE U-2

| | OT1.0002 | OT1.0003 | OT1.0004 | OT1.0001 | OT1.0016 | OT1.0012 |
|---|---|---|---|---|---|---|
| | (0.6/0.01) | (0.6/0.01) | (0.6/0.01) | (0.6/0.01) | (0.6/0.01) | (0.6/0.01) |
| Ingredient | % w/w | | | | | |
| Tofacitinib Citrate | 1.0 | 1.0 | 1.0 | 1.0 | 1.0 | 1.0 |
| Fingolimod HCl | 0.01 | 0.01 | 0.01 | 0.01 | 0.01 | 0.01 |
| ST-Elastomer 10 | 48.99 | 68.99 | 78.99 | 84.99 | 86.99 | 88.99 |
| MCT oil | 50 | 30 | 20 | 14 | 12 | 10 |
| Total | 100 | 100 | 100 | 100 | 100 | 100 |

### Example V. Physical Properties of Elastomer-Based Carrier Formulations with Different Amounts of Alternative Oils, Such as Isopropyl Palmitate and Isopropyl Myristate

Formulations with various proportions of either isopropyl palmitate or isopropyl myristate in ST-elastomer 10 comprising tofacitinib citrate and/or fingolimod hydrochloride are prepared as shown in Tables Va1-Va3 and are evaluated for their physical properties such as homogenous/phase separation, aggregation, sedimentation, viscosity and visual appearance (e.g., transparent or translucent or cloudy solid or flowing), homogenous distribution of API crystals.

TABLE V-a1
Isopropyl Palmitate

| | OT1.0006 | OT1.0008 | OT1.0007 | OT1.0005 | OT1.0013 |
|---|---|---|---|---|---|
| | (0.6/0.0) | (0.6/0.0) | (0.6/0.0) | (0.6/0.0) | (0.6/0.0) |
| Ingredient | % w/w | | | | |
| Tofacitinib | 1.0 | 1.0 | 1.0 | 1.0 | 1.0 |
| Citrate | | | | | |
| Fingolimod HCl | 0.0 | 0.0 | 0.0 | 0.0 | 0.0 |
| ST-Elastomer 10 | 49 | 69 | 79 | 85 | 89 |
| Isopropyl | 50 | 30 | 20 | 14 | 10 |
| Palmitate | | | | | |
| Total | 100 | 100 | 100 | 100 | 100 |

TABLE V-a2
Isopropyl Palmitate

| | OT1.0006 | OT1.0008 | OT1.0007 | OT1.0005 | OT1.0013 |
|---|---|---|---|---|---|
| | (0.0/0.01) | (0.0/0.01) | (0.0/0.01) | (0.0/0.01) | (0.0/0.01) |
| Ingredient | % w/w | | | | |
| Tofacitinib | 0.0 | 0.0 | 0.0 | 0.0 | 0.0 |
| Citrate | | | | | |
| Fingolimod HCl | 0.01 | 0.01 | 0.01 | 0.01 | 0.01 |
| ST-Elastomer 10 | 49.99 | 69.99 | 79.99 | 85.99 | 89.99 |
| Isopropyl | 50 | 30 | 20 | 14 | 10 |
| Palmitate | | | | | |
| Total | 100 | 100 | 100 | 100 | 100 |

TABLE V-a3
Isopropyl Palmitate

| | OT1.0006 | OT1.0008 | OT1.0007 | OT1.0005 | OT1.0013 |
|---|---|---|---|---|---|
| | (0.6/0.01) | (0.6/0.01) | (0.6/0.01) | (0.6/0.01) | (0.6/0.01) |
| Ingredient | % w/w | | | | |
| Tofacitinib | 1.0 | 1.0 | 1.0 | 1.0 | 1.0 |
| Citrate | | | | | |
| Fingolimod HCl | 0.01 | 0.01 | 0.01 | 0.01 | 0.01 |
| ST-Elastomer 10 | 48.99 | 68.99 | 78.99 | 84.99 | 88.99 |
| Isopropyl | 50 | 30 | 20 | 14 | 10 |
| Palmitate | | | | | |
| Total | 100 | 100 | 100 | 100 | 100 |

TABLE V-b1
Isopropyl myristate

| | OT1.0011 | OT1.0010 | OT1.0009 | OT1.0014 |
|---|---|---|---|---|
| | (0.6/0.0) | (0.6/0.0) | (0.6/0.0) | (0.6/0.0) |
| Ingredient | % w/w | | | |
| Tofacitinib Citrate | 1.0 | 1.0 | 1.0 | 1.0 |
| Fingolimod HCl | 0.0 | 0.0 | 0.0 | 0.0 |
| ST-Elastomer 10 | 69 | 79 | 85 | 89 |
| isopropyl myristate | 30 | 20 | 14 | 10 |
| Total | 100 | 100 | 100 | 100 |

TABLE V-b2
Isopropyl myristate

| | OT1.0011 | OT1.0010 | OT1.0009 | OT1.0014 |
|---|---|---|---|---|
| | (0.0/0.01) | (0.0/0.01) | (0.0/0.01) | (0.0/0.01) |
| Ingredient | % w/w | | | |
| Tofacitinib Citrate | 0.0 | 0.0 | 0.0 | 0.0 |
| Fingolimod HCl | 0.01 | 0.01 | 0.01 | 0.01 |
| ST-Elastomer 10 | 69.99 | 79.99 | 85.99 | 89.99 |
| Isopropyl myristate | 30 | 20 | 14 | 10 |
| Total | 100 | 100 | 100 | 100 |

TABLE V-b3
Isopropyl myristate

| | OT1.0011 | OT1.0010 | OT1.0009 | OT1.0014 |
|---|---|---|---|---|
| | (0.6/0.01) | (0.6/0.01) | (0.6/0.01) | (0.6/0.01) |
| Ingredient | % w/w | | | |
| Tofacitinib Citrate | 1.0 | 1.0 | 1.0 | 1.0 |
| Fingolimod HCl | 0.01 | 0.01 | 0.01 | 0.01 |
| ST-Elastomer 10 | 68.99 | 78.99 | 84.99 | 88.99 |
| Isopropyl myristate | 30 | 20 | 14 | 10 |
| Total | 100 | 100 | 100 | 100 |

### Example W. HET-CAM Assay (Hen's Egg-Chorioallantoic Membrane Test) for Elastomer-Based Formulations and Emulsion-Based Formulations

Elastomer-based formulations comprising MCT oil and emulsion-based formulations with fingolimod hydrochloride or a combination of fingolimod hydrochloride and tofacitinib citrate (see Table W) are tested in a HET-CAM assay. Formulations are compared with NaOH 0.1% solution as a positive control and saline 0.9% as a negative control.

Note that methodology is also described in Experimental Method “G” and the results of a placebo and tofacitinib elastomer-based formulations comprising MCT oil and placebo and active emulsion-based formulations are described in Example 14 above (see Table 13).

In some embodiment elastomer-based formulations comprising fingolimod hydrochloride alone or a combination of fingolimod hydrochloride and tofacitinib citrate have a better tolerability potential than the emulsion-based formulations.

TABLE W

| | TOF055 | TOF013 | TOF055 | TOF013 |
|---|---|---|---|---|
| Ingredients | (0/0.01) | (0/0.01) | (0.6/0.01) | (0.15/0.01) |
| MCT oil | 13.00 | 10.00 | 13.00 | 10.00 |
| ST-Elastomer 10 | 86.99 | 0 | 85.99 | 0 |
| Tofacitinib citrate* | 0 | 0 | 1.00 | 1.00 |
| Fingolimod HCl** | 0.01 | 0.01 | 0.01 | 0.01 |
| Stearyl alcohol | 0 | 2.00 | 0 | 2.00 |
| Ceteareth 20 | 0 | 3.50 | 0 | 3.50 |
| Glyceryl monostearate | 0 | 1.50 | 0 | 1.50 |
| Hypromellose K100M | 0 | 0.50 | 0 | 0.50 |
| Citrate buffer, 50 mM, pH 4.5 | 0 | 76.49 | 0 | 75.49 |
| Glycerin | 0 | 5.00 | 0 | 5.00 |
| Benzyl alcohol | 0 | 1.00 | 0 | 1.00 |
| Total | 100.00 | 100.00 | 100.00 | 100.00 |

*0.6% tofacitinib corresponds to 1% tofacitinib citrate (non-micronized); 0.15% tofacitinib corresponds to 0.25% tofacitinib citrate (non-micronized)
**0.01% fingolimod corresponds to 0.0112% of fingolimod HCl

### Example X. Day 14 Plasma Levels in Minipigs Applied an Elastomer-Based Formulation with Fingolimod Alone or in Combination with Tofacitinib

Two minipigs are treated topically once-daily for 14 days with a formulation of 0.01% fingolimod alone or 0.01% fingolimod in combination 0.3% of tofacitinib (as citrate) (Table X). On day 14, blood samples are collected and plasmas are analyzed for their tofacitinib or fingolimod content as described in the Methods section. Note that methodology is also described in Experimental Method “O” and that plasma levels in minipigs dosed with a tofacitinib alone formulation are described in Example 15 and Tables 16a and b above.

TABLE X

| | T-F | T-F 0.0-0.01 |
|---|---|---|
| | (0.0/0.01) | (0.3/0.01) |
| Ingredients | % w/w | % w/w |
| MCT oil | 13.00 | 13.00 |
| ST-Elastomer 10 | 86.99 | 86.49 |
| Tofacitinib citrate* | 0.0 | 0.50 |
| Fingolimod HCl** | 0.01 | 0.01 |
| Total | 100.00 | 100.00 |

*0.3% tofacitinib corresponds to 0.5% tofacitinib citrate (non-micronized)
**0.01% fingolimod corresponds to 0.0112% of fingolimod HCl

### Example Y. Active Agent Interfacial Tension with Stainless Steel

Different elastomer-based formulations comprising fingolimod alone or in combination with tofacitinib citrate are evaluated for accumulation of the active agent on the stainless mixing propellers are manufactured (see Table Y and Table Y1). Note that methodology is also described in Experimental Method “J” and that interfacial tension with a tofacitinib alone formulation is described in Example 17 and Tables 18a and b above.

TABLE Y

| | OT1.0016A | OT1.0018A | OT1.0019A | OT1.0020A | OT1.0021A | OT1.0022A |
|---|---|---|---|---|---|---|
| | (0/0.01) | (0/0.01) | (0/0.01) | (0/0.01) | (0/0.01) | (0/0.01) |
| Ingredients | % w/w | | | | | |
| Fingolimod HCl | 0.01 | 0.01 | 0.01 | 0.01 | 0.01 | 0.01 |
| ST-Elastomer 10 | 87.99 | 87.99 | 87.99 | 87.99 | 87.99 | 87.99 |
| MCT oil | 12 | 6 | 10 | 10 | 8 | 8 |
| Isopropyl | | 6 | 2 | | | |
| Palmitate | | | | | | |
| Oleyl alcohol | | | | 2 | | 2 |
| Squalane | | | | | 2 | 2 |
| Isopropyl | | | | | 2 | |
| isostearate | | | | | | |
| Total | 100 | 100 | 100 | 100 | 100 | 100 |

TABLE Y1

| | OT1.0016A | OT1.0018A | OT1.0019A | OT1.0020A | OT1.0021A | OT1.0022A |
|---|---|---|---|---|---|---|
| | (0.3/0.01) | (0.3/0.01) | (0.3/0.01) | (0.3/0.01) | (0.3/0.01) | (0.3/0.01) |
| Ingredients | % w/w | | | | | |
| Tofacitinib | 0.5 | 0.5 | 0.5 | 0.5 | 0.5 | 0.5 |
| citrate* | | | | | | |
| Fingolimod HCl | 0.01 | 0.01 | 0.01 | 0.01 | 0.01 | 0.01 |
| ST-Elastomer 10 | 87.49 | 87.49 | 87.49 | 87.49 | 87.49 | 87.49 |
| MCT oil | 12 | 6 | 10 | 10 | 8 | 8 |
| Isopropyl | | 6 | 2 | | | |
| Palmitate | | | | | | |
| Oleyl alcohol | | | | 2 | | 2 |
| Squalane | | | | | 2 | 2 |
| Isopropyl | | | | | 2 | |
| isostearate | | | | | | |
| Total | 100 | 100 | 100 | 100 | 100 | 100 |

*0.3% Tofacitinib corresponds to 0.5% tofacitinib citrate (non-micronized)
**0.01% fingolimod corresponds to 0.0112% of fingolimod HCl

### Example Z. Microscopic Evaluation of an Elastomer-Based Formulation Comprising MCT Oil and Additional Emollients with a Micronized Tofacitinib Salt or Free Base or Fingolimod Salt or Free Base

Elastomer-based formulations comprising a micronized tofacitinib free base or fingolimod free base or fingolimod HCL (Table Z) are prepared and examined under a microscope. Microscopic evaluation of tofacitinib citrate elastomer based formulations is described above in Example 21.

TABLE Z

| | OT1.0031A | OT1.0031A | OT1.0031A |
|---|---|---|---|
| Ingredients | (1.2-fb) | (0.01 Fhcl) | (0.001 -Ffb) |
| Tofacitinib free base | 1.2 | | |
| Fingolimod HCl** | | 0.01 | |
| Fingolimod free base | | | 0.01 |
| ST-Elastomer 10 | 86.8 | 87.99 | 87.99 |
| MCT oil | 8 | 8 | 8 |
| Squalane | 2 | 2 | 2 |
| Isopropyl isostearate | 2 | 2 | 2 |
| Total | 100 | 100 | 100 |

**0.01% fingolimod corresponds to 0.0112% of fingolimod HCl

### Example i. Formulations with Alternative Elastomers

Investigation of the effect of the different elastomers (Table i and i-1) on various physical parameters of fingolimod alone or in combination with tofacitinib is undertaken in accordance with the protocol set out in Methods sections I. M and P. The investigation examines, amongst other things, the effect of the different elastomers on the physical properties of the formulation, including homogenous/phase separation, aggregation, sedimentation, viscosity and visual appearance (e.g., transparent or translucent or cloudy solid or flowing), homogenous distribution of API crystals. In one or more embodiments other elastomers are swollen or expanded in a silicone oil. In one or more embodiments other elastomers are suspended or dispersed in a silicone oil.

TABLE i

| | OT1.0030A | OT1.00ELA | OT1.0030ELB | OT1.0030ELC |
|---|---|---|---|---|
| | (0/0.01) | (0/0.01) | (0/0.01) | (0/0.01) |
| Ingredients | % w/w | | | |
| Fingolimod | 0.01 | 0.01 | 0.01 | 0.01 |
| HCl | | | | |
| ST-Elastomer | 87.99 | | | |
| 10 | | | | |
| ST-Elastomer | | 87.99 | | |
| 1148 | | | | |
| Gransil | | | 87.99 | |
| DMG-6 | | | | |
| Gransil DM-5 | | | | 87.99 |
| Elastomer | | | | |
| Squalane | 2 | 2 | 2 | 2 |
| Isopropyl | 2 | 2 | 2 | 2 |
| isostearate | | | | |
| MCT oil | 8 | 8 | 8 | 8 |
| Total | 100 | 100 | 100 | 100 |

TABLE i-1

| | OT1.0030A | OT1.00ELA | OT1.0030ELB | OT1.0030ELC |
|---|---|---|---|---|
| | (0.6/0.01) | (0.6/0.01) | (0.6/0.01) | (0.6/0.01) |
| Ingredients | % w/w | | | |
| Tofacitinib | 1 | 1 | 1 | 1 |
| Citrate - m* | | | | |
| Fingolimod | 0.01 | 0.01 | 0.01 | 0.01 |
| HCl | | | | |
| ST-Elastomer | 86.99 | | | |
| 10 | | | | |
| ST-Elastomer | | 86.99 | | |
| 1148 | | | | |
| Gransil | | | 86.99 | |
| DMG-6 | | | | |
| Gransil DM-5 | | | | 86.99 |
| Elastomer | | | | |
| Squalane | 2 | 2 | 2 | 2 |
| Isopropyl | 2 | 2 | 2 | 2 |
| isostearate | | | | |
| MCT oil | 8 | 8 | 8 | 8 |
| Total | 100 | 100 | 100 | 100 |

*0.6% tofacitinib corresponds to 1% tofacitinib citrate; m = micronized API

### Example ii. Water Activity

The water activity of formulation OT1.0021 with 0.01 fingolimod and for the corresponding formulation 1.2% oftofacitinib and 0.01 fingolimod (see Table ii below) is measured in accordance with USP<1112>. Note that the results of a placebo and tofacitinib elastomer-based formulations are described in Example 32. In one or more embodiments water activity is low in one or more active formulations.

TABLE ii

| Ingredients | OT1.0021(0/0.01) | OT1.0021(1.2/0.01) |
|---|---|---|
| Tofacitinib | 0 | 2.0 |
| Citrate - m* | | |
| Fingolimod HCl | 0.01 | 0.01 |
| ST-Elastomer 10 | 87.99 | 85.99 |
| MCT oil | 8 | 8 |
| Squalane | 2 | 2 |
| Isopropyl isostearate | 2 | 2 |
| Total | 100 | 100 |
| Water activity | | |

*1.2% tofacitinib corresponds to 2% tofacitinib citrate; m = micronized API

### Example iii. Formulations Comprising Fingolimod HCl Dissolved (Partially or Entirely)

Formulations comprising fingolimod HCL alone or in combination with tofacitinib citrate in a dissolved state (partially or entirely) are prepared as shown in tables iii and iii-1 below. In some embodiments there is provided a formulation in which tofacitinib and/or fingolimod has some solubility. In some embodiments the tofacitinib salt is solubilized in DMSO or another known solvent for tofacitinib salt. In some embodiments the fingolimod salt is solubilized in DMSO or another known solvent for fingolimod salt, such as water or ethanol. In some embodiments the DMSO is about 5% to about 15%, e.g., about 50, about 7.5%, about 10%, about 12.5% or about 15% by weight of the composition. In some embodiments, ethanol is about 5% to about 15%, e.g., about 5%, about 7.5%, about 10%, about 12.5% or about 15% by weight of the composition. In some embodiments water is about 5% to about 15%. e.g., about 5%, about 7.5%, about 10%, about 12.5% or about 15% by weight of the composition.

TABLE iii

| | FR1 | FR2 | FR3 |
|---|---|---|---|
| | (Fing 0.01) | (Fing 0.01) | (Fing 0.01) |
| Ingredients | % w/w | | |
| Fingolimod HCl | 0.01 | 0.01 | 0.01 |
| ST-Elastomer 10 | 77.99 | 67.99 | 77.99 |
| MCT oil | 8 | 8 | 8 |
| Squalane | 2 | 2 | 2 |
| Isopropyl isostearate | 2 | 7 | 2 |
| Dimethyl Sulfoxide | 10 | 20 | |
| Propylene Glycol | | | 10 |
| Total | 100 | 100 | 100 |

TABLE iii-1

| | FR1 | FR2 | FR3 |
|---|---|---|---|
| | (0.6/0.01) | (0.6/0.01) | (0.6/0.01) |
| Ingredients | % w/w | | |
| Tofacitinib Citrate - m* | 1.0 | 1.0 | 1.0 |
| Fingolimod HCl | 0.01 | 0.01 | 0.01 |
| ST-Elastomer 10 | 76.99 | 66.99 | 76.99 |
| MCT oil | 8 | 8 | 8 |
| Squalane | 2 | 2 | 2 |
| Isopropyl isostearate | 2 | 2 | 2 |
| Dimethyl Sulfoxide | 10 | 20 | |
| Propylene Glycol | | | 10 |
| Total | 100 | 100 | 100 |

*0.6% tofacitinib corresponds to 1% tofacitinib citrate; m = micronized API

### Example iv. Formulations Comprising Gelled Oil

Formulations comprising fingolimod HCL alone or in combination with tofacitinib citrate comprising a versagel with different oil combinations are prepared and evaluated for visual appearance as shown in Table iv and Table iv-1. In one or more embodiments, the formulations provided herein comprise a gelled mineral oil. In one or more embodiments, the formulations provided herein comprise a versagel.

TABLE iv

| | OT1.00VG | OT1.00VMSI | OT100VC | OT100VCSI |
|---|---|---|---|---|
| | (0/0.01) | (0/0.01) | (0/0.01) | (0/0.01) |
| Ingredients | % w/w | | | |
| Fingolimod HCl | 0.01 | 0.01 | 0.01 | 0.01 |
| Versagel | 99.99 | 87.99 | 87.99 | 87.99 |
| MCT oil | | 8 | | |
| Cyclomethicone | | | 12 | 8 |
| Squalane | | 2 | | 2 |
| Isopropyl isostearate | | 2 | | 2 |
| Total | 100 | 100 | 100 | 100 |

*0.6% tofacitinib corresponds to 1% tofacitinib citrate: m = micronized API

TABLE iv-1

| | OT1.00VG | OT1.00VMSI | OT1.00VC | OT1.00VCSI |
|---|---|---|---|---|
| | (0.6/0.01) | (0.6/0.01) | (0.6/0.01) | (0.6/0.01) |
| Ingredients | % w/w | | | |
| Tofacitinib Citrate - | 1 | 1 | 1 | 1 |
| m* | | | | |
| Fingolimod HCl | 0.01 | 0.01 | 0.01 | 0.01 |
| Versagel | 98.99 | 86.99 | 86.99 | 86.99 |
| MCT oil | | 8 | | |
| Cyclomethicone | | | 12 | 8 |
| Squalane | | 2 | | 2 |
| Isopropyl isostearate | | 2 | | 2 |
| Total | 100 | 100 | 100 | 100 |

*0.6% tofacitinib corresponds to 1% tofacitinib citrate; m = micronized API

### Example v. Formulations Comprising Different Concentrations of APIs

The amounts of API are varied by increasing or decreasing the amount of elastomer. Formulations comprising fingolimod HCL and tofacitinib citrate at different concentrations are shown in Tables v and v-1 below.

TABLE v

| | TOF/FIN | TOF/FIN | TOF/FIN | TOF/FIN | TOF/FIN | TOF/FIN | TOF/FIN | TOF/FIN |
|---|---|---|---|---|---|---|---|---|
| | 0.6*/ | 0.6*/ | 0.6*/ | 0.6*/ | 0.6*/ | 0.6*/ | 0.6*/ | 0.6*/ |
| | 0.0001** | 0.001** | 0.005** | 0.01** | 0.025** | 0.05 | 0.075 | 0.01** |
| Fingolimod | 0.0001 | 0.001 | 0.005 | 0.01 | 0.025 | 0.05 | 0.075 | 0.1 |
| HCl | | | | | | | | |
| Tofacitinib | 1 | 1 | 1 | 1 | 1 | 1 | 1 | 1 |
| Citrate** | | | | | | | | |
| ST-Elastomer | 86.0099 | 86.099 | 86.095 | 86.99 | 86.075 | 86.95 | 86.025 | 86.9 |
| 10 | | | | | | | | |
| MCT oil | 8 | 8 | 8 | 8 | 8 | 8 | 8 | 8 |
| Squalane | 2 | 8 | 8 | 8 | 8 | 8 | 8 | 8 |
| Isopropyl | 2 | 2 | 2 | 2 | 2 | 2 | 2 | 9 |
| isostearate | | | | | | | | |
| Total | 100 | 100 | 100 | 100 | 100 | 100 | 100 | 100 |

TABLE v-1

| | TOF/FIN | TOF/FIN | TOF/FIN | TOF/FIN | TOF/FIN | TOF/FIN | TOF/FIN |
|---|---|---|---|---|---|---|---|
| | 0.15*/0.02 | 0.3*/0.02 | 0.4*/0.02 | 0.5*/0.02** | 0.6*/0.02 | 0.8*/0.02 | 1.2*/0.02 |
| Fingolimod | 0.02 | 0.02 | 0.02 | 0.02 | 0.02 | 0.02 | 0.02 |
| HCl | | | | | | | |
| Tofacitinib | 0.25 | 0.5 | 0.67 | 6.83 | 1 | 1.33 | 2 |
| Citrate** | | | | | | | |
| ST- | 87.23 | 87.48 | 87.31 | 87.15 | 86.98 | 86.65 | 85.98 |
| Elastomer | | | | | | | |
| 10 | | | | | | | |
| MCT oil | 8 | 8 | 8 | 8 | 8 | 8 | 8 |
| Squalane | 2 | 8 | 8 | 8 | 8 | 8 | 8 |
| Isopropyl | 2 | 2 | 2 | 2 | 2 | 2 | 2 |
| isostearate | | | | | | | |
| Total | 100 | 100 | 100 | 100 | 100 | 100 | 100 |

### Example vi. Alternative Formulations with a Reduced Amount of Elastomer

Some examples of the present disclosure include formulations comprising a reduced amount of an elastomer component with or without active agents, are prepared as shown in Tables vi-1-vi-3.

TABLE vi-1

| | OT2.0002 | OT2.0003 | OT2.0004 | OT2.0005 | OT2.0007 |
|---|---|---|---|---|---|
| | (0.6/0) | (0.6/0) | (0.6/0) | (0.6/0) | (0.6/0) |
| Tofa- | 1.0 | 1.0 | 1.0 | 1.0 | 1.0 |
| citinib | | | | | |
| Citrate* | | | | | |
| ST-Elas- | 14 | 14.5 | 16 | 16 | 16 |
| tomer 10 | | | | | |
| MCT oil | 36 | 36 | 36 | 20 | 20 |
| Isopropyl | 21 | 21 | 21 | 7 | 7 |
| Palmitate | | | | | |
| Isopropyl | | | | 15 | 10 |
| myristate | | | | | |
| Glyceryl | 16 | 16 | 14 | 16 | 16 |
| Behenate | | | | | |
| Cetearyl | 10 | 10 | 10 | | |
| Isono- | | | | | |
| nanoate | | | | | |
| Hydro- | 2 | 1.5 | 2 | 2 | 2 |
| genated | | | | | |
| Castor | | | | | |
| Oil | | | | | |
| Cyclo- | | | | 13 | 13 |
| methicone | | | | | |
| Oleyl | | | | 10 | 5 |
| alcohol | | | | | |
| White | | | | | 10 |
| mineral | | | | | |
| oil | | | | | |
| Total | 100 | 100 | 100 | 100 | 100 |

*0.6% Tofacitinib corresponds to 1% Tofacitinib citrate (non-micronized);

TABLE vi-2

| | OT2.0002 | OT2.0003 | OT2.0004 | OT2.0005 | OT2.0007 |
|---|---|---|---|---|---|
| | (0/0.01) | (0/0.01) | (0/0.01) | (0/0.01) | (0/0.01) |
| Fingolimod | 0.01 | 0.01 | 0.01 | 0.01 | 0.01 |
| HCl** | | | | | |
| ST-Elas- | 14.99 | 15.49 | 16.99 | 16.99 | 16.99 |
| tomer 10 | | | | | |
| MCT oil | 36 | 36 | 36 | 20 | 20 |
| Isopropyl | 21 | 21 | 21 | 7 | 7 |
| Palmitate | | | | | |
| Isopropyl | | | | 15 | 10 |
| myristate | | | | | |
| Glyceryl | 16 | 16 | 14 | 16 | 16 |
| Behenate | | | | | |
| Cetearyl | 10 | 10 | 10 | | |
| Isono- | | | | | |
| nanoate | | | | | |
| Hydro- | 2 | 1.5 | 2 | 2 | 2 |
| genated | | | | | |
| Castor Oil | | | | | |
| Cyclo- | | | | 13 | 13 |
| methicone | | | | | |
| Oleyl | | | | 10 | 5 |
| alcohol | | | | | |
| White | | | | | 10 |
| mineral | | | | | |
| oil | | | | | |
| Total | 100 | 100 | 100 | 100 | 100 |

**0.01% fingolimod corresponds to 0.0112% of fingolimod HCl

TABLE vi-3

| | OT2.0002 | OT2.0003 | OT2.0004 | OT2.0005 | OT2.0007 |
|---|---|---|---|---|---|
| | (0.6/0.01) | (0.6/0.01) | (0.6/0.01) | (0.6/0.01) | (0.6/0.01) |
| Tofacitinib | 1.0 | 1.0 | 1.0 | 1.0 | 1.0 |
| Citrate* | | | | | |
| Fingolimod | 0.01 | 0.01 | 0.01 | 0.01 | 0.01 |
| HCl** | | | | | |
| ST-Elas- | 13.99 | 14.49 | 15.99 | 15.99 | 15.99 |
| tomer 10 | | | | | |
| MCT oil | 36 | 36 | 36 | 20 | 2.0 |
| Isopropyl | 21 | 21 | 21 | 7 | 7 |
| Palmitate | | | | | |
| Isopropyl | | | | 15 | 10 |
| myristate | | | | | |
| Glyceryl | 16 | 16 | 14 | 16 | 16 |
| Behenate | | | | | |
| Cetearyl | 10 | 10 | 10 | | |
| Isono- | | | | | |
| nanoate | | | | | |
| Hydro- | 2 | 1.5 | 2 | 2 | 2 |
| genated | | | | | |
| Castor Oil | | | | | |
| Cyclo- | | | | 13 | 13 |
| methicone | | | | | |
| Oleyl | | | | 10 | 5 |
| alcohol | | | | | |
| White | | | | | 10 |
| mineral oil | | | | | |
| Total | 100 | 100 | 100 | 100 | 100 |

*0.6% Tofacitinib corresponds to 1% Tofacitinib citrate (non-micronized);
**0.01% fingolimod corresponds to 0.0112% of fingolimod HCl

### Example vii. Alternative Oleogel-Based Carrier and Active Formulations

Formulations comprising fingolimod alone or in combination with tofacitinib in oleogel-based carrier are prepared as shown in Table vii.

TABLE vii

| | OT3.0005A | OT3.0005A |
|---|---|---|
| | (0/0.01) | (0.3/0.01) |
| Tofacitinib* | 0 | 0.5 |
| Citrate | | |
| Fingolimod** | 0.01 | 0.01 |
| HCl | | |
| MCT oil (Migliol | 19.99 | 19.49 |
| 812N) | | |
| Isopropyl | 5 | 5 |
| Palmitate | | |
| (Crodamol IPP) | | |
| Isopropyl | | |
| myristate | | |
| (Crodamol IPM) | | |
| Glyceryl | 16 | 16 |
| Behenate | | |
| (Compritol 888) | | |
| Hydrogenated | 2 | 2 |
| Castor Oil | | |
| (Kaliwax HCO) | | |
| Cyclomethicone | 13 | 13 |
| (ST- | | |
| Cyclomethicone | | |
| 5NF) | | |
| Oleyl alcohol | | |
| (Kallicream OA) | | |
| White mineral | 37 | 37 |
| oil (Blandol) | | |
| Zea Mays Starch | 7 | 7 |
| (Amidon De | | |
| Mais Extra | | |
| Blank) | | |
| Total | 100 | 100 |

*0.3% Tofacitinib corresponds to 0.5% Tofacitinib citrate
**0.01% fingolimod corresponds to 0.0112% of fingolimod HCl

### Example viii. In-Vitro Human Skin Model for Atopic Dermatitis for Formulations Containing Tofacitnib Citrate and/or Fingolimod Hydrochloride

Tofacitnib citrate and/or fingolimod hydrochloride elastomer-based formulations (Table viii) are evaluated in Reconstructed Human Epidermis Th2 AD model by StratiCELL, Isnes, Belgium as described Experimental section Method S.

TABLE viii

| | | | 3 | 4 | 5 | 6 | |
|---|---|---|---|---|---|---|---|
| | 1 | 2 | TOF 0.6 + | TOF 0.6 + | TOF 0.6 + | TOF 0.3 + | 7 |
| Formulation # | Fingolimod | Fingolimod | Fingolimod | Fingolimod | Fingolimod | Fingolimod | TOF |
| Ingredients | 0.005 | 0.02 | 0.005 | 0.01 | 0.02 | 0.02 | 0.3 |
| Fingolimod HCl | 0.0056 | 0.0224 | 0.0056 | 0.0112 | 0.0224 | 0.0224 | |
| Tofacitinib Citrate | | | 1 | 1 | 1 | 0.5 | 0.5 |
| ST-Elastomer 10 | 87.9944 | 87.9776 | 86.9944 | 86.9888 | 86.9776 | 87.4776 | 87.5 |
| Squalese | 2 | 2 | 2 | 2 | 2 | 2 | 2 |
| Isopropyl | 2 | 2 | 2 | 2 | 2 | 2 | 2 |
| Isostearate | | | | | | | |
| MCT oil | 8 | 8 | 8 | 8 | 8 | 8 | 8 |
| Total | 100 | 100 | 100 | 100 | 100 | 100 | 100 |

### Embodiments of Tofacitinib:

1. In one or more embodiments there is provided a topical composition comprising tofacitinib and a carrier in which the tofacitinib is suspended or substantially suspended.
2. The composition of embodiment 1, wherein at least about 99.9% of the tofacitinib is suspended.
3. The composition of embodiments 1 or 2, wherein the tofacitinib is a pharmaceutically acceptable salt.
4. The composition of embodiment 3, wherein the tofacitinib salt is a citrate salt, hydrochloride salt, hydrobromide salt, oxalate salt, nitrate salt, sulfate salt, phosphate salt, fumarate salt, succinate salt, maleate salt, besylate salt, tosylate salt, palmitate salt, tartrate salt, adipate salt, laurate salt, or myristate salt.
5. The composition of embodiment 3, wherein the tofacitinib salt is tofacitinib citrate.
6. The composition of embodiment 3, wherein the tofacitinib salt is tofacitinib adipate.
7. The composition of embodiment 3, wherein the tofacitinib salt is tofacitinib laurate.
8. The composition of embodiment 3, wherein the tofacitinib salt is tofacitinib myristate.
9. The composition of embodiment 1, wherein the tofacitinib is tofacitinib base.
10. The composition of any of the preceding embodiments, wherein the tofacitinib is homogeneously suspended.
11. The composition of any of the preceding embodiments, wherein the tofacitinib is about 0.3% to about 5%, e.g., about 0.3% to about 1.2%, or about 0.6% to about 2.2%, or about 1% to about 2%, or about 1.2% to about 1.8% by weight of the composition.
12. The composition of any of the preceding embodiments, wherein the tofacitinib is about 0.5% to about 1.6%, e.g., about 0.5% to about 0.7% or about 0.8% to about 1.6% by weight of the composition.
13. The composition of any of the preceding embodiments, wherein the tofacitinib is about 0.5%, about 0.6%, about 0.7%, about 0.8%, about 0.9%, about 1% about 1.1%, about 1.2%, about 1.3%, about 1.4%, about 1.5%, about 1.6% by weight of the composition.
14. The composition of any of the preceding embodiments, wherein the tofacitinib is tofacitinib citrate in an amount to provide about 0.3% to 5% e.g., 0.6%, 1.2% or 1.5% tofacitinib by weight of the composition.
15. The composition of any of the preceding embodiments, wherein the tofacitinib is micronized.
16. The composition of any of the preceding embodiments, wherein the tofacitinib is suspended as nanoparticles.
17. The composition of any of the preceding embodiments, wherein the carrier comprises nanoparticles of the tofacitinib.
18. The composition of any of the preceding embodiments, wherein the tofacitinib is of an average uniform size range.
19. The composition of embodiment 18, wherein the average uniform size range is expressed as D90 between about 2 μm to about 50 μm or between about 5 μm to about 50 μm.
20. The composition of embodiment 18, wherein the average uniform size is expressed as D90 of less than about 25 μm, or less than about 10 μm, or is about 9 μm, or about 8 μm, or about 7.5 μm, or about 7 μm, or about 6 μm, or about 5 μm or about 4 μm, or about 3 μm.
21. The composition of embodiments of 18, wherein the average uniform size range is expressed as D90 of less than about 1p m or less than about 0.75 μm, or less than about 0.5 μm, or less than about 0.25 μm, or less than about 0.2 μm, or is about 0.9 μm, or is about 0.8 μm, or is about 0.7 μm, or is about 0.6 μm, or is about 0.5 μm, or is about 0.4 μm, or is about 0.3 μm, or is about 0.25 μm, or is about 0.2 μm, or is about 0.15 μm, or is about 0.1 μm.
22. The composition of any of the preceding embodiments, wherein the carrier reduces the potential for agglomeration of suspended tofacitinib.
23. The composition of embodiment 22, wherein the reduction is in frequency of agglomerates, number of agglomerates, and/or size of agglomerates.
24. The composition of embodiment 22, wherein the average number of tofacitinib particles in the size range between about 40 μm to about 100 μm and is less than about 50 per mg, wherein the average number of particles in the size range between about 100 μm and 200 μm and is less than about 10 per mg and wherein no or almost no particles larger than 200 μm are detected.
25. The composition of embodiment 22, wherein the average size of agglomerates is less than about 175 μm, or is less than about 150 μm, or is less than about 125 μm, or is less than about 100 μm, or is less than about 75 μm, or is less than about 50 μm.
26. The composition of embodiment 22, wherein at least about 95% of the tofacitinib is not present as agglomerates.
27. The composition of embodiment 22, wherein less than about 3% of the composition comprises agglomerates.
28. The composition of embodiment 22, wherein less than about 1% of the composition comprises agglomerates.
29. The composition of embodiment 22, wherein the composition is free or substantially free of agglomerates.
30. The composition of any of embodiment 22 to 29, wherein the carrier comprises at least one elastomer and at least one emollient.
31. The composition of embodiment 30, wherein the emollient includes one or more of a glyceride oil, a branched chain ester, or a branched hydrocarbon oil.
32. The composition of embodiment 30, wherein the emollient is a triglyceride oil, an isopropyl ester, or a saturated or branched hydrocarbon oil.
33. The composition of any of the preceding embodiments, wherein the carrier is not hydrophilic.
34. The composition of any of the preceding embodiments, wherein the carrier is free of or substantially free of hydrophilic compounds.
35. The composition of any of the preceding embodiments, wherein the hydrophilic compound is volatile.
36. The composition of any of the preceding embodiments, wherein the volatile compound is a propellant.
37. The composition of any of the preceding embodiments, wherein the carrier is free or substantially free of a surfactant.
38. The composition of any of the preceding embodiments, wherein the carrier is free or substantially free of water.
39. The composition of any of the preceding embodiments, wherein the carrier is free or substantially free of preservatives.
40. The composition of any of the preceding embodiments, wherein the carrier is free or substantially free of anti-oxidants.
41. The composition of any of the preceding embodiments, wherein the carrier is free or substantially free of scavengers.
42. The composition of any of embodiments 39 to 41, wherein the carrier is free or substantially free of additional stabilizers.
43. The composition of any preceding embodiments, wherein the carrier is free or substantially free of a penetration enhancer that dissolves or substantially dissolves a proportion of the tofacitinib.
44. The composition of any preceding embodiments, wherein the carrier is free or substantially free of a compound that essentially dissolves a proportion of the tofacitinib.
45. The composition of embodiment 43, wherein the carrier is free, or essentially free, or substantially free of a compound that dissolves a proportion of the tofacitinib.
46. The composition of embodiment 45, wherein the compound is water, HCl, transcutol, dimethyl isosorbate, a glycol, a polyethylene glycol, polyethylene glycol 200, polyethylene glycol 400, propylene glycol, glycerol, sulphoxides, dimethyl sulfoxide, dimethylacetamide, or dimethylformamide.
47. The composition of any preceding embodiments, wherein the carrier is free or substantially free of a penetration enhancer that dissolves a proportion of the tofacitinib, wherein the proportion is at least about 0.1%.
48. The composition of any preceding embodiments, wherein the carrier is free or substantially free of a penetration enhancer that dissolves a proportion of the tofacitinib, wherein the proportion is at least about 0.5%.
49. The composition of any preceding embodiments, wherein the carrier is free or substantially free of a penetration enhancer that dissolves a proportion of the tofacitinib, wherein the proportion is at least about 1%.
50. The composition of any preceding embodiments, wherein the carrier is free or substantially free of a penetration enhancer that dissolves a proportion of the tofacitinib, wherein the proportion is at least about 2%.
51. The composition of any preceding embodiments, wherein the carrier is free or substantially free of a penetration enhancer that dissolves a proportion of the tofacitinib, wherein the proportion is at least about 5%.
52. The composition of any preceding embodiments, wherein the carrier is free or substantially free of a penetration enhancer that dissolves a proportion of the tofacitinib, wherein the proportion is at least about 10%.
53. The composition of any preceding embodiments, wherein less than about 1% of tofacitinib present in the composition is dissolved.
54. The composition of any preceding embodiments, wherein less than about 0.5% of tofacitinib present in the composition is dissolved.
55. The composition of any preceding embodiments, wherein less than about 0.1% of tofacitinib present in the composition is dissolved.
56. The composition of any preceding embodiments, wherein the composition is non-occlusive or substantially non-occlusive.
57. The composition of any preceding embodiments, wherein the composition is partially occlusive.
58. The composition of any preceding embodiments, wherein the carrier is free or substantially free of an occlusive agent.
59. The composition of any preceding embodiments, wherein the carrier is free or substantially free of a petrolatum.
60. The composition of any preceding embodiments, wherein the carrier is free or substantially free of a solid wax having a melting temperature greater than about 45° C.
61. The composition of any preceding embodiments, wherein the carrier is free or substantially free of compounds to which tofacitinib is not inert.
62. The composition of any preceding embodiments, wherein the carrier is lipophilic.
63. The composition of embodiment 62, wherein the lipophilic carrier comprises at least one oil that is liquid at room temperature.
64. The composition of embodiment 62, wherein the lipophilic carrier comprises at least one oil that is solid at room temperature.
65. The composition of embodiment 62, wherein the lipophilic carrier comprises at least one oil that is liquid at room temperature, or at least one oil that is solid at room temperature.
66. The composition of any preceding embodiments, wherein the carrier comprises a polymeric agent.
67. The composition of embodiment 66, wherein the polymeric agent is a gelling agent.
68. The composition of any preceding embodiment, wherein the carrier comprises a gelling agent and a hydrophobic agent or oil.
69. The composition of any preceding embodiments, wherein the carrier comprises at least one elastomer.
70. The composition of embodiment 69, wherein the at least one elastomer comprises one or more of cyclopentasiloxane (and) polysilicone-11 (Grant MGS-Elastomer 1100), dimethicone (and) polysilicone-11 (Gransil DMG-3), a cyclopentasiloxane (and) petrolatum (and) polysilicone-11 (MGS-Elastomer 1148P), cyclopentasiloxane (and) dimethicone cross polymer (ST-Elastomer 10), or dimethicone (and) dimethicone crosspolymer (DOWSIL™ 9041).
71. The composition of embodiments 69 to 70 comprising a tofacitinib salt, wherein the salt is more stable than tofacitinib base.
72. The composition of embodiments 69 to 70, wherein the viscosity of the composition is stable or substantially stable from about 8° C. to about 40° C.
73. The composition of embodiments 69 to 70, wherein the viscosity of the composition is stable or substantially stable from about 10° C. to about 35° C.
74. The composition of embodiments 69 to 70, wherein the viscosity of the composition is stable or substantially stable from about 15° C. to about 30° C.
75. The composition of embodiments 69 to 70, wherein the viscosity of the composition is stable or substantially stable from about 20° C. to about 25° C.
76. The composition of any preceding embodiments, wherein the carrier comprises a gelled oil.
77. The composition of any preceding embodiments, wherein the carrier comprises a gelled mineral oil.
78. The composition of any preceding embodiments, wherein the carrier comprises a gelled mineral oil and an elastomer.
79. The composition of any preceding embodiments, wherein the carrier comprises an elastomer and an emollient.
80. The composition of any preceding embodiments, wherein the carrier comprises a gelled oil and an emollient.
81. The composition of any preceding embodiments, wherein the carrier comprises an elastomer, a gelled oil, and an emollient.
82. The composition of embodiments 80 to 81, wherein the gelled oil comprises a mineral oil.
83. The composition of any preceding embodiments, wherein the emollient is one or more of a glyceride oil, a branched alkyl ester, or a branched hydrocarbon oil.
84. The composition of embodiment 83, wherein if present the glyceride oil comprises a triglyceride oil, or a branched alky ester comprising an isopropyl ester and a saturated branched hydrocarbon oil.
85. The composition of embodiment 84 wherein the triglyceride oil comprises an MCT oil.
86. In one or more embodiments there is provided a topical composition comprising a tofacitinib and a carrier in which the tofacitinib is suspended or substantially suspended, wherein the carrier comprises:

(i) a carrier base comprising at least one elastomer, a gelled mineral oil, at least one gelling agent in at least one lipophilic solvent or oil, or mixtures of any two or more thereof; and

(ii) at least one emollient; and

wherein the carrier is free or substantially free of a penetration enhancer that dissolves a proportion of the tofacitinib.
87. The composition of embodiment 86, wherein at least about 99.9% of the tofacitinib is suspended.
88. The composition of embodiment 87, wherein the tofacitinib is a pharmaceutically acceptable salt.
89. The composition of embodiment 88, wherein the tofacitinib salt includes one or more of tofacitinib citrate, tofacitinib adipate, tofacitinib laurate, or tofacitinib myristate.
90. The composition of embodiment 89, wherein the tofacitinib salt is tofacitinib citrate.
91. The composition of any preceding embodiments, wherein the tofacitinib is at least about 0.3% by weight of the composition.
92. The composition of any preceding embodiments, wherein the tofacitinib is about is about 0.3% to about 5%, e.g., 0.3% to about 3%, about 0.3% to about 2%, about 0.3% to about 1.2%, about 0.6% to about 2.2%, about 1% to about 2%, about 1.2% to about 1.8%, about 0.5% to about 1.6%, about 0.5% to about 0.7%, about 0.8% to about 1.6%, about 0.4% to about 1.0%, about 0.45% to about 0.8%, or about 0.5% to about 0.7% by weight of the composition.
93. The composition of any of embodiments 87 to 92, wherein the tofacitinib is about 0.4%, about 0.45%, about 0.5%, about 0.55%, about 0.6%, about 0.65%, about 0.7%, about 0.75%, about 0.8%, about 0.85%, about 0.9%, about 1% about 1.1%, about 1.2%, about 1.3%, about 1.4%, about 1.5%, or about 1.6% by weight of the composition.
94. The composition of any of embodiments 87 to 93, wherein the tofacitinib is tofacitinib citrate in an amount to provide about 0.3% to 5% e.g., 0.6%, 1.2% or 1.5% tofacitinib by weight of the composition.
95. The composition of embodiment 86, wherein the elastomer comprises one or more of cyclopentasiloxane (and) polysilicone-11 (Grant MGS-Elastomer 1100), dimethicone and polysilicone-11 (Gransil DMG-3), a cyclopentasiloxane (and) petrolatum (and) polysilicone-11 (MGS-Elastomer I148P), cyclopentasiloxane (and) dimethicone cross polymer (ST-Elastomer 10), or dimethicone (and) dimethicone Crosspolymer (DOWSIL™ 9041).
96. The composition of embodiment 95, wherein the elastomer comprises ST-Elastomer 10.
97. The composition of embodiment 86, wherein the gelled mineral oil comprises a mineral oil and ethylene/propylene/styrene copolymer and butylene/ethylene/styrene copolymer.
98. The composition of embodiment 86, wherein the emollient comprises one or more of a glyceride, a triglyceride, a diglyceride, a monoglyceride, an MCT oil, a branched hydrocarbon oil, a saturated and branched hydrocarbon oil, squalene, squalane, a branched alkyl ester, isopropyl isostearate, isopropyl palmitate, isopropyl myristate, oleyl alcohol, a mineral oil, a vegetable oil, a liquid fatty acid, a liquid fatty alcohol, a branched liquid fatty acid, a branched liquid fatty alcohol, glyceryl monooleate, glyceryl isostearate, glyceryl dicaprate, a polypropylene glycerol alkyl ether, a polypropylene glycerol stearyl ether, polypropylene glycerol 15 stearyl ether, polypropylene glycerol 11 stearyl ether, glycerol behenate, diisopropyl adipate, cetearyl ethylhexanoate, or cetearyl isononanoate.
99. The composition of embodiment 98, wherein the emollient comprises a triglyceride oil.
100. The composition of embodiment 99, wherein the triglyceride oil comprises MCT oil.
101. The composition of embodiment 98, wherein the emollient comprises a branched alkyl ester.
102. The composition of embodiment 101, wherein the branched alkyl ester comprises an isopropyl ester or a glycerol iso-ester.
103. The composition of embodiment 102, wherein the isopropyl ester comprises isopropyl isostearate, isopropyl palmitate, isopropyl myristate or mixtures of two or more thereof.
104. The composition of embodiment 103, wherein the isopropyl ester comprises isopropyl isostearate.
105. The composition of embodiment 98, wherein the emollient comprises a branched hydrocarbon oil.
106. The composition of embodiment 104, wherein the branched hydrocarbon oil comprises squalane or squalene.
107. The composition of embodiment 98, wherein the emollient comprises a branched and saturated hydrocarbon oil.
108. The composition of embodiment 86 to 107, wherein the emollient comprises at least two of a triglyceride oil, an isopropyl ester and a saturated, or a branched hydrocarbon oil.
109. The composition of embodiment 108, wherein the emollient comprises at least two of isopropyl isostearate, squalane, squalene, or an MCT oil.
110. The composition of embodiments 86 to 106, wherein the emollient comprises a triglyceride oil, an isopropyl ester, or a saturated and branched hydrocarbon oil.
111. The composition of embodiment 109, wherein the emollient comprises MCT oil, an isopropyl ester, or squalane.
112. The composition of embodiment 108, wherein the isopropyl ester comprises isopropyl isostearate.
113. The composition of embodiment 100, wherein the composition provides at least two, three, or four of the following characteristics:

an increase in the chemical stability of tofacitinib salt;

a reduction or elimination of balling;

when applied topically to skin or mucosa an increased delivery of tofacitinib into the skin or mucosa;

when applied topically to skin or mucosa a reduced delivery of tofacitinib through the skin or mucosa; and

when applied topically to skin an increased delivery of tofacitinib into the epidermis and reduced delivery through the skin.

114. The composition of embodiment 100, wherein the tofacitinib is a pharmaceutically acceptable salt and wherein the salt is tofacitinib citrate.
115. The composition of embodiments 86 to 114, wherein the carrier comprises a silicone oil in addition to the elastomer.
116. The composition of embodiment 115, wherein the silicone oil is a cyclomethicone or a dimethicone.
117. The composition of embodiments 86 to 116, wherein the elastomer is about 75% to about 97% by weight of the composition.
118. The composition of embodiments 86 to 116, wherein the elastomer is about 80% to about 93% by weight of the composition.
119. The composition of embodiments 86 to 116, wherein the elastomer is about 86% to about 89% by weight of the composition.
120. The composition of embodiments 86 to 119, wherein the emollient is about 3% to about 25% by weight of the composition.
121. The composition of embodiments 86 to 119, wherein the emollient is about 7% to about 20% by weight of the composition.
122. The composition of embodiments 86 to 119, wherein the emollient is about 11% to about 14% by weight of the composition.
123. The composition of embodiments 6 to 122, wherein the emollient is about 12%, about 13%, or about 14% by weight of the composition.
124. The composition of embodiments 115 to 116, wherein the silicone oil is about 1% to about 75%, or about 5% to about 50%, or about 7% to about 30%, or about 10% to about 15% by weight of the composition.
125. The composition of embodiments 86 to 124, wherein the gelling agent is about 0.5% to about 15%, or about 1% to about 13%, or about 5% to about 12%, or about 8% to about 11%, by weight of the composition.
126. The composition of embodiments 86 to 125, wherein the carrier comprises, an elastomer, and at least two emollients, wherein the ratio of emollient to elastomer is from about 1:30 to about 1:3.
127. The composition of embodiments 86 to 125, wherein the carrier comprises an elastomer and at least two emollients, wherein the ratio of emollient to elastomer is between about 1:9 to about 1:6, between about 1:8 and about 1:7, about 1:7, about 3:22, or about 1:8.
128. The composition of embodiments 86 to 127, wherein the emollient is liquid at room temperature.
129. The composition of embodiment 128, wherein the temperature is about 25° C.
130. The composition of any preceding embodiments of, wherein the tofacitinib is in a concentration sufficient to bind to Janus Kinase (JAK) receptors in the dermis or epidermis in an applied area of skin of a mammal.
131. The composition of embodiment 130, wherein the skin is of a human subject.
132. The composition of embodiment 130, wherein the receptors are JAK 3 receptors.
133. The composition of embodiment 130, wherein the receptors are JAK 1 receptors.
134. The composition of embodiment 131, wherein the receptors are JAK 3 receptors.
135. The composition of embodiment 131, wherein the receptors are JAK 1 receptors.
136. The composition of any of embodiments 130 to 135, wherein the tofacitinib is in an effective concentration sufficient to reach an apparent maximum inhibition of JAK receptors in the dermis or epidermis in the applied area of a mammal as indicated when a significant additional increase in tofacitinib concentration by weight % in the composition does not result in a significant increase in effect in treating a disorder.
137. The composition of any of embodiments 130 to 135, wherein the tofacitinib is in an effective concentration sufficient to reach an apparent maximum inhibition of JAK receptors in the dermis or epidermis in the applied area of a human subject as indicated when a significant additional increase in tofacitinib concentration by weight % in the composition does not result in a significant increase in effect in treating a disorder.
138. The composition of any of embodiments 86 to 135, wherein the tofacitinib is in a concentration sufficient to reach a plateau effect in the dermis or epidermis in the applied area of skin of a mammal.
139. The composition of any preceding embodiments, wherein the tofacitinib is in a concentration sufficient to reach a plateau effect in the dermis or epidermis in the applied area of a human subject.
140. The composition of any of embodiments 136 to 139, wherein the disorder is atopic dermatitis and the effective concentration is about 0.6% or more, e.g., 0.8%, 1.2% or 1.5% by weight of the composition.
141. The composition of any of the preceding embodiments 86 to 140, wherein the carrier is free or substantially free of one or more of water, surfactants, hydrophilic compounds, preservatives, anti-oxidants, scavengers, chelating agents, or additional stabilizers.
142. The composition of embodiment 141, wherein the composition is anhydrous or substantially anhydrous.
143. The composition of embodiment 141, wherein the composition has an Aw value of less than 0.9.
144. The composition of embodiment 141, wherein the composition has an Aw value of less than 0.8.
145. The composition of embodiment 141, wherein the composition has an Aw value of less than 0.7.
146. The composition of embodiment 141, wherein the composition has an Aw value of less than 0.6.
147. The composition of any of embodiment 86 to 146, wherein the tofacitinib is chemically stable.
148. The composition of embodiment 147, wherein the tofacitinib is chemically stable for at least 3 months at 25° C.
149. The composition of embodiment 147, wherein the tofacitinib is chemically stable for at least 6 months at 25° C.
150. The composition of embodiment 147, wherein at least 90% by mass of the tofacitinib or salt thereof is present in the composition when stored for 3 months at 25° C.
151. The composition of embodiment 147, wherein at least about 90% by mass of the tofacitinib or salt thereof is present in the composition when stored for 6 months at 25° C.
152. The composition of embodiment 147, wherein at least about 95% by mass of the tofacitinib or salt thereof is present in the composition when stored for 3 months at 25° C.
153. The composition of embodiment 147, wherein at least about 95% by mass of the tofacitinib or salt thereof is present in the composition when stored for 6 months at 25° C.
154. The composition of embodiment 147, wherein at least about 98% by mass of the tofacitinib or salt thereof is present in the composition when stored for 3 months at 25° C.
155. The composition of embodiment 147, wherein at least about 98% by mass of the tofacitinib or salt thereof is present in the composition when stored for 6 months at 25° C.
156. The composition of embodiment 147, wherein at least about 99% by mass of the tofacitinib or salt thereof is present in the composition when stored for 3 months at 25° C.
157. The composition of embodiment 147, wherein at least about 99% by mass of the tofacitinib or salt thereof is present in the composition when stored for 6 months at 25° C.
158. The composition of any of embodiments 147 to 157, wherein the composition is stored at 40° C.
159. The composition of any of embodiments 147 to 157, wherein less than about 0.1% by mass of Impurity B is measured when the composition is stored for 3 months at 25° C. compared to time 0.
160. The composition of any preceding embodiments, wherein less than about 0.1% by mass of Impurity B is measured when the composition is stored for 6 months at 25° C. compared to time 0.
161. The composition of any of embodiments 159 to 160, wherein the composition is stored at 40° C.
162. The composition of any of embodiments 107 to 112, wherein one or more of the adhesiveness, surface energy, surface tension, or interfacial tension of the composition is reduced.
163. The composition of embodiment 162 wherein the reduction is sufficient to discourage significant adhesion to a metal surface.
164. The composition of embodiment 162, wherein the reduction is sufficient to discourage significant adhesion to a moving metal surface.
165. The composition of embodiments 163 to 164, wherein the metal is stainless steel.
166. The composition of embodiment 162, wherein the reduction is sufficient to discourage significant adhesion to a plastic surface.
167. The composition of embodiment 162, wherein the reduction is sufficient to discourage significant adhesion to a moving plastic surface.
168. The composition of embodiment 162, wherein the surface energy of the carrier and tofacitinib is below that of tofacitinib and a metal.
169. The composition of embodiment 162, wherein the interfacial tension (mN/m) of the carrier and tofacitinib is below that of tofacitinib and a metal.
170. The composition of embodiment 162, wherein the interfacial tension (mN/m) of the carrier and tofacitinib is at least about 5% below that of tofacitinib and a metal.
171. The composition of embodiment 162, wherein the interfacial tension (mN/m) of the carrier and tofacitinib is at least about 10% below that of tofacitinib and a metal.
172. The composition of embodiment 162, wherein the interfacial tension (mN/m) of the carrier and tofacitinib is at least about 15% below that of tofacitinib and a metal.
173. The composition of any of embodiments 168 to 172, wherein the metal is stainless steel.
174. The composition of embodiment 162, wherein the surface energy of the carrier and tofacitinib is below that oftofacitinib and a plastic.
175. The composition of embodiment 162, wherein the interfacial tension (mN/m) of the carrier and tofacitinib is below that of tofacitinib and a plastic.
176. The composition of embodiment 162, wherein the interfacial tension (mN/m) of the carrier and tofacitinib is at least about 5% below that of tofacitinib and a plastic.
177. The composition of embodiment 162, wherein the interfacial tension (mN/m) of the carrier and tofacitinib is at least about 10% below that of tofacitinib and a plastic.
178. The composition of embodiment 162, wherein the interfacial tension (mN/m) of the carrier and tofacitinib is at least about 15% below that of tofacitinib and a plastic.
179. The composition of embodiments 166 to 167, and 174 to 178, wherein the plastic is PTFE (polytetrafluorethylene).
180. The composition of 162, wherein the surface energy of the composition is below that of the tofacitinib with a metal
181. The composition of embodiment 162, wherein the interfacial tension between non-micronized tofacitinib and the composition is less than about 1.6 mN/m or between about 1.5 mN/m and about 1.1 nM/m.
182. The composition of embodiment 162, wherein the interfacial tension between micronized tofacitinib and the composition is less than about 2.5 mN/m, or between about 1.8 mN/m and about 2.3 mN/m.
183. The composition of embodiment 162, wherein the surface tension of the composition is sufficient to discourage adhesion of tofacitinib to a surface.
184. The composition of embodiment 183, wherein the surface is a metal such as stainless steel.
185. The composition of embodiment 183, wherein the surface is a plastic.
186. The composition of embodiments 162 to 185, wherein the emollient comprises one or more of a branched hydrocarbon oil, a branched alkyl ester, a liquid fatty alcohol, or a liquid fatty acid.
187. The composition of embodiments 162 to 185, wherein the emollient comprises one or more of a branched and saturated hydrocarbon oil, an isopropyl ester, a liquid fatty alcohol, or a liquid fatty acid.
188. The composition of embodiments 162 to 185, wherein the emollient comprises one or more of squalane, isopropyl isostearate, or oleyl alcohol.
189. The composition of any of embodiments 101 to 112, wherein the ratio of carrier base to emollient is less than about 9:1.
190. The composition of any of embodiments 101 to 112, wherein the ratio of carrier base to emollient is between about 9:1 and about 6:1.
191. The composition of any of embodiments 101 to 112, wherein the ratio of carrier base to emollient is between about 8:1 and about 7:1, or is about 8:1, or about 22:3, or about 7:1.
192. The composition of any of embodiments 101 to 112, wherein the carrier base is about 83% to about 90% by weight of the composition.
193. The composition of any of embodiments 101 to 112, wherein the carrier base is about 86% to about 88% by weight of the composition.
194. The composition of any of embodiments 101 to 112, wherein the carrier base is about 87% by weight of the composition.
195. The composition of any of embodiments 101 to 112, wherein the emollient is about 10% to about 16% by weight of the composition.
196. The composition of any of embodiments 101 to 112, wherein the emollient is about 11% to about 14% by weight of the composition.
197. The composition of any of embodiments 101 to 112, wherein the emollient is about 12% by weight of the composition.
198. The composition of any of embodiments 189 to 197, wherein the tofacitinib is about 0.3% to about 5%, e.g., 0.3% to about 3%, about 0.3% to about 2%, about 0.3% to about 1.2%, about 0.6% to about 2.2%, about 1% to about 2%, about 1.2% to about 1.8%, about 0.5% to about 1.6%, about 0.5% to about 0.7%, about 0.8% to about 1.6%, about 0.4% to about 1.0%, about 0.45% to about 0.8%, or about 0.5% to about 0.7%; or about 0.4%, about 0.45%, about 0.5%, about 0.55%, about 0.6%, about 0.65%, about 0.7%, about 0.75%, about 0.8%, about 0.85%, about 0.9%, about 1% about 1.1%, about 1.2%, about 1.3%, about 1.4%, about 1.5%, or about 1.6% by weight of the composition.
199. The composition of embodiment 198, wherein the carrier base comprises elastomer and is about 83% to about 90% by weight of the composition and the emollient is about 10% to about 16% by weight of the composition.
200. The composition of embodiment 198, wherein the carrier base comprises elastomer and is about 86% to about 88% by weight of the composition and the emollient is about 11% to about 14% by weight of the composition.
201. The composition of any of embodiments 32, 84, 98, 99, 108, and 110, wherein the emollient comprises a triglyceride oil comprising one or more of a MCT oil, an olive oil, a coconut oil, a palm oil, a sunflower oil, a rapeseed oil, a soybean oil, a groundnut oil, a peanut oil, a corn oil, a walnut oil, a soya oil, a fish oil, a tallow, a fraction of any of the aforesaid, or mixtures of any two or more thereof.
202. The composition of any preceding embodiments, wherein the tofacitinib is the sole active agent in the composition.
203. The composition of any preceding embodiments other than embodiment 202, wherein the composition further comprises a second active agent.
204. The composition of embodiment 203, wherein the second active agent comprises a JAK inhibitor.
205. The composition of embodiment 203, wherein the second active agent comprises an antipruritic agent.
206. The composition of embodiment 203, wherein the second active agent comprises an anesthetic agent.
207. The composition of any embodiment 203, wherein the second active agent comprises an antibiotic.
208. The composition of embodiment 203, wherein the second active agent comprises a steroid.
209. The composition of embodiment 203, wherein the second active agent comprises a nonsteroidal anti-inflammatory drug (NSAID).
210. The composition of embodiment 203, wherein the second active agent comprises a retinoid.
211. The composition of embodiment 203, wherein the second active agent comprises a dicarboxylic acid.
212. The composition of any preceding embodiments, wherein the carrier or composition is a gel or a semi-solid or a liquid at room temperature.
213. The composition of embodiment 212, wherein the composition is a gel at room temperature.
214. The composition of embodiment 212, wherein the composition is a semi-solid at room temperature.
215. The composition of embodiment 212, wherein the composition is a liquid at room temperature.
216. The composition of any preceding embodiments, wherein composition is foamable.
217. The composition of any preceding embodiments, wherein foamable composition comprises a foam adjuvant.
218. The composition of any preceding embodiments, wherein foamable composition comprises a propellant.
219. The composition of any preceding embodiments, wherein foamable composition upon release from a pressurized canister forms a foam.
220. The composition of any preceding embodiments, wherein the composition when applied to a surface does not run.
221. The composition of any preceding embodiments, wherein the composition when applied to a skin or mucosal surface has a bioadhesive or mucoadhesive quality.
222. The composition of any preceding embodiments, wherein the composition forms a quasi-layer.
223. The composition of any preceding embodiments, wherein the quasi-layer facilitates absorption of the tofacitinib into epidermal and dermal layers of skin.
224. The composition of any preceding embodiments, wherein the quasi-layer facilitates absorption of the tofacitinib into a mucosal membrane.
225. The composition of any preceding embodiments, wherein the quasi-layer facilitates absorption of the tofacitinib into a lining of a body cavity.
226. The composition of any of embodiments 222 to 225, wherein delivery of tofacitinib salt to the skin, mucosa, or body cavity lining is higher than with tofacitinib base.
227. The composition of any of embodiments 222 to 225, wherein delivery of tofacitinib salt in the skin, mucosal and body cavity lining is more than about 50%, or more than about 100%, or more than about 200% higher than with tofacitinib base.
228. The composition of any of embodiments 222 to 225, wherein delivery of tofacitinib salt through the skin, mucosal and body cavity lining is comparable to or lower than with tofacitinib base.
229. The composition of any of embodiments 226 to 228, wherein the carrier base and emollient act synergistically to enhance delivery even though the tofacitinib is not soluble or substantially not soluble in the carrier base and emollient.
230. The composition of embodiment 229 where the carrier base comprises ST-Elastomer 10 and the emollient comprises MCT oil.
231. The composition of any preceding embodiments (other than embodiments providing that the composition is free of one or more of the following) further comprising at least one of a fragrance agent, a masking agent, a buffering agent, a pH agent, a preservative, a chelating agent, an anti-oxidant, a scavenger agent, a thickener, a diluent, or any mixtures of two or more thereof.
232. The composition of any preceding embodiments of (other than embodiments providing that the composition is free of one or more of the following) further comprising at least one of a preservative, a chelating agent, an anti-oxidant, a scavenger agent, or any mixtures of two or more thereof.
233. In one or more embodiments there is provided a kit comprising the composition of any of the preceding embodiments in a container and a disposable applicator connectable to the container.
234. The kit of any of the preceding embodiments, wherein the container comprises a unit dose means suitable for delivery of a measured unit dose.
235. The kit of embodiment 234, wherein the unit dose is about 0.1 g, or about 0.2 g, or about 0.3 g or about 0.4 g, or about 0.5 g. or about 0.6 g, or about 0.7 g or about 0.8 g, or about 0.9 g, or about 1.0 g.
236. The kit of any of the preceding embodiments, wherein the disposable applicator is adapted for delivery of the composition to a body cavity.
237. The kit of any of the preceding embodiments, wherein the disposable applicator is adapted for delivery of the composition to a skin surface.
238. The kit of any of the preceding embodiments, wherein the disposable applicator is adapted for delivery of the composition to a mucosal surface.
239. In one or more embodiments there is provided a method of treating a skin disorder comprising applying to the skin of a subject the composition of any of the proceeding embodiments.
240. In one or more embodiments there is provided a method of treating a mucosal disorder comprising applying to the mucosa of a subject the composition of any of the proceeding embodiments.
241. In one or more embodiments there is provided a method of treating a body cavity disorder comprising applying to a body cavity of a subject the composition of any of the proceeding embodiments.
242. The method of embodiments of 239, wherein the disorder includes dermatitis, atopic dermatitis, psoriasis, or eczema.
243. In one or more embodiments there is provided a method of treating or preventing a dermatological disorder or a deterioration thereof comprising applying to the skin of a subject topical composition comprising a tofacitinib salt and a carrier in which the tofacitinib salt is suspended or substantially suspended, wherein the carrier comprises:

(i) a carrier base comprising at least one elastomer, a gelled mineral oil, at least one gelling agent in at least one lipophilic solvent or oil, and mixtures of any two or more thereof; and

(ii) at least one emollient; and

wherein the carrier is free or substantially free of a penetration enhancer that dissolves a proportion of the tofacitinib salt; and

wherein at least about 99.9% of the tofacitinib salt is suspended.

244. The method of embodiment 243, where in the disorder is a dermatological disorder, a mucosal disorder, or a body cavity disorder.
245. The method of embodiment 244, wherein the dermatological disorder is an eczema.
246. The method of embodiment 245 wherein the eczema is atopic dermatitis, contact dermatitis, dyshidrotic eczema, nummular eczema, seborrheic dermatitis or stasis dermatitis.
247. The method of embodiment 244, wherein the dermatological disorder is a dermatitis.
248. The method of embodiment 245, wherein the dermatological disorder is atopic dermatitis.
249. The method of embodiment 244, wherein the dermatological disorder is psoriasis.
250. The method of any of embodiments 243 to 249, wherein the tofacitinib is delivered into the epidermis and the dermis.
251. The method of embodiment 250, wherein the delivery to the epidermis is greater than to the dermis.
252. The method of embodiment 250, wherein the delivery to the epidermis is at least about 20% or, at least about 50% or, at least about 100% or, at least about 150% or, at least about 200% or, at least about 250%, or at least about 300% greater than to the dermis.
253. The method of embodiment 252, wherein the delivery to the epidermis is expressed as a percentage of applied dose.
254. The method of embodiment 250, wherein the delivery to the epidermis as a percentage of applied dose is at least about 100% greater than to the dermis.
255. The method of embodiment 250, wherein the topical delivery of the tofacitinib to the dermis and epidermis is about or greater than 20-fold the delivery of the tofacitinib through the skin.
256. The method of any of embodiments 243 to 255 embodiments of, wherein the tofacitinib salt in the composition is in an effective concentration sufficient to reach a plateau effect in the dermis or epidermis of a human subject to treat the disorder.
257. The method of embodiment 256, wherein the concentration of the tofacitinib salt corresponds to tofacitinib at about 0.5% to about 0.7% by weight of the composition.
258. The method of embodiment 257, wherein the concentration of the tofacitinib salt corresponds to tofacitinib at about 0.3% to about 5%, e.g., 0.3% to about 3%, about 0.3% to about 2%, about 0.3% to about 1.2%, about 0.6% to about 2.2%, about 1% to about 2%, about 1.2% to about 1.8%, about 0.5% to about 1.6%, about 0.5% to about 0.7%, about 0.8% to about 1.6%, about 0.4% to about 1.0%, about 0.45% to about 0.8%, or about 0.5% to about 0.7%; or about 0.4%, about 0.45%, about 0.5%, about 0.55%, about 0.6%, about 0.65%, about 0.7%, about 0.75%, about 0.8%, about 0.85%, about 0.9%, about 1% about 1.1%, about 1.2%, about 1.3%, about 1.4%, about 1.5%, or about 1.6% by weight of the composition.
259. The method of any of embodiments 239 to 258, wherein the carrier is free or substantially free of a penetration enhancer that dissolves a proportion of the tofacitinib, wherein that proportion is at least 0.1% by weight.
260. The method of any of embodiments 239 to 259, wherein the carrier is free or substantially free of hydrophilic solvents.
261. The method of any of embodiments 239 to 259, wherein the carrier base is about 83% to about 89% by weight of the composition and the emollient is about 10% to about 16% by weight of the composition.
262. The method of embodiment 261, wherein the carrier base comprises ST-Elastomer 10 and the emollient comprises MCT oil.
263. The method of embodiment 262, wherein the emollient further comprises one or more of squalane, an isopropyl ester and oleyl alcohol.
264. The method of any of embodiments 239 to 263, wherein the composition is applied to the area of the disorder.
265. The method any of embodiments 239 to 263, wherein the composition is applied to the area surrounding the area of the disorder.
266. The method of any of embodiments 239 to 264, wherein the composition is applied to the area of the disorder and the area surrounding the disorder.
267. The method of any of embodiments 239 to 266, wherein the composition is applied once daily.
268. The method of any of embodiments 239 to 266, wherein the composition is applied twice daily.
269. The method of any of embodiments 239 to 266, wherein the composition is applied at least once per day for at least 7 days.
270. The method of any of embodiments 239 to 266, wherein the composition is applied at least once per day for at least 14 days.
271. The method of any of embodiments 239 to 266, wherein the composition is applied at least once per day for at least 4 weeks.
272. The method of any of embodiments 239 to 266, wherein the composition is applied at least once per day for at least 8 weeks.
273. The method of any of embodiments 239 to 266, wherein the composition is applied at least once per day for at least 12 weeks.
274. The method of any of embodiments 239 to 266, wherein the composition is applied as a maintenance dose following an initial treatment period.
275. The method of any of embodiments 239 to 266, wherein the maintenance dose is applied on non-consecutive days.
276. The method of embodiment 275, wherein the maintenance dose is applied on alternative days.
277. The method of embodiment 275, wherein the maintenance dose is applied twice weekly.
278. The method of any of embodiment 239 to 277, wherein systemic exposure to tofacitinib applied topically is much less than when the same amount is applied orally.
279. The method of embodiment 278, wherein the systemic exposure is at least 20-fold less.
280. The method of embodiment 278, wherein the systemic exposure is at least 50-fold less.
281. The method of embodiment 278, wherein the systemic exposure is at least 100-fold less.
282. The method of embodiment 278, wherein the systemic exposure is at least 200-fold less.
283. The method of embodiment 278, wherein the systemic exposure is at least 400-fold less.
284. The method of embodiment 278, wherein the systemic exposure is at least 500-fold less.
285. The method of any of embodiment 242, 246, and 248, wherein the topic dermatitis index is reduced by about 25% compared to placebo.
286. The method of embodiment 285, wherein the index is less than three.
287. The method of embodiment 285, wherein the index is about 2.5.
288. The method of any of embodiments 239 to 287, wherein the carrier is free or substantially free of one or more of water, surfactants, hydrophilic compounds, preservatives, anti-oxidants, scavengers, chelating agents and additional stabilizers.
289. The method of any of embodiments 239 to 287, wherein the carrier is not an emulsion.
290. The method of embodiment 243, wherein the composition is a gel.
291. In one or more embodiments there is provided a composition comprising a tofacitinib and a carrier in which part of the tofacitinib is suspended, wherein the carrier comprises:

(i) a carrier base comprising at least one elastomer, a gelled mineral oil, at least one gelling agent in at least one lipophilic solvent or oil, and mixtures of any two or more thereof; and

(ii) at least one emollient; and

(iii) at least one compound in which the tofacitinib has some solubility; and wherein less than about 99.9% of the tofacitinib is suspended.

292. The composition of embodiment 291, wherein less than about 99.8%, or less than about 99.7% less than about 99.6%, or less than about 99.5% less than about 99.3%, or less than about 99% of the tofacitinib is suspended.
293. The composition of 292, wherein the tofacitinib is a salt.
294. The composition of any of embodiments 291 to 292, wherein the tofacitinib is about 0.3% to about 5%, e.g., 0.3% to about 3%, about 0.3% to about 2%, about 0.3% to about 1.2%, about 0.6% to about 2.2%, about 1% to about 2%, about 1.2% to about 1.8%, about 0.5% to about 1.6%, about 0.5% to about 0.7%, about 0.8% to about 1.6%, about 0.4% to about 1.0%, about 0.45% to about 0.8%, or about 0.5% to about 0.7%; or about 0.4%, about 0.45%, about 0.5%, about 0.55%, about 0.6%, about 0.65%, about 0.7%, about 0.75%, about 0.8%, about 0.85%, about 0.9%, about 1% about 1.1%, about 1.2%, about 1.3%, about 1.4%, about 1.5%, or about 1.6% by weight of the composition by weight of the composition.
295. In one or more embodiments there is provided a method of treating or preventing a dermatological disorder, or a deterioration thereof, comprising applying to the skin of a subject the topical composition of any of embodiments of 293 to 294
296. The method of embodiment 295, wherein the disorder includes, an eczema, a dermatitis or psoriasis, wherein the eczema is atopic dermatitis, contact dermatitis, dyshidrotic eczema, nummular eczema, seborrheic dermatitis, or stasis dermatitis.
297. In one or more embodiments there is provided a topical composition comprising a JAK inhibitor and a carrier in which the JAK inhibitor is suspended or substantially suspended, wherein the carrier comprises:

(i) a carrier base comprising at least one elastomer, a gelled mineral oil, at least one gelling agent in at least one lipophilic solvent or oil, and mixtures of any two or more thereof; and

(ii) at least one emollient; and

wherein the carrier is free or substantially free of a penetration enhancer that dissolves a proportion of the JAK inhibitor; and

298, wherein at least about 99.9% of the JAK inhibitor is suspended.
299. The composition of embodiment 297, wherein the JAK inhibitor is a salt.
300. The composition of embodiment 297, wherein the JAK inhibitor is about 0.3% to about 5%, e.g., 0.3% to about 3%, about 0.3% to about 2%, about 0.3% to about 1.2%, about 0.6% to about 2.2%, about 1% to about 2%, about 1.2% to about 1.8%, about 0.5% to about 1.6%, about 0.5% to about 0.7%, about 0.8% to about 1.6%, about 0.4% to about 1.0%, about 0.45% to about 0.8%, or about 0.5% to about 0.7%; or about 0.4%, about 0.45%, about 0.5%, about 0.55%, about 0.6%, about 0.65%, about 0.7%, about 0.75%, about 0.8%, about 0.85%, about 0.9%, about 1% about 1.1%, about 1.2%, about 1.3%, about 1.4%, about 1.5%, or about 1.6% by weight of the composition.
301. A method of treating or preventing a dermatological disorder or a deterioration thereof comprising applying to the skin of a subject the topical composition of any of embodiments 297 to 299.
302. The method of embodiment 300, wherein the disorder includes, an eczema, a dermatitis or psoriasis, wherein the eczema is atopic dermatitis, contact dermatitis, dyshidrotic eczema, nummular eczema, seborrheic dermatitis, or stasis dermatitis.
303. In one or more embodiments there is provided a composition comprising a JAK inhibitor and a carrier in which part of the JAK inhibitor is suspended, wherein the carrier comprises:

(i) a carrier base comprising at least one elastomer, a gelled mineral oil, at least one gelling agent in at least one lipophilic solvent or oil, and mixtures of any two or more thereof; and

(ii) at least one emollient; and

(iii) at least one compound in which the JAK inhibitor has some solubility; and

wherein less than about 99.9% of the JAK inhibitor is suspended.

304. The composition of embodiment 302, wherein less than about 99.8%, or less than about 99.7% less than about 99.6%, or less than about 99.5% less than about 99.3%, or less than about 99% of the JAK inhibitor is suspended.
305. The composition of embodiment 302, wherein the JAK inhibitor is a salt.
306. The composition of embodiment 302, wherein the JAK inhibitor is about 0.3% to about 5%, e.g., 0.3% to about 3%, about 0.3% to about 2%, about 0.3% to about 1.2%, about 0.6% to about 2.2%, about 1% to about 2%, about 1.2% to about 1.8%, about 0.5% to about 1.6%, about 0.5% to about 0.7%, about 0.8% to about 1.6%, about 0.4% to about 1.0%, about 0.45% to about 0.8%, or about 0.5% to about 0.7%; or about 0.4%, about 0.45%, about 0.5%, about 0.55%, about 0.6%, about 0.65%, about 0.7%, about 0.75%, about 0.8%, about 0.85%, about 0.9%, about 1% about 1.1%, about 1.2%, about 1.3%, about 1.4%, about 1.5%, or about 1.6% by weight of the composition.
307. In one or more embodiments there is provided a method of treating or preventing a dermatological disorder or a deterioration thereof comprising applying to the skin of a subject the topical composition of any of embodiments 302 to 305.
308. The method of embodiment 306, wherein the disorder includes, an eczema, a dermatitis or psoriasis, wherein the eczema is atopic dermatitis, contact dermatitis, dyshidrotic eczema, nummular eczema, seborrheic dermatitis, or stasis dermatitis.
309. In one or more embodiments there is provided a topical composition comprising an active agent in a pharmaceutically effective amount and a carrier in which the active agent is suspended or substantially suspended, wherein the carrier comprises:

(i) a carrier base comprising at least one elastomer, a gelled mineral oil, at least one gelling agent in at least one lipophilic solvent or oil, and mixtures of any two or more thereof; and

(ii) at least one emollient;

wherein the carrier is free or substantially free of a penetration enhancer that dissolves a proportion of the active agent; and

wherein at least about 99.9% of the active agent is suspended.

310. The composition of 308, wherein the active agent is a salt.
311. In one or more embodiments there is provided a method of treating or preventing a dermatological disorder or a deterioration thereof comprising applying to the skin of a subject the topical composition of any of embodiments 308 to 309
312. The method of embodiment 310, wherein the disorder includes, an eczema, a dermatitis or psoriasis, wherein the eczema is atopic dermatitis, contact dermatitis, dyshidrotic eczema, nummular eczema, seborrheic dermatitis or stasis dermatitis.
313. In one or more embodiments there is provided a composition comprising an active agent and a carrier in which part of the active agent is suspended, wherein the carrier comprises:

(i) a carrier base comprising at least one elastomer, a gelled mineral oil, at least one gelling agent in at least one lipophilic solvent or oil, and mixtures of any two or more thereof; and

(ii) at least one emollient; and

(iii) at least one compound in which the active agent has some solubility; and

wherein less than about 99.9% of the active agent is suspended.

314. The composition of embodiment 312, wherein less than about 99.8%, or less than about 99.7% less than about 99.6%, or less than about 99.5% less than about 99.3%, or less than about 99% of the active agent is suspended.
315. The composition of 312, wherein the active agent is a salt.
316. In one or more embodiments there is provided a method of treating or preventing a dermatological disorder, or a deterioration thereof, comprising applying to the skin of a subject the topical composition of any of embodiments 312 to 314.
317. The method of embodiment 315, wherein the disorder includes, an eczema, a dermatitis or psoriasis, wherein the eczema is atopic dermatitis, contact dermatitis, dyshidrotic eczema, nummular eczema, seborrheic dermatitis, or stasis dermatitis.
318. In one or more embodiments there is provided a topical carrier composition for suspending or substantially suspending at least about 99.9% of an active agent, wherein the carrier comprises:

(i) a carrier base comprising at least one elastomer, a gelled mineral oil, at least one gelling agent in at least one lipophilic solvent or oil, and mixtures of any two or more thereof; and

(ii) at least one emollient; and

wherein the carrier is free or substantially free of a penetration enhancer that can dissolve a proportion of the active agent.

319. The composition of embodiment 317, further comprising a second active agent wherein the second active agent comprises a JAK inhibitor.
320. The composition of embodiment 318, wherein the JAK inhibitor is a salt.
321. The composition of embodiment 318, wherein the JAK inhibitor is a tofacitinib.
322. The composition of embodiment 320, wherein the tofacitinib is a salt.
323. The composition of embodiment 321, wherein the salt is tofacitinib citrate.
324. The composition of embodiment 321, wherein the tofacitinib is about 0.3% to about 5%, e.g., 0.3% to about 3%, about 0.3% to about 2%, about 0.3% to about 1.2%, about 0.6% to about 2.2%, about 1% to about 2%, about 1.2% to about 1.8%, about 0.5% to about 1.6%, about 0.5% to about 0.7%, about 0.8% to about 1.6%, about 0.4% to about 1.0%, about 0.45% to about 0.8%, or about 0.5% to about 0.7%; or about 0.4%, about 0.45%, about 0.5%, about 0.55%, about 0.6%, about 0.65%, about 0.7%, about 0.75%, about 0.8%, about 0.85%, about 0.9%, about 1% about 1.1%, about 1.2%, about 1.3%, about 1.4%, about 1.5%, or about 1.6% by weight of the composition.
325. In one or more embodiments there is provided a method of treating or preventing a dermatological disorder, or a deterioration thereof, comprising applying to the skin of a subject the topical carrier composition of any of embodiments 317-323.
326. The method of embodiment 324, wherein the disorder includes, an eczema, a dermatitis or psoriasis, wherein the eczema is atopic dermatitis, contact dermatitis, dyshidrotic eczema, nummular eczema, seborrheic dermatitis, or stasis dermatitis.
327. In one or more embodiments there is provided a carrier composition for suspending part and dissolving part of an active agent, wherein the carrier comprises:

(i) a carrier base comprising at least one elastomer, a gelled mineral oil, at least one gelling agent in at least one lipophilic solvent or oil, and mixtures of any two or more thereof; and

(ii) at least one emollient; and

(iii) at least one compound in which the active agent has some solubility; and

wherein the part to be suspended less than about 99.9% of the active agent.

328. The composition of embodiment 326, wherein the part to be dissolved is up to about 15%.
329. The composition of embodiment 327, wherein the part to be dissolved is more than about 0.2%, more than about 0.3%, more than about 0.4%, more than about 0.5%, more than about 0.7%, or more than about 1%.
330. The composition of 327, wherein the active agent is a salt.
331. In one or more embodiments there is provided a method of treating or preventing a dermatological disorder, or a deterioration thereof, comprising applying to the skin of a subject the topical composition of any of embodiments 326 to 329.
332. The method of embodiment 330, wherein the disorder includes, an eczema, a dermatitis or psoriasis, wherein the eczema is atopic dermatitis, contact dermatitis, dyshidrotic eczema, nummular eczema, seborrheic dermatitis or stasis dermatitis.
333. The composition of embodiment 203, wherein the second active agent comprises a coal tar.

### ADDITIONAL EMBODIMENTS

334. The composition of any preceding embodiments, wherein the carrier comprises at least one emollient that does not dissolve an active agent, e.g. a JAK inhibitor, e.g. tofacitinib citrate, wherein said at least one emollient improves the penetration of the active agent into the skin.
335. The composition of embodiment 333, wherein penetration into the epidermis is improved.
336. The composition of embodiment 333, wherein penetration into the dermis is improved.
337. The composition of embodiment 333, wherein penetration into the epidermis and dermis is improved.
338. The composition of embodiment 333, wherein penetration into the epidermis is improved.
339. The composition of embodiment 333, wherein the ratio of penetration into the skin to penetration through the skin is about at least 50:1, or about at least 75:1, or about at least 100:1, or about at least 125:1, or about at least 150:1, or about at least 175:1, or about at least 200:1, or about at least 225:1, or about at least 250:1, or about at least 275:1, or about at least 300:1, or about at least 325:1, or about at least 350:1, or about at least 375:1, or about at least 400:1, or about at least 425:1, or about at least 450:1, or about at least 475:1, or about at least 500:1.
340. The composition of embodiment 333, wherein the ratio of penetration into the skin to penetration through the skin is about 50:1 to about 500:1, or about 100:1 to about 500:1, or about 150:1 to about 500:1, or about 200:1 to about 500:1, or about 250:1 to about 500:1, or about 300:1 to about 500:1, or about 350:1 to about 500:1, or about 400:1 to about 500:1, or about 450:1 to about 500:1, or about 75:1 to about 450:1, or about 100:1 to about 425:1, or about 75:1 to about 400:1, or about 75:1 to about 375:1, or about 75:1 to about 350:1, or about 100:1 to about 400:1, or about 100:1 to about 375:1, or about 100:1 to about 350:1, or about 125:1 to about 400:1, or about 125:1 to about 375:1, or about 125:1 to about 350:1, or about 150:1 to about 375:1, or about 50:1 to about 50:100, or about 50:1 to about 50:100, or about 50:1 to about 50:150, or about 50:1 to about 50:200, or about 50:1 to about 50:250, or about 50:300 to about 50:350, or about 50:1 to about 400:1, or about 50:1 to about 450:1, or about 50:1 to about 500:1.
341. The composition of embodiment 333, wherein the pK of the active ingredient in the blood is low.
342. The composition of any of embodiments 333-340, wherein the at least one emollient comprises isopropyl isostearate and or squalene.
343. The composition of any of embodiments 333-341, wherein by altering the amounts of said emollients the penetration of the active ingredient into the skin is improved.
344. The composition of any of embodiments 333-342, wherein by altering the amounts of said emollients the ratio of penetration of the active ingredient into the skin to penetration through the skin is improved.
345. The composition of any of embodiments 333-343, wherein the emollients comprise isopropyl isostearate and squalene.
346. The composition of embodiment 344, wherein the emollient further comprises at least MCT oil, mineral oil, or IPP.
347. The composition of embodiment 345, wherein the emollient further comprises two or more of MCT oil, mineral oil, or IPP.
348. The composition of any of embodiments 333-346, wherein the emollient is at least about 4%, or at least about 6%, or at least about 8%, or at least about 10%, or at least about 12%, or at least about 14%, or at least about 16%, or at least about 18%, or at least about 20%, or at least about 22%, or at least about 24% by weight of the composition.
349. The composition of any of embodiments 333-347, wherein the emollient is less than about 30%, or less than about 28%, or less than about 26%, or less than about 24%, or less than about 22%, or less than about 20%, or less than about 18%, or less than about 16%, or less than about 14% by weight of the composition.
350. The composition of any of embodiments 333-348, wherein the emollient is about 4% to about 30%, or about 5% to about 28%, or about 6% to about 26%, or about 7% to about 24%, or about 8% to about 22%, or about 8% to about 20%, or about 8% to about 18%, or about 8% to about 16% or about 9% to about 20%, or about 9% to about 19%, or about 9% to about 17%, or about 9% to about 15%, or about 10% to about 18%, or about 10% to about 16%, or about 10% to about 14% by weight of the composition.
351. The composition of any of embodiments 333-349, wherein the emollient is about 4%, or about 5%, or about 6% or about 7% or about 8% or about 9%, or about 10%, or about 11% or about 12% or about 13% or about 14%, or about 15%, or about 16%, or about 17%, or about 18%, or about 19%, about 20%, or about 21% or about 22%, or about 23%, or about 24% or about 25% by weight of the composition.
352. The composition of any of embodiments 333-350, wherein the active agent comprises a JAK inhibitor that acts on one or more JAK receptors.
353. The composition of any of embodiments 333-351, wherein the JAK inhibitor acts on two or more JAK receptors.
354. The composition of any of embodiments 333-352, wherein the JAK inhibitor comprises a tofacitinib.
355. The composition of embodiment 353, wherein the JAK inhibitor comprises tofacitinib citrate.
356. The composition of any of embodiments 353-354, wherein the tofacitinib is about 0.3% to about 5% by weight of the composition.
357. The composition of any of embodiments 353-355, wherein the tofacitinib is about 0.3% to about 5%, or 0.4% to about 4.5%, or 0.5% to about 4%, or 0.6% to about 3.5%, or about 0.6% to about 3%, or 0.6% to about 3.5%, or about 0.6% to about 3%, or about 0.6% to about 2.5%, or about 0.6% to about 2%, or about 0.6% to about 2.5%, or 0.8% to about 3.5%, or about 0.8% to about 3%, or about 0.8% to about 2.5%, or about 0.8% to about 2%, or about 0.8% to about 1.8%, or about 0.8% to about 1.5%, or about 1% to about 3.5%, or about 1% to about 3%, or about 1% to about 2.5%, or about 1% to about 2%, or about 1% to about 1.8%, or about 1% to about 1.5% by weight of the composition.
358. The composition of any of embodiments 353-356, wherein the tofacitinib is about 0.3%, or about 0.4%, or about 0.5%, or about 0.6%, or about 0.7%, or about 0.8%, or about 0.9%, or about 1%, or about 1.1%, or about 1.2%, or about 1.3%, or about 1.4%, or about 1.5%, or about 1.6%, or about 1.7%, or about 1.8%, or about 1.9%, or about 2.0%, or about 2.1%, or about 2.2%, or about 2.3%, or about 2.4%, or about 2.5%, or about 2.6%, or about 2.7%, or about 2.8%, or about 2.9%, or about 3%, or about 3.1%, or about 3.2%, or about 3.3%, or about 3.4%, or about 3.5%, or about 3.6%, or about 3.7%, or about 3.8%, or about 3.9%, or about 4%, or about 4.1%, or about 4.2%, or about 4.3%, or about 4.4%, or about 4.5%, or about 4.6%, or about 4.7%, or about 4.8%, or about 4.9%, or about 5% by weight of composition.
359. The composition of any of embodiments 353-357, wherein the tofacitinib is about 1%, or about 1.1%, or about 1.2%, or about 1.3%, or about 1.4%, or about 1.5%, or about 1.6%, or about 1.7%, or about 1.8% by weight of the composition.
360. The composition of any of embodiments 353-358, wherein the tofacitinib is about 1.2%, or about 1.3%, or about 1.4%, or about 1.5% by weight of the composition.
361. The composition of any of embodiments 353-359 further comprising a second active agent.
362. The composition of embodiment 360 wherein the second active agent comprises one or more of a JAK inhibitor, an antipruritic, an anesthetic, an antibiotic, an anti-atopic dermatitis agent, an anti-alopecia agent, an antihistamine, an anti-fibrinolytic agent, an anti-scarring agent, a cysteine protease inhibitor, a serine protease inhibitor, an anti-vitiligo agent, an anti-psoriasis agent, a MEK inhibitor, an immunosuppressive agent, a sphingosine-1-phosphate receptor modulator or agonist, a steroid, a NSAID, a retinoid, or a dicarboxylic acid.
363. The composition of embodiment 360 wherein the second active agent comprises seliforant and or fingolimod.
364. The composition of embodiment 360 wherein the second active agent comprises, aminocaproic acid and or trametinib dimethylsulfoxide.
365. In one or more embodiments there is provided a method of treating a JAK related condition comprising applying to the skin or mucosa of a subject the composition of any of embodiments 333 to 363.
366. In one or more embodiments there is provided a method of treating or preventing a dermatological disorder or a deterioration thereof comprising applying to the skin or mucosa of a subject the composition of any of embodiments 333 to 364.
367. In one or more embodiments there is provided a method of treating or preventing a dermatological disorder or the deterioration thereof comprising orally administering serlopitant and topically administering a composition of any of the preceding embodiments wherein the composition comprises tofacitinib or a pharmaceutically acceptable salt thereof, or the composition comprises tofacitinib or a pharmaceutically acceptable salt thereof and fingolimod or a pharmaceutically acceptable salt thereof.

### Water Activity Embodiments

368. In one or more embodiments there is provided a composition or a method according to any of the preceding embodiments, wherein the composition has an Aw value of less than about 0.9.
369. The composition or method according to embodiment 366, wherein the composition has an Aw value of less than about 0.7, or less than about 0.6, or less than about 0.5, or less than about 0.4, or less than about 0.3.
370. The composition or method according to embodiments 366-367, wherein the composition has an Aw value of about 0.7, or about 0.6, or about 0.55, or about 0.5, or about 0.45, or about 0.4, or about 0.35, or about 0.3, or about 0.25, or about 2.

### Diagnostic and Treatment Embodiments

371. In one or more embodiments there is provided a method of treating or preventing a dermatological disorder or the deterioration thereof comprising any of the preceding compositions further comprising diagnosing the dermatological disorder, wherein the dermatological disorder is atopic dermatitis or psoriasis.
372. In some embodiments, the diagnosis of atopic dermatitis is based on historical features, morphology and distribution of skin lesions, and related clinical signs, in other embodiments, the diagnosis of atopic dermatitis may include evaluation of (1) atypical vascular responses (e.g. facial pallor, white dermographism, delayed blanch response), (2) keratosis pilaris/pityriasis alba/hyperlinear palms/ichthyosis, (3) ocular/periorbital changes, (4) other regional findings (e.g., perioral changes/periauricular lesions), (5) perifollicular accentuation/lichenification/prurigo lesions and (6) folliculitis/keratosis pilaris, hidradentitis suppurativa, pyoderma gangrenosum, lichenification disorders e.g., lichen planus/sclerosus, lichen simplex chronicus/neurodermatitis, primary cicatricial alopecias such as lichen planopilaris and frontal fibrosing alopecia, and cellulitis.
373. In some embodiments, the diagnosis of atopic dermatitis is based on exclusion of other conditions comprising scabies, seborrheic dermatitis, contact dermatitis (irritant or allergic), ichthyoses, cutaneous T-cell lymphoma, psoriasis, photosensitivity dermatoses, immune deficiency diseases, and erythroderma of other causes.
374. In some embodiments, the diagnosis of psoriasis is based on pattern recognition involving morphologic evaluation of skin lesions and joints.
375. In certain embodiments, the psoriasis is guttate psoriasis or pustular psoriasis.

### Fingolimod Embodiments

376. In some embodiments, any of the preceding compositions comprises fingolimod, either alone or in combination with a JAK inhibitor. In certain embodiments the JAK inhibitor is a tofacitinib. In some embodiments, any of the preceding compositions is a combination of a tofacitinib and a fingolimod. In some embodiments the fingolimod is fingolimod hydrochloride and the tofacitinib is tofacitinib citrate. In some embodiments, the fingolimod is fingolimod free base and the tofacitinib is tofacitinib free base. In some embodiments, if the fingolimod is fingolimod free base the tofacitinib is a salt e.g., tofacitinib citrate. In some embodiments, if the fingolimod is a salt e.g., fingolimod hydrochloride the tofacitinib is the free base. In some embodiments, any of the proceeding compositions comprising a tofacitinib without a fingolimod are instead provided with a fingolimod in place of a tofacitinib. In one or more embodiments any such fingolimod formulations are provided with a therapeutically effective amount of a fingolimod. In some embodiments, a therapeutically effective amount of a fingolimod can be between about 0.001% to about 1% by weight of composition e.g., the fingolimod is at a concentration of about 0.001% to about 0.01% by weight of the composition, or about 0.005% to about 0.01% by weight of the composition, or about 0.005% to about 0.02% by weight of the composition, or about 0.01% to about 0.02% by weight of the composition, or about 0.01% to about 0.1%, or about 0.001% to about 0.1% by weight of the composition, or such other amounts or ranges as described or illustrated elsewhere herein
377. In one or more embodiments there is provided a method of treating or ameliorating a dermatological disorder comprising administering a composition comprising a therapeutically effective amount of fingolimod, or a pharmaceutically acceptable salt thereof, wherein the dermatological disorder is atopic dermatitis, ichthyosis vulgaris, psoriasis, dermatitis, eczema, vitiligo, alopecia, alopecia totalis, alopecia universalis, autoimmune bullous skin disorder such as pemphigus vulgaris (PV) or bullous pemphigoid (BP), skin rash, skin irritation, skin sensitization (e.g., contact dermatitis or allergic contact dermatitis), chronic atypical neutrophilic dermatosis with lipodystrophy and elevated temperature (CANDLE), pustulosis palmoplantaris, ichtyosis, actinic keratosis, pruritus, rosacea, lupus erythematosus, skin inflammation, skin itch, skin infection, skin scars (such as hypertrophic scars, keloid scars, post-surgery scars), acne, or acne vulgaris. In one or more embodiments a fingolimod is used in combination with one or more other active pharmaceutical agents. In one or more embodiments there is provided a method of treating or ameliorating a dermatological disorder comprising administering a composition comprising a therapeutically effective amount of fingolimod, or a pharmaceutically acceptable salt thereof in combination with another active pharmaceutical agent, wherein the dermatological disorder is any of the aforesaid disorders. In one or more embodiments the dermatological disorder involves inflammation as one of its etiological symptoms. In one or more embodiments the fingolimod contributes to treating or ameliorating the inflammation or inflammatory response.
378. In some embodiments, the amount of a fingolimod applied topically is about 0.0001% to about 0.1% by weight of the composition, about 0.0002% to about 0.1% by weight of the composition, about 0.0005% to about 0.05% by weight of the composition, about 0.001% to about 0.01% by weight of the composition, about 0.005% to about 0.01% by weight of the composition, or about 0.001% to about 0.05% by weight of the composition.
379. In one or more embodiments there is provided a method of treating a dermatological disorder comprising administering a composition comprising a therapeutically effective amount of fingolimod, or a pharmaceutically acceptable salt thereof, in combination with tofacitinib, or a pharmaceutically acceptable salt thereof, wherein the dermatological disorder is atopic dermatitis, ichthyosis vulgaris, psoriasis, dermatitis, eczema, vitiligo, alopecia, alopecia totalis, alopecia universalis, autoimmune bullous skin disorder such as pemphigus vulgaris (PV) or bullous pemphigoid (BP), skin rash, skin irritation, skin sensitization (e.g., contact dermatitis or allergic contact dermatitis), chronic atypical neutrophilic dermatosis with lipodystrophy and elevated temperature (CANDLE), pustulosis palmoplantaris, ichtyosis, actinic keratosis, pruritus, rosacea, lupus erythematosus, skin inflammation, skin itch, skin infection, skin scars (such as hypertrophic scars, keloid scars, post-surgery scars), acne, or acne vulgaris. In one or more embodiments there is provided a method of treating a dermatological disorder comprising administering a composition comprising a therapeutically effective amount of fingolimod, or a pharmaceutically acceptable salt thereof, in combination with tofacitinib, or a pharmaceutically acceptable salt thereof, wherein the dermatological disorder is folliculitis, furunculosis, keratosis pilaris, hidradentitis suppurativa, pyoderma gangrenosum, a lichenification disorder e.g., lichen planus, sclerosus, lichen simplex chronicus, neurodermatitis, primary cicatricial alopecias, such as lichen planopilaris and frontal fibrosing alopecia, and cellulitis.
380. In some embodiments the amount of a fingolimod applied topically is about 0.0001% to about 10% by weight of the composition and the amount of a tofacitinib is about 0.01% to about 10% by weight of the composition, the amount of a fingolimod applied topically is about 0.001% to about 1% by weight of the composition and the amount of a tofacitinib is about 0.05% to about 3.05% by weight of the composition, the amount of a fingolimod applied topically is about 0.002% to about 0.1% by weight of the composition and the amount of a tofacitinib is about 0.1% to about 1% by weight of the composition, or the amount of a fingolimod applied topically is about 0.005% to about 0.01% by weight of the composition and the amount of a tofacitinib is about 0.3% to about 0.6% by weight of the composition.
381. In one or more embodiments there is provided a composition for use in the manufacture of a medicament comprising a JAK inhibitor (e.g. a tofacitinib) and or a S1 PR modulator or agonist (e.g. a fingolimod) having an effect of ameliorating or treating a dermatological disorder. In one or more embodiments there is provided the use of a composition in the manufacture of a medicament comprising a JAK inhibitor (e.g. a tofacitinib) and or a S1PR modulator or agonist (e.g. a fingolimod) having an effect of ameliorating or treating a dermatological disorder. In one or more embodiments the dermatological disorder is a JAK related disorder, a S1 PR modulator or agonist, related disorder, an inflammatory disorder, or one or more of the disorders or conditions described or detailed elsewhere herein.
382. In one or more embodiments compositions comprising tofacitinib or a pharmaceutically acceptable salt thereof may also be read as including fingolimod or a pharmaceutically acceptable salt thereof. In one or more embodiments compositions comprising fingolimod or a pharmaceutically acceptable salt thereof may also be read as including tofacitinib or a pharmaceutically acceptable salt thereof.

### FURTHER EMBODIMENTS

383. A topical composition comprising tofacitinib and a carrier in which the tofacitinib is suspended or substantially suspended.

384. The topical composition of embodiment 382, wherein the carrier comprises a carrier base comprising at least one elastomer and at least one emollient.
385. The topical composition of any of embodiments 382-383, wherein the carrier is free or substantially free of a penetration enhancer that dissolves a proportion of the tofacitinib.
386. The topical composition of any preceding embodiments, wherein the at least one emollient comprises one or more emollients that enhances the penetration of tofacitinib when the composition is applied to the skin or mucosa.
387. The topical composition of any preceding embodiments, wherein the emollients comprise an isopropyl ester, e.g., isopropyl isostearate and a saturated or branched hydrocarbon oil e.g., squalane.
388. The composition of any preceding embodiments, wherein the emollient further comprises at least MCT oil, mineral oil, or IPP.
389. The composition of any preceding embodiments, wherein the emollient further comprises two or more of MCT oil, mineral oil, or IPP.
390. The composition of any of embodiments 386-388, wherein one or more of the adhesiveness, surface energy, surface tension, or interfacial tension of the composition is reduced e.g., to discourage or reduce adhesion.
391. The composition of any of any preceding embodiments, wherein the at least one emollient that is capable of enhancing penetration of the tofacitinib comprises isopropyl isostearate and/or squalane.
392. The composition of any preceding embodiments, wherein by altering the amounts and/or ratios of said emollients the penetration of the tofacitinib into the skin is improved.
393. The composition of any preceding embodiments, wherein by altering the amounts and/or ratios of said emollients the ratio of penetration of the tofacitinib into the skin to penetration through the skin is improved.
394. The composition of embodiments 391 or 392 wherein the ratio of isopropyl isostearate to other emollients is about 12:1 to about 1:12, e.g., about 10:1, about 8:1, about 6:1, about 5:1, about 4:1, about, 3:1, about 2:1, about 1:1, about 1:2, about 1:3, about 1:4, about 1:5, about 1:6, about, 1:7, about 1:8, or about 1:10.
395. The composition of embodiments 391 or 392, wherein the ratio of squalane to other emollients is about 12:1 to about 1:12, e.g., about 10:1, about 8:1, about 6:1, about 5:1, about 4:1, about, 3:1, about 2:1, about 1:1, about 1:2, about 1:3, about 1:4, about 1:5, about 1:6 about, 1:7, about 1:8, or about 1:10.
396. The composition of embodiments 391 or 392, wherein the at least one emollient comprises 3 emollients in the ratios of about, 1:1:1, or about 1:4:1, or about 1:1:4, or about 4:1:1
397. The composition of embodiment 395, wherein one of the emollients is isopropyl isostearate or squalane.
398. The composition of embodiment 395, wherein two of the emollients are isopropyl isostearate or squalane.
399. The composition of any of embodiments 395-398, wherein the other emollients (other than isopropyl isostearate and squalane) comprise one or more of a MCT oil, a mineral oil, or IPP.
400. The composition of any preceding embodiment, wherein the emollient comprises one or more of a glyceride, a triglyceride, a diglyceride, a monoglyceride, an MCT oil, a branched hydrocarbon oil, a saturated and branched hydrocarbon oil, squalene, squalane, a branched alkyl ester, an isopropyl ester, a glycerol iso-ester, isopropyl isostearate, isopropyl palmitate, isopropyl myristate, oleyl alcohol, a mineral oil, a vegetable oil, a liquid fatty acid, a liquid fatty alcohol, a branched liquid fatty acid, a branched liquid fatty alcohol, glyceryl monooleate, glyceryl isostearate, glyceryl dicaprate, a polypropylene glycerol alkyl ether, a polypropylene glycerol stearyl ether, polypropylene glycerol 15 stearyl ether, polypropylene glycerol 11 stearyl ether, glycerol behenate, diisopropyl adipate, cetearyl ethylhexanoate, or cetearyl isononanoate or mixtures of two or more thereof.
401. The topical composition of any preceding embodiments, wherein the emollient includes one or more of a glyceride oil, a branched chain ester, or a branched hydrocarbon oil.
402. The composition of any preceding embodiments, wherein the emollient is a triglyceride oil, an isopropyl ester, or a saturated or branched hydrocarbon oil.
403. The composition of any preceding embodiments, wherein the emollient is at least about 4%, or at least about 6%, or at least about 8%, or at least about 10%, or at least about 12%, or at least about 14%, or at least about 16%, or at least about 18%, or at least about 20%, or at least about 22%, or at least about 24% by weight of the carrier.
404. The composition of any preceding embodiments, wherein the emollient is less than about 30%, or less than about 28%, or less than about 26%, or less than about 24%, or less than about 22%, or less than about 20%, or less than about 18%, or less than about 16%, or less than about 14% by weight of the carrier.
405. The composition of any preceding embodiments, wherein the emollient is about 4% to about 30%, or about 5% to about 28%, or about 6% to about 26%, or about 7% to about 24%, or about 8% to about 22%, or about 8% to about 20%, or about 8% to about 18%, or about 8% to about 16% or about 9% to about 20%, or about 9% to about 19%, or about 9% to about 17%, or about 9% to about 15%, or about 10% to about 18%, or about 10% to about 16%, or about 10% to about 14% by weight of the carrier.
406. The composition of any preceding embodiments, wherein the emollient is about 4%, or about 5%, or about 6% or about 7% or about 8% or about 9%, or about 10%, or about 11% or about 12% or about 13% or about 14%, or about 15%, or about 16%, or about 17%, or about 18%, or about 19%, about 20%, or about 21% or about 22%, or about 23%, or about 24% or about 25% by weight of the carrier.
407. The composition of any preceding embodiments, wherein the emollient is about 9% to about 15%, e.g., about 9%, about 10%, about 11%, about 12/6, about 13%, about, 14%, or about 15% by weight of the carrier.
408. The composition of any preceding embodiments, wherein penetration of the tofacitinib into the epidermis is improved.
409. The composition of any preceding embodiments, wherein penetration of the tofacitinib into the dermis is improved.
410. The composition of any preceding embodiments, wherein penetration of the tofacitinib into the epidermis and dermis is improved.
411. The composition of any preceding embodiments, wherein the ratio of penetration into the skin to penetration through the skin is about at least 50:1, or about at least 75:1, or about at least 100:1, or about at least 125:1, or about at least 150:1, or about at least 200:1, or about at least 225:1, or about at least 250:1, or about at least 275:1, or about at least 300:1, or about at least 325:1, or about at least 350:1, or about at least 375:1, or about at least 400:1, or about at least 425:1, or about at least 450:1, or about at least 475:1, or about at least 500:1.
412. The composition of any preceding embodiments, wherein the ratio of penetration into the skin to penetration through the skin is about 50:1 to about 500:1, or about 75:1 to about 450:1, or about 100:1, to about 425:1, or about 75:1 to about 400:1, or about 75:1 to about 375:1, or about 75:1 to about 350:1, or about 100:1, to about 400:1, or about 100:1 to about 375:1, or about 100:1 to about 350:1, or about 125:1, to about 400:1, or about 125:1, or about 375:1, or about 125:1 to about 350:1, or about 150:1, to about 375:1.
413. The composition of any preceding embodiments, wherein the pK of the tofacitinib in the blood is low.
414. The composition of any preceding embodiments, wherein the tofacitinib is a JAK inhibitor that acts on one or more JAK receptors and is in a concentration sufficient to bind to one or more Janus Kinase (JAK) receptors in the dermis or epidermis in an applied area of skin of a mammal e.g., human subject.
415. The composition of any preceding embodiments, wherein the tofacitinib provides a dose dependent effect when applied to the skin or mucosa of a subject.
416. The composition of any preceding embodiments, wherein the tofacitinib is a pharmaceutically acceptable salt.
417. The composition of any preceding embodiments, wherein the tofacitinib salt is a citrate salt, hydrochloride salt, hydrobromide salt, oxalate salt, nitrate salt, sulfate salt, phosphate salt, fumarate salt, succinate salt, maleate salt, besylate salt, tosylate salt, palmitate salt, tartrate salt, adipate salt, laurate salt, or myristate salt.
418. The composition of any preceding embodiments, wherein the tofacitinib comprises tofacitinib citrate.
419. The composition of any preceding embodiments, wherein the tofacitinib comprises tofacitinib base.
420. The composition of an embodiment 417 or 418 wherein the penetration of tofacitinib base in the epidermis is higher than that of tofacitinib citrate, when equivalent amounts are applied to the skin of a subject in the composition.
421. The composition of any preceding embodiments, wherein the tofacitinib is about 0.3% to about 5% by weight of the composition.
422. The composition of any preceding embodiments, wherein the tofacitinib is about 0.3% to about 5%, or 0.4% to about 4.5%, or 0.5% to about 4%, or 0.6% to about 3.5%, or about 0.6% to about 3%, or 0.6% to about 3.5%, or about 0.6% to about 3%, or about 0.6% to about 2.5%, or about 0.6% to about 2%, or about 0.6% to about 2.5%, or 0.8% to about 3.5%, or about 0.8% to about 3%, or about 0.8% to about 2.5%, or about 0.8% to about 2%, or about 0.8% to about 1.8%, or about 0.8% to about 1.5%, or about 1% to about 3.5%, or about 1% to about 3%, or about 1% to about 2.5%, or about 1% to about 2%, or about 1% to about 1.8%, or about 1% to about 1.5% by weight of the composition.
423. The composition of any preceding embodiments, wherein the tofacitinib is about 0.3%, or about 0.4%, or about 0.5%, or about 0.6%, or about 0.7%, or about 0.8%, or about 0.9%, or about 1%, or about 1.1%, or about 1.2%, or about 1.3%, or about 1.4%, or about 1.5%, or about 1.6%, or about 1.7%, or about 1.8%, or about 1.9%, or about 2.0%, or about 2.1%, or about 2.2%, or about 2.3%, or about 2.4%, or about 2.5%, or about 2.6%, or about 2.7%, or about 2.8%, or about 2.9%, or about 3%, or about 3.1%, or about 3.2%, or about 3.3%, or about 3.4%, or about 3.5%, or about 3.6%, or about 3.7%, or about 3.8%, or about 3.9%, or about 4%, or about 4.1%, or about 4.2%, or about 4.3%, or about 4.4%, or about 4.5%, or about 4.6%, or about 4.7%, or about 4.8%, or about 4.9%, or about 5% by weight of composition.
424. The composition of any preceding embodiments, wherein the tofacitinib is about 1%, or about 1.1%, or about 1.2%, or about 1.3%, or about 1.4%, or about 1.5%, or about 1.6%, or about 1.7%, or about 1.8% by weight of the composition.
425. The composition of any preceding embodiments, wherein the tofacitinib is about 1.2%, or about 1.3%, or about 1.4%, or about 1.5% by weight of the composition.
426. The composition of any preceding embodiments further comprising a second active agent.
427. The composition of any preceding embodiments, wherein the second active agent comprises one or more of a JAK inhibitor, an antipruritic, an anesthetic, an antibiotic, an anti-atopic dermatitis agent, an anti-alopecia agent, an antihistamine, an anti-fibrinolytic agent, an anti-scarring agent, a serine protease inhibitor, a cysteine protease inhibitor, an anti-vitiligo agent, an anti-psoriasis agent, a MEK, inhibitor, an immunosuppressive agent, a sphingosine-1-phosphate receptor modulator or agonist, a steroid, a NSAID, a retinoid, or a dicarboxylic acid.
428. The composition of embodiment 425, wherein the second active agent comprises seliforant and/or fingolimod.
429. The composition of embodiment 425, wherein the second active agent comprises, aminocaproic acid and/or trametinib dimethylsulfoxide.
430. The composition of any preceding embodiment, wherein at least about 99.9% of the tofacitinib is suspended.
431. The composition of any of the preceding embodiments, wherein the tofacitinib is homogeneously suspended.
432. The composition of any of the preceding embodiments, wherein the tofacitinib is micronized.
433. The composition of any of the preceding embodiments, wherein the tofacitinib is suspended as nanoparticles.
434. The composition of any of the preceding embodiments, wherein the carrier comprises nanoparticles of the tofacitinib.
435. The composition of embodiment 431, wherein the D90 is between about 2 μm to about 50 μm e.g., about 2 μm to about 20 μm.
436. The composition of embodiment 431, wherein the D90 is less than about 22 μm, or less than about 10 μm, or is about 9 μm, or about 8 μm, or about 7.5 μm, or about 7 μm, or about 6 μm, or about 5 μm or about 4 μm, or about 3 μm.
437. The composition of embodiments 432 or 433, wherein the D90 is less than about 1 μm or less than about 0.75 μm, or less than about 0.5 μm, or less than about 0.25 μm, or less than about 0.2 μm, or is about 0.9 μm, or is about 0.8 μm, or is about 0.7 μm, or is about 0.6 μm, or is about 0.5 μm, or is about 0.4 μm, or is about 0.3 μm, or is about 0.25 μm, or is about 0.2 μm, or is about 0.15 μm, or is about 0.1 μm.
438. The composition of any of the preceding embodiments, wherein the carrier reduces the potential for agglomeration of suspended tofacitinib.
439. The composition of embodiment 437, wherein the reduction is in frequency of agglomerates, number of agglomerates, and/or size of agglomerates.
440. The composition of embodiment 437, wherein the average size of agglomerates is less than about 175 μm, or is less than about 150 μm, or is less than about 125 μm, or is less than about 100 μm, or is less than about 75 μm, or is less than about 50 μm.
441. The composition of embodiment 437, wherein at least about 95% of the tofacitinib is not present as agglomerates.
442. The composition of embodiment 437, wherein less than about 3% of the composition comprises agglomerates.
443. The composition of embodiment 437, wherein less than about 1% of the composition comprises agglomerates.
444. The composition of embodiment 437, wherein the composition is free or substantially free of agglomerates.
445. The composition of any preceding embodiments further comprising one or more surfactants.
446. The composition of embodiment 444, wherein at least one surfactant is non-ionic and has a HLB of less than about 9.
447. The composition of embodiment 444, wherein at least one surfactant is non-ionic and has a HLB of less than about 7, e.g., about 6-4.
448. The composition of embodiment 444, wherein the surfactants are non-ionic and have an average HLB of less than about 9.
449. The composition of embodiment 444, wherein the surfactants are non-ionic and have an average HLB of less than about 7 e.g., about 6-4.
450. The composition of any of the preceding embodiments, wherein the carrier is not hydrophilic.
451. The composition of any of the preceding embodiments, wherein the carrier is free of or substantially free of hydrophilic compounds.
452. The composition of any of the preceding embodiments, wherein the hydrophilic compound is volatile.
453. The composition of any of the preceding embodiments, wherein the volatile hydrophilic compound is a propellant.
454. The composition of any of the preceding embodiments, wherein the carrier is free or substantially free of a surfactant.
455. The composition of any of the preceding embodiments, wherein the carrier is free or substantially free of water.
456. The composition of any of the preceding embodiments, wherein the carrier is free or substantially free of preservatives.
457. The composition of any of the preceding embodiments, wherein the carrier is free or substantially free of anti-oxidants.
458. The composition of any of the preceding embodiments, wherein the carrier is free or substantially free of scavengers.
459. The composition of any of embodiments 455 to 457, wherein the carrier is free or substantially free of additional stabilizers e.g., chelating agents.
460. The composition of any preceding embodiment, wherein the carrier is free or substantially free of a compound that essentially dissolves a proportion of the tofacitinib.
461. The composition of embodiment 459, wherein the carrier is free, or essentially free, or substantially free of a compound that dissolves a proportion of the tofacitinib.
462. The composition of embodiment 460, wherein the compound is water, HCl, transcutol, dimethyl isosorbate, a glycol, a polyethylene glycol, polyethylene glycol 200, polyethylene glycol 400, propylene glycol, glycerol, sulphoxides, dimethyl sulfoxide, dimethylacetamide, or dimethylformamide.
463. The composition of any preceding embodiment, wherein the carrier is free or substantially free of a penetration enhancer that dissolves a proportion of the tofacitinib, wherein the proportion is at least about 0.1%, or about 0.5%.
464. The composition of any preceding embodiment, wherein the carrier is free or substantially free of a penetration enhancer that dissolves a proportion of the tofacitinib, wherein the proportion is at least about 1%, about 2%, about 5% or about 10%.
465. The composition of any preceding embodiment, wherein less than about 1%, e.g., 0.5% or 0.1% of tofacitinib present in the composition is dissolved.
466. The composition of any preceding embodiment, wherein the composition is non-occlusive or substantially non-occlusive.
467. The composition of any preceding embodiment, wherein the composition is partially occlusive.
468. The composition of any preceding embodiment, wherein the carrier is free or substantially free of an occlusive agent.
469. The composition of any preceding embodiment, wherein the carrier is free or substantially free of a petrolatum.
470. The composition of any preceding embodiment, wherein the carrier is free or substantially free of a solid wax having a melting temperature greater than about 45° C.
471. The composition of any preceding embodiment, wherein the carrier is free or substantially free of compounds to which tofacitinib is not inert.
472. The composition of any preceding embodiment, wherein the carrier is lipophilic.
473. The composition of embodiment 471, wherein the lipophilic carrier comprises at least one oil that is liquid at room temperature.
474. The composition of embodiment 471, wherein the lipophilic carrier comprises at least one oil that is solid at room temperature.
475. The composition of embodiment 471, wherein the lipophilic carrier comprises at least one oil that is liquid at room temperature, or at least one oil that is solid at room temperature.
476. The composition of any preceding embodiment, wherein the carrier comprises a polymeric agent.
477. The composition of embodiment 475, wherein the polymeric agent is a gelling agent.
478. The composition of any preceding embodiment, wherein the carrier comprises a gelling agent and a hydrophobic agent or oil.
479. The composition of any preceding embodiment, wherein the at least one elastomer comprises one or more of cyclopentasiloxane (and) polysilicone-11 (Grant MGS-Elastomer 1100), dimethicone (and) polysilicone-11 (Gransil DMG-3), a cyclopentasiloxane (and) petrolatum (and) polysilicone-11 (MGS-Elastomer 1148P), cyclopentasiloxane (and) dimethicone cross polymer (ST-Elastomer 10), or dimethicone (and) dimethicone crosspolymer (DOWSIL™ 9041).
480. The composition of any preceding embodiment, wherein the elastomer comprises ST-Elastomer 10.
481. The composition of embodiments 478 or 479 comprising a tofacitinib salt, wherein the salt is more stable than tofacitinib base.
482. The composition of embodiments 478 or 479, wherein the viscosity of the composition is stable or substantially stable from about 8° C. to about 40° C.
483. The composition of embodiments 478 or 479, wherein the viscosity of the composition is stable or substantially stable from about 10° C. to about 35° C.
484. The composition of embodiments 478 or 479, wherein the viscosity of the composition is stable or substantially stable from about 15° C. to about 30° C.
485. The composition of embodiments 478 or 479, wherein the viscosity of the composition is stable or substantially stable from about 20° C. to about 25° C.
486. The composition of any preceding embodiment, wherein the carrier further comprises a gelled oil.
487. The composition of embodiment 485, wherein the gelled mineral oil comprises a mineral oil and ethylene/propylene/styrene copolymer and butylene/ethylene/styrene copolymer.
488. The composition of any preceding embodiments, wherein the carrier base comprises a silicone oil e.g., a cyclomethicone or a dimethicone in addition to the elastomer.
489. The composition of embodiment 487, wherein the silicone oil is about 1% to about 75%, or about 5% to about 50%, or about 7% to about 30%, or about 10% to about 15% by weight of the carrier base.
490. The composition of embodiments 476 or 477, wherein the gelling agent is about 0.4% to about 15% e.g., about 0.5% to about 5% or about 1% to about 13%, or about 5% to about 12%, or about 8% to about 11%, by weight of the composition.
491. The composition of any preceding embodiments, wherein the ratio of emollient to elastomer is from about 1:30 to about 1:3.
492. The composition of any preceding embodiments, wherein the ratio of emollient to elastomer is between about 1:9 to about 1:6, between about 1:8 and about 1:7, about 1:7, about 3:22, or about 1:8.
493. The composition of embodiment 491, wherein the ratio of emollient to elastomer is about 1:4, about 2:9, about 4:18, about 1:5, about 4:21, about 2:11, or about 1:6.
494. The composition of any preceding embodiment, wherein the composition is anhydrous or substantially anhydrous.
495. The composition of any preceding embodiment, wherein the composition has an Aw value of less than 0.9.
496. The composition of embodiment 494, wherein the composition has an Aw value of less than or about 0.8, 0.7, 0.6, 0.5, 0.4 or 0.3.
497. The composition of embodiment 495, wherein the composition has an Aw value of less than about 0.3.
498. The composition of any preceding embodiments, wherein the tofacitinib is chemically stable.
499. The composition of embodiment 116, wherein the tofacitinib is chemically stable for at least 3 months, e.g., 6 months at 25° C.
500. The composition of embodiments 497 or 498, wherein at least 90%, e.g., about 95%, or about 98%, or about 99% by mass of the tofacitinib or salt thereof is present in the composition when stored for 3 or 6 months at 25° C.
501. The composition of any of embodiments 497 to 499, wherein the composition is stored at 40° C.
502. The composition of any of embodiments 497 to 499, wherein less than about 0.1% by mass of Impurity B is measured when the composition is stored for 3 or 6 months at 25° C. compared to time 0.
503. The composition of embodiment 501, wherein the composition is stored at 40° C.
504. The composition of any of embodiments 389 to 502 wherein the reduction is sufficient to discourage significant adhesion to a surface, e.g., a metal surface e.g., a moving metal surface.
505. The composition embodiment 503, wherein the metal is stainless steel.
506. The composition of any of embodiments 389 to 504, wherein the surface energy of the carrier and tofacitinib is below that of tofacitinib and a metal.
507. The composition of any of embodiments 389 to 505, wherein the interfacial tension (mN/m) of the carrier and tofacitinib is below that of tofacitinib and a metal.
508. The composition of any of embodiments 389 to 506, wherein the interfacial tension (mN/m) of the carrier and tofacitinib is at least about 5% below e.g., 10% or 15% below that oftofacitinib and a metal.
509. The composition of embodiments 506 or 507, wherein the metal is stainless steel.
510. The composition of any of embodiments 389 to 500, wherein the reduction is sufficient to discourage significant adhesion to a plastic surface e.g., a moving plastic surface.
511. The composition of any of embodiments 389 to 500 or 509, wherein the surface energy of the carrier and tofacitinib is below that of tofacitinib and a plastic.
512. The composition of any of embodiments 389 to 500, 509, or 510, wherein the interfacial tension (mN/m) of the carrier and tofacitinib is below that of tofacitinib and a plastic.
513. The composition of any of embodiments 389 to 500, 509, or 510, wherein the interfacial tension (mN/m) of the carrier and tofacitinib is at least about 5% below e.g., 10% or 15% below that of tofacitinib and a plastic.
514. The composition of any of embodiments 389 to 500, 510, or 512, wherein the plastic is PTFE (polytetrafluorethylene).
515. The composition of any of embodiments 389 to 504, wherein the surface energy of the composition is below that of the tofacitinib with a metal
516. The composition of any of embodiments 389 to 505, wherein the interfacial tension between non-micronized tofacitinib and the composition is less than about 1.6 mN/m or between about 1.5 mN/m and about 1.1 nM/m.
517. The composition of any of embodiments 389 to 503, wherein the interfacial tension between micronized tofacitinib and the composition is less than about 2.5 mN/m, or between about 1.8 mN/m and about 2.3 mN/m.
518. The composition of any preceding embodiments, wherein the ratio of carrier base to emollient is about or less than 9:1, e.g., between about 9:1 and about 6:1.
519. The composition of any preceding embodiments, wherein the ratio of carrier base to emollient is between about 8:1 and about 7:1, or is about 8:1, or about 22:3, or about 7:1.
520. The composition of any preceding embodiments, wherein the carrier base is about 83% to about 90% e.g., about 86% to about 88%, e.g., about 87% by weight of the composition.
521. The composition of any preceding embodiment, wherein the carrier base comprises elastomer and is about 83% to about 90% by weight of the composition and the emollient is about 10% to about 16% by weight of the composition.
522. The composition of embodiment 520, wherein the carrier base comprises elastomer and is about 86% to about 88% by weight of the composition and the emollient is about 11% to about 14% by weight of the composition.
523. The composition of any preceding embodiments, wherein the emollient comprises a triglyceride oil comprising one or more of a MCT oil, an olive oil, a coconut oil, a palm oil, a sunflower oil, a rapeseed oil, a soybean oil, a groundnut oil, a peanut oil, a coin oil, a walnut oil, a soya oil, a fish oil, a tallow, a fraction of any of the aforesaid, or mixtures of any two or more thereof.
524. The composition of any preceding embodiment, wherein the tofacitinib is the sole active agent in the composition.
525. The composition of any preceding embodiment, wherein the carrier or composition is a gel, or a semi-solid, or a liquid at room temperature.
526. The composition of embodiment 524, wherein the composition is a gel at room temperature.
527. The composition of embodiment 524, wherein the composition is a semi-solid at mom temperature.
528. The composition of embodiment 524, wherein the composition is a liquid at room temperature.
529. The composition of any preceding embodiment, wherein composition is foamable and comprises a foam adjuvant and/or a surfactant.
530. The composition of embodiment 528, wherein foamable composition comprises a propellant.
531. The composition of embodiment 529, wherein the foamable composition upon release from a pressurized canister forms a foam.
532. The composition of any preceding embodiment, wherein the composition when applied to a surface does not run.
533. The composition of any preceding embodiment, wherein the composition when applied to a skin or mucosal surface has a bioadhesive or mucoadhesive quality.
534. The composition of any preceding embodiment, wherein the composition forms a quasi-layer.
535. The composition of any preceding embodiment, wherein the quasi-layer facilitates absorption of the tofacitinib into epidermal and dermal layers of skin.
536. The composition of any preceding embodiment, wherein the quasi-layer facilitates absorption of the tofacitinib into a mucosal membrane.
537. The composition of any preceding embodiment, wherein the quasi-layer facilitates absorption of the tofacitinib into a lining of a body cavity.
538. The composition of any preceding embodiments, wherein the carrier base and emollient act synergistically to enhance delivery even though the tofacitinib is not soluble or substantially not soluble in the carrier base and emollient.
539. The composition of embodiment 479, wherein the composition provides at least two, three, or four of the following characteristics:

an increase in the chemical stability oftofacitinib salt:

a reduction or elimination of balling;

when applied topically to skin or mucosa an increased delivery of tofacitinib into the skin or mucosa;

when applied topically to skin or mucosa a reduced delivery of tofacitinib through the skin or mucosa; and

when applied topically to skin an increased delivery of tofacitinib into the epidermis and reduced delivery through the skin.

540. The composition of embodiment 382, wherein the tofacitinib is a pharmaceutically acceptable salt and wherein the salt is tofacitinib citrate.
541. The composition of any preceding embodiment (other than embodiments providing that the composition is free of the following) further comprising at least one of a fragrance agent, a masking agent, a buffering agent, a pH agent, a preservative, a chelating agent, an anti-oxidant, a scavenger agent, a thickener, a diluent, or any mixtures of two or more thereof.
542. A kit comprising the composition of any of the preceding embodiments in a container and a disposable applicator connectable to the container.
543. The kit of embodiment 541, wherein the container comprises a unit dose means suitable for delivery of a measured unit dose.
544. The kit of embodiment 542, wherein the unit dose is about 0.1 g, or about 0.2 g, or about 0.3 g or about 0.4 g, or about 0.5 g, or about 0.6 g, or about 0.7 g or about 0.8 g, or about 0.9 g, or about 1.0 g.
545. The kit embodiments 541-543, wherein the disposable applicator is adapted for delivery of the composition to a body cavity.
546. The kit of any of embodiments 541-544, wherein the disposable applicator is adapted for delivery of the composition to a skin surface.
547. The kit of any of embodiments 541-545, wherein the disposable applicator is adapted for delivery of the composition to a mucosal surface.
548. A method of treating a skin disorder comprising applying to the skin of a subject the composition of any of the proceeding embodiments.
549. A method of treating a mucosal disorder comprising applying to the mucosa of a subject the composition of any of the proceeding embodiments.
550. A method of treating a body cavity disorder comprising applying to a body cavity of a subject the composition of any of the proceeding embodiments.
551. A method of treating a JAK related condition comprising applying to the skin or mucosa or body cavity of a subject the composition of any of the preceding embodiments.
552. A method of treating or preventing a dermatological disorder or a deterioration thereof comprising applying to the skin of a subject the composition of any of the preceding embodiments
553. The method of embodiment 551, wherein the disorder includes a dermatitis, atopic dermatitis, or psoriasis.
554. The method of embodiment 551, wherein the dermatological disorder is an eczema.
555. The method of embodiment 553 wherein the eczema is atopic dermatitis, contact dermatitis, dyshidrotic eczema, nummular eczema, seborrheic dermatitis, or stasis dermatitis.
556. The method of embodiment 551, wherein the dermatological disorder is atopic dermatitis.
557. The method of embodiment 551, wherein the dermatological disorder is psoriasis.
558. The method of any of embodiments 550 to 556, wherein the tofacitinib is delivered into the epidermis and the dermis.
559. The method of embodiment 557, wherein the delivery to the epidermis is greater than to the dermis.
560. The method of embodiment 557, wherein the delivery to the epidermis is at least about 20% or, at least about 50% or, at least about 100% or, at least about 150% or, at least about 200% or, at least about 250%, or at least about 300% greater than to the dermis.
561. The method of embodiment 557, wherein the delivery to the epidermis is expressed as a percentage of applied dose.
562. The method of embodiment 559, wherein the delivery to the epidermis as a percentage of applied dose is at least about 100% greater than to the dermis.
563. The method of embodiment 559, wherein the topical delivery of the tofacitinib to the dermis and epidermis is about or greater than 20-fold the delivery of the tofacitinib through the skin.
564. The method of any of embodiments 550 to 562, wherein the composition is applied to the area of the disorder.
565. The method any of embodiments 550 to 563, wherein the composition is applied to the area surrounding the area of the disorder.
566. The method of any of embodiments 550 to 564, wherein the composition is applied to the area of the disorder and the area surrounding the disorder.
567. The method of any of embodiments 550 to 565, wherein the composition is applied once daily.
568. The method of any of embodiments 550 to 565, wherein the composition is applied twice daily.
569. The method of any of embodiments 550 to 565, wherein the composition is applied at least once per day for at least 7 days.
570. The method of any of embodiments 550 to 565, wherein the composition is applied at least once per day for at least 14 days.
571. The method of any of embodiments 550 to 565, wherein the composition is applied at least once per day for at least 4 weeks.
572. The method of any of embodiments 550 to 565, wherein the composition is applied at least once per day for at least 8 weeks.
573. The method of any of embodiments 550 to 565, wherein the composition is applied at least once per day for at least 12 weeks.
574. The method of any of embodiments 550 to 565, wherein the composition is applied as a maintenance dose following an initial treatment period.
575. The method of any of embodiments 550 to 565, wherein the maintenance dose is applied on non-consecutive days.
576. The method of embodiment 574, wherein the maintenance dose is applied on alternative days.
577. The method of embodiment 575, wherein the maintenance dose is applied twice weekly.
578. The method of any of embodiments 550 to 576, wherein systemic exposure to tofacitinib applied topically is much less than when the same amount is applied orally.
579. The method of embodiment 577, wherein the systemic exposure is at least 20-fold less.
580. The method of embodiment 577, wherein the systemic exposure is at least 50-fold less.
581. The method of embodiment 577, wherein the systemic exposure is at least 100-fold less.
582. The method of embodiment 577, wherein the systemic exposure is at least 200-fold less.
583. The method of embodiment 577, wherein the systemic exposure is at least 400-fold less.
584. The method of embodiment 577, wherein the systemic exposure is at least 500-fold less.
585. The method of any of embodiments 555, wherein the atopic dermatitis index is reduced by about 20%, about 25%, or about 30% compared to placebo.
586. The method of embodiment 584, wherein the index is less than four.
587. The method of embodiment 584, wherein the index is about 3.
588. The method of any of embodiments 556, wherein the psoriasis index is reduced by about 20%, about 25%, or about 30% compared to placebo.
589. The method of embodiment 587, wherein the index is less than four.
590. The method of embodiment 587, wherein the index is about 3.4.
591. The method of any preceding embodiments, wherein the carrier is not an emulsion.
592. The method of embodiment 550, wherein the JAK related condition or disorder comprises alopecia totalis, alopecia universalis, vitiligo, autoimmune bullous skin disorder such as pemphigus vulgaris (PV) or bullous pemphigoid (BP), skin rash, skin irritation, skin sensitization (e.g., contact dermatitis or allergic contact dermatitis), chronic atypical neutrophilic dermatosis with lipodystrophy and elevated temperature (CANDLE), pustulosis palmoplantaris, ichtyosis, eczema, actinic keratosis, pruritus, rosacea and acne.
593. The method of embodiment 550, wherein the JAK related condition or disorder comprises vitiligo and the composition is applied topically to the area of skin lacking pigment and its surrounds.
594. The method of embodiment 550, wherein the JAK related condition or disorder comprises alopecia and the composition is applied topically to the skin and hair.
595. A topical composition comprising tofacitinib or a pharmaceutically acceptable salt thereof, and fingolimod or a pharmaceutically acceptable salt thereof, and a carrier in which the fingolimod and tofacitinib are suspended or substantially suspended.
596. The topical composition of embodiment 594, wherein the carrier comprises a carrier base and at least one emollient, wherein the carrier base comprises at least one elastomer.
597. The topical composition of any of embodiment 594 or 595, wherein the carrier is free or substantially free of a penetration enhancer that dissolves a proportion of the tofacitinib or the fingolimod.
598. The topical composition of any preceding embodiment, wherein the at least one emollient comprises one or more emollients that enhances the penetration of tofacitinib when the composition is applied to the skin or mucosa.
599. The topical composition of any preceding embodiment, wherein the emollients comprise an isopropyl ester and a saturated or branched hydrocarbon oil.
600. The topical composition of embodiment 598, wherein the isopropyl ester is isopropyl isostearate and the saturated or branched hydrocarbon oil is squalane.
601. The topical composition of any preceding embodiment, wherein the emollient further comprises one or more of MCT oil, mineral oil, or isopropyl palmitate.
602. The topical composition of any preceding embodiment, wherein the emollient further comprises two or more of MCT oil, mineral oil, or isopropyl palmitate.
603. The topical composition of any of embodiments 598-602, wherein one or more of the adhesiveness, surface energy, surface tension, or interfacial tension of the composition is reduced to reduce adhesion of the composition to a surface.
604. The topical composition of any preceding embodiment, wherein by altering the amounts and/or ratios of said emollients the penetration of the tofacitinib into the skin is improved.
605. The topical composition of any preceding embodiment, wherein by altering the amounts and/or ratios of said emollients the ratio of penetration of the tofacitinib into the skin to penetration of the tofacitinib through the skin is improved.
606. The topical composition of embodiments 603 or 604, wherein the ratio of isopropyl isostearate to other emollients is about 12:1 to about 1:12, e.g., about 10:1, about 8:1, about 6:1, about 5:1, about 4:1, about, 3:1, about 2:1, about 1:1, about 1:2, about 1:3, about 1:4, about 1:5, about 1:6, about, 1:7, about 1:8, or about 1:10.
607. The topical composition of embodiments 603 or 604, wherein the ratio of squalane to other emollients is about 12:1 to about 1:12, e.g., about 10:1, about 8:1, about 6:1, about 5:1, about 4:1, about, 3:1, about 2:1, about 1:1, about 1:2, about 1:3, about 1:4, about 1:5, about 1:6 about, 1:7, about 1:8, or about 1:10.
608. The topical composition of embodiment 603 or 604, wherein the at least one emollient comprises 3 emollients in the ratios of about, 1:1:1, or about 1:4:1, or about 1:1:4, or about 4:1:1.
609. The topical composition of embodiment 607, wherein one of the emollients is isopropyl isostearate or squalane.
610. The topical composition of embodiment 607, wherein two of the emollients are isopropyl isostearate or squalane.
611. The topical composition of any of embodiments 607-609, wherein the emollients in addition to isopropyl isostearate and squalane comprise one or more of an MCT oil, a mineral oil, or isopropyl palmitate.
612. The topical composition of any preceding embodiment, wherein the emollient comprises a glyceride, a triglyceride, a diglyceride, a monoglyceride, an MCT oil, a branched hydrocarbon oil, a saturated and branched hydrocarbon oil, squalene, squalane, a branched alkyl ester, an isopropyl ester, a glycerol iso-ester, isopropyl isostearate, isopropyl palmitate, isopropyl myristate, oleyl alcohol, a mineral oil, a vegetable oil, a liquid fatty acid, a liquid fatty alcohol, a branched liquid fatty acid, a branched liquid fatty alcohol, glyceryl monooleate, glyceryl isostearate, glyceryl dicaprate, a polypropylene glycerol alkyl ether, a polypropylene glycerol stearyl ether, polypropylene glycerol 15 stearyl ether, polypropylene glycerol 11 stearyl ether, glycerol behenate, diisopropyl adipate, cetearyl ethylhexanoate, or cetearyl isononanoate, or mixtures of two or more thereof.
613. The topical composition of any preceding embodiment, wherein the emollient includes one or more of a glyceride oil, a branched chain ester, or a branched hydrocarbon oil.
614. The topical composition of any preceding embodiment, wherein the emollient is a triglyceride oil, an isopropyl ester, or a saturated or branched hydrocarbon oil.
615. The topical composition of any preceding embodiment, wherein the emollient is at least about 4%, or at least about 6%, or at least about 8%, or at least about 10%, or at least about 12%, or at least about 14%, or at least about 16%, or at least about 18%, or at least about 20%, or at least about 22%, or at least about 24% by weight of the composition.
616. The topical composition of any preceding embodiment, wherein the emollient is less than about 30%, or less than about 28%, or less than about 26%, or less than about 24%, or less than about 22%, or less than about 20%, or less than about 18%, or less than about 16%, or less than about 14%, or less than about 12% by weight of the composition.
617. The topical composition of any preceding embodiment, wherein the emollient is about 4% to about 30%, or about 5% to about 28%, or about 6% to about 26%, or about 7% to about 24%, or about 8% to about 22%, or about 8% to about 20%, or about 8% to about 18%, or about 8% to about 16% or about 9% to about 20%, or about 9% to about 19%, or about 9% to about 17%, or about 9% to about 15%, or about 10% to about 18%, or about 10% to about 16%, or about 10% to about 14% by weight of the composition.
618. The topical composition of any preceding embodiment, wherein the emollient is about 4%, or about 5%, or about 6% or about 7% or about 8% or about 9%, or about 10%, or about 11% or about 12% or about 13% or about 14%, or about 15%, or about 16%, or about 17%, or about 18%, or about 19%, about 20%, or about 21% or about 22%, or about 23%, or about 24% or about 25% by weight of the composition.
619. The topical composition of any preceding embodiment, wherein the emollient is about 9% to about 15%, e.g., about 9%, about 10%, about 11%⁰%, about 12%, about 13%, about, 14%, or about 15% by weight of the composition.
620. The topical composition of any preceding embodiment, wherein penetration of the tofacitinib into the epidermis is improved.
621. The topical composition of any preceding embodiment, wherein penetration of the tofacitinib into the dermis is improved.
622. The topical composition of any preceding embodiment, wherein penetration of the tofacitinib into the epidermis and dermis is improved.
623. The topical composition of any preceding embodiment, wherein the ratio of penetration into the skin to penetration through the skin is about at least 50:1, or about at least 75:1, or about at least 100:1, or about at least 125:1, or about at least 150:1, or about at least 175:1, or about at least 200:1, or about at least 225:1, or about at least 250:1, or about at least 275:1, or about at least 300:1, or about at least 325:1, or about at least 350:1, or about at least 375:1, or about at least 400:1, or about at least 425:1, or about at least 450:1, or about at least 475:1, or about at least 500:1.
624. The topical composition of any preceding embodiment, wherein the ratio of penetration into the skin to penetration through the skin is about 50:1 to about 500:1, or about 100:1 to about 500:1, or about 150:1 to about 500:1, or about 200:1 to about 500:1, or about 250:1 to about 500:1, or about 300:1 to about 500:1, or about 350:1 to about 500:1, or about 400:1 to about 500:1, or about 450:1 to about 500:1, or about 75:1 to about 450:1, or about 100:1 to about 425:1, or about 75:1 to about 400:1, or about 75:1 to about 375:1, or about 75:1 to about 350:1, or about 100:1 to about 400:1, or about 100:1 to about 375:1, or about 100:1 to about 350:1, or about 125:1 to about 400:1, or about 125:1 to about 375:1, or about 125:1 to about 350:1, or about 150:1 to about 375:1, or about 50:1 to about 50:100, or about 50:1 to about 50:100, or about 50:1 to about 50:150, or about 50:1 to about 50:200, or about 50:1 to about 50:250, or about 50:300 to about 50:350, or about 50:1 to about 400:1, or about 50:1 to about 450:1, or about 50:1 to about 500:1.
625. The topical composition of any preceding embodiment, wherein the pK of the tofacitinib in the blood is low.
626. The topical composition of any preceding embodiment, wherein the tofacitinib is a JAK inhibitor that acts on one or more JAK receptors and is in a concentration sufficient to bind to one or more Janus Kinase (JAK) receptors in the dermis or epidermis in an applied area of skin of a mammal e.g., human subject.
627. The topical composition of any preceding embodiment, wherein the tofacitinib provides a dose dependent effect when applied to the skin or mucosa of a subject.
628. The topical composition of any preceding embodiment, wherein the tofacitinib is a pharmaceutically acceptable salt.
629. The topical composition of any preceding embodiment, wherein the tofacitinib salt is a citrate salt, hydrochloride salt, hydrobromide salt, oxalate salt, nitrate salt, sulfate salt, phosphate salt, fumarate salt, succinate salt, maleate salt, besylate salt, tosylate salt, palmitate salt, tartrate salt, adipate salt, laurate salt, or myristate salt.
630. The topical composition of any preceding embodiment, wherein the tofacitinib comprises tofacitinib citrate.
631. The topical composition of embodiment 626, wherein the tofacitinib comprises tofacitinib base.
632. The topical composition of embodiment 626 or 627, wherein the penetration of tofacitinib base in the epidermis is higher than that of tofacitinib citrate, when equivalent amounts are applied to the skin of a subject in the otherwise same composition.
633. The topical composition of any preceding embodiment, wherein the tofacitinib is about 0.3% to about 5% by weight of the composition.
634. The topical composition of any preceding embodiment, wherein the tofacitinib is about 0.3% to about 5%, or 0.4% to about 4.5%, or 0.5% to about 4%, or 0.6% to about 3.5%, or about 0.6% to about 3%, or 0.6% to about 3.5%, or about 0.6% to about 3%, or about 0.6% to about 2.5%, or about 0.6% to about 2%, or about 0.6% to about 2.5%, or 0.8% to about 3.5%, or about 0.8% to about 3%, or about 0.8% to about 2.5%, or about 0.8% to about 2%, or about 0.8% to about 1.8%, or about 0.8% to about 1.5%, or about 1% to about 3.5%, or about 1% to about 3%, or about 1% to about 2.5%, or about 1% to about 2%, or about 1% to about 1.8%, or about 1% to about 1.5% by weight of the composition.
635. The topical composition of any preceding embodiment, wherein the tofacitinib is about 0.3%, or about 0.4%, or about 0.5%, or about 0.6%, or about 0.7%, or about 0.8%, or about 0.9%, or about 1%, or about 1.1%, or about 1.2%, or about 1.3%, or about 1.4%, or about 1.5%, or about 1.6%, or about 1.7%, or about 1.8%, or about 1.9%, or about 2.0%, or about 2.1%, or about 2.2%, or about 2.3%, or about 2.4%, or about 2.5%, or about 2.6%, or about 2.7%, or about 2.8%, or about 2.9%, or about 3%, or about 3.1%, or about 3.2%, or about 3.3%, or about 3.4%, or about 3.5%, or about 3.6%, or about 3.7%, or about 3.8%, or about 3.9%, or about 4%, or about 4.1%, or about 4.2%, or about 4.3%, or about 4.4%, or about 4.5%, or about 4.6%, or about 4.7%, or about 4.8%, or about 4.9%, or about 5% by weight of composition.
636. The topical composition of any preceding embodiment, wherein the tofacitinib is about 1%, or about 1.1%, or about 1.2%, or about 1.3%, or about 1.4%, or about 1.5%, or about 1.6%, or about 1.7%, or about 1.8% by weight of the composition.
637. The topical composition of any preceding embodiment, wherein the tofacitinib is about 0.3%, or about 0.35%, or about 0.4%, or about 0.45%, or about 0.5%, or about 0.55%, or about 0.6%, or about 0.65%, or about 0.7%, or about 0.75%, or about 0.8%, or about 0.85%, or about 0.9%, or about 0.95%, or about 1.0%, or about 1.05%, or about 1.1%, or about 1.15%, or about 1.25%, or about 1.35%, or about 1.45%, or about 1.55% by weight of the composition.
638. The topical composition of any preceding embodiment further comprising a third active agent.
639. The topical composition of any preceding embodiment, wherein the third active agent comprises one or more of a JAK inhibitor, an antipruritic, an anesthetic, an antibiotic, an anti-atopic dermatitis agent, an anti-alopecia agent, an antihistamine, an anti-fibrinolytic agent, an anti-scarring agent, a serine protease inhibitor, a cysteine protease inhibitor, an anti-vitiligo agent, an anti-psoriasis agent, a MEK inhibitor, an immunosuppressive agent, a sphingosine-1-phosphate receptor modulator or agonist, a steroid, a NSAID, a retinoid, or a dicarboxylic acid.
640. The topical composition of embodiment 637, wherein the third active agent is seliforant.
641. The topical composition of embodiment 637, wherein the third active agent is aminocaproic acid or trametinib dimethylsulfoxide.
642. The topical composition of any preceding embodiment, wherein at least about 99.9% of the tofacitinib is suspended.
643. The topical composition of any of the preceding embodiment, wherein the tofacitinib is homogeneously suspended.
644. The topical composition of any of the preceding embodiment, wherein the tofacitinib is micronized.
645. The topical composition of any of the preceding embodiment, wherein the tofacitinib is suspended as nanoparticles.
646. The topical composition of any of the preceding embodiment, wherein the wherein the fingolimod is homogeneously suspended, optionally the fingolimod is micronized or is suspended as nanoparticles.
647. The topical composition of embodiment 643, wherein the D90 is between about 2 μm to about 50 μm e.g., about 2 μm to about 20 μm.
648. The topical composition of embodiment 643, wherein the D90 is less than about 22 μm, or less than about 10 μm, or is about 9 μm, or about 8 μm, or about 7.5 μm, or about 7 μm, or about 6 μm, or about 5 μm or about 4 μm, or about 3 μm.
649. The topical composition of embodiment 633 or 645, wherein the D90 is less than about 1 μm or less than about 0.75 μm, or less than about 0.5 μm, or less than about 0.25 μm, or less than about 0.2 μm, or is about 0.9 μm, or is about 0.8 μm, or is about 0.7 μm, or is about 0.6 μm, or is about 0.5 μm, or is about 0.4 μm, or is about 0.3 μm, or is about 0.25 μm, or is about 0.2 μm, or is about 0.15 μm, or is about 0.1 μm.
650. The topical composition of any of the preceding embodiment, wherein the carrier reduces the potential for agglomeration of suspended tofacitinib or fingolimod.
651. The topical composition of embodiment 649, wherein the reduction is in one or more of frequency of agglomerates, number of agglomerates, or size of agglomerates.
652. The topical composition of embodiment 649, wherein the average size of agglomerates is less than about 175 μm, less than about 150 μm, less than about 125 μm, less than about 100 μm, less than about 75 μm, or less than about 50 μm.
653. The topical composition of embodiment 649, wherein at least about 95% of the tofacitinib is not present as agglomerates.
654. The topical composition of embodiment 649, wherein less than about 3% of the composition comprises agglomerates.
655. The topical composition of embodiment 649, wherein less than about 1% of the composition comprises agglomerates.
656. The topical composition of embodiment 649, wherein the composition is free or substantially free of agglomerates.
657. The topical composition of any preceding embodiment further comprising one or more surfactants.
658. The topical composition of embodiment 656, wherein at least one surfactant is non-ionic and has an HLB of less than about 9.
659. The topical composition of embodiment 656, wherein at least one surfactant is non-ionic and has an HLB of less than about 7.
660. The topical composition of embodiment 656, wherein the surfactants are non-ionic and have an average HLB of less than about 9.
661. The topical composition of embodiment 656, wherein the surfactants are non-ionic and have an average HLB of less than about 7.
662. The topical composition of any of the preceding embodiment, wherein the carrier is not hydrophilic.
663. The topical composition of any of the preceding embodiment, wherein the carrier is free of or substantially free of hydrophilic compounds.
664. The topical composition of any of the preceding embodiment, wherein the hydrophilic compound is volatile.
665. The topical composition of any of the preceding embodiment, wherein the volatile hydrophilic compound is a propellant.
666. The topical composition of any of the preceding embodiment, wherein the carrier is free or substantially free of a surfactant.
667. The topical composition of any of the preceding embodiment, wherein the carrier is free or substantially free of water.
668. The topical composition of any of the preceding embodiment, wherein the carrier is free or substantially free of preservatives.
669. The topical composition of any of the preceding embodiment, wherein the carrier is free or substantially free of anti-oxidants.
670. The topical composition of any of the preceding embodiment, wherein the carrier is free or substantially free of scavengers.
671. The topical composition of any of embodiment 667-669, wherein the carrier is free or substantially free of additional stabilizers.
672. The topical composition of any preceding embodiment, wherein the carrier is free or substantially free of a compound that essentially dissolves a proportion of the tofacitinib.
673. The topical composition of embodiment 671, wherein the carrier is free, or essentially free, or substantially free of a compound that dissolves a proportion of the tofacitinib.
674. The topical composition of embodiment 672, wherein the compound is water, HCl, transcutol, dimethyl isosorbate, a glycol, a polyethylene glycol, polyethylene glycol 200, polyethylene glycol 400, propylene glycol, glycerol, sulphoxides, dimethyl sulfoxide, dimethylacetamide, or dimethylformamide.
675. The topical composition of any preceding embodiment, wherein the carrier is free or substantially free of a penetration enhancer that dissolves a proportion of the tofacitinib, wherein the proportion is at least about 0.1%, or about 0.5%.
676. The topical composition of any preceding embodiment, wherein the carrier is free or substantially free of a penetration enhancer that dissolves a proportion of the tofacitinib, wherein the proportion is at least about 1%, about 2%, about 5% or about 10%.
677. The topical composition of any preceding embodiment, wherein less than about 1%, e.g., 0.5% or 0.1% of tofacitinib present in the composition is dissolved.
678. The topical composition of any preceding embodiment, wherein the composition is non-occlusive or substantially non-occlusive.
679. The topical composition of any preceding embodiment, wherein the composition is partially occlusive.
680. The topical composition of any preceding embodiment, wherein the carrier is free or substantially free of an occlusive agent.
681. The topical composition of any preceding embodiment, wherein the carrier is free or substantially free of a petrolatum.
682. The topical composition of any preceding embodiment, wherein the carrier is free or substantially free of a solid wax having a melting temperature greater than about 45° C.
683. The topical composition of any preceding embodiment, wherein the carrier is free or substantially free of compounds to which the tofacitinib or the fingolimod is not inert.
684. The topical composition of any preceding embodiment, wherein the carrier is lipophilic.
685. The topical composition of embodiment 683, wherein the lipophilic carrier comprises at least one oil that is liquid at room temperature.
686. The topical composition of embodiment 683, wherein the lipophilic carrier comprises at least one oil that is solid at room temperature.
687. The topical composition of embodiment 683, wherein the lipophilic carrier comprises at least one oil that is liquid at room temperature, or at least one oil that is solid at room temperature.
688. The topical composition of any preceding embodiment, wherein the carrier comprises a polymeric agent.
689. The topical composition of embodiment 687, wherein the polymeric agent is a gelling agent.
690. The topical composition of any preceding embodiment, wherein the carrier comprises a gelling agent and a hydrophobic agent or oil.
691. The topical composition of any preceding embodiment, wherein the at least one elastomer comprises one or more of cyclopentasiloxane (and) polysilicone-11 (Grant MGS-Elastomer 1100), dimethicone (and) polysilicone-11 (Gransil DMG-3), a cyclopentasiloxane (and) petrolatum (and) polysilicone-11 (MGS-Elastomer 1148P), cyclopentasiloxane (and) dimethicone cross polymer (ST-Elastomer 10), or dimethicone (and) dimethicone crosspolymer (DOWSIL™ 9041).
692. The topical composition of any preceding embodiment, wherein the elastomer comprises ST-Elastomer 10.
693. The topical composition of embodiment 690 or 691 comprising a tofacitinib salt, wherein the salt is more stable than tofacitinib base.
694. The topical composition of embodiment 690 or 691, wherein the viscosity of the composition is stable or substantially stable from about 8° C. to about 40° C.
695. The topical composition of embodiment 690 or 691, wherein the viscosity of the composition is stable or substantially stable from about 10° C. to about 35° C.
696. The topical composition of embodiment 690 or 691, wherein the viscosity of the composition is stable or substantially stable from about 15° C. to about 30° C.
697. The topical composition of embodiment 690 or 691, wherein the viscosity of the composition is stable or substantially stable from about 20° C. to about 25° C.
698. The topical composition of any preceding embodiment, wherein the carrier further comprises a gelled oil.
699. The topical composition of embodiment 697, wherein the gelled mineral oil comprises a mineral oil and ethylene/propylene/styrene copolymer and butylene/ethylene/styrene copolymer.
700. The topical composition of any preceding embodiment, wherein the carrier base comprises a silicone oil in addition to the elastomer.
701. The topical composition of embodiment 699, wherein the silicone oil is a cyclomethicone or a dimethicone.
702. The topical composition of embodiment 700, wherein the silicone oil is about 1% to about 75%, or about 5% to about 50%, or about 7% to about 30%, or about 10% to about 15% by weight of the carrier base.
703. The topical composition of embodiment 688 or 689, wherein the gelling agent is about 0.4% to about 15%, about 0.5% to about 5% about 1% to about 13%, about 5% to about 12%, or about 8% to about 11% by weight of the composition.
704. The topical composition of any preceding embodiment, wherein the ratio of emollient to elastomer is from about 1:30 to about 1:3.
705. The topical composition of any preceding embodiment, wherein the ratio of emollient to elastomer is between about 1:9 to about 1:6, between about 1:8 to about 1:7, about 1:7, about 3:22, or about 1:8.
706. The topical composition of embodiment 703, wherein the ratio of emollient to elastomer is about 1:4, about 2:9, about 4:18, about 1:5, about 4:21, about 2:11, or about 1:6.
707. The topical composition of any preceding embodiment, wherein the composition is anhydrous or substantially anhydrous.
708. The topical composition of any preceding embodiment, wherein the composition has an Aw value of less than 9.
709. The topical composition of embodiment 707, wherein the composition has an Aw value of less than or about 0.8, 0.7, or 0.6.
710. The topical composition of embodiment 707, wherein the composition has an Aw value of less than about 0.5, 0.4 or 0.3.
711. The topical composition of any preceding embodiment, wherein the tofacitinib is chemically stable.
712. The topical composition of embodiment 710, wherein the tofacitinib is chemically stable for at least 3 months at 25° C.
713. The topical composition of embodiment 710 or 711, wherein at least 90%, about 95%, about 98%, or about 99% by mass of the tofacitinib or pharmaceutically acceptable salt thereof is present in the composition when stored for 3 or 6 months at 25° C.
714. The topical composition of any of embodiment 710-712, wherein the composition is stored at 40° C.
715. The topical composition of any of embodiment 710-712, wherein less than about 0.1% by mass of Impurity B is measured when the composition is stored for 3 or 6 months at 25° C. compared to time 0.
716. The topical composition of embodiment 714, wherein the composition is stored at 40° C.
717. The topical composition of any of embodiment 601-714, wherein the reduction is sufficient to discourage significant adhesion to a surface, a metal surface, or a moving metal surface.
718. The topical composition of embodiment 716, wherein the metal is stainless steel.
719. The topical composition of any of embodiment 601-717, wherein the surface energy of the carrier and tofacitinib is below that of tofacitinib and a metal.
720. The topical composition of any of embodiment 601-718, wherein the interfacial tension (mN/m) of the carrier and tofacitinib is below that of tofacitinib and a metal.
721. The topical composition of any of embodiment 601-719, wherein the interfacial tension (mN/m) of the carrier and tofacitinib is at least about 5% below e.g., 10% or 15% below that of tofacitinib and a metal.
722. The topical composition of embodiment 719 or 720, wherein the metal is stainless steel.
723. The topical composition of any of embodiment 601-715, wherein the reduction is sufficient to discourage significant adhesion to a plastic surface e.g., a moving plastic surface.
724. The topical composition of any of embodiment 601-715 or 722, wherein the surface energy of the carrier and tofacitinib is below that of tofacitinib and a plastic.
725. The topical composition of any of embodiment 601-715, 722, or 723, wherein the interfacial tension (mN/m) of the carrier and tofacitinib is below that of tofacitinib and a plastic.
726. The topical composition of any of embodiment 601-715, 722, or 723, wherein the interfacial tension (mN/m) of the carrier and tofacitinib is at least about 5% below e.g., 10% or 15% below that of tofacitinib and a plastic.
727. The topical composition of any of embodiment 601-715 or 722-725, wherein the plastic is PTFE (polytetrafluorethylene).
728. The topical composition of any of embodiment 601-721, wherein the surface energy of the composition is below that of the tofacitinib with a metal.
729. The topical composition of any of embodiment 601-715, wherein the interfacial tension between non-micronized tofacitinib and the composition is less than about 1.6 mN/m or between about 1.5 mN/m and about 1.1 nM/m.
730. The topical composition of any of embodiment 601-715, wherein the interfacial tension between micronized tofacitinib and the composition is less than about 2.5 mN/m, or between about 1.8 mN/m and about 2.3 mN/m.
731. The topical composition of any preceding embodiment, wherein the ratio of carrier base to emollient is about or less than 9:1, or between about 9:1 and about 6:1.
732. The topical composition of any preceding embodiment, wherein the ratio of carrier base to emollient is between about 8:1 and about 7:1, about 8:1, about 22:3, or about 7:1.
733. The topical composition of any preceding embodiment, wherein the carrier base is about 83% to about 90%, about 86% to about 88%, or about 87% by weight of the composition.
734. The topical composition of any preceding embodiment, wherein the carrier base comprises elastomer and is about 83% to about 90% by weight of the composition and the emollient is about 10% to about 16% by weight of the composition.
735. The topical composition of embodiment 733, wherein the carrier base comprises elastomer and is about 86% to about 88% by weight of the composition and the emollient is about 11% to about 14% by weight of the composition.
736. The topical composition of any preceding embodiment, wherein the emollient comprises a triglyceride oil comprising one or more of an MCT oil, an olive oil, a coconut oil, a palm oil, a sunflower oil, a rapeseed oil, a soybean oil, a groundnut oil, a peanut oil, a corn oil, a walnut oil, a soya oil, a fish oil, a tallow, a fraction of any of the aforesaid, or mixtures of any two or more thereof.
737. The topical composition of any preceding embodiment, wherein the tofacitinib and the fingolimod are the sole active agents in the composition.
738. The topical composition of any preceding embodiment, wherein the carrier or composition is a gel, or a semi-solid, or a liquid at room temperature.
739. The topical composition of embodiment 737, wherein the composition is a gel at room temperature.
740. The topical composition of embodiment 737, wherein the composition is a semi-solid at room temperature.
741. The topical composition of embodiment 737, wherein the composition is a liquid at room temperature.
742. The topical composition of any preceding embodiment, wherein composition is foamable and comprises a foam adjuvant and/or a surfactant.
743. The topical composition of embodiment 741, wherein foamable composition comprises a propellant.
744. The topical composition of embodiment 742, wherein the foamable composition upon release from a pressurized canister forms a foam.
745. The topical composition of any preceding embodiment, wherein the composition when applied to a surface does not run.
746. The topical composition of any preceding embodiment, wherein the composition when applied to a skin or mucosal surface has a bioadhesive or mucoadhesive quality.
747. The topical composition of any preceding embodiment, wherein the composition forms a quasi-layer.
748. The topical composition of any preceding embodiment, wherein the quasi-layer facilitates absorption of the tofacitinib into epidermal and dermal layers of skin.
749. The topical composition of any preceding embodiment, wherein the quasi-layer facilitates absorption of the tofacitinib into a mucosal membrane.
750. The topical composition of any preceding embodiment, wherein the quasi-layer facilitates absorption of the tofacitinib into a lining of a body cavity.
751. The topical composition of any preceding embodiment, wherein the carrier base and emollient act synergistically to enhance delivery even though the tofacitinib or the fingolimod are not soluble or substantially not soluble in the carrier base and emollient.
752. The topical composition of embodiment 691, wherein the composition provides at least two, three, or four of the following characteristics:

an increase in the chemical stability of tofacitinib salt,

a reduction or elimination of balling;

when applied topically to skin or mucosa an increased delivery of tofacitinib into the skin or mucosa;

when applied topically to skin or mucosa a reduced delivery of tofacitinib through the skin or mucosa; and

when applied topically to skin an increased delivery of tofacitinib into the epidermis and reduced delivery through the skin.

753. The topical composition of any preceding embodiment further comprising at least one of a fragrance agent, a masking agent, a buffering agent, a pH agent, a preservative, a chelating agent, an anti-oxidant, a scavenger agent, a thickener, a diluent, or any mixtures of two or more thereof.
754. The topical composition of any preceding embodiment, wherein the fingolimod is a pharmaceutically acceptable salt.
755. The topical composition of any preceding embodiment, wherein the fingolimod salt is a citrate salt, hydrochloride salt, hydrobromide salt, oxalate salt, nitrate salt, sulfate salt, phosphate salt, fumarate salt, succinate salt, maleate salt, besylate salt, tosylate salt, palmitate salt, tartrate salt, adipate salt, laurate salt, or myristate salt.
756. The topical composition of any preceding embodiment, wherein the fingolimod comprises fingolimod hydrochloride.
757. The topical composition of any preceding embodiment, wherein the fingolimod comprises fingolimod base.
758. The topical composition of any preceding embodiment, wherein the fingolimod is present in an amount sufficient to restore the skin barrier.
759. The topical composition of embodiment 756, wherein the fingolimod increases the level of filaggrin in the skin.
760. The topical composition of any preceding embodiment, wherein the fingolimod is present in an amount of about 0.0001% to about 10% by weight of the composition and the tofacitinib is present in an amount of about 0.01% to about 10% by weight of the composition, the fingolimod is about 0.001% to about 1% by weight of the composition and the tofacitinib is about 0.05% to about 3.05% by weight of the composition, the fingolimod is about 0.002% to about 0.1% by weight of the composition and the tofacitinib is about 0.1% to about 1% by weight of the composition, or the fingolimod is about 0.005% to about 0.01% by weight of the composition and the tofacitinib is about 0.3% to about 0.6% by weight of the composition, or fingolimod is about 0.001% to about 0.01% by weight of the composition and the tofacitinib is about 0.3% to about 0.6% by weight of the composition, or fingolimod is about 0.005% to about 0.01% by weight of the composition and the tofacitinib is about 0.3% to about 0.6% by weight of the composition, or fingolimod is about 0.005% to about 0.02% by weight of the composition and the tofacitinib is about 0.3% to about 0.6% by weight of the composition, or fingolimod is about 0.01% to about 0.02% by weight of the composition and the tofacitinib is about 0.3% to about 0.6% by weight of the composition, or fingolimod is about 0.01% to about 0.1% by weight of the composition and the tofacitinib is about 0.3% to about 0.6% by weight of the composition, or fingolimod is about 0.001% to about 0.1% by weight of the composition and the tofacitinib is about 0.3% to about 0.6% by weight of the composition.
761. The topical composition of any preceding embodiment, wherein the fingolimod is at a concentration of about 0.001% to about 0.01% by weight of the composition, or about 0.005% to about 0.01% by weight of the composition, or about 0.005% to about 0.02% by weight of the composition, or about 0.01% to about 0.02% by weight of the composition, or about 0.01% to about 0.1%, or about 0.001% to about 0.1% by weight of the composition.
762. A kit comprising the composition of any of preceding embodiment in a container and a disposable applicator connectable to the container.
763. The kit of embodiment 761, wherein the container comprises a unit dose means suitable for delivery of a measured unit dose of the tofacitinib and the fingolimod.
764. The kit of embodiment 762, wherein the unit dose is about 0.1 g, about 0.2 g, about 0.3 g, about 0.4 g, about 0.5 g, about 0.6 g, about 0.7 g, about 0.8 g, about 0.9 g, or about 1.0 g of composition.
765. The kit any of embodiment 761-763, wherein the disposable applicator is adapted for delivery of the composition to a body cavity.
766. The kit of any of embodiment 761-764, wherein the disposable applicator is adapted for delivery of the composition to a skin surface.
767. The kit of any of embodiment 761-765, wherein the disposable applicator is adapted for delivery of the composition to a mucosal surface.
768. A method of treating or ameliorating a skin disorder comprising applying to the skin of a subject the composition of any of embodiments 594-760.
769. A method of treating or ameliorating a mucosal disorder comprising applying to the mucosa of a subject the composition of any of embodiments 594-760.
770. A method of treating or ameliorating a body cavity disorder comprising applying to a body cavity of a subject the composition of any of embodiments 594-760.
771. A method of treating a JAK related condition comprising applying to the skin or mucosa or body cavity of a subject the composition of any of embodiments 594-760.
772. A method of treating or preventing a dermatological disorder, or a deterioration thereof, comprising applying to the skin of a subject the composition of any of embodiments 594-760.
773. The method of embodiment 771, wherein the disorder is dermatitis, atopic dermatitis, psoriasis, hypertrophic scars, keloid scars, post-surgery scars, or eczema.
774. The method of embodiment 772, wherein the eczema is atopic dermatitis, contact dermatitis, dyshidrotic eczema, nummular eczema, seborrheic dermatitis, or stasis dermatitis.
775. The method of embodiment 772, wherein the dermatological disorder is atopic dermatitis.
776. The method of embodiment 772, wherein the dermatological disorder is psoriasis.
777. The method of embodiment 772, wherein the dermatological disorder is scarring.
778. The method of any of embodiments 771-776, wherein the tofacitinib is delivered into the epidermis and the dermis.
779. The method of embodiment 777, wherein the delivery to the epidermis is greater than to the dermis.
780. The method of embodiment 777, wherein the delivery to the epidermis is at least about 20%, at least about 50%, at least about 100%, at least about 150%, at least about 200%, at least about 250%, or at least about 300% greater than to the dermis.
781. The method of embodiment 777, wherein the delivery to the epidermis is expressed as a percentage of applied dose.
782. The method of embodiment 780, wherein the delivery to the epidermis as a percentage of applied dose is at least about 100% greater than to the dermis.
783. The method of embodiment 780, wherein the topical delivery of the tofacitinib to the dermis and epidermis is about or greater than 20-fold the delivery of the tofacitinib through the skin.
784. The method of any of embodiment 767-782, wherein the composition is applied to the area of the disorder.
785. The method any of embodiment 767-782, wherein the composition is applied to the area surrounding the area of the disorder.
786. The method of any of embodiment 767-782, wherein the composition is applied to the area of the disorder and the area surrounding the disorder.
787. The method of any of embodiment 767-782, wherein the composition is applied once daily.
788. The method of any of embodiment 767-782, wherein the composition is applied twice daily.
789. The method of any of embodiment 767-782, wherein the composition is applied at least once per day for at least 7 days.
790. The method of any of embodiment 767-782, wherein the composition is applied at least once per day for at least 14 days.
791. The method of any of embodiment 767-782, wherein the composition is applied at least once per day for at least 4 weeks.
792. The method of any of embodiment 767-782, wherein the composition is applied at least once per day for at least 8 weeks.
793. The method of any of embodiment 767-782, wherein the composition is applied at least once per day for at least 12 weeks.
794. The method of any of embodiment 767-782, wherein the composition is applied as a maintenance dose following an initial treatment period.
795. The method of any of embodiment 767-782, wherein the maintenance dose is applied on non-consecutive days.
796. The method of embodiment 794, wherein the maintenance dose is applied on alternative days.
797. The method of embodiment 794, wherein the maintenance dose is applied twice weekly.
798. The method of any of embodiment 767-796, wherein systemic exposure to tofacitinib applied topically is much less than when the same amount is applied orally.
799. The method of embodiment 797, wherein the systemic exposure is at least 20-fold less.
800. The method of embodiment 797, wherein the systemic exposure is at least 50-fold less.
801. The method of embodiment 797, wherein the systemic exposure is at least 100-fold less.
802. The method of embodiment 797, wherein the systemic exposure is at least 200-fold less.
803. The method of embodiment 797, wherein the systemic exposure is at least 400-fold less.
804. The method of embodiment 797, wherein the systemic exposure is at least 500-fold less.
805. The method of embodiment 774, wherein the atopic dermatitis index is reduced by about 20%, about 25%, or about 30% compared to placebo.
806. The method of embodiment 804, wherein the index is less than four.
807. The method of embodiment 804, wherein the index is about 3.
808. The method of embodiment 774, wherein the psoriasis index is reduced by about 20%, about 25%, or about 30% compared to placebo.
809. The method of embodiment 807, wherein the index is less than four.
810. The method of embodiment 807, wherein the index is about 3.4.
811. The method of any of embodiment 767-809, wherein the carrier is not an emulsion.
812. The method of embodiment 770, wherein the JAK related condition or disorder comprises alopecia totalis, alopecia universalis, vitiligo, autoimmune bullous skin disorder, pemphigus vulgaris (PV), bullous pemphigoid (BP), skin rash, skin irritation, skin sensitization, contact dermatitis, allergic contact dermatitis, chronic atypical neutrophilic dermatosis with lipodystrophy and elevated temperature (CANDLE), pustulosis palmoplantaris, ichtyosis, hypertrophic scars, keloid scars, post-surgery scars, eczema, actinic keratosis, pruritus, rosacea, or acne.
813. The method of embodiment 770, wherein the JAK related condition or disorder comprises vitiligo and the composition is applied topically to the area of skin lacking pigment and its surrounds.
814. The method of embodiment 770, wherein the JAK related condition or disorder comprises alopecia and the composition is applied topically to the skin and hair.
815. The method of embodiment 771, wherein the disorder comprises a dermatitis, atopic dermatitis, dermatomyositis, eczema, psoriasis, rosacea, acne, disorder of the pilosebaccous unit, alopecia, alopecia totalis, alopecia universalis, vitiligo, autoimmune bullous skin disorder, skin rash, skin irritation, skin sensitization, chronic atypical neutrophilic dermatosis with lipodystrophy and elevated temperature (CANDLE), pustulosis palmoplantaris, ichtyosis, actinic keratosis, pruritus, contact dermatitis, dyshidrotic eczema, nummular eczema, seborrheic dermatitis, stasis dermatitis, lupus erythematosus, skin inflammation, skin itch, skin infection, skin scars, folliculitis/keratosis pilaris, hidradentitis suppurativa, pyoderma gangrenosum, lichenification disorders, primary cicatricial alopecias, or cellulitis.
816. The topical composition of any preceding embodiment, wherein the fingolimod is chemically stable.
817. The topical composition of embodiment 770, wherein the fingolimod is chemically stable for at least 3 weeks at 5° C.
818. The topical composition of embodiment 770, wherein the fingolimod is chemically stable for at least 3 weeks at 40° C.
819. The topical composition of embodiment 770, wherein the fingolimod is chemically stable for at least 3 weeks at 50° C.
820. The topical composition of embodiment 770, wherein the fingolimod is chemically stable for at least 2 months at 5° C.
821. The topical composition of embodiment 770, wherein the fingolimod is chemically stable for at least at least 2 months at 40° C.
822. The topical composition of embodiment 770, wherein the fingolimod is chemically stable for at least at least 2 months at 50° C.
823. The topical composition of any of embodiment 815-821, wherein at least about 90%, about 95%, about 98%, or about 99% by mass of the fingolimod is present in the composition when stored for 3 weeks or 2 months at 5° C.
824. The topical composition of any of embodiment 815-821, wherein at least about 90%, about 95%, about 98%, or about 99% by mass of the fingolimod is present in the composition when stored for 3 weeks or 2 months at 25° C.
825. The topical composition of any proceeding embodiment, wherein the tofacitinib and the fingolimod are chemically stable in the composition.
826. The topical composition of embodiment 822, wherein at least about 90%, about 95%, about 98%, or about 99% by mass of the tofacitinib and of the fingolimod is present in the composition when stored for 3 or 6 months at 5° C.
827. The topical composition of embodiment 823, wherein at least about 90%, about 95%, about 98%, or about 99% by mass of the tofacitinib and of the fingolimod is present in the composition when stored for 3 or 6 months at 25° C.
828. Use of the topical composition of any preceding embodiment as a medicament for preventing, treating or ameliorating a disorder or a deterioration thereof comprising applying to the skin of a subject the topical composition.
829. Use of the topical composition of embodiment 827, wherein the disorder comprises a dermatitis, atopic dermatitis, dermatomyositis, eczema, psoriasis, rosacea, acne, disorder of the pilosebaceous unit, alopecia, alopecia totalis, alopecia universalis, vitiligo, autoimmune bullous skin disorder, skin rash, skin irritation, skin sensitization, chronic atypical neutrophilic dermatosis with lipodystrophy and elevated temperature (CANDLE), pustulosis palmoplantaris, ichtyosis, actinic keratosis, pruritus, contact dermatitis, dyshidrotic eczema, nummular eczema, seborrheic dermatitis, stasis dermatitis, lupus erythematosus, skin inflammation, skin itch, skin infection, skin scars, folliculitis/keratosis pilaris, furunculosis, hidradentitis suppurativa, pyoderma gangrenosum, lichenification disorders, primary cicatricial alopecias, or cellulitis.
830. Use of the topical composition of embodiment 827, wherein the dermatological disorder involves inflammation. A dual chamber kit comprising two containers,
831, wherein a first container comprises a first composition comprising tofacitinib or a pharmaceutically acceptable salt thereof and a first carrier and a second container comprising a second composition comprising a second carrier and fingolimod or a pharmaceutically acceptable salt thereof, wherein each composition is stored separately and upon dispensing the compositions are combined with a mixer connectable to each container and optionally comprising a unit dose means for dispensing a measured unit dose from each container.
832. The method of embodiment 814, wherein the autoimmune bullous skin disorder is pemphigus vulgaris (PV) or bullous pemphigoid (BP).
833. The method of embodiment 814, wherein the skin sensitization is contact dermatitis or allergic contact dermatitis.
834. The method of embodiment 814, wherein the skin scars are hypertrophic scars, keloid scars, or post-surgery scars.
835. The method of embodiment 814, wherein the lichenification disorders are lichen planus/sclerosus or lichen simplex chronicus/neurodermatitis.
836. The method of embodiment 814, wherein the primary cicatricial alopecias are lichen planopilaris or frontal fibrosing alopecia.
837. The use of embodiment 828, wherein the autoimmune bullous skin disorder is pemphigus vulgaris (PV) or bullous pemphigoid (BP).
838. The use of embodiment 828, wherein the skin sensitization is contact dermatitis or allergic contact dermatitis.
839. The use of embodiment 828, wherein the skin scars are hypertrophic scars, keloid scars, or post-surgery scars.
840. The use of embodiment 828, wherein the lichenification disorders are lichen planus/sclerosus or lichen simplex chronicus/neurodermatitis.
841. The use of embodiment 828, wherein the primary cicatricial alopecias are lichen planopilaris or frontal fibrosing alopecia.
842. In one or more embodiments the penetration of tofacitinib base in the epidermis is higher than that of tofacitinib citrate when equivalent amounts are applied to the skin of a subject in the otherwise same elastomer based composition. In one or more embodiments when equivalent amounts are applied to the skin of a subject the penetration of tofacitinib base in the epidermis when delivered in a PEG based ointment formulation is lower than the penetration oftofacitinib citrate when delivered in an elastomer based formulation. Thus, in one or more embodiments a PEG based carrier is less effective in delivering the tofacitinib that an elastomer based formulation. In one or more embodiments tofacitinib base does not deliver into the skin of a subject as well in a PEG based carrier compared to a elastomer based carrier.

We claim:

1. A topical composition comprising tofacitinib or a pharmaceutically acceptable salt thereof, and fingolimod or a pharmaceutically acceptable salt thereof, and a carrier in which the fingolimod and tofacitinib are suspended or substantially suspended.

2. The topical composition of claim 1, wherein the carrier comprises a carrier base and at least one emollient, wherein the carrier base comprises at least one elastomer.

3. The topical composition of any of claim 1 or 2, wherein the carrier is free or substantially free of a penetration enhancer that dissolves a proportion of the tofacitinib or the fingolimod.

4. The topical composition of any preceding claim, wherein the at least one emollient comprises one or more emollients that enhances the penetration of tofacitinib when the composition is applied to the skin or mucosa.

5. The topical composition of any preceding claim, wherein the emollients comprise an isopropyl ester and a saturated or branched hydrocarbon oil.

6. The topical composition of claim 5, wherein the isopropyl ester is isopropyl isostearate and the saturated or branched hydrocarbon oil is squalane.

7. The topical composition of any preceding claim, wherein the emollient further comprises one or more of MCT oil, mineral oil, or isopropyl palmitate.

8. The topical composition of any preceding claim, wherein the emollient further comprises two or more of MCT oil, mineral oil, or isopropyl palmitate.

9. The topical composition of any of claims 5-8, wherein one or more of the adhesiveness, surface energy, surface tension, or interfacial tension of the composition is reduced to reduce adhesion of the composition to a surface.

10. The topical composition of any preceding claim, wherein by altering the amounts and/or ratios of said emollients the penetration of the tofacitinib into the skin is improved.

11. The topical composition of any preceding claim, wherein by altering the amounts and/or ratios of said emollients the ratio of penetration of the tofacitinib into the skin to penetration of the tofacitinib through the skin is improved.

12. The topical composition of claim 10 or 11, wherein the ratio of isopropyl isostearate to other emollients is about 12:1 to about 1:12, e.g., about 10:1, about 8:1, about 6:1, about 5:1, about 4:1, about, 3:1, about 2:1, about 1:1, about 1:2, about 1:3, about 1:4, about 1:5, about 1:6, about, 1:7, about 1:8, or about 1:10.

13. The topical composition of claim 10 or 11, wherein the ratio of squalane to other emollients is about 12:1 to about 1:12, e.g., about 10:1, about 8:1, about 6:1, about 5:1, about 4:1, about, 3:1, about 2:1, about 1:1, about 1:2, about 1:3, about 1:4, about 1:5, about 1:6 about, 1:7, about 1:8, or about 1:10.

14. The topical composition of claim 10 or 11, wherein the at least one emollient comprises 3 emollients in the ratios of about, 1:1:1, or about 1:4:1, or about 1:1:4, or about 4:1:1.

15. The topical composition of claim 14, wherein one of the emollients is isopropyl isostearate or squalane.

16. The topical composition of claim 14, wherein two of the emollients are isopropyl isostearate or squalane.

17. The topical composition of any of claims 14-16, wherein the emollients in addition to isopropyl isostearate and squalane comprise one or more of an MCT oil, a mineral oil, or isopropyl palmitate.

18. The topical composition of any preceding claim, wherein the emollient comprises a glyceride, a triglyceride, a diglyceride, a monoglyceride, an MCT oil, a branched hydrocarbon oil, a saturated and branched hydrocarbon oil, squalene, squalane, a branched alkyl ester, an isopropyl ester, a glycerol iso-ester, isopropyl isostearate, isopropyl palmitate, isopropyl myristate, oleyl alcohol, a mineral oil, a vegetable oil, a liquid fatty acid, a liquid fatty alcohol, a branched liquid fatty acid, a branched liquid fatty alcohol, glyceryl monooleate, glyceryl isostearate, glyceryl dicaprate, a polypropylene glycerol alkyl ether, a polypropylene glycerol stearyl ether, polypropylene glycerol 15 stearyl ether, polypropylene glycerol 11 stearyl ether, glycerol behenate, diisopropyl adipate, cetearyl ethylhexanoate, or cetearyl isononanoate, or mixtures of two or more thereof.

19. The topical composition of any preceding claim, wherein the emollient includes one or more of a glyceride oil, a branched chain ester, or a branched hydrocarbon oil.

20. The topical composition of any preceding claim, wherein the emollient is a triglyceride oil, an isopropyl ester, or a saturated or branched hydrocarbon oil.

21. The topical composition of any preceding claim, wherein the emollient is at least about 4%, or at least about 6%, or at least about 8%, or at least about 10%, or at least about 12%, or at least about 14%, or at least about 16%, or at least about 18%, or at least about 20%, or at least about 22%, or at least about 24% by weight of the composition.

22. The topical composition of any preceding claim, wherein the emollient is less than about 30%, or less than about 28%, or less than about 26%, or less than about 24%, or less than about 22%, or less than about 20%, or less than about 18%, or less than about 16%, or less than about 14%, or less than about 12% by weight of the composition.

23. The topical composition of any preceding claim, wherein the emollient is about 4% to about 30%, or about 5% to about 28%, or about 6% to about 26%, or about 7% to about 24%, or about 8% to about 22%, or about 8% to about 20%, or about 8% to about 18%, or about 8% to about 16% or about 9% to about 20%, or about 9% to about 19%, or about 9% to about 17%, or about 9% to about 15%, or about 10% to about 18%, or about 10% to about 16%, or about 10% to about 14% by weight of the composition.

24. The topical composition of any preceding claim, wherein the emollient is about 4%, or about 5%, or about 6% or about 7% or about 8% or about 9%, or about 10%, or about 11% or about 12% or about 13% or about 14%, or about 15%, or about 16%, or about 17%, or about 18%, or about 19%, about 20%, or about 21% or about 22%, or about 23%, or about 24% or about 25% by weight of the composition.

25. The topical composition of any preceding claim, wherein the emollient is about 9% to about 15%, e.g., about 9%, about 10%/6, about 11%, about 12%, about 13%, about, 14%, or about 15% by weight of the composition.

26. The topical composition of any preceding claim, wherein penetration of the tofacitinib into the epidermis is improved.

27. The topical composition of any preceding claim, wherein penetration of the tofacitinib into the dermis is improved.

28. The topical composition of any preceding claim, wherein penetration of the tofacitinib into the epidermis and dermis is improved.

29. The topical composition of any preceding claim, wherein the ratio of penetration into the skin to penetration through the skin is about at least 50:1, or about at least 75:1, or about at least 100:1, or about at least 125:1, or about at least 150:1, or about at least 175:1, or about at least 200:1, or about at least 225:1, or about at least 250:1, or about at least 275:1, or about at least 300:1, or about at least 325:1, or about at least 350:1, or about at least 375:1, or about at least 400:1, or about at least 425:1, or about at least 450:1, or about at least 475:1, or about at least 500:1.

30. The topical composition of any preceding claim, wherein the ratio of penetration into the skin to penetration through the skin is about 50:1 to about 500:1, or about 100:1 to about 500:1, or about 150:1 to about 500:1, or about 200:1 to about 500:1, or about 250:1 to about 500:1, or about 300:1 to about 500:1, or about 350:1 to about 500:1, or about 400:1 to about 500:1, or about 450:1 to about 500:1, or about 75:1 to about 450:1, or about 100:1 to about 425:1, or about 75:1 to about 400:1, or about 75:1 to about 375:1, or about 75:1 to about 350:1, or about 100:1 to about 400:1, or about 100:1 to about 375:1, or about 100:1 to about 350:1, or about 125:1 to about 400:1, or about 125:1 to about 375:1, or about 125:1 to about 350:1, or about 150:1 to about 375:1, or about 50:1 to about 50:100, or about 50:1 to about 50:100, or about 50:1 to about 50:150, or about 50:1 to about 50:200, or about 50:1 to about 50:250, or about 50:300 to about 50:350, or about 50:1 to about 400:1, or about 50:1 to about 450:1, or about 50:1 to about 500:1.

31. The topical composition of any preceding claim, wherein the pK of the tofacitinib in the blood is low.

32. The topical composition of any preceding claim, wherein the tofacitinib is a JAK inhibitor that acts on one or more JAK receptors and is in a concentration sufficient to bind to one or more Janus Kinase (JAK) receptors in the dermis or epidermis in an applied area of skin of a mammal e.g., human subject.

33. The topical composition of any preceding claim, wherein the tofacitinib provides a dose dependent effect when applied to the skin or mucosa of a subject.

34. The topical composition of any preceding claim, wherein the tofacitinib is a pharmaceutically acceptable salt.

35. The topical composition of any preceding claim, wherein the tofacitinib salt is a citrate salt, hydrochloride salt, hydrobromide salt, oxalate salt, nitrate salt, sulfate salt, phosphate salt, fumarate salt, succinate salt, maleate salt, besylate salt, tosylate salt, palmitate salt, tartrate salt, adipate salt, laurate salt, or myristate salt.

36. The topical composition of any preceding claim, wherein the tofacitinib comprises tofacitinib citrate.

37. The topical composition of claim 33, wherein the tofacitinib comprises tofacitinib base.

38. The topical composition of claim 36 or 37, wherein the penetration of tofacitinib base in the epidermis is higher than that of tofacitinib citrate, when equivalent amounts are applied to the skin of a subject in the otherwise same composition.

39. The topical composition of any preceding claim, wherein the tofacitinib is about 0.3% to about 5% by weight of the composition.

40. The topical composition of any preceding claim, wherein the tofacitinib is about 0.3% to about 5%, or 0.4% to about 4.5%, or 0.5% to about 4%, or 0.6% to about 3.5%, or about 0.6% to about 3%, or 0.6% to about 3.5%, or about 0.6% to about 3%, or about 0.6% to about 2.5%, or about 0.6% to about 2%, or about 0.6% to about 2.5%, or 0.8% to about 3.5%, or about 0.8% to about 3%, or about 0.8% to about 2.5%, or about 0.8% to about 2%, or about 0.8% to about 1.8%, or about 0.8% to about 1.5%, or about 1% to about 3.5%, or about 1% to about 3%, or about 1% to about 2.5%, or about 1% to about 2%, or about 1% to about 1.8%, or about 1% to about 1.5% by weight of the composition.

41. The topical composition of any preceding claim, wherein the tofacitinib is about 0.3%, or about 0.4%, or about 0.5%, or about 0.6%, or about 0.7%, or about 0.8%, or about 0.9%, or about 1%, or about 1.1%, or about 1.2%, or about 1.3%, or about 1.4%, or about 1.5%, or about 1.6%, or about 1.7%, or about 1.8%, or about 1.9%, or about 2.0%, or about 2.1%, or about 2.2%, or about 2.3%, or about 2.4%, or about 2.5%, or about 2.6%, or about 2.7%, or about 2.8%, or about 2.9%, or about 3%, or about 3.1%, or about 3.2%, or about 3.3%, or about 3.4%, or about 3.5%, or about 3.6%, or about 3.7%, or about 3.8%, or about 3.9%, or about 4%, or about 4.1%, or about 4.2%, or about 4.3%, or about 4.4%, or about 4.5%, or about 4.6%, or about 4.7%, or about 4.8%, or about 4.9%, or about 5% by weight of composition.

42. The topical composition of any preceding claim, wherein the tofacitinib is about 1%, or about 1.1%, or about 1.2%, or about 1.3%, or about 1.4%, or about 1.5%, or about 1.6%, or about 1.7%, or about 1.8% by weight of the composition.

43. The topical composition of any preceding claim, wherein the tofacitinib is about 0.3%, or about 0.35%, or about 0.4%, or about 0.45%, or about 0.5%, or about 0.55%, or about 0.6%, or about 0.65%, or about 0.7%, or about 0.75%, or about 0.8%, or about 0.85%, or about 0.9%, or about 0.95%, or about 1.0%, or about 1.05%, or about 1.1%, or about 1.15%, or about 1.25%, or about 1.35%, or about 1.45%, or about 1.55% by weight of the composition.

44. The topical composition of any preceding claim further comprising a third active agent.

45. The topical composition of any preceding claim, wherein the third active agent comprises one or more of a JAK inhibitor, an antipruritic, an anesthetic, an antibiotic, an anti-atopic dermatitis agent, an anti-alopecia agent, an antihistamine, an anti-fibrinolytic agent, an anti-scarring agent, a serine protease inhibitor, a cysteine protease inhibitor, an anti-vitiligo agent, an anti-psoriasis agent, a MEK inhibitor, an immunosuppressive agent, a sphingosine-1-phosphate receptor modulator or agonist, a steroid, a NSAID, a retinoid, or a dicarboxylic acid.

46. The topical composition of claim 44, wherein the third active agent is seliforant.

47. The topical composition of claim 44, wherein the third active agent is aminocaproic acid or trametinib dimethylsulfoxide.

48. The topical composition of any preceding claim, wherein at least about 99.9% of the tofacitinib is suspended.

49. The topical composition of any of the preceding claim, wherein the tofacitinib is homogeneously suspended.

50. The topical composition of any of the preceding claim, wherein the tofacitinib is micronized.

51. The topical composition of any of the preceding claim, wherein the tofacitinib is suspended as nanoparticles.

52. The topical composition of any of the preceding claim, wherein the wherein the fingolimod is homogeneously suspended, optionally the fingolimod is micronized or is suspended as nanoparticles.

53. The topical composition of claim 50, wherein the D90 is between about 2 μm to about 50 μm e.g., about 2 μm to about 20 μm.

54. The topical composition of claim 50, wherein the D90 is less than about 22 μm, or less than about 10 μm, or is about 9 μm, or about 8 μm, or about 7.5 μm, or about 7 μm, or about 6 μm, or about 5 μm or about 4 μm, or about 3 μm.

55. The topical composition of claim 51 or 52, wherein the D90 is less than about 1 μm or less than about 0.75 μm, or less than about 0.5 μm, or less than about 0.25 μm, or less than about 0.2 μm, or is about 0.9 μm, or is about 0.8 μm, or is about 0.7 μm, or is about 0.6 μm, or is about 0.5 μm, or is about 0.4 μm, or is about 0.3 μm, or is about 0.25 μm, or is about 0.2 μm, or is about 0.15 μm, or is about 0.1 μm.

56. The topical composition of any of the preceding claim, wherein the carrier reduces the potential for agglomeration of suspended tofacitinib or fingolimod.

57. The topical composition of claim 56, wherein the reduction is in one or more of frequency of agglomerates, number of agglomerates, or size of agglomerates.

58. The topical composition of claim 56, wherein the average size of agglomerates is less than about 175 μm, less than about 150 μm, less than about 125 μm, less than about 100 μm, less than about 75 μm, or less than about 50 μm.

59. The topical composition of claim 56, wherein at least about 95% of the tofacitinib is not present as agglomerates.

60. The topical composition of claim 56, wherein less than about 3% of the composition comprises agglomerates.

61. The topical composition of claim 56, wherein less than about 1% of the composition comprises agglomerates.

62. The topical composition of claim 56, wherein the composition is free or substantially free of agglomerates.

63. The topical composition of any preceding claim further comprising one or more surfactants.

64. The topical composition of claim 63, wherein at least one surfactant is non-ionic and has an HLB of less than about 9.

65. The topical composition of claim 63, wherein at least one surfactant is non-ionic and has an HLB of less than about 7.

66. The topical composition of claim 63, wherein the surfactants are non-ionic and have an average HLB of less than about 9.

67. The topical composition of claim 63, wherein the surfactants are non-ionic and have an average HLB of less than about 7.

68. The topical composition of any of the preceding claim, wherein the carrier is not hydrophilic.

69. The topical composition of any of the preceding claim, wherein the carrier is free of or substantially free of hydrophilic compounds.

70. The topical composition of any of the preceding claim, wherein the hydrophilic compound is volatile.

71. The topical composition of any of the preceding claim, wherein the volatile hydrophilic compound is a propellant.

72. The topical composition of any of the preceding claim, wherein the carrier is free or substantially free of a surfactant.

73. The topical composition of any of the preceding claim, wherein the carrier is free or substantially free of water.

74. The topical composition of any of the preceding claim, wherein the carrier is free or substantially free of preservatives.

75. The topical composition of any of the preceding claim, wherein the carrier is free or substantially free of anti-oxidants.

76. The topical composition of any of the preceding claim, wherein the carrier is free or substantially free of scavengers.

77. The topical composition of any of claims 74-76, wherein the carrier is free or substantially free of additional stabilizers.

78. The topical composition of any preceding claim, wherein the carrier is free or substantially free of a compound that essentially dissolves a proportion of the tofacitinib.

79. The topical composition of claim 78, wherein the carrier is free, or essentially free, or substantially free of a compound that dissolves a proportion of the tofacitinib.

80. The topical composition of claim 79, wherein the compound is water, HCl, transcutol, dimethyl isosorbate, a glycol, a polyethylene glycol, polyethylene glycol 200, polyethylene glycol 400, propylene glycol, glycerol, sulphoxides, dimethyl sulfoxide, dimethylacetamide, or dimethylformamide.

81. The topical composition of any preceding claim, wherein the carrier is free or substantially free of a penetration enhancer that dissolves a proportion of the tofacitinib, wherein the proportion is at least about 0.1%, or about 0.5%.

82. The topical composition of any preceding claim, wherein the carrier is free or substantially free of a penetration enhancer that dissolves a proportion of the tofacitinib, wherein the proportion is at least about 1%, about 2%, about 5% or about 10%.

83. The topical composition of any preceding claim, wherein less than about 1%, e.g., 0.5% or 0.1% of tofacitinib present in the composition is dissolved.

84. The topical composition of any preceding claim, wherein the composition is non-occlusive or substantially non-occlusive.

85. The topical composition of any preceding claim, wherein the composition is partially occlusive.

86. The topical composition of any preceding claim, wherein the carrier is free or substantially free of an occlusive agent.

87. The topical composition of any preceding claim, wherein the carrier is free or substantially free of a petrolatum.

88. The topical composition of any preceding claim, wherein the carrier is free or substantially free of a solid wax having a melting temperature greater than about 45° C.

89. The topical composition of any preceding claim, wherein the carrier is free or substantially free of compounds to which the tofacitinib or the fingolimod is not inert.

90. The topical composition of any preceding claim, wherein the carrier is lipophilic.

91. The topical composition of claim 90, wherein the lipophilic carrier comprises at least one oil that is liquid at room temperature.

92. The topical composition of claim 90, wherein the lipophilic carrier comprises at least one oil that is solid at room temperature.

93. The topical composition of claim 90, wherein the lipophilic carrier comprises at least one oil that is liquid at room temperature, or at least one oil that is solid at room temperature.

94. The topical composition of any preceding claim, wherein the carrier comprises a polymeric agent.

95. The topical composition of claim 94, wherein the polymeric agent is a gelling agent.

96. The topical composition of any preceding claim, wherein the carrier comprises a gelling agent and a hydrophobic agent or oil.

97. The topical composition of any preceding claim, wherein the at least one elastomer comprises one or more of cyclopentasiloxane (and) polysilicone-11 (Grant MGS-Elastomer 1100), dimethicone (and) polysilicone-11 (Gransil DMG-3), a cyclopentasiloxane (and) petrolatum (and) polysilicone-11 (MGS-Elastomer I148P), cyclopentasiloxane (and) dimethicone cross polymer (ST-Elastomer 10), or dimethicone (and) dimethicone crosspolymer (DOWSIL™ 9041).

98. The topical composition of any preceding claim, wherein the elastomer comprises ST-Elastomer 10.

99. The topical composition of claim 97 or 98 comprising a tofacitinib salt, wherein the salt is more stable than tofacitinib base.

100. The topical composition of claim 97 or 98, wherein the viscosity of the composition is stable or substantially stable from about 8° C. to about 40° C.

101. The topical composition of claim 97 or 98, wherein the viscosity of the composition is stable or substantially stable from about 10° C. to about 35° C.

102. The topical composition of claim 97 or 98, wherein the viscosity of the composition is stable or substantially stable from about 15° C. to about 30° C.

103. The topical composition of claim 97 or 98, wherein the viscosity of the composition is stable or substantially stable from about 20° C. to about 25° C.

104. The topical composition of any preceding claim, wherein the carrier further comprises a gelled oil.

105. The topical composition of claim 104, wherein the gelled mineral oil comprises a mineral oil and ethylene/propylene/styrene copolymer and butylene/ethylene/styrene copolymer.

106. The topical composition of any preceding claim, wherein the carrier base comprises a silicone oil in addition to the elastomer.

107. The topical composition of claim 106, wherein the silicone oil is a cyclomethicone or a dimethicone.

108. The topical composition of claim 107, wherein the silicone oil is about 1% to about 75%, or about 5% to about 50%, or about 7% to about 30%, or about 10% to about 15% by weight of the carrier base.

109. The topical composition of claim 95 or 96, wherein the gelling agent is about 0.4% to about 15%, about 0.5% to about 5% about 1% to about 13%, about 5% to about 12%, or about 8% to about 11% by weight of the composition.

110. The topical composition of any preceding claim, wherein the ratio of emollient to elastomer is from about 1:30 to about 1:3.

111. The topical composition of any preceding claim, wherein the ratio of emollient to elastomer is between about 1:9 to about 1:6, between about 1:8 to about 1:7, about 1:7, about 3:22, or about 1:8.

112. The topical composition of claim 110, wherein the ratio of emollient to elastomer is about 1:4, about 2:9, about 4:18, about 1:5, about 4:21, about 2:11, or about 1:6.

113. The topical composition of any preceding claim, wherein the composition is anhydrous or substantially anhydrous.

114. The topical composition of any preceding claim, wherein the composition has an Aw value of less than 9.

115. The topical composition of claim 114, wherein the composition has an Aw value of less than or about 0.8, 0.7, or 0.6.

116. The topical composition of claim 114, wherein the composition has an Aw value of less than about 0.5, 0.4 or 0.3.

117. The topical composition of any preceding claim, wherein the tofacitinib is chemically stable.

118. The topical composition of claim 117, wherein the tofacitinib is chemically stable for at least 3 months at 25° C.

119. The topical composition of claim 117 or 118, wherein at least 90%, about 95%, about 98%, or about 99% by mass of the tofacitinib or pharmaceutically acceptable salt thereof is present in the composition when stored for 3 or 6 months at 25° C.

120. The topical composition of any of claims 117-119, wherein the composition is stored at 40° C.

121. The topical composition of any of claims 117-119, wherein less than about 0.1% by mass of Impurity B is measured when the composition is stored for 3 or 6 months at 25° C. compared to time 0.

122. The topical composition of claim 121, wherein the composition is stored at 40° C.

123. The topical composition of any of claims 8-121 wherein the reduction is sufficient to discourage significant adhesion to a surface, a metal surface, or a moving metal surface.

124. The topical composition of claim 123, wherein the metal is stainless steel.

125. The topical composition of any of claims 8-124, wherein the surface energy of the carrier and tofacitinib is below that of tofacitinib and a metal.

126. The topical composition of any of claims 8 to 125, wherein the interfacial tension (mN/m) of the carrier and tofacitinib is below that oftofacitinib and a metal.

127. The topical composition of any of claims 8-126, wherein the interfacial tension (mN/m) of the carrier and tofacitinib is at least about 5% below e.g., 10% or 15% below that of tofacitinib and a metal.

128. The topical composition of claim 126 or 127, wherein the metal is stainless steel.

129. The topical composition of any of claims 8-122, wherein the reduction is sufficient to discourage significant adhesion to a plastic surface e.g., a moving plastic surface.

130. The topical composition of any of claims 8-122 or 129, wherein the surface energy of the carrier and tofacitinib is below that of tofacitinib and a plastic.

131. The topical composition of any of claims 8-122, 129, or 130, wherein the interfacial tension (mN/m) of the carrier and tofacitinib is below that of tofacitinib and a plastic.

132. The topical composition of any of claims 8-122, 129, or 130, wherein the interfacial tension (mN/m) of the carrier and tofacitinib is at least about 5% below e.g., 10% or 15% below that of tofacitinib and a plastic.

133. The topical composition of any of claims 8-122 or 129-132, wherein the plastic is PTFE (polytetrafluorethylene).

134. The topical composition of any of claims 8-128, wherein the surface energy of the composition is below that of the tofacitinib with a metal.

135. The topical composition of any of claims 8-122, wherein the interfacial tension between non-micronized tofacitinib and the composition is less than about 1.6 mN/m or between about 1.5 mN/m and about 1.1 nM/m.

136. The topical composition of any of claims 8-122, wherein the interfacial tension between micronized tofacitinib and the composition is less than about 2.5 mN/m, or between about 1.8 mN/m and about 2.3 mN/m.

137. The topical composition of any preceding claim, wherein the ratio of carrier base to emollient is about or less than 9:1, or between about 9:1 and about 6:1.

138. The topical composition of any preceding claim, wherein the ratio of carrier base to emollient is between about 8:1 and about 7:1, about 8:1, about 22:3, or about 7:1.

139. The topical composition of any preceding claim, wherein the carrier base is about 83% to about 90%, about 86% to about 88%, or about 87% by weight of the composition.

140. The topical composition of any preceding claim, wherein the carrier base comprises elastomer and is about 83% to about 90% by weight of the composition and the emollient is about 10% to about 16% by weight of the composition.

141. The topical composition of claim 140, wherein the carrier base comprises elastomer and is about 86% to about 88% by weight of the composition and the emollient is about 11% to about 14% by weight of the composition.

142. The topical composition of any preceding claim, wherein the emollient comprises a triglyceride oil comprising one or more of an MCT oil, an olive oil, a coconut oil, a palm oil, a sunflower oil, a rapeseed oil, a soybean oil, a groundnut oil, a peanut oil, a corn oil, a walnut oil, a soya oil, a fish oil, a tallow, a fraction of any of the aforesaid, or mixtures of any two or more thereof

143. The topical composition of any preceding claim, wherein the tofacitinib and the fingolimod are the sole active agents in the composition.

144. The topical composition of any preceding claim, wherein the carrier or composition is a gel, or a semi-solid, or a liquid at room temperature.

145. The topical composition of claim 144, wherein the composition is a gel at room temperature.

146. The topical composition of claim 144, wherein the composition is a semi-solid at room temperature.

147. The topical composition of claim 144, wherein the composition is a liquid at room temperature.

148. The topical composition of any preceding claim, wherein composition is foamable and comprises a foam adjuvant and/or a surfactant.

149. The topical composition of claim 148, wherein foamable composition comprises a propellant.

150. The topical composition of claim 149, wherein the foamable composition upon release from a pressurized canister forms a foam.

151. The topical composition of any preceding claim, wherein the composition when applied to a surface does not run.

152. The topical composition of any preceding claim, wherein the composition when applied to a skin or mucosal surface has a bioadhesive or mucoadhesive quality.

153. The topical composition of any preceding claim, wherein the composition forms a quasi-layer.

154. The topical composition of any preceding claim, wherein the quasi-layer facilitates absorption of the tofacitinib into epidermal and dermal layers of skin.

155. The topical composition of any preceding claim, wherein the quasi-layer facilitates absorption of the tofacitinib into a mucosal membrane.

156. The topical composition of any preceding claim, wherein the quasi-layer facilitates absorption of the tofacitinib into a lining of a body cavity.

157. The topical composition of any preceding claim, wherein the carrier base and emollient act synergistically to enhance delivery even though the tofacitinib or the fingolimod are not soluble or substantially not soluble in the carrier base and emollient.

158. The topical composition of claim 98, wherein the composition provides at least two, three, or four of the following characteristics:
a. an increase in the chemical stability of tofacitinib salt;
b. a reduction or elimination of balling;
c. when applied topically to skin or mucosa an increased delivery of tofacitinib into the skin or mucosa;
d. when applied topically to skin or mucosa a reduced delivery of tofacitinib through the skin or mucosa; and
e. when applied topically to skin an increased delivery oftofacitinib into the epidermis and reduced delivery through the skin.

159. The topical composition of any preceding claim further comprising at least one of a fragrance agent, a masking agent, a buffering agent, a pH agent, a preservative, a chelating agent, an anti-oxidant, a scavenger agent, a thickener, a diluent, or any mixtures of two or more thereof.

160. The topical composition of any preceding claim, wherein the fingolimod is a pharmaceutically acceptable salt.

161. The topical composition of any preceding claim, wherein the fingolimod salt is a citrate salt, hydrochloride salt, hydrobromide salt, oxalate salt, nitrate salt, sulfate salt, phosphate salt, fumarate salt, succinate salt, maleate salt, besylate salt, tosylate salt, palmitate salt, tartrate salt, adipate salt, laurate salt, or myristate salt.

162. The topical composition of any preceding claim, wherein the fingolimod comprises fingolimod hydrochloride.

163. The topical composition of any preceding claim, wherein the fingolimod comprises fingolimod base.

164. The topical composition of any preceding claim, wherein the fingolimod is present in an amount sufficient to restore the skin barrier.

165. The topical composition of claim 163, wherein the fingolimod increases the level of filaggrin in the skin.

166. The topical composition of any preceding claim, wherein the fingolimod is present in an amount of about 0.0001% to about 10% by weight of the composition and the tofacitinib is present in an amount of about 0.01% to about 10% by weight of the composition, the fingolimod is about 0.001% to about 1% by weight of the composition and the tofacitinib is about 0.05% to about 3.05% by weight of the composition, the fingolimod is about 0.002% to about 0.1% by weight of the composition and the tofacitinib is about 0.1% to about 1% by weight of the composition, or the fingolimod is about 0.005% to about 0.01% by weight of the composition and the tofacitinib is about 0.3% to about 0.6% by weight of the composition, or fingolimod is about 0.001% to about 0.01% by weight of the composition and the tofacitinib is about 0.3% to about 0.6% by weight of the composition, or fingolimod is about 0.005% to about 0.01% by weight of the composition and the tofacitinib is about 0.3% to about 0.6% by weight of the composition, or fingolimod is about 0.005% to about 0.02% by weight of the composition and the tofacitinib is about 0.3% to about 0.6% by weight of the composition, or fingolimod is about 0.01% to about 0.02% by weight of the composition and the tofacitinib is about 0.3% to about 0.6% by weight of the composition, or fingolimod is about 0.01% to about 0.1% by weight of the composition and the tofacitinib is about 0.3% to about 0.6% by weight of the composition, or fingolimod is about 0.001% to about 0.1% by weight of the composition and the tofacitinib is about 0.3% to about 0.6% by weight of the composition.

167. The topical composition of any preceding claim, wherein the fingolimod is at a concentration of about 0.001% to about 0.01% by weight of the composition, or about 0.005% to about 0.01% by weight of the composition, or about 0.005% to about 0.02% by weight of the composition, or about 0.01% to about 0.02% by weight of the composition, or about 0.01% to about 0.1%, or about 0.001% to about 0.1% by weight of the composition.

168. A kit comprising the composition of any of preceding claim in a container and a disposable applicator connectable to the container.

169. The kit of claim 168, wherein the container comprises a unit dose means suitable for delivery of a measured unit dose of the tofacitinib and the fingolimod.

170. The kit of claim 169, wherein the unit dose is about 0.1 g, about 0.2 g, about 0.3 g, about 0.4 g, about 0.5 g, about 0.6 g, about 0.7 g, about 0.8 g, about 0.9 g, or about 1.0 g of composition.

171. The kit any of claims 168-170, wherein the disposable applicator is adapted for delivery of the composition to a body cavity.

172. The kit of any of claims 168-171, wherein the disposable applicator is adapted for delivery of the composition to a skin surface.

173. The kit of any of claims 168-172, wherein the disposable applicator is adapted for delivery of the composition to a mucosal surface.

174. A method of treating or ameliorating a skin disorder comprising applying to the skin of a subject the composition of any of claims 1-167.

175. A method of treating or ameliorating a mucosal disorder comprising applying to the mucosa of a subject the composition of any of claims 1-167.

176. A method of treating or ameliorating a body cavity disorder comprising applying to a body cavity of a subject the composition of any of claims 1-167.

177. A method of treating a JAK related condition comprising applying to the skin or mucosa or body cavity of a subject the composition of any of claims 1-167.

178. A method of treating or preventing a dermatological disorder, or a deterioration thereof, comprising applying to the skin of a subject the composition of any of claims 1-167.

179. The method of claim 178, wherein the disorder is dermatitis, atopic dermatitis, psoriasis, hypertrophic scars, keloid scars, post-surgery scars, or eczema.

180. The method of claim 179 wherein the eczema is atopic dermatitis, contact dermatitis, dyshidrotic eczema, nummular eczema, seborrheic dermatitis, or stasis dermatitis.

181. The method of claim 179, wherein the dermatological disorder is atopic dermatitis.

182. The method of claim 179, wherein the dermatological disorder is psoriasis.

183. The method of claim 179, wherein the dermatological disorder is scarring.

184. The method of any of claims 178-183, wherein the tofacitinib is delivered into the epidermis and the dermis.

185. The method of claim 184, wherein the delivery to the epidermis is greater than to the dermis.

186. The method of claim 184, wherein the delivery to the epidermis is at least about 20%, at least about 50%, at least about 100%, at least about 150%, at least about 200%, at least about 250%, or at least about 300% greater than to the dermis.

187. The method of claim 184, wherein the delivery to the epidermis is expressed as a percentage of applied dose.

188. The method of claim 187, wherein the delivery to the epidermis as a percentage of applied dose is at least about 100% greater than to the dermis.

189. The method of claim 187, wherein the topical delivery of the tofacitinib to the dermis and epidermis is about or greater than 20-fold the delivery of the tofacitinib through the skin.

190. The method of any of claims 174-189, wherein the composition is applied to the area of the disorder.

191. The method any of claims 174-189, wherein the composition is applied to the area surrounding the area of the disorder.

192. The method of any of claims 174-189, wherein the composition is applied to the area of the disorder and the area surrounding the disorder.

193. The method of any of claims 174-191, wherein the composition is applied once daily.

194. The method of any of claims 174-191, wherein the composition is applied twice daily.

195. The method of any of claims 174-191, wherein the composition is applied at least once per day for at least 7 days.

196. The method of any of claims 174-191, wherein the composition is applied at least once per day for at least 14 days.

197. The method of any of claims 174-191, wherein the composition is applied at least once per day for at least 4 weeks.

198. The method of any of claims 174-191, wherein the composition is applied at least once per day for at least 8 weeks.

199. The method of any of claims 174-191, wherein the composition is applied at least once per day for at least 12 weeks.

200. The method of any of claims 174-191, wherein the composition is applied as a maintenance dose following an initial treatment period.

201. The method of any of claims 174-191, wherein the maintenance dose is applied on non-consecutive days.

202. The method of claim 201, wherein the maintenance dose is applied on alternative days.

203. The method of claim 201, wherein the maintenance dose is applied twice weekly.

204. The method of any of claims 174-203, wherein systemic exposure to tofacitinib applied topically is much less than when the same amount is applied orally.

205. The method of claim 204, wherein the systemic exposure is at least 20-fold less.

206. The method of claim 204, wherein the systemic exposure is at least 50-fold less.

207. The method of claim 204, wherein the systemic exposure is at least 100-fold less.

208. The method of claim 204, wherein the systemic exposure is at least 200-fold less.

209. The method of claim 204, wherein the systemic exposure is at least 400-fold less.

210. The method of claim 204, wherein the systemic exposure is at least 500-fold less.

211. The method of claim 181, wherein the atopic dermatitis index is reduced by about 20%, about 25%, or about 30% compared to placebo.

212. The method of claim 211, wherein the index is less than four.

213. The method of claim 211, wherein the index is about 3.

214. The method of claim 181, wherein the psoriasis index is reduced by about 20%, about 25%, or about 30% compared to placebo.

215. The method of claim 214, wherein the index is less than four.

216. The method of claim 214, wherein the index is about 3.4.

217. The method of any of claims 174-216, wherein the carrier is not an emulsion.

218. The method of claim 177, wherein the JAK related condition or disorder comprises alopecia totalis, alopecia universalis, vitiligo, autoimmune bullous skin disorder, pemphigus vulgaris (PV), bullous pemphigoid (BP), skin rash, skin irritation, skin sensitization, contact dermatitis, allergic contact dermatitis, chronic atypical neutrophilic dermatosis with lipodystrophy and elevated temperature (CANDLE), pustulosis palmoplantaris, ichtyosis, hypertrophic scars, keloid scars, post-surgery scars, eczema, actinic keratosis, pruritus, rosacea, or acne.

219. The method of claim 177, wherein the JAK related condition or disorder comprises vitiligo and the composition is applied topically to the area of skin lacking pigment and its surrounds.

220. The method of claim 177, wherein the JAK related condition or disorder comprises alopecia and the composition is applied topically to the skin and hair.

221. The method of claim 178, wherein the disorder comprises a dermatitis, atopic dermatitis, dermatomyositis, eczema, psoriasis, rosacea, acne, disorder of the pilosebaceous unit, alopecia, alopecia totalis, alopecia universalis, vitiligo, autoimmune bullous skin disorder, skin rash, skin irritation, skin sensitization, chronic atypical neutrophilic dermatosis with lipodystrophy and elevated temperature (CANDLE), pustulosis palmoplantaris, ichtyosis, actinic keratosis, pruritus, contact dermatitis, dyshidrotic eczema, nummular eczema, seborrheic dermatitis, stasis dermatitis, lupus erythematosus, skin inflammation, skin itch, skin infection, skin scars, folliculitis/keratosis pilaris, hidradentitis suppurativa, pyoderma gangrenosum, lichenification disorders, primary cicatricial alopecias, or cellulitis.

222. The topical composition of any preceding claim, wherein the fingolimod is chemically stable.

223. The topical composition of claim 117, wherein the fingolimod is chemically stable for at least 3 weeks at 5° C.

224. The topical composition of claim 117, wherein the fingolimod is chemically stable for at least 3 weeks at 40° C.

225. The topical composition of claim 117, wherein the fingolimod is chemically stable for at least 3 weeks at 50° C.

226. The topical composition of claim 117, wherein the fingolimod is chemically stable for at least 2 months at 5° C.

227. The topical composition of claim 117, wherein the fingolimod is chemically stable for at least at least 2 months at 40° C.

228. The topical composition of claim 117, wherein the fingolimod is chemically stable for at least at least 2 months at 50° C.

229. The topical composition of any of claims 222 to 228, wherein at least about 90%, about 95%, about 98%, or about 99% by mass of the fingolimod is present in the composition when stored for 3 weeks or 2 months at 5° C.

230. The topical composition of any of claims 222 to 228, wherein at least about 90%, about 95%, about 98%, or about 99% by mass of the fingolimod is present in the composition when stored for 3 weeks or 2 months at 25° C.

231. The topical composition of any proceeding claim, wherein the tofacitinib and the fingolimod are chemically stable in the composition.

232. The topical composition of claim 229, wherein at least about 90%, about 95%, about 98%, or about 99% by mass of the tofacitinib and of the fingolimod is present in the composition when stored for 3 or 6 months at 5° C.

233. The topical composition of claim 230, wherein at least about 90%, about 95%, about 98%, or about 99% by mass of the tofacitinib and of the fingolimod is present in the composition when stored for 3 or 6 months at 25° C.

234. Use of the topical composition of any preceding claim as a medicament for preventing, treating or ameliorating a disorder or a deterioration thereof comprising applying to the skin of a subject the topical composition.

235. Use of the topical composition of claim 234, wherein the disorder comprises a dermatitis, atopic dermatitis, dermatomyositis, eczema, psoriasis, rosacea, acne, disorder of the pilosebaceous unit, alopecia, alopecia totalis, alopecia universalis, vitiligo, autoimmune bullous skin disorder, skin rash, skin irritation, skin sensitization, chronic atypical neutrophilic dermatosis with lipodystrophy and elevated temperature (CANDLE), pustulosis palmoplantaris, ichtyosis, actinic keratosis, pruritus, contact dermatitis, dyshidrotic eczema, nummular eczema, seborrheic dermatitis, stasis dermatitis, lupus erythematosus, skin inflammation, skin itch, skin infection, skin scars, folliculitis/keratosis pilaris, furunculosis, hidradentitis suppurativa, pyoderma gangrenosum, lichenification disorders, primary cicatricial alopecias, or cellulitis.

236. Use of the topical composition of claim 234, wherein the dermatological disorder involves inflammation. A dual chamber kit comprising two containers, wherein a first container comprises a first composition comprising tofacitinib or a pharmaceutically acceptable salt thereof and a first carrier and a second container comprising a second composition comprising a second carrier and fingolimod or a pharmaceutically acceptable salt thereof, wherein each composition is stored separately and upon dispensing the compositions are combined with a mixer connectable to each container and optionally comprising a unit dose means for dispensing a measured unit dose from each container.

237. The method of claim 221, wherein the autoimmune bullous skin disorder is pemphigus vulgaris (PV) or bullous pemphigoid (BP).

238. The method of claim 221, wherein the skin sensitization is contact dermatitis or allergic contact dermatitis.

239. The method of claim 221, wherein the skin scars are hypertrophic scars, keloid scars, or post-surgery scars.

240. The method of claim 221, wherein the lichenification disorders are lichen planus/sclerosus or lichen simplex chronicus/neurodermatitis.

241. The method of claim 221, wherein the primary cicatricial alopecias are lichen planopilaris or frontal fibrosing alopecia.

242. The use of claim 235, wherein the autoimmune bullous skin disorder is pemphigus vulgaris (PV) or bullous pemphigoid (BP).

243. The use of claim 235, wherein the skin sensitization is contact dermatitis or allergic contact dermatitis.

244. The use of claim 235, wherein the skin scars are hypertrophic scars, keloid scars, or post-surgery scars.

245. The use of claim 235, wherein the lichenification disorders are lichen planus/sclerosus or lichen simplex chronicus/neurodermatitis.

246. The use of claim 235, wherein the primary cicatricial alopecias are lichen planopilaris or frontal fibrosing alopecia.
